SEQUENCE LISTING (13 sequences)

SEQ ID NO: 1 - DNA, 1296 bp, Homo sapiens
atgcaggcgcttaacattaccccggagcagttctctcggctgctgcgggaccacaacctgacgcgggagcagttcatcgcagttcatcgcctgcgaccgctcgtctacaccccagagctgccgggacgcgccaagctggccctcgtgctcaccggcgtgctcatcttcgccctggcgctctttggcaatgctctggtgttctacgtggtgacccgcagcaaggccatgcgcaccgtcaccaacatctttatctgctccttggcgctcagtgacctgctcatcaccttcttctgcattcccgtcaccatgctccagaacatttccgacaactggctggggggtgctttcatttgcaagatggtgccatttgtccagtctaccgctgttgtgacagaaatcctcactatgacctgcattgctgtggaaaggcaccagggacttgtgcatccttttaaaatgaagtggcaatacaccaaccgaagggctttcacaatgctaggtgtggtctggctggtggcagtcatcgtaggatcacccatgtggcacgtgcaacaacttgagatcaaatatgacttcctatatgaaaaggaacacatctgctgcttagaagagtggaccagccctgtgcaccagaagatctacaccaccttcatccttgtcatcctcttcctcctgcctcttatggtgatgcttattctgtacagtaaaattggttatgaactttggataaagaaaagagttggggatggttcagtgcttcgaactattcatggaaaagaaatgtccaaaatagccaggaagaagaaacgagctgtcattatgatggtgacagtggtggctctctttgctgtgtgctgggcaccattccatgttgtccatatgatgattgaatacagtaattttgaaaaggaatatgatgatgtcacaatcaagatgatttttgctatcgtgcaaattattggattttccaactccatctgtaatcccattgtctatgcatttatgaatgaaaacttcaaaaaaaatgttttgtctgcagtttgttattgcatagtaaataaaaccttctctccagcacaaaggcatggaaattcaggaattacaatgatgcggaagaaagcaaagttttccctcagagagaatccagtggaggaaaccaaaggagaagcattcagtgatggcaacattgaagtcaaattgtgtgaacagacagaggagaagaaaaagctcaaacgacatcttgctctctttaggtctgaactggctgagaattctcctttagacagtgggcattaa

SEQ ID NO: 2 - DNA, 1816 bp, Mus musculus
gccagagcaggaggctggcaggactctgcgcacagcatgcaggcgctcaacatcaccgcggagcagttttcccggctgctgagcgcgcacaacctgactcgggaacagttcattcatcgctatgggctgcgaccgctggtctacactccggagctgcccgcgcgcgctaaactggcctttgcgctggctggagcactcatttttgccctggcgctctttggcaactctctggtcatctatgtggtgacccgcagcaaggccatgcgcaccgtcaccaacatcttcatctgctctctggcactcagtgatctgctcattgccttcttctgcatccccgtcacgatgctccagaacatctccgacaagtggctgggtggtgccttcatctgcaagatggtgcccttcgtccagtccactgctgttgtgacggaaatcctcaccatgacttgcatcgctgttgagaggcaccaaggactcatccatccttttaaaatgaagtggcagtacactacccgaagggctttcacaatcttgggtgtggtctggttggcagccatcatcgtaggatcacccatgtggcacgtacaacgcctcgagattaagtatgacttcctctatgagaaagaacatgtctgctgtttggaagagtgggccagccccatgcaccagagaatctacaccaccttcatcctcgtcatcctcttcctcctgccgcttgtggtgatgcttgtcctctacagcaagattggctatgaactgtggatcaagaagagagttggagacagttcagcacttcagactatccacgggaaagaaatgtccaaaatagccaggaagaagaagcgggctgtcgttatgatggtgacagtggtggctctcttcgctgcgtgctgggcacctttccatgttgttcacatgatggttgagtacagtaactttgaaaaagagtatgatgatgtcacaatcaagatggtttttgctgttgcacaaacaattggctttttcaactccatctgtaatccctttgtgtatgcatttatgaatgaaaacttcaaaaagaattttttgtctgcggtttgttattgcatagtaagagaaaccttctccccaggacagaagcctggaaattctgggatttcaatgatgcaaaagagagcaaagttatcacgatcacagcgtccagtggcggaagccaaaggagacttattcagcgatgccaacgttgatgtcaaattgtgtgagcagccaggggagaaaaggcaactcaagcgacagcttgccttctttagttctgaactttctgaaaactctactttcggcagtggacatgaactgtaatgatatcctcatagctaatatcatttgtatggaaagttattttaagcaaaggtcaggactattttttttaaatgacaagaagagaaacaagacatgttttccatttaaatgaacataatacataacactgtaactttgaaaaattattataacagctttgtagatgataaaagtagatttttgaaagtcttcgtacataataaagcagtggttttggcagcagttttatccatgtagtcaatgtaatgtgacttttatgtattgctacactggatgaaaattattaaaattgtgtcatcatccttgaatattaaacatctgaacatcataatgtagtttgtagtgtgctgtaaacgtttgtaaaatcagcctttggaactgacatctgtgccataattaaaaaatcaaggaggatgaagaatcaggcaagtgacaactaataaaagttaagagataaatgtcaaaaaaaaaaaaaaaa

SEQ ID NO: 3 - DNA, 1772 bp, Mus musculus
atgtcgtggaacttgaccgcggagcagctctcggcgctgcttcggctgcacaacctgacgcgcgctcagttcatcgcgcactatgggctgcggccactggtgctcaccccgcagcttcccgcgcgcgccaggctggccctcctgctggtcggcatgctcatctttgccctggcgctcttcggcaacgccctggtagtctatgtggtgacccgcagcaaggccatgcgcaccgtcaccaacatcttcatctgctccctggcactcagcgacctgctcatcgtcttcttctgcatcccggtcaccatgctccagaacgtctcggacacctggctggggggtgccttcatttgcaaaatggtcccatttgtccagtgcactgccattgtgacagaaatccttactatgacctgcattgctgtggaaaggcaccagggacttgtccatccttttaaaatgaagcggcagtacaccaatcaaagagctttcacaatgctaggtgtggtgtggctggtggccatcatcataggatcacccatgtggcatgtgcagcgacttgagattaagtatgacttcctatatgaaaaagaacacatctgctgcctggaagagtggagcagccccgtgcaccagaagatctacaccaccttcatccttgtcaccctcttcctgctaccactgttgctgctctctgtcctctacgggaaaatcggttatgagctttggatcaagaaaagaatcggggatggctcagtgctccgaactattcatggaaaagaaatgttcaaaatagccagaaagaagaagcgagctgtgatcatgatggtgacagtcgtggttctctttgctgtgtgctgggcacctttccacatcgttcacatgatgattgaatacagtaattttgaaaaggaatatgatgaagtcacaatcaagatgatttttgctatagtgcaaataattggatttttcaactccatctgtaatcccattatttatgcacttatgaatgaaaacttcaaaaaaaactttgtgtctgccgtttgctattgcattgtgaaggaaacaccttcttcagcacggaagcatggaagttcaggagctatggtgatgcacaggagggcaaagttagctgcaagagagaatcctgtagagatcaaaggagaagcatttgggggcagcaacatcgatatcaagtggtgtgaacagccagaaaagaagaagaggagatcaaaagtggcatcttgtcctctttagttccgaatttctgagagctctgctgtagacgtgaacactgtaccaatgtcttcagaatgagtatctgtcatactgtaatcgaaagaaaatgattttgagaaaaagccagagagctttcatattaaaaatgttgacaaacactcagaaggcagggacaggggattcaagagtttaaagtcatccttagctgcacgataagtttgaggataacctgggctacaagagaccctgtctcaagaagccataataattaaaacaaccatccttaactaatgataatgacaaagtatttttccattgaaaatacatgtaagctgcaattttgaaaaattattgaaccacccttgtgattaatagatgaagtttaaaaaaatttaaatgtgtttttattgtatgtatatggttggtttacctgtgtatatgtctttcagtaacttgtataaaactcaatgatctcagctagtaactttcttctgtgtggtcaatgtgatatgatttcctatatattgctaaattgaatg

SEQ ID NO: 4 - DNA, 1101 bp, Homo sapiens
atgtggaacgcgacgcccagcgaagagccggggttcaacctcacactggccgacctggactgggatgcttcccccggcaacgactcgctgggcgacgagctgctgcagctcttccccgcgccgctgctggcgggcgtcacagccacctgcgtggcactcttcgtggtgggtatcgctggcaacctgctcaccatgctggtggtgtcgcgcttccgcgagctgcgcaccaccaccaacctctacctgtccagcatggccttctccgatctgctcatcttcctctgcatgcccctggacctcgttcgcctctggcagtaccggccctggaacttcggcgacctcctctgcaaactcttccaattcgtcagtgagagctgcacctacgccacggtgctcaccatcacagcgctgagcgtcgagcgctacttcgccatctgcttcccactccgggccaaggtggtggtcaccaaggggcgggtgaagctggtcatcttcgtcatctgggccgtggccttctgcagcgccgggcccatcttcgtgctagtcggggtggagcacgagaacggcaccgacccttgggacaccaacgagtgccgccccaccgagtttgcggtgcgctctggactgctcacggtcatggtgtgggtgtccagcatcttcttcttccttcctgtcttctgtctcacggtcctctacagtctcatcggcaggaagctgtggcggaggaggcgcggcgatgctgtcgtgggtgcctcgctcagggaccagaaccacaagcaaaccgtgaaaatgctggctgtagtggtgtttgccttcatcctctgctggctccccttccacgtagggcgatatttattttccaaatcctttgagcctggctccttggagattgctcagatcagccagtactgcaacctcgtgtcctttgtcctcttctacctcagtgctgccatcaaccccattctgtacaacatcatgtccaagaagtaccgggtggcagtgttcagacttctgggattcgaacccttctcccagagaaagctctccactctgaaagatgaaagttctcgggcctggacagaatctagtattaatacatga

SEQ ID NO: 5 - DNA, 870 bp, Homo sapiens
atgtggaacgcgacgcccagcgaagagccggggttcaacctcacactggccgacctggactgggatgcttcccccggcaacgactcgctgggcgacgagctgctgcagctcttccccgcgccgctgctggcgggcgtcacagccacctgcgtggcactcttcgtggtgggtatcgctggcaacctgctcaccatgctggtggtgtcgcgcttccgcgagctgcgcaccaccaccaacctctacctgtccagcatggccttctccgatctgctcatcttcctctgcatgcccctggacctcgttcgcctctggcagtaccggccctggaacttcggcgacctcctctgcaaactcttccaattcgtcagtgagagctgcacctacgccacggtgctcaccatcacagcgctgagcgtcgagcgctacttcgccatctgcttcccactccgggccaaggtggtggtcaccaaggggcgggtgaagctggtcatcttcgtcatctgggccgtggccttctgcagcgccgggcccatcttcgtgctagtcggggtggagcacgagaacggcaccgacccttgggacaccaacgagtgccgccccaccgagtttgcggtgcgctctggactgctcacggtcatggtgtgggtgtccagcatcttcttcttccttcctgtcttctgtctcacggtcctctacagtctcatcggcaggaagctgtggcggaggaggcgcggcgatgctgtcgtgggtgcctcgctcagggaccagaaccacaagcaaaccgtgaaaatgctgggtgggtctcagcgcgcgctcaggctttctctcgcgggtcctatcctctccctgtgccttctcccttctctctga

SEQ ID NO: 6 - DNA, 23 bp, Artificial Sequence
  synthetic oligonucleotide
tctttggcaactctctggtcatc

SEQ ID NO: 7 - DNA, 22 bp, Artificial Sequence
  synthetic oligonucleotide
cttcgggtagtgtactgccact

SEQ ID NO: 8 - DNA, 24 bp, Artificial Sequence
  synthetic oligonucleotide
cgatatcaagtggtgtgaacagcc

SEQ ID NO: 9 - DNA, 20 bp, Artificial Sequence
  synthetic oligonucleotide
gggtctcttgtagcccaggt

SEQ ID NO: 10 - DNA, 21 bp, Artificial Sequence
  synthetic oligonucleotide
tctgccgtccttaccatctca

SEQ ID NO: 11 - DNA, 22 bp, Artificial Sequence
  synthetic oligonucleotide
tctcaggactgtcccaaaggag

SEQ ID NO: 12 - DNA, 23 bp, Artificial Sequence
  synthetic oligonucleotide
ggaccagagcgaaagcatttgcc

SEQ ID NO: 13 - DNA, 22 bp, Artificial Sequence
  synthetic oligonucleotide
tcaatctcgggtggctgaacgc

(19) United States
(12) Patent Application Publication
(10) Pub. No.: US 2009/0042905 A1
(43) Pub. Date: Feb. 12, 2009

(54) TRIAZOLE DERIVATIVES AS LIGANDS OF G-PROTEIN COUPLED RECEPTORS

(75) Inventors: Daniel Perrissoud, Bad Homburg (DE); Huy Ong, Cambridge (CA); Mukandila Mulumba, Longueuil (CA); Christian Jossart, Montreal (CA); Yves Lachance, St-Nicolas (CA)

Correspondence Address: OBLON, SPIVAK, MCCLELLAND MAIER & NEUSTADT, P.C., 1940 DUKE STREET, ALEXANDRIA, VA, 22314, US

(73) Assignee: AETERNA ZENTARIS GmbH, Frankfurt (DE)

(21) Appl. No.: 12/031,089

(22) Filed: Feb. 14, 2008

Related U.S. Application Data
(60) Provisional application No. 60/889,801, filed on Feb. 14, 2007.

(30) Foreign Application Priority Data
Feb. 14, 2007 (EP) 07102360.0

(51) Int. Cl. A61K 31/4196; A61K 31/454; A61K 31/4439; A61K 31/497; A61K 31/496; A61P 43/00

(52) U.S. Cl. 514/254.05; 514/383; 514/326; 514/339; 514/255.05

(57) ABSTRACT

The present invention provides triazole derivatives according to formula (I) as ligands of G-protein coupled receptors (GPCR), which are useful for the study of and/or the treatment or prophylaxis of physiological and/or pathophysiological conditions in mammals, preferably humans, which are mediated by GPCR.

### FIELD OF THE INVENTION

The invention relates to triazole derivatives that act as ligands of G-protein coupled receptors. These compounds are useful in the study of and/or treatment or prophylaxis of various physiological and pathophysiological conditions, such as adipogenesis, adiposity, obesity, alteration of food intake and energy balance, cachexia, hyperlipidemia and/or hyperphagia.

### DESCRIPTION OF THE RELATED ART

Many medically significant biological processes are mediated by signal transduction pathways that involve G proteins (Lefkowitz R J, Nature 1992, 358(6385):372). The family of G protein-coupled receptors (GPCR) includes receptors for hormones, neuro-transmitters, growth factors, and viruses. Specific examples of GPCRs include receptors for such diverse agents as calcitonin, adrenergic hormones, endothelin, cAMP, adenosine, acetylcholine, serotonin, dopamine, histamine, thrombin, kinin, follicle stimulating hormone, opsins, endothelial differentiation gene-1, rhodopsins, odorants, cytomegalovirus, G proteins themselves, effector proteins such as phospholipase C, adenyl cyclase, and phosphodiesterase, and actuator proteins such as protein kinase A and protein kinase C.

The GPCR protein superfamily now contains over 250 types of paralogs, receptors that represent variants generated by gene duplications (or other processes), as opposed to orthologs, the same receptor from different species. The superfamily can be broken down into five families:

Family I, receptors typified by rhodopsin and the beta-adrenergic receptor and currently represented by over 200 unique members (reviewed by Dohlman H G et al., Ann. Rev. Biochem. 1991, 60: 653-88, and references cited therein);

Family II, the parathyroid hormone/calcitonin/-secretin receptor family (Juppner H et al., Science 1991, 254: 1024-26; Lin H Y et al., Science 1991, 254: 1022-24);

Family III, the metabotropic glutamate receptor family in mammals (Nakanishi S, Science 1992, 258: 597-603);

Family IV, the cAMP receptor family, important in the chemotaxis and development of Dictyostelium discoideum (Klein P S et al., Science 1988, 241: 1467-72); and

Family V, the fingal mating pheromone receptors such as STE2 (reviewed by Kurjan J, Ann. Rev. Biochem. 1992, 61: 1097-1129).

GPCRs possess seven conserved membrane-spanning domains connecting at least eight divergent hydrophilic loops. GPCRs (also known as 7TM receptors) have been characterized as including these seven conserved hydrophobic stretches of about 20 to 30 amino acids, connecting at least eight divergent hydrophilic loops. Most GPCRs have single conserved cysteine residues in each of the first two extracellular loops, which form disulfide bonds that are believed to stabilize functional protein structure. The seven transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some GPCRs. Most GPCRs contain potential phosphorylation sites within the third cytoplasmic loop and/or the carboxy terminus. For several GPCRs, phosphorylation by protein kinase A and/or specific receptor kinases mediates receptor desensitization.

For some receptors, the ligand binding sites of GPCRs are believed to comprise hydrophilic sockets formed by several GPCR transmembrane domains. The hydrophilic sockets are surrounded by hydrophobic residues of the GPCRs. The hydrophilic side of each GPCR transmembrane helix is postulated to face inward and form a polar ligand binding site. TM3 has been implicated in several GPCRs as having a ligand binding site, such as the TM3 aspartate residue. TM5 serines, a TM6 asparagine, and TM6 or TM7 phenylalanines or tyrosines also are implicated in ligand binding.

GPCRs are coupled inside the cell by heterotrimeric G proteins to various intracellular enzymes, ion channels, and transporters (see Johnson G L et al., Endocr. Rev. 1989, 10: 317-31). Different G protein alpha subunits preferentially stimulate particular effectors to modulate various biological functions in a cell. Phosphorylation of cytoplasmic residues of GPCRs is an important mechanism for the regulation of some GPCRs. For example, in one form of signal transduction, the effect of hormone binding is the activation inside the cell of the enzyme, adenylate cyclase. Enzyme activation by hormones is dependent on the presence of the nucleotide GTP. GTP also influences hormone binding. A G protein connects the hormone receptor to adenylate cyclase. G protein exchanges GTP for bound GDP when activated by a hormone receptor. The GTP-carrying form then binds to activated adenylate cyclase. Hydrolysis of GTP to GDP, catalyzed by the G protein itself, returns the G protein to its basal, inactive form. Thus, the G protein serves a dual role, as an intermediate that relays the signal from receptor to effector, and as a clock that controls the duration of the signal.

Over the past decades, many hundreds of therapeutic agents targeting GPCRs receptors have been successfully introduced into the market. This indicates that these receptors have an established, proven history as therapeutic targets.

Further references dealing with G-protein coupled receptors comprise: WO 02/42458, WO 2004/000883.

Obesity and overweight are defined as an excess of body fat relative to lean body mass. An increase in caloric intake or a decrease in energy expenditure or both can bring about this imbalance leading to surplus energy being stored as fat. Obesity is associated with important medical morbidities and an increase in mortality. The causes of obesity are poorly understood and may be due to genetic factors, environmental factors or a combination of the two to cause a positive energy balance. In contrast, anorexia and cachexia are characterized by an imbalance in energy intake versus energy expenditure leading to a negative energy balance and weight loss. An interesting commentary review was published not long ago in Nature Medicine (Friedman J M, Nature Medicine 2004, 10(6): 563-569).

Agents that either increase energy expenditure and/or decrease energy intake, absorption or storage would be useful for treating obesity, overweight, and associated comorbidities. Agents that either increase energy intake and/or decrease energy expenditure or increase the amount of lean tissue would be useful for treating cachexia, anorexia and wasting disorders. In general the following exemplary physiological and/or pathophysiological states can be targeted: obesity, overweight, overweight-associated comorbidities including hypertension, type 2 diabetes, coronary artery disease, hyperlipidemia, stroke, gallbladder disease, gout, osteoarthritis, sleep apnea and respiratory problems, anorexia, cachexia, wasting disorders, appetite suppression, appetite enhancement, increases or decreases in satiety, modulation of body weight, and/or other eating disorders such as bulimia.

As pointed out supra, G protein-coupled receptors (GPCRs) are integral membrane proteins characterized by seven transmembrane spanning helical domains that mediate the actions of many extracellular signals. GPCRs interact with heterotrimeric guanine nucleotide binding regulatory proteins (G proteins) that modulate a variety of second messenger systems or ionic conductances to effect physiological responses. In fact, almost 50% of 30 currently marketed drugs elicit their therapeutic effects by interacting with GPCRs (Kirkpatrick, Nat. Rev. Drug Disc. 2002, 1(7): 488).

A number of peripherally and centrally acting signaling molecules produce a sense of hunger/satiety or produce elevation in lipid mobilization/oxidation through their interactions with GPCRs. There are numerous examples of neurotransmitters and hormones acting on central satiety pathways. Endocannabinoids, melanin concentrating hormone, serotonin, dopamine, NPY, alpha-MSH, GLP-I, ghrelin and orexin serve as few examples of neurotransmitters/hormones that modulate satiety and/or energy expenditure through GPCRs (Di Marzo V et al., Nature 2001, 410: 822-25; Marsh D J et al., Proc. Natl. Acad. Sci. USA 2002, 99(5): 3240-45; Nonogaki K et al., Nat. Med. 1998, 4(10): 1152-56; Gadde K M et al., Obes. Res. 2001, 9: 544-51; Daniels A J et al., Peptides 2001, 22: 483-91; Hinney A et al., J. Clin. Endocrin. Metabol. 1999, 84: 1483-86; Meier J J et al., Eur. J. Pharmacol. 2002, 440: 269-79; Nakazato M et al., Nature 2001, 409: 194-98; Haynes A C et al., Regul. Pept. 2002,104: 153-59).

In addition to modulation of central pathways, GPCRs also play a critical role in regulating energy expenditure in the periphery. For example, selective agonist ligands of beta3-adrenergic receptors (AR) induce increase in lipolysis and lipid oxidation in rodents resulting in a decrease in body weight (Arch J R S, Eur. J. Pharmacol. 2002, 440: 99-107).

Further relevant literature concerning GPCRs and obesity as well as related physiological states comprise: Melanin-concentrating-hormone (MCH) receptor (SLC-1; Chambers J et al., Nature 1999, 400: 261-265; Saito Y et al., Nature 1999, 400: 265-269; Saito Y et al., TEM 2000, 11(8): 299-303); NPY receptors (Fetissov S O et al., Neuropeptides 2004, 38: 175-188); general overview about GPCRs involved in metabolic diseases (Bagnol D, Curr. Opin. Drug. Disc. Dev. 2004, 7(5): 665-682); GPR103 receptors (AQ27, SP9155, Fukusumi S et al., J. Biol. Chem. 2003, 278(47): 46387-46395; Moriya R et al., Endocrinology 2006, 147(6): 2916-2922; Takayasu S et al., PNAS 2006, 103(19): 7438-7443; Kampe J et al., Brain Res. 2006, 1119(1): 133-149; Jiang Y et al., J. Biol. Chem. 2003, 278(30): 27652-27657; EP 1 207 201; Egido E M et al., “26RFa, a novel orexigenic neuropeptide, inhibits insulin secretion in the rat pancreas.”, Peptides, in press, available online 13 Jun. 2006; Thuau R et al., Peptides 2005, 26(5):779-89; Baribault H et al., Mol Cell Biol 2006, 26(2):709-17; Lee D K et al., Gene 2001, 275(1):83-91), cortistatin receptor (MrgX2, Robas N et al., J. Biol. Chem. 2003, 278(45): 44400-44404; Allia E et al., J. Pathol. 2005, 207(3): 336-345).

Compounds containing triazole moieties have been widely recognized in the medicinal chemistry due to their various biological activities. The following patent families are all directed to heterocyclic compounds that are said to show certain biological action for use in different medicinal indications. Triazole moieties are implicitly or explicitly contained.

WO 2004/111015 discloses modulators of the glucocorticoid receptor. WO 2004/052280 describes anti-agiogenic compounds as inhibitors of tyrosine kinase activity of VEGF receptors and their use in cancer. WO 2004/096795 also discloses tyrosine kinase inhibitors, preferably C-FMS inhibitors. WO 03/011831 and WO 03/011210 both describe heteroarylheteroalkylamine derivatives as inhibitors of nitric oxide synthase. WO 02/00651 is directed to Factor XA inhibitors for use in thromboembolic disorders. WO 01/94318 and WO 01/94317 both describe chemical libraries of substituted azole derivatives and methods of their synthesis for use in drug discovery high-throughput screening. However, they fail to provide any biological activity or any medicinal use nor do they name specific compounds. WO 00/76971 and WO 00/76970 both claim serine protease inhibitors useful as antithrombotic agents. WO 01/36395 discloses triazole derivatives as farnesyl transferase inhibitors. WO 96/33176 and U.S. Pat. No. 5,703,092 are directed to hydroxamic acid compounds as metalloprotease and TNF inhibitors. WO 93/09095 describes 2-heterocyclicethylamine derivatives and their use in neurological and neurodegenerative disorders. WO 2004/103270 claims compounds for the treatment of thrombosis, in particular FactorXIa inhibitors. WO 98/38177, U.S. Pat. No. 6,506,782, U.S. Pat. No. 6,849,650 and US 2003/0130188 all describe heterocyclic compounds as inhibitors of beta-amyloid peptide release or its synthesis for use in Alzheimer's disease. WO 00/54729 discloses heterocyclic aromatic compounds as GH secretagogues which are said to stimulate endogenous production and/or release of GH and can also contain triazole moieties. In addition, a method for increasing levels of endogenous GH or increasing the endogenous production or release of GH administering such GHS is described. Furthermore, a method is provided for preventing or treating osteoporosis (improving bone density and/or strength), or treating obesity, or increasing muscle mass and/or muscle strength and function in elderly humans, or reversal or prevention of frailty in elderly humans administering such GHS. U.S. Pat. No. 6,525,203, U.S. Pat. No. 6,518,292 U.S. Pat. No. 6,660,760 are members of the same patent family as WO 00/54729. WO 2004/021984 describes heterocyclic aromatic compounds GH secretagogues which are said to be useful in stimulating endogenous production or release of GH. However, claimed compounds consists of bi- to tetracylic aromatic rings and do not contain triazoles. WO 97/23508 claims compounds of peptide mimetic nature as GHS and are said to act directly on pituitary cells in vitro to release GH there-from and show improved properties, such as improved resistance to proteolytic degradation and improved bioavailability. In addition, claimed compounds could also be administered in vivo to increase GH release. The compounds are peptide derivatives and do not explicitly contain triazole moieties. U.S. Pat. No. 6,127,391, U.S. Pat. No. 5,977,178 and U.S. Pat. No. 6,555,570 are members of the same patent family as WO 97/23508. WO 01/36395 discloses triazole compounds for the treatment of tumor cell proliferation.

### SUMMARY OF THE INVENTION

The present invention has the object to provide compounds which can be employed for the study of and/or treatment or prophylaxis of physiological and/or pathophysiological conditions in mammals, in particular humans, that are mediated by G-protein coupled receptors (GPCR). It is another object of the underlying invention to provide compounds for the above treatment where the treatment is achieved by modulation of GPCR receptors. A further object of the present invention is to provide antagonists of GPCR receptors for those treatments. It is yet another object of the underlying invention to provide agonists of GPCR receptors for those treatments.

### BRIEF DESCRIPTION OF THE DRAWINGS

FIG. 1 depicts the nucleotide sequence (5′→3′) of human GPR103 (GenBank accession no. NM_{—}198179) (SEQ ID NO:1).

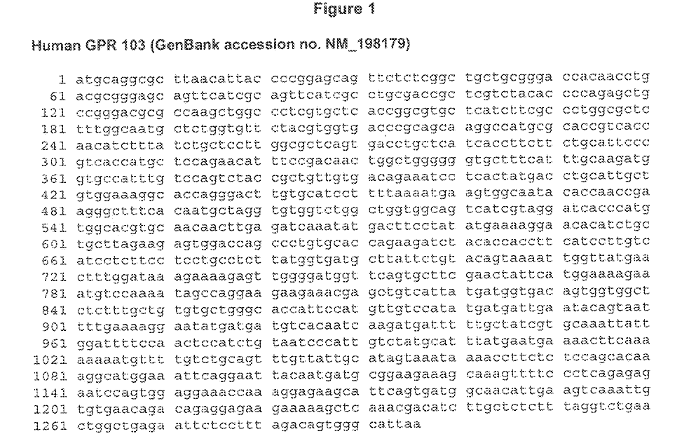

FIG. 2 depicts the nucleotide sequence (5′→3′) of mouse GPR103A (GenBank accession no. NM_{—}198192) (SEQ ID NO:2).

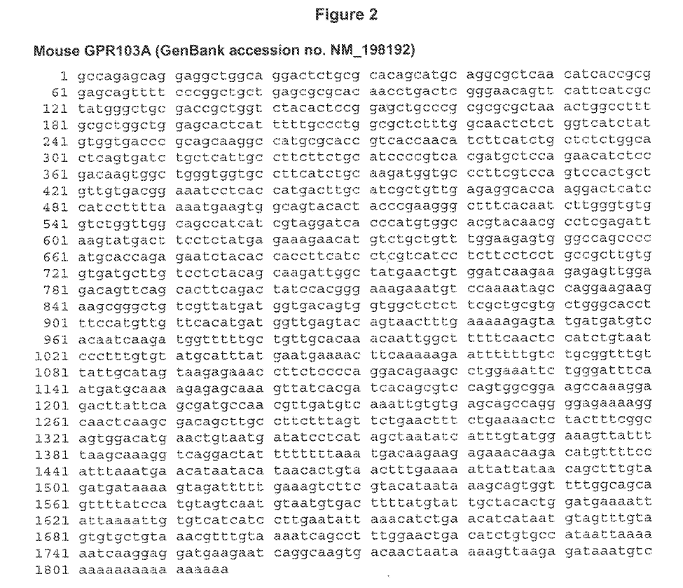

FIG. 3 depicts the nucleotide sequence (5′→3′) of mouse GPR103B (GenBank accession no. AX665940) (SEQ ID NO:3).

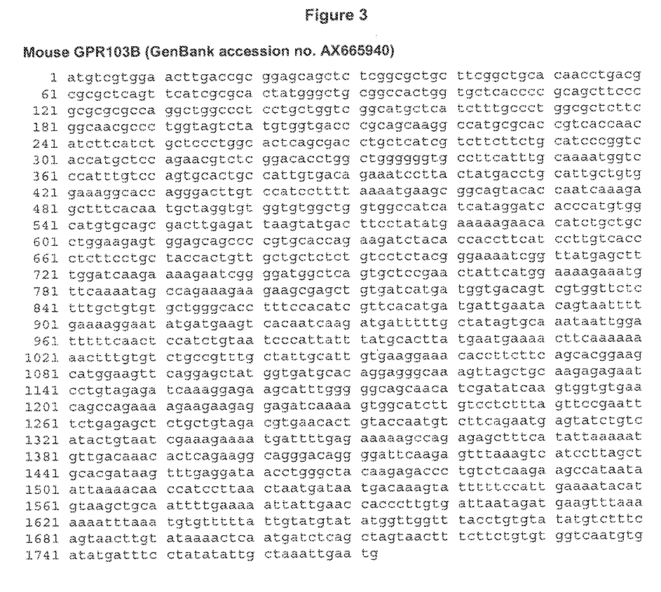

FIG. 4 depicts the nucleotide sequence (5′→3′) of human GHS-R1a (GenBank accession no. NM_{—}198407) (SEQ ID NO:4).

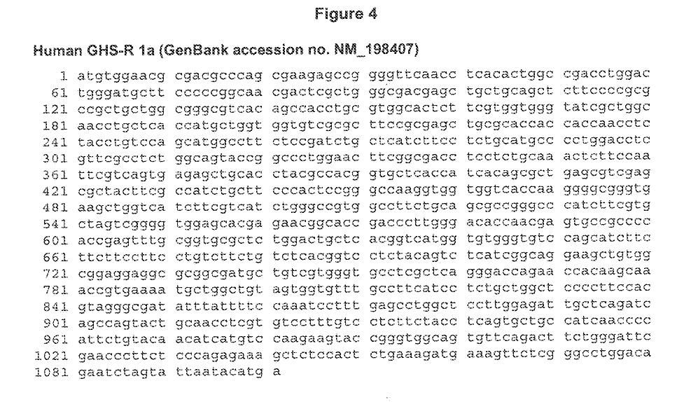

FIG. 5 depicts the nucleotide sequence (5′→3′) of human GHS-R1b (GenBank accession no. NM_{—}004122) (SEQ ID NO:5).

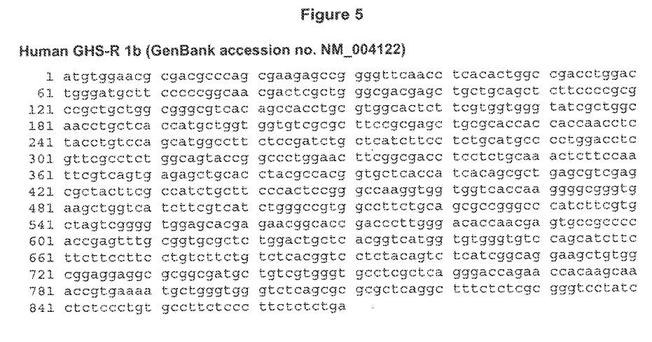

FIG. 6 shows the analysis of the mRNA expression of GPR103 receptors and QRFP peptide in mice adipocytes.

FIG. 7 shows the inhibitory effect of QRFP-43 and QRFP-26 on isoproterenol-induced lipolysis.

FIG. 8 shows the inhibitory effects of compound 79 on the inhibitory effect of QRFP-26 on isoproterenol-induced lipolysis.

FIG. 9 shows the inhibitory effects of compound 39 on the inhibitory effect of QRFP-26 on isoproterenol-induced lipolysis.

### DETAILED DESCRIPTION OF THE INVENTION

The object of the invention has been surprisingly solved in one aspect by providing compounds according to formula (I)

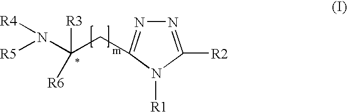

wherein:
R1 and R2 are independently of one another selected from the group consisting of “hydrogen atom, alkyl, alkenyl, alkynyl, cycloalkyl, cycloalkylalkyl, aryl, heteroaryl, arylalkyl, heteroarylalkyl, heterocyclyl, heterocyclylalkyl, alkylsulfonyl, arylsulfonyl, arylalkylsulfonyl” which are optionally substituted in the alkyl, cycloalkyl, cycloalkylalkyl, aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably are selected from the group consisting of “alkyl, aryl, heteroaryl, arylalkyl, heteroarylalkyl” optionally being substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
one of radicals R3 and R4 is a hydrogen atom, whereas the other radical is selected from the group consisting of “hydrogen atom, alkyl, aryl, heteroaryl, arylalkyl, heteroarylalkyl, heterocyclyl, heterocyclylalkyl, -alkyl-O-aryl, -alkyl-O-arylalkyl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, -alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO-NH₂, -alkyl-CO-OH, -alkyl-N H₂, -alkyl-NH-C(NH)-NH₂, alkylsulfonyl, arylsulfonyl, arylalkylsulfonyl, alkyl-S-alkyl, alkyl-S—H” which are optionally substituted in the aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably are selected from the group consisting of “arylalkyl, heteroarylalkyl, heterocyclylalkyl, -alkyl-O-aryl, -alkyl-O-arylalkyl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO—N—H₂, -alkyl-CO—OH, -alkyl-NH₂, -alkyl-NH—C(NH)NH₂,” optionally being substituted in the aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
R5 is selected from the group consisting of “hydrogen atom, alkyl, cycloalkyl, cycloalkylalkyl, aryl, heteroaryl, arylalkyl, heteroarylalkyl, heterocyclyl, heterocyclylalkyl, —CO-alkyl, —CO-cycloalkyl, —CO-cycloalkylalkyl, —CO-aryl, —CO-arylalkyl, —CO-heteroaryl, —CO-heteroarylalkyl, —CO-heterocyclyl, —CO-heterocyclylalkyl, —CO—C*(R9R10)—NH₂, —CO—CH₂—C*(R9R10)—NH₂, —CO—C*(R9R10)CH₂—NH₂, alkylsulfonyl, arylsulfonyl, arylalkylsulfonyl” which are optionally substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably is selected from the group consisting of “hydrogen atom, —CO-alkyl, —CO-cycloalkyl, —CO-aryl, —CO-heteroaryl, —CO-arylalkyl, —CO-heteroarylalkyl, —CO-heterocyclyl, —CO—C*(R9R10NH₂, —CO—CH₂—C*(R9R10)—NH₂, —CO—C*(R9R10)—CH₂—NH₂, optionally being substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
R6 is selected from the group consisting of “hydrogen atom, alkyl, cycloalkyl, cycloalkylalkyl” and preferably is a hydrogen atom;
R7 and R8 are independently of one another selected from the group consisting of “hydrogen atom, alkyl, cycloalkyl, cycloalkylalkyl” and preferably are a hydrogen atom;
R9 and R10 are independently of one another selected from the group consisting of “hydrogen atom, alkyl, natural alpha-amino acid side chain, unnatural alpha-amino acid side chain” and preferably are selected from the group consisting of “hydrogen atom, alkyl”;
m is 0, 1 or 2 and preferably is 0; and
* means a carbon atom of R or S configuration when chiral;
that can be used for the manufacture of a medicament for the treatment or prophylaxis of physiological and/or pathophysiological conditions in mammals, where the physiological and/or pathophysiological conditions are mediated by at least one G-protein coupled receptor (GPCR);
with the proviso that the following G-protein coupled receptors (GPCR) are excluded: “GHS receptors, GHS type 1 receptor, GHS-R1a, GHS-R1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

In a preferred embodiment compounds according to above formula (I) are provided, where
R3 is selected from the group consisting of “-alkyl-CO-aryl, -alkyl-CO-arylalkyl, -alkyl-CO-heteroaryl, -alkyl-CO-heteroarylalkyl, -alkyl-CO-heterocyclyl, alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO-NH₂, -alkyl-CO—OH, -alkyl-NH—C(NH)—NH₂, alkyl-S-alkyl, alkyl-S—H”, and preferably is selected from the group consisting of “-alkyl-CO-arylalkyl, -alkyl-C(O)O-arylalkyl, -alkyl-CO—NH₂, -alkyl-CO—OH”;

that can be used for the manufacture of a medicament for the treatment or prophylaxis of physiological and/or pathophysiological conditions in mammals, where the physiological and/or pathophysiological conditions are mediated by at least one G-protein coupled receptor (GPCR);

with the proviso that the following G-protein coupled receptors (GPCR) are excluded: “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

In another preferred embodiment compounds according to above formula (I) are provided, where
R4 is a hydrogen atom;
R5 is selected from the group consisting of “hydrogen atom, alkyl, cycloalkyl, cycloalkylalkyl, aryl, heteroaryl, arylalkyl, heteroarylalkyl, heterocyclyl, heterocyclylalkyl, alkylsulfonyl, arylsulfonyl, arylalkylsulfonyl, —CO-cycloalkyl, —CO-cycloalkyl-alkyl, —CO-aryl, —CO-arylalkyl, —CO-heteroaryl, —CO-heteroarylalkyl, —CO-heterocyclyl, —CO-heterocyclylalkyl”;
with the proviso that if R5 is “—CO-heteroarylalkyl”, “heteroaryl” is not imidazole; and
with the proviso that if R5 is “—CO-heterocyclyl” and “heterocyclyl” contains only nitrogen atoms as heteroatoms, that at least two nitrogen atoms are contained in “heterocyclyl”; and
with the proviso that if R5 is “—CO-heterocyclylalkyl” and “heterocyclyl” contains only nitrogen atoms as heteroatoms that in the case that one or two nitrogen atoms are contained in “heterocyclyl” no nitrogen atom is positioned at position 1 of “heterocyclyl” that is the atom directly linking “heterocyclyl” to the carbonyl group “—CO—”;
where “alkyl, cycloalkyl, cycloalkylalkyl, aryl, heteroaryl, arylalkyl, heteroarylalkyl, heterocyclyl, heterocyclylalkyl, alkylsulfonyl, arylsulfonyl, arylalkylsulfonyl, —CO-cycloalkyl, —CO-cycloalkylalkyl, —CO-aryl, —CO-arylalkyl, —CO-heteroaryl, —CO-heteroarylalkyl, —CO-heterocyclyl, and/or —CO-heterocyclylalkyl” are optionally substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
with the proviso that if R5 is “—CO-cycloalkyl” or “—CO-cycloalkylalkyl”, R5 is not substituted with NR7R8 at position 1 of “cycloalkyl”, that is the C atom directly linking “cycloalkyl” to the carbonyl group “—CO—” in case of R5=“—CO-cycloalkyl” or to the “alkyl” in case R5=“—CO-cycloalkylalkyl”; and
with the proviso that if R5 is “—CO-aryl” or “—CO-arylalkyl” and “aryl” is phenyl/benzene and is only substituted with one substituent, this one substituent is not —NR7R8;
R6 is a hydrogen atom;
R7 and R8 are independently of one another selected from the group consisting of “hydrogen atom, alkyl, cycloalkyl, cycloalkylalkyl” and preferably are a hydrogen atom; and
m is 0, 1 or 2, and more preferably is 0;

that can be used for the manufacture of a medicament for the treatment or prophylaxis of physiological and/or pathophysiological conditions in mammals, where the physiological and/or pathophysiological conditions are mediated by at least one G-protein coupled receptor (GPCR);

with the proviso that the following G-protein coupled receptors (GPCR) are excluded: “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

In a further aspect, the object of the invention has surprisingly been achieved by providing compounds according to formula (I), where
R1 is selected from the group consisting of “hydrogen, methyl, (2-methoxyphenyl)-methyl, (3-methoxyphenyl)-methyl, (4-methoxyphenyl)-methyl, (3-methoxyphenyl)-ethyl, (4-methoxyphenyl)-ethyl, phenyl, phenyl-methyl, phenyl-ethyl, (4-ethylphenyl)-methyl, (4-methylphenyl)-methyl, (4-fluorophenyl)-methyl, (4-bromophenyl)-methyl, (2,4-dimethoxyphenyl)-methyl, (3,5-dimethoxyphenyl)-methyl, 2,2-diphenyl-ethyl, naphthaline-1-yl-methyl, 1H-indole-3-yl-methyl, 2-(1H-indole-3-yl)-ethyl, 3-(1H-indole-3-yl)-propyl, 4-methyl-phenyl, 4-ethyl-phenyl, n-hexyl, (3,4-dichlorophenyl)-methyl, (4-nitro-phenyl)-methyl, (pyridine-2-yl)-methyl, (pyridine-3-yl)-methyl, (pyridine-4-yl)-methyl, (thiophene-2-yl)-methyl, (thiophene-3-yl)-methyl, (furan-2-yl)-methyl, (furan-3-yl)-methyl”;
R2 is selected from the group consisting of “methyl, 1H-indole-3-yl-methyl, 2-(1H-indole-3-yl)-ethyl, 3-(1H-indole-3-yl)-propyl, 2-phenyl-ethyl, 3-phenyl-propyl, 4-phenyl-butyl, 2-methoxy-phenylmethyl, 3-methoxy-phenylmethyl, 4-methoxy-phenylmethyl, 2-methoxy-phenylethyl, 3-methoxy-phenylethyl, 4-methoxy-phenylethyl”;
R3 is selected from the group consisting of “hydrogen atom, methyl, propan-2-yl, 2-methyl-propan-1-yl, butan-2-yl, butan-1-yl, —CH₂—SH, —(CH₂)₂—S—CH₃, 1H-indole-3-yl-methyl, phenyl-methyl, 2-phenyl-ethyl, —CH₂—O—CH₂-phenyl, —CH₂—CO—CH₂-phenyl, —(CH₂)₂—CO—CH₂-phenyl, —CH₂—C(O)O-phenyl, —(CH₂)₂—C(O)O-phenyl, hydroxy-methyl, 1-hydroxy-ethan-1-yl, —CH₂—CO—NH₂, —(CH₂)₂—CO—NH₂, (1-hydroxy-benzene-4-yl)-methyl, —CH₂—CO—OH, —(CH₂)₂—CO—OH, —(CH₂)₄—NH₂, (1H-imidazol-5-yl)-methyl, —(CH₂)₃—NH—C(NH)—NH₂, —(CH₂)₃—NH₂, —(CH₂)₃—NH—CO—NH₂”, and preferably is selected from the group consisting of “1H-indole-3-yl-methyl, —CH₂—CO—CH₂-phenyl, —(CH₂)₂—CO—CH₂-phenyl, —CH₂—C(O)O-phenyl, —(CH₂)₂—C(O)O-phenyl”;
R4 is a hydrogen atom;
R5 is selected from the group consisting of “hydrogen atom, —CO—CH₂—NH₂ (Gly residue), —CO—CH₂—CH₂—NH₂ (beta-Ala residue), —CO—CHCH₃—NH₂ (D- and/or L-alpha-Ala residue), —CO-(pyrrolidine-2-yl) (D- and/or L-Pro residue), 2-amino-2-carbonyl-propane (2-amino-isobutyric acid/Aib residue), 4-carbonyl-1H-piperidine, 3-carbonyl-1H-piperidine, R-(3-carbonyl-1H-piperidine), S-(3-carbonyl-1H-piperidine), 2-carbonyl-1H-piperidine, R-(2-carbonyl-1H-piperidine), S-(2-carbonyl-1H-piperidine), 1-amino-2-carbonyl-benzene, carbonyl-cyclohexane, 2-acetyl-pyridine, 3-acetyl-pyridine, 4-acetyl-pyridine, 2-propionyl-pyridine, 3-propionyl-pyridine, 4-propionyl-pyridine, (R-1-amino)-2-carbonyl-cyclohexane, (S-1-amino)-2-carbonyl-cyclohexane, 2-carbonyl-1H-imidazole, 2-carbonyl-pyridine, 3-carbonyl-pyridine, 4-carbonyl-pyridine, 2-amino-3-carbonyl-pyridine, 2-carbonyl-pyrazine, 2-carbonyl-4-hydroxy-1H-pyrrolidine, 4-carbonyl-1H,3H-diazacyclohexane, methyl-sulfonyl, phenylsulfonyl, 1-carbonyl-1-amino-2-phenylethane, phenylmethyl, 1-carbonyl-4-azide-benzene, 2-carbonyl-2,5-dihydro-1H-pyrrole, 2-carbonyl-piperazine, 2-carbonyl-1H-pyrrolidine, 2-aminoethane, carbonyl-benzene, 2-carbonyl-pyrazine, 3-carbonyl-pyrazine, 4-carbonyl-oxacyclohexane, 4-methyl-phenylsulfonyl, phenylmethyl-sulfonyl”;
R6 is a hydrogen atom; and
m is 0;

that can be used for the manufacture of a medicament for the treatment or prophylaxis of physiological and/or pathophysiological conditions in mammals, where the physiological and/or pathophysiological conditions are mediated by at least one G-protein coupled receptor (GPCR);

with the proviso that the following G-protein coupled receptors (GPCR) are excluded: “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

In a preferred embodiment compounds according to above formula (I) are provided, where
R3 is selected from the group consisting of “—CH₂—CO—CH₂-phenyl, —(CH₂)₂—CO—CH₂-phenyl, —CH₂—CO—NH₂, —(CH₂)₂—CO—NH₂, —CH₂—CO—OH, —(CH₂)₂—CO—OH, —(CH₂)₃—NH—C(NH)NH₂, —CH₂—SH, —(CH₂)₂—S—CH₃”;

that can be used for the manufacture of a medicament for the treatment or prophylaxis of physiological and/or pathophysiological conditions in mammals, where the physiological and/or pathophysiological conditions are mediated by at least one G-protein coupled receptor (GPCR);

with the proviso that the following G-protein coupled receptors (GPCR) are excluded: “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

In another preferred embodiment compounds according to above formula (I) are provided, where
R5 is selected from the group consisting of “hydrogen atom, methylsulfonyl, phenylsulfonyl, carbonyl-cyclohexane, (R-1-amino)-2-carbonyl-cyclohexane, (S-1-amino)-2-carbonyl-cyclohexane, 2-carbonyl-pyridine, 3-carbonyl-pyridine, 4-carbonyl-pyridine, 2-acetyl-pyridine, 3-acetyl-pyridine, 4-acetyl-pyridine, 2-propionyl-pyridine, 3-propionyl-pyridine, 4-propionyl-pyridine, 2-amino-3-carbonyl-pyridine, 2-carbonyl-1H-imidazole, 2-carbonyl-pyrazine, 4-carbonyl-1H,3H-diazacyclohexane”;

that can be used for the manufacture of a medicament for the treatment or prophylaxis of physiological and/or pathophysiological conditions in mammals, where the physiological and/or pathophysiological conditions are mediated by at least one G-protein coupled receptor (GPCR);

with the proviso that the following G-protein coupled receptors (GPCR) are excluded: “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

In a further aspect, the object of the invention has surprisingly been achieved by providing triazole compounds selected from the group consisting of:

### compound 1 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,

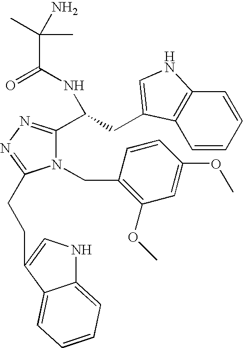

### compound 2 (R)-N-(1-(5-(2-(1H-indol-3-yl)ethyl)-4-phenethyl-4H-1,2,4-triazol-3-yl)-2-(1H-indol-3-yl)ethyl)-2-amino-2-methylpropanamide,

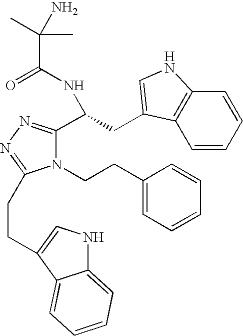

### compound 3 (R)-N-(1-(5-(3-(1H-indol-3-yl)propyl)-4-phenethyl-4H-1,2,4-triazol-3-yl)-2-(1H-indol-3-yl)ethyl)-2-amino-2-methylpropanamide,

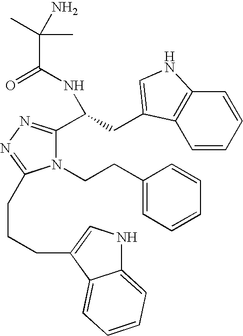

### compound 4 (R)-N-(1-(5-benzyl-4-(naphthalen-1-ylmethyl)-4H-1,2,4-triazol-3-yl)-2-(1H-indol-3-yl)ethyl)-2-amino-2-methylpropanamide,

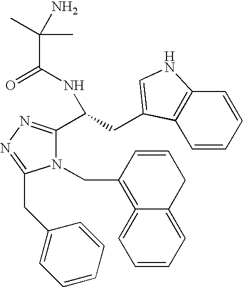

### compound 5 (R)-N-(1-(5-(2-(1H-indol-3-yl)ethyl)-4-(naphthalen-1-ylmethyl)-4H-1,2,4-triazol-3-yl)-2-(1H-indol-3-yl)ethyl)-2-amino-2-methylpropanamide,

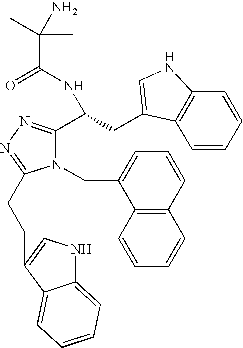

### compound 6 (R)-N-(1-(5-(2-(1H-indol-3-yl)ethyl)-4-(3-methoxybenzyl)-4H-1,2,4-triazol-3-yl)-2-(1H-indol-3-yl)ethyl)-2-amino-2-methylpropanamide,

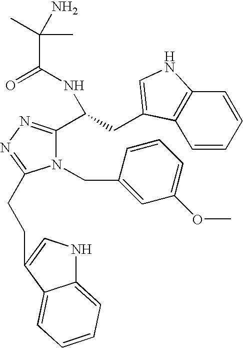

### compound 7 (R)-N-(1-(4-(3-methoxybenzyl)-5-benzyl-4H-1,2,4-triazol-3-yl)-2-(1H-indol-3-yl)ethyl)-2-amino-2-methylpropanamide,

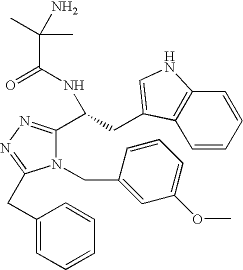

### compound 8 (R)-N-(1-(5-(2-(1H-indol-3-yl)ethyl)-4-benzyl-4H-1,2,4-triazol-3-yl)-2-(1H-indol-3-yl)ethyl)-2-amino-2-methylpropanamide,

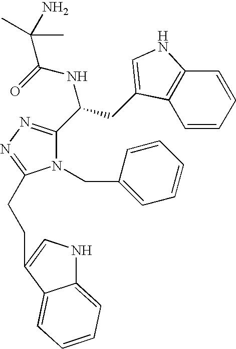

### compound 9 (R)-N-(1-(5-(3-(1H-indol-3-yl)propyl)-4-benzyl-4H-1,2,4-triazol-3-yl)-2-(1H-indol-3-yl)ethyl)-2-amino-2-methylpropanamide,

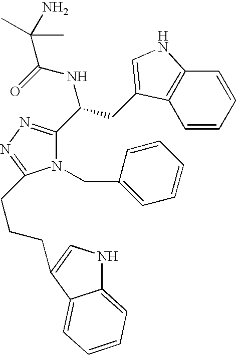

### compound 10 (R)-N-(1-(5-(3-(1H-indol-3-yl)propyl)-4-(3-methoxybenzyl)-4H-1,2,4-triazol-3-yl)-2-(1H-indol-3-yl)ethyl)-2-amino-2-methylpropanamide,

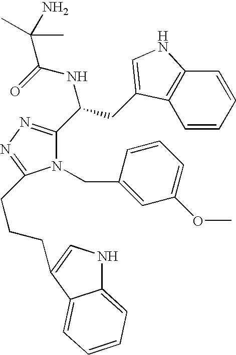

### compound 11 (R)-N-(1-(5-(3-(1H-indol-3-yl)propyl)-4-(naphthalen-1-ylmethyl)-4H-1,2,4-triazol-3-yl)-2-(1H-indol-3-yl)ethyl)-2-amino-2-methylpropanamide,

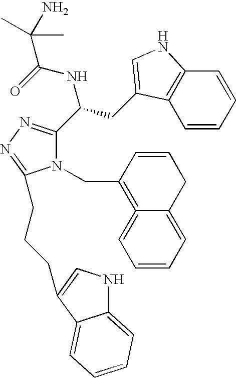

### compound 12 (R)-N-(1-(5-(2-(1H-indol-3-yl)ethyl)-4-(4-methoxybenzyl)-4H-1,2,4-triazol-3-yl)-2-(1H-indol-3-yl)ethyl)-2-amino-2-methylpropanamide,

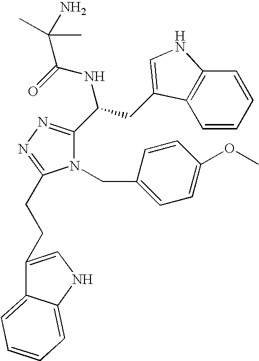

### compound 13 (R)-N-(1-(4-(4-methoxybenzyl)-5-benzyl-4H-1,2,4-triazol-3-yl)-2-(1H-indol-3-yl)ethyl)-2-amino-2-methylpropanamide,

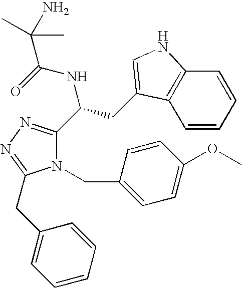

### compound 14 (R)-N-(1-(5-(3-(1H-indol-3-yl)propyl)-4-(4-bromobenzyl)-4H-1,2,4-triazol-3-yl)-2-(1H-indol-3-yl)ethyl)-2-amino-2-methylpropanamide,

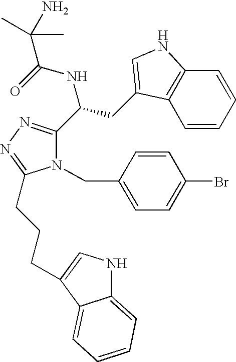

### compound 15 (R)-N-(1-(5-(2-(1H-indol-3-yl)ethyl)-4-hexyl-4H-1,2,4-triazol-3-yl)-2-(1H-indol-3-yl)ethyl)-2-amino-2-methylpropanamide,

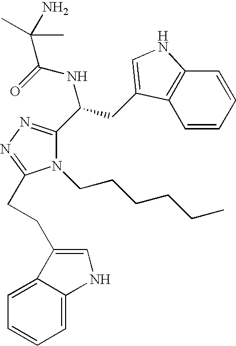

### compound 16 (R)-N-(1-(5-(3-(1H-indol-3-yl)propyl)-4-hexyl-4H-1,2,4-triazol-3-yl)-2-(1H-indol-3-yl)ethyl)-2-amino-2-methylpropanamide,

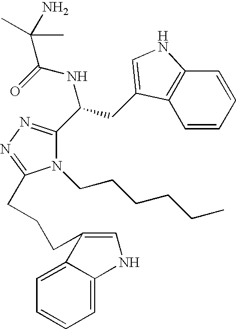

### compound 17 (R)-N-(1-(4,5-bis(2-(1H-indol-3-yl)ethyl)-4H-1,2,4-triazol-3-yl)-2-(1H-indol-3-yl)ethyl)-2-amino-2-methylpropanamide,

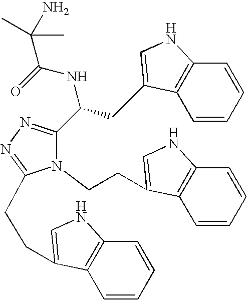

### compound 18 (S)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,

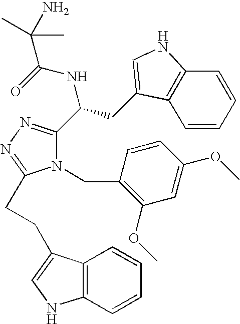

### compound 19 (R)-N-(1-(4-(3-methoxybenzyl)-5-phenethyl-4H-1,2,4-triazol-3-yl)-2-(1H-indol-3-yl)ethyl)-2-amino-2-methylpropanamide,

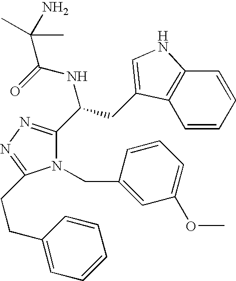

### compound 20 (R)-N-(1-(4-(4-methoxybenzyl)-5-phenethyl-4H-1,2,4-triazol-3-yl)-2-(1H-indol-3-yl)ethyl)-2-amino-2-methylpropanamide,

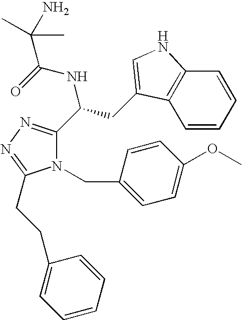

### compound 21 (R)-N-(1-(5-(2-(1H-indol-3-yl)ethyl)-4-(3,5-dimethoxybenzyl)-4H-1,2,4-triazol-3-yl)-2-(1H-indol-3-yl)ethyl)-2-amino-2-methylpropanamide,

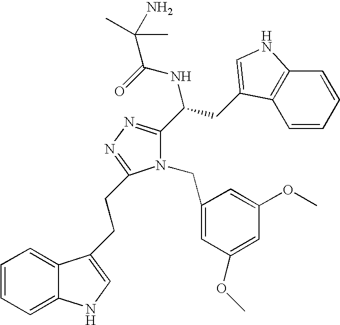

### compound 22 (R)-N-(1-(4-(4-methoxybenzyl)-5-(3-phenylpropyl)-4H-1,2,4-triazol-3-yl)-2-(1H-indol-3-yl)ethyl)-2-amino-2-methylpropanamide,

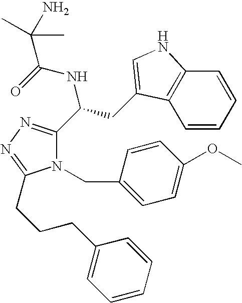

### compound 23 (R)-N-(1-(5-(3-(1H-indol-3-yl)propyl)-4-(4-methoxybenzyl)-4H-1,2,4-triazol-3-yl)-2-(1H-indol-3-yl)ethyl)-2-amino-2-methylpropanamide,

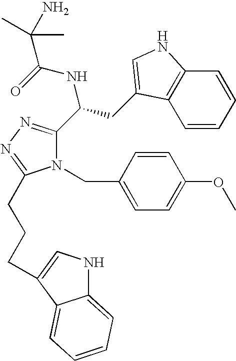

### compound 24 (R)-N-(1-(4-(2-(1H-indol-3-yl)ethyl)-5-(3-(1H-indol-3-yl)propyl)-4H-1,2,4-triazol-3-yl)-2-(1H-indol-3-yl)ethyl)-2-amino-2-methylpropanamide,

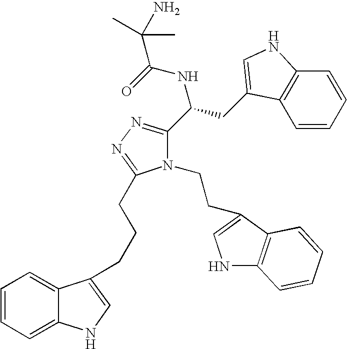

### compound 25 (R)-N-(1-(5-(2-(1H-indol-3-yl)ethyl)-4-(2-methoxy)benzyl)-4H-1,2,4-triazol-3-yl)-2-(1H-indol-3-yl)ethyl)-2-amino-2-methylpropanamide,

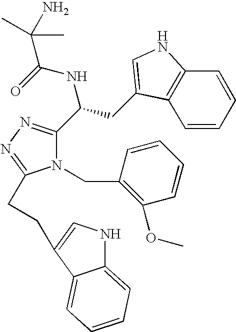

### compound 26 (R)-N-(1-(4-(2-methoxybenzyl)-5-phenethyl-4H-1,2,4-triazol-3-yl)-2-(1H-indol-3-yl)ethyl)-2-amino-2-methylpropanamide,

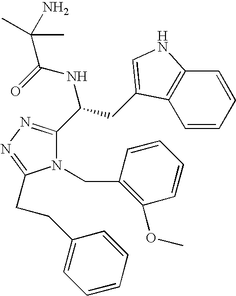

### compound 27 (R)-N-(2-(1H-indol-3-yl)-1-(4-(naphthalen-1-ylmethyl)-5-phenethyl-4H-1,2,4-triazol-3-yl)ethyl)-2-amino-2-methylpropanamide,

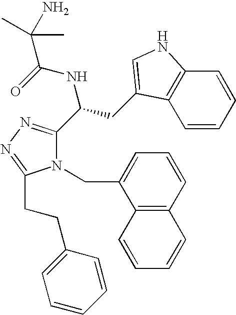

### compound 28 (R)-N-(1-(5-(2-(1H-indol-3-yl)ethyl)-4-(3,4-dichlorobenzyl)-4H-1,2,4-triazol-3-yl)-2-(1H-indol-3-yl)ethyl)-2-amino-2-methylpropanamide,

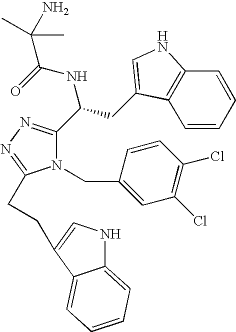

### compound 29 (R)-N-(1-(5-(2-(1H-indol-3-yl)ethyl)-4-(4-fluorobenzyl)-4H-1,2,4-triazol-3-yl)-2-(1H-indol-3-yl)ethyl)-2-amino-2-methylpropanamide,

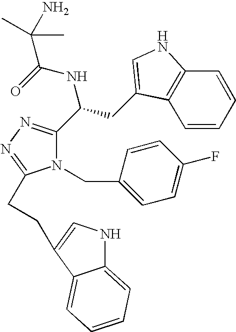

### compound 30 (R)-N-(1-(4-(4-fluorobenzyl)-5-benzyl-4H-1,2,4-triazol-3-yl)-2-(1H-indol-3-yl)ethyl)-2-amino-2-methylpropanamide,

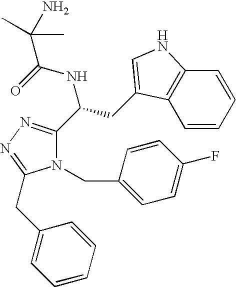

### compound 31 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-4-carboxamide,

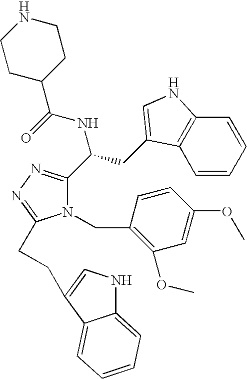

### compound 32 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-3-carboxamide,

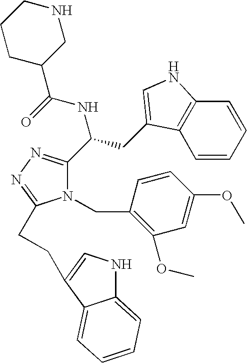

### compound 33 (R)-N-(1-(4-(4-methylbenzyl)-5-(3-phenylpropyl)-4H-1,2,4-triazol-3-yl)-2-(1H-indol-3-yl)ethyl)-2-amino-2-methylpropanamide,

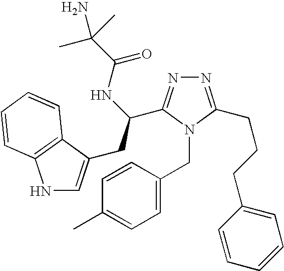

### compound 34 (R)-N-(1-(5-(2-(1H-indol-3-yl)ethyl)-4-(4-methylbenzyl)-4H-1,2,4-triazol-3-yl)-2-(1H-indol-3-yl)ethyl)-2-amino-2-methylpropanamide,

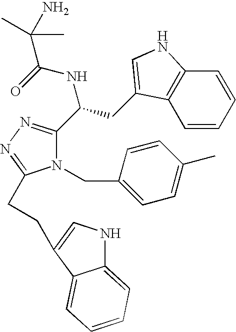

### compound 36 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-2-carboxamide,

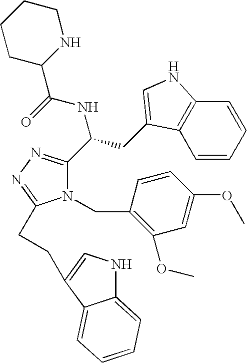

### compound 37 (R)-N-(1-(4-(4-methylbenzyl)-5-phenethyl-4H-1,2,4-triazol-3-yl)-2-(1H-indol-3-yl)ethyl)-2-amino-2-methylpropanamide,

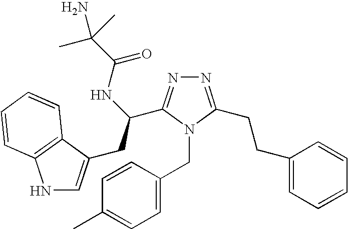

### compound 38 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminobenzamide,

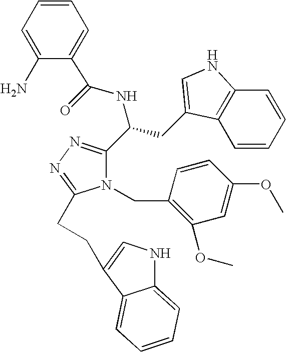

### compound 39 (R)-N-(1-(5-benzyl-4-(pyridin-2-ylmethyl)-4H-1,2,4-triazol-3-yl)-2-(1H-indol-3-yl)ethyl)-2-amino-2-methylpropanamide,

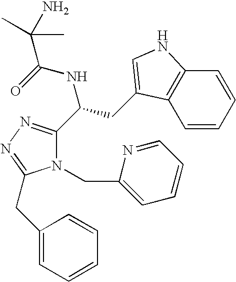

### compound 40 (2S,4R)-N-((R)-1-(4-(4-methoxybenzyl)-5-phenethyl-4H-1,2,4-triazol-3-yl)-2-(1H-indol-3-yl)ethyl)-4-hydroxypyrrolidine-2-carboxamide,

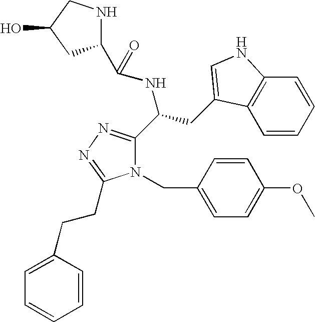

### compound 41 (S)-N-((R)-1-(4-(4-methoxybenzyl)-5-phenethyl-4H-1,2,4-triazol-3-yl)-2-(1H-indol-3-yl)ethyl)piperidine-3-carboxamide,

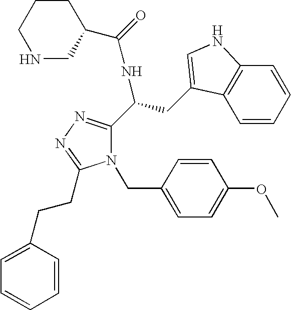

### compound 42 (R)-N-((R)-1-(4-(4-methoxybenzyl)-5-phenethyl-4H-1,2,4-triazol-3-yl)-2-(1H-indol-3-yl)ethyl)piperidine-3-carboxamide,

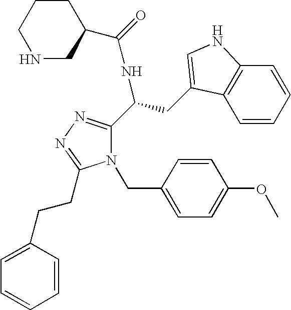

### compound 43 (R)-N-(1-(4-(4-ethylbenzyl)-5-phenethyl-4H-1,2,4-triazol-3-yl)-2-(1H-indol-3-yl)ethyl)-2-amino-2-methylpropanamide,

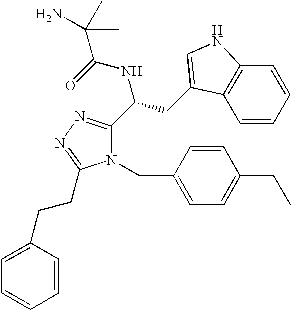

### compound 44 (R)-N-(1-(4-(4-methoxybenzyl)-5-phenethyl-4H-1,2,4-triazol-3-yl)-2-(1H-indol-3-yl)ethyl)piperidine-4-carboxamide,

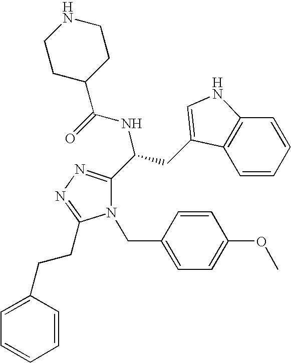

### compound 45 (R)-N-(1-(5-(2-(1H-indol-3-yl)ethyl)-4-(4-methoxybenzyl)-4H-1,2,4-triazol-3-yl)-2-(1H-indol-3-yl)ethyl)piperidine-4-carboxamide,

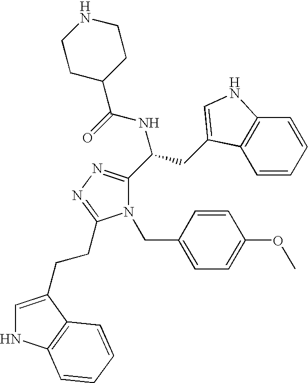

### compound 46 (S)-N-((R)-1-(4-(4-methoxybenzyl)-5-phenethyl-4H-1,2,4-triazol-3-yl)-2-(1H-indol-3-yl)ethyl)pyrrolidine-2-carboxamide,

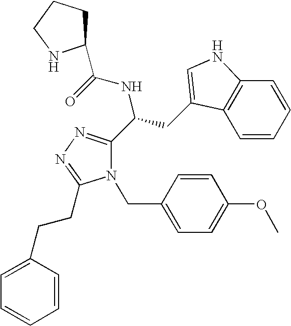

### compound 47 (R)-N-((R)-1-(4-(4-methoxybenzyl)-5-phenethyl-4H-1,2,4-triazol-3-yl)-2-(1H-indol-3-yl)ethyl)pyrrolidine-2-carboxamide,

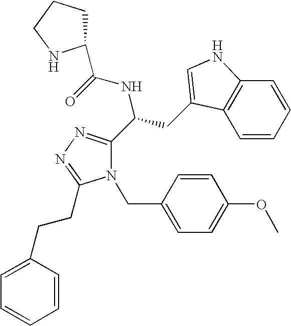

### compound 48 (S)-N-((R)-1-(4-(4-methoxybenzyl)-5-phenethyl-4H-1,2,4-triazol-3-yl)-2-(1H-indol-3-yl)ethyl)piperidine-2-carboxamide,

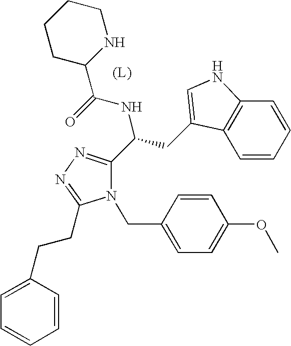

### compound 49 (R)-N-((R)-1-(4-(4-methoxybenzyl)-5-phenethyl-4H-1,2,4-triazol-3-yl)-2-(1H-indol-3-yl)ethyl)piperidine-2-carboxamide,

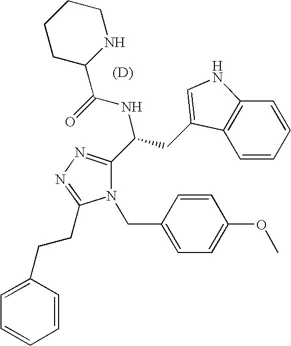

### compound 50 (R)-N-(1-(4-(4-methoxybenzyl)-5-phenethyl-4H-1,2,4-triazol-3-yl)-2-(1H-indol-3-yl)ethyl)-2-aminoacetamide,

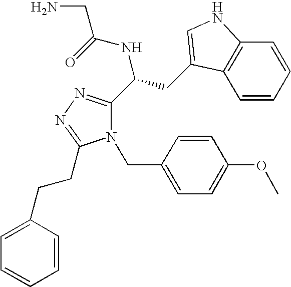

### compound 51 (R)-N-(1-(4-(4-methoxybenzyl)-5-phenethyl-4H-1,2,4-triazol-3-yl)-2-(1H-indol-3-yl)ethyl)-2-(pyridin-2-yl)acetamide,

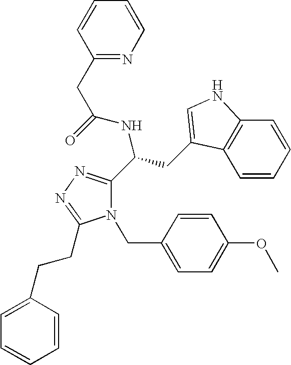

### compound 52 (R)-N-(1-(4-(4-methoxybenzyl)-5-phenethyl-4H-1,2,4-triazol-3-yl)-2-(1H-indol-3-yl)ethyl)-2-(pyridin-4-yl)acetamide,

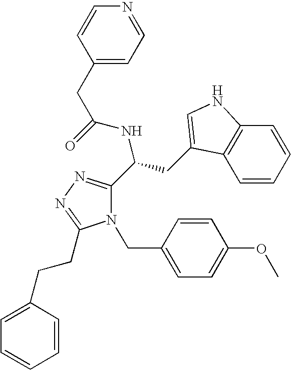

### compound 53 (R)-N-(1-(4-(4-methoxybenzyl)-5-phenethyl-4H-1,2,4-triazol-3-yl)-2-(1H-indol-3-yl)ethyl)cyclohexanecarboxamide,

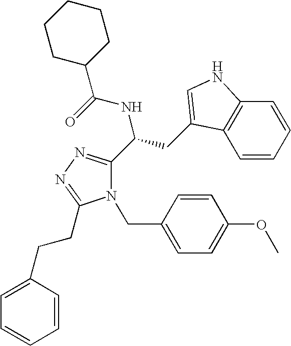

### compound 54 (R)-N-(1-(5-(2-(1H-indol-3-yl)ethyl)-4-benzyl-4H-1,2,4-triazol-3-yl)-2-(1H-indol-3-yl)ethyl)piperidine-4-carboxamide,

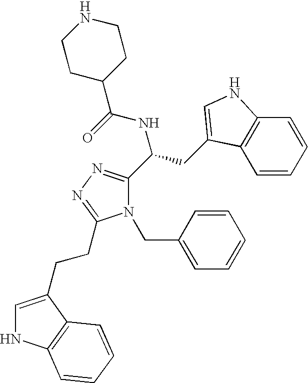

### compound 55 (R)-N-(1-(5-(2-(1H-indol-3-yl)ethyl)-4-benzyl-4H-1,2,4-triazol-3-yl)-2-(1H-indol-3-yl)ethyl)piperidine-3-carboxamide,

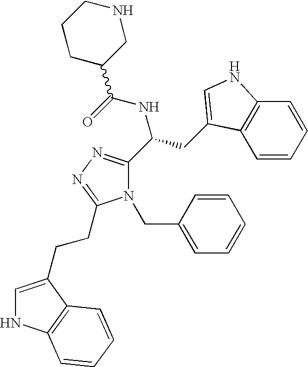

### compound 56 (R)-N-(1-(4-(4-methoxybenzyl)-5-phenethyl-4H-1,2,4-triazol-3-yl)-2-(1H-indol-3-yl)ethyl)-3-aminopropanamide,

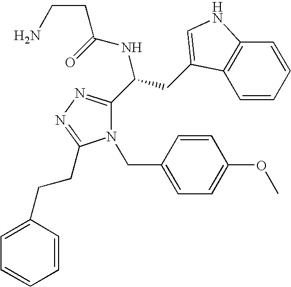

### compound 57 (S)-N-((R)-1-(4-(4-methoxybenzyl)-5-phenethyl-4H-1,2,4-triazol-3-yl)-2-(1H-indol-3-yl)ethyl)-2-aminopropanamide,

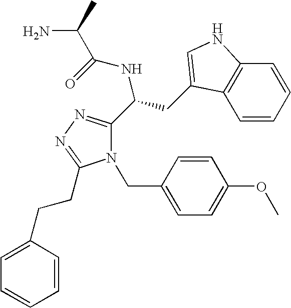

### compound 58 (R)-N-(1-(4-(4-methoxybenzyl)-5-phenethyl-4H-1,2,4-triazol-3-yl)-2-(1H-indol-3-yl)ethyl)-2-(pyridin-3-yl)acetamide,

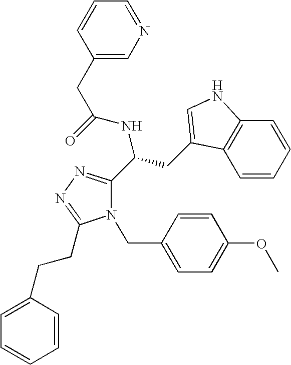

### compound 59 (R)-N-(1-(4-(4-methoxybenzyl)-5-phenethyl-4H-1,2,4-triazol-3-yl)-2-(1H-indol-3-yl)ethyl)-3-(pyridin-3-yl)propanamide,

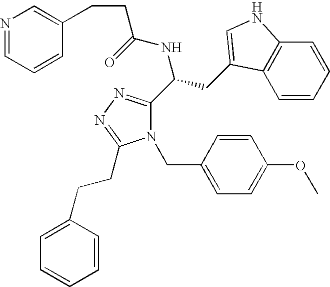

### compound 60 (R)-N-(1-(5-(2-(1H-indol-3-yl)ethyl)-4-benzyl-4H-1,2,4-triazol-3-yl)-2-(1H-indol-3-yl)ethyl)-2-(pyridin-2-yl)acetamide,

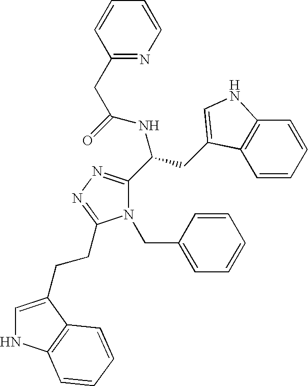

### compound 61 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-(pyridin-2-yl)acetamide,

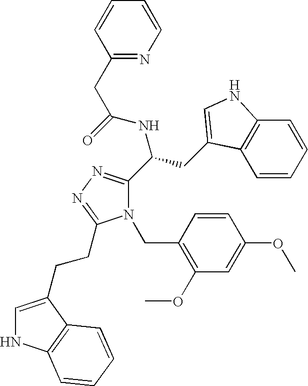

### compound 62 (R)-N-(1-(4-(2,4-dimethoxybenzyl)-5-phenethyl-4H-1,2,4-triazol-3-yl)-2-(1H-indol-3-yl)ethyl)piperidine-4-carboxamide,

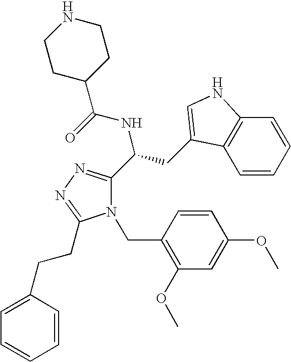

### compound 63 (R)-N-((R)-1-(4-(2,4-dimethoxybenzyl)-5-phenethyl-4H-1,2,4-triazol-3-yl)-2-(1H-indol-3-yl)ethyl)piperidine-2-carboxamide,

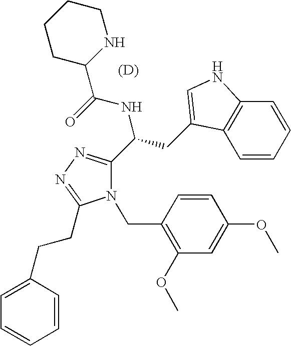

### compound 64 (R)-N-(1-(4-(2,4-dimethoxybenzyl)-5-phenethyl-4H-1,2,4-triazol-3-yl)-2-(1H-indol-3-yl)ethyl)picolinamide,

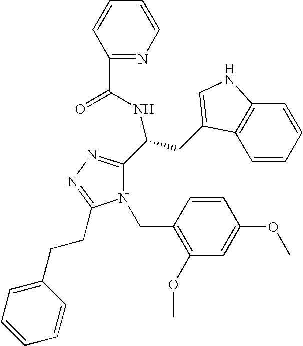

### compound 65 (R)-N-(1-(4-(2,4-dimethoxybenzyl)-5-phenethyl-4H-1,2,4-triazol-3-yl)-2-(1H-indol-3-yl)ethyl)isonicotinamide,

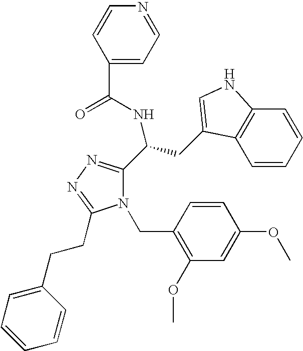

### compound 66 (R)-N-(1-(4-(2,4-dimethoxybenzyl)-5-phenethyl-4H-1,2,4-triazol-3-yl)-2-(1H-indol-3-yl)ethyl)pyrazine-2-carboxamide,

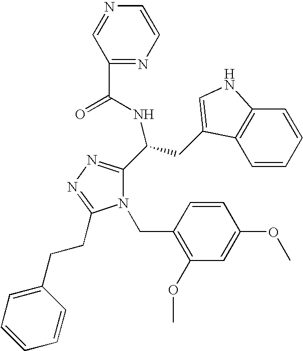

### compound 67 (R)-N-1-(4-(2,4-dimethoxybenzyl)-5-phenethyl-4H-1,2,4-triazol-3-yl)-2-(1H-indol-3-yl)ethyl)piperazine-2-carboxamide,

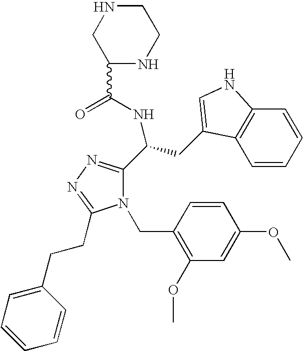

### compound 68 (S)-N-((R)-1-(4-(2,4-dimethoxybenzyl)-5-phenethyl-4H-1,2,4-triazol-3-yl)-2-(1H-indol-3-yl)ethyl)pyrrolidine-2-carboxamide,

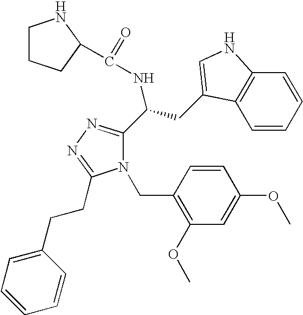

### compound 69 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminoacetamide,

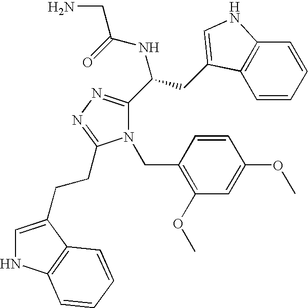

### compound 70 (S)-N-((R)-1-(5-(2-(1H-indol-3-yl)ethyl)-4-(2,4-dimethoxybenzyl)-4H-1,2,4-triazol-3-yl)-2-(1H-indol-3-yl)ethyl)pyrrolidine-2-carboxamide,

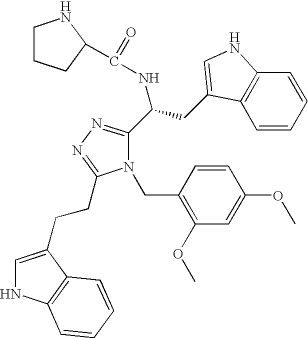

### compound 71 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)pyrazine-2-carboxamide,

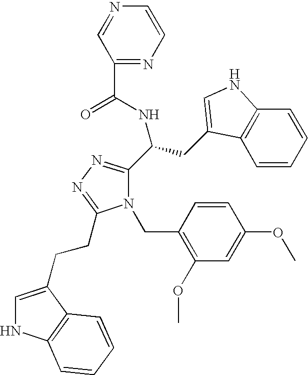

### compound 72 (R)-N-1-(5-(2-(1H-indol-3-yl)ethyl)-4-(2,4-dimethoxybenzyl)-4H-1,2,4-triazol-3-yl)-2-(1H-indol-3-yl)ethyl)piperazine-2-carboxamide,

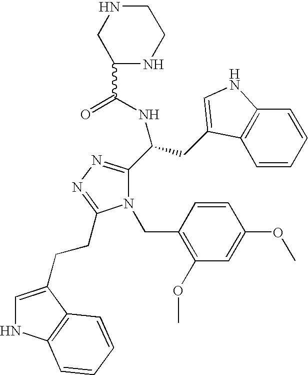

### compound 73 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)picolinamide,

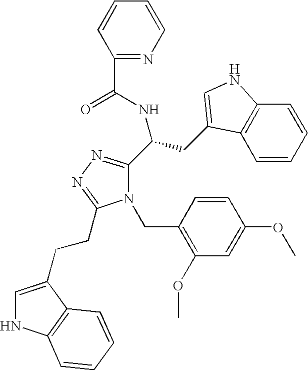

### compound 74 (R)-1-(5-(2-(1H-indol-3-yl)ethyl)-4-(2,4-dimethoxybenzyl)-4H-1,2,4-triazol-3-yl)-2-(1H-indol-3-yl)ethanamine,

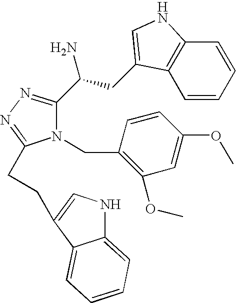

### compound 75 (R)-N-(1-(4-(2,4-dimethoxybenzyl)-5-phenethyl-4H-1,2,4-triazol-3-yl)-2-(1H-indol-3-yl)ethyl)-2-aminoacetamide,

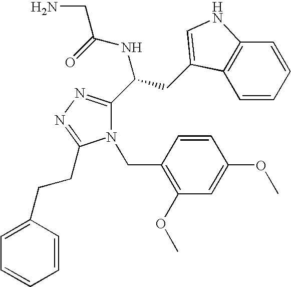

### compound 76 (R)-N-(1-(4-(4-methoxybenzyl)-5-phenethyl-4H-1,2,4-triazol-3-yl)-2-(1H-indol-3-yl)ethyl)pyrazine-2-carboxamide,

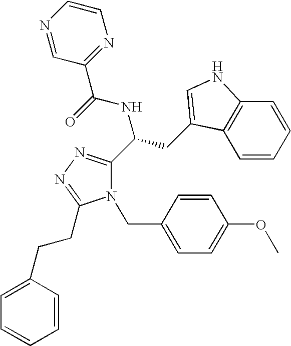

### compound 77 (R)-N-(1-(4-(4-methoxybenzyl)-5-phenethyl-4H-1,2,4-triazol-3-yl)-2-(1H-indol-3-yl)ethyl)isonicotinamide,

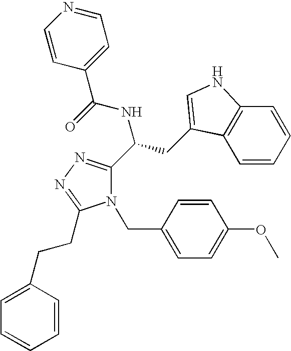

### compound 78 (R)-N-1-(4-(4-methoxybenzyl)-5-phenethyl-4H-1,2,4-triazol-3-yl)-2-(1H-indol-3-yl)ethyl)piperazine-2-carboxamide,

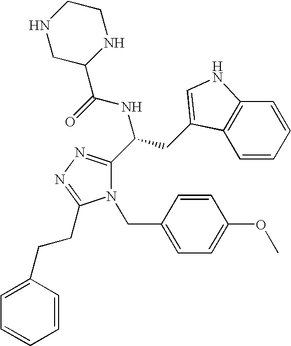

### compound 79 (R)-N-(1-(4-(4-methoxybenzyl)-5-phenethyl-4H-1,2,4-triazol-3-yl)-2-(1H-indol-3-yl)ethyl)picolinamide,

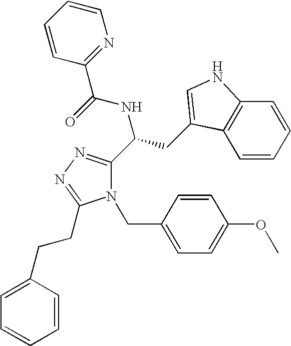

### compound 80 (R)-N-(1-(5-(2-(1H-indol-3-yl)ethyl)-4-(4-methoxybenzyl)-4H-1,2,4-triazol-3-yl)-2-(1H-indol-3-yl)ethyl)picolinamide,

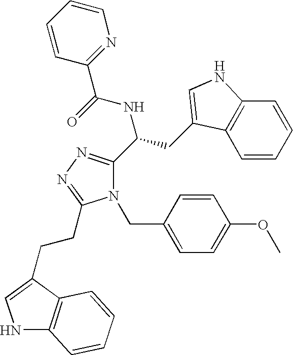

### compound 81 (R)-N-1-(5-(2-(1H-indol-3-yl)ethyl)-4-(4-methoxybenzyl)-4H-1,2,4-triazol-3-yl)-2-(1H-indol-3-yl)ethyl)piperazine-2-carboxamide,

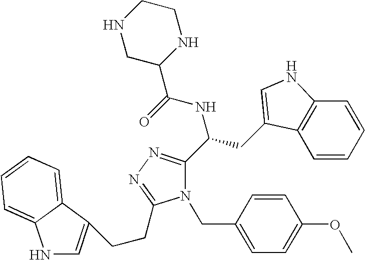

### compound 82 (R)-N-(1-(4-(4-ethylbenzyl)-5-phenethyl-4H-1,2,4-triazol-3-yl)-2-(1H-indol-3-yl)ethyl)-2-(pyridin-2-yl)acetamide,

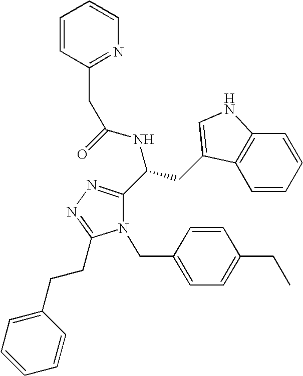

### compound 83 (R)-N-(1-(4-(4-ethylbenzyl)-5-phenethyl-4H-1,2,4-triazol-3-yl)-2-(1H-indol-3-yl)ethyl)piperidine-4-carboxamide,

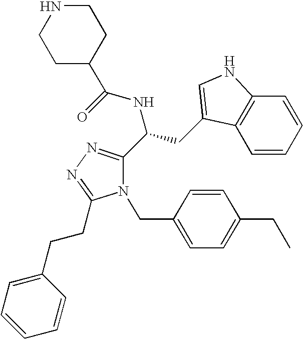

### compound 84 (R)-N-(1-(4-(4-ethylbenzyl)-5-phenethyl-4H-1,2,4-triazol-3-yl)-2-(1H-indol-3-yl)ethyl)piperazine-2-carboxamide,

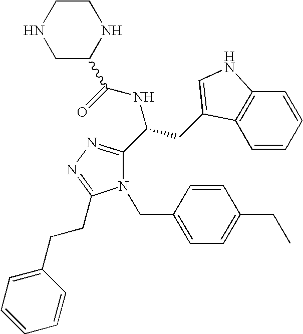

### compound 85 (R)-N-(1-(4-(4-ethylbenzyl)-5-phenethyl-4H-1,2,4-triazol-3-yl)-2-(1H-indol-3-yl)ethyl)pyrazine-2-carboxamide,

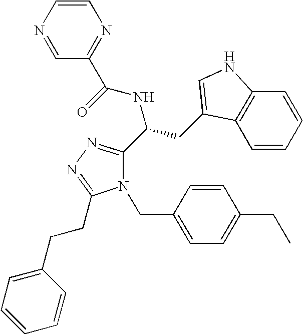

### compound 86 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-cis-aminocyclohexanecarboxamide,

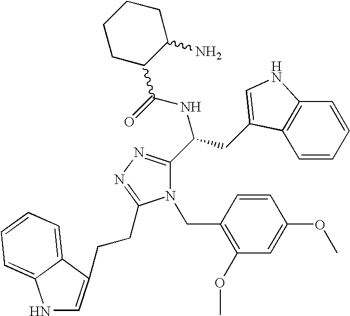

### compound 87 (S)-N-((R)-1-(5-(2-(1H-indol-3-yl)ethyl)-4-(4-methoxybenzyl)-4H-1,2,4-triazol-3-yl)-2-(1H-indol-3-yl)ethyl)piperidine-3-carboxamide,

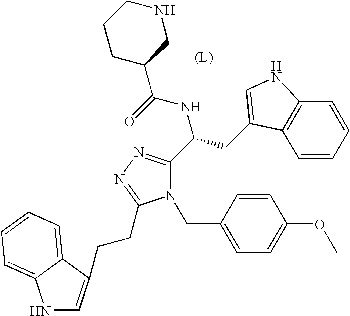

### compound 88 (R)-N-((R)-1-(5-(2-(1H-indol-3-yl)ethyl)-4-(4-methoxybenzyl)-4H-1,2,4-triazol-3-yl)-2-(1H-indol-3-yl)ethyl)piperidine-2-carboxamide,

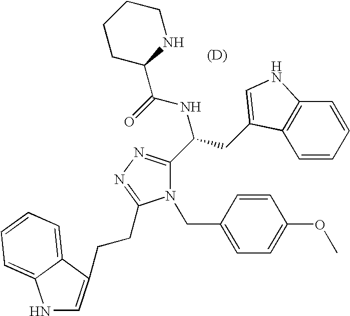

### compound 89 (S)-N-((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,

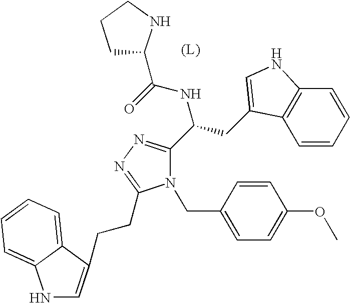

### compound 90 (R)-N-((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,

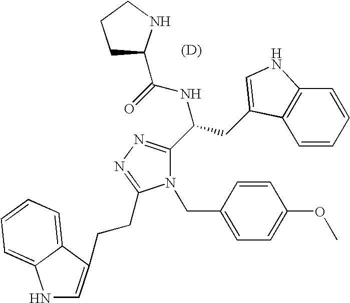

### compound 91 (R)-N-(1-(5-(2-(1H-indol-3-yl)ethyl)-4-(4-methoxybenzyl)-4H-1,2,4-triazol-3-yl)-2-(1H-indol-3-yl)ethyl)-2-(pyridin-2-yl)acetamide,

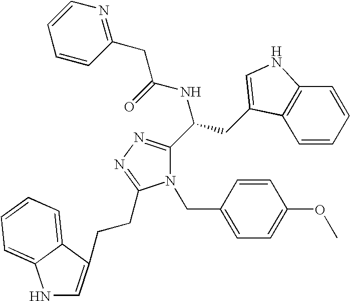

### compound 92 (R)-N-(1-(5-(2-(1H-indol-3-yl)ethyl)-4-(4-bromobenzyl)-4H-1,2,4-triazol-3-yl)-2-(1H-indol-3-yl)ethyl)-2-amino-2-methylpropanamide,

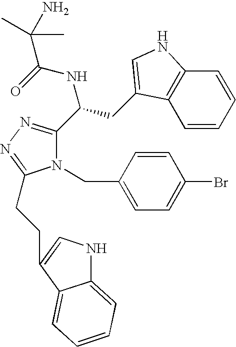

### compound 93 (R)-N-(1-(5-(2-(1H-indol-3-yl)ethyl)-4-(4-methoxybenzyl)-4H-1,2,4-triazol-3-yl)-2-phenylethyl)-2-amino-2-methylpropanamide,

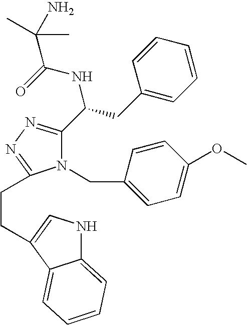

### compound 94 (R)-N-(2-(1H-indol-3-yl)-1-(5-phenethyl-4-(thiophen-2-ylmethyl)-4H-1,2,4-triazol-3-yl)ethyl)piperidine-4-carboxamide,

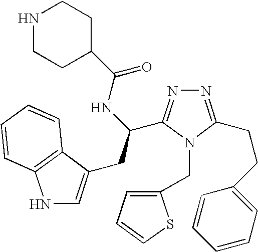

### compound 95 (R)-N-(1-(4-(2-(1H-indol-3-yl)ethyl)-4H-1,2,4-triazol-3-yl)-2-(1H-indol-3-yl)ethyl)-2-amino-2-methylpropanamide,

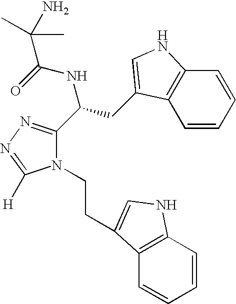

### compound 96 (R)-N-(1-(5-((1H-indol-3-yl)methyl)-4-methyl-4H-1,2,4-triazol-3-yl)-2-(1H-indol-3-yl)ethyl)-2-amino-2-methylpropanamide,

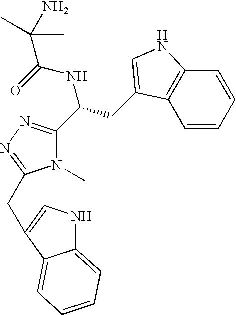

### compound 97 (R)-N-(1-(5-(2-(1H-indol-3-yl)ethyl)-4-methyl-4H-1,2,4-triazol-3-yl)-2-(1H-indol-3-yl)ethyl)-2-amino-2-methylpropanamide,

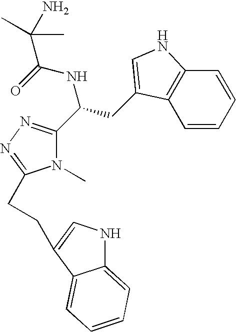

### compound 98 (R)-N-(1-(5-((1H-indol-3-yl)methyl)-4H-1,2,4-triazol-3-yl)-2-(1H-indol-3-yl)ethyl)-2-amino-2-methylpropanamide,

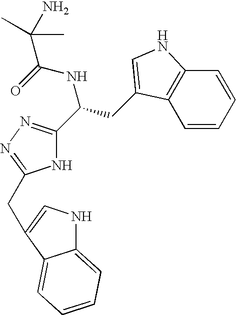

### compound 99 (R)-N-(1-(5-((1H-indol-3-yl)methyl)-4-(2,4-dimethoxybenzyl)-4H-1,2,4-triazol-3-yl)-2-(1H-indol-3-yl)ethyl)-2-amino-2-methylpropanamide,

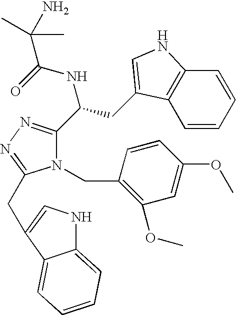

### compound 100 (R)-N-(1-(4-(2,4-dimethoxybenzyl)-5-methyl-4H-1,2,4-triazol-3-yl)-2-(1H-indol-3-yl)ethyl)-2-amino-2-methylpropanamide,

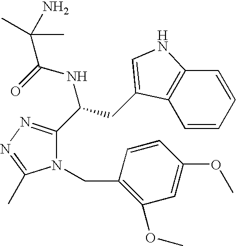

### compound 101 (R)-N-(1-(5-((1H-indol-3-yl)methyl)-4-(4-methoxybenzyl)-4H-1,2,4-triazol-3-yl)-2-(1H-indol-3-yl)ethyl)-2-amino-2-methylpropanamide,

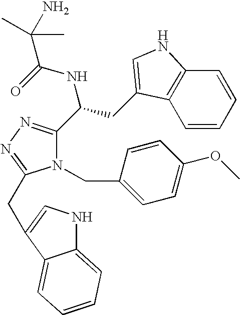

### compound 102 (R)-N-(1-(4-(2,4-dimethoxybenzyl)-5-benzyl-4H-1,2,4-triazol-3-yl)-2-(1H-indol-3-yl)ethyl)-2-amino-2-methylpropanamide,

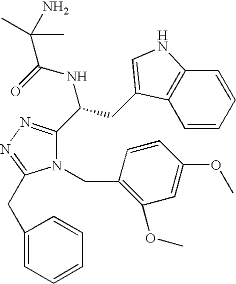

### compound 103 (R)-N-(1-(5-(3-(1H-indol-3-yl)propyl)-4-(2,4-dimethoxybenzyl)-4H-1,2,4-triazol-3-yl)-2-(1H-indol-3-yl)ethyl)-2-amino-2-methylpropanamide,

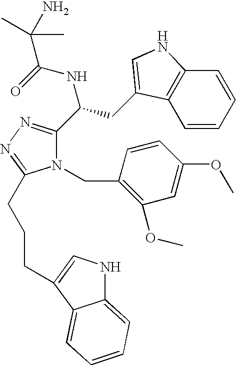

### compound 104 (R)-N-(1-(5-((1H-indol-3-yl)methyl)-4-phenethyl-4H-1,2,4-triazol-3-yl)-2-(1H-indol-3-yl)ethyl)-2-amino-2-methylpropanamide,

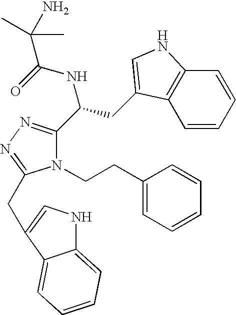

### compound 105 (R)-N-(1-(5-benzyl-4-phenethyl-4H-1,2,4-triazol-3-yl)-2-(1H-indol-3-yl)ethyl)-2-amino-2-methylpropanamide,

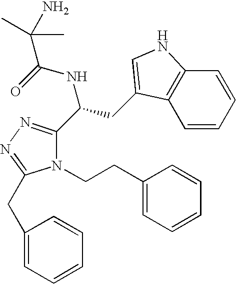

### compound 106 (R)-N-(1-(5-benzyl-4-(2,2-diphenylethyl)-4H-1,2,4-triazol-3-yl)-2-(1H-indol-3-yl)ethyl)-2-amino-2-methylpropanamide,

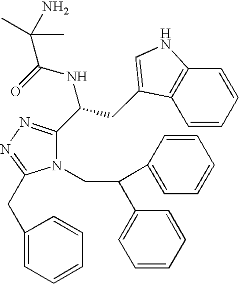

### compound 107 (R)-N-(1-(5-(2-(1H-indol-3-yl)ethyl)-4-(2,2-diphenylethyl)-4H-1,2,4-triazol-3-yl)-2-(1H-indol-3-yl)ethyl)-2-amino-2-methylpropanamide,

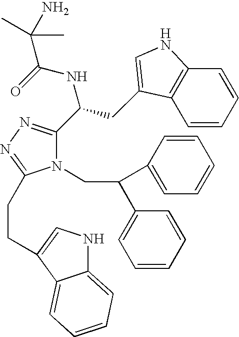

### compound 108 (R)-N-(1-(4-(3,5-dimethoxybenzyl)-5-benzyl-4H-1,2,4-triazol-3-yl)-2-(1H-indol-3-yl)ethyl)-2-amino-2-methylpropanamide,

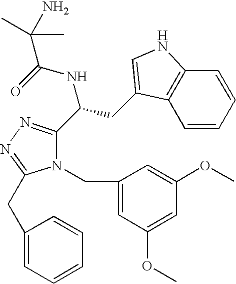

### compound 109 (R)-N-(1-(4,5-dibenzyl-4H-1,2,4-triazol-3-yl)-2-(1H-indol-3-yl)ethyl)-2-amino-2-methylpropanamide,

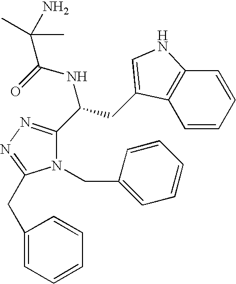

### compound 110 (R)-N-(1-(5-benzyl-4-hexyl-4H-1,2,4-triazol-3-yl)-2-(1H-indol-3-yl)ethyl)-2-amino-2-methylpropanamide,

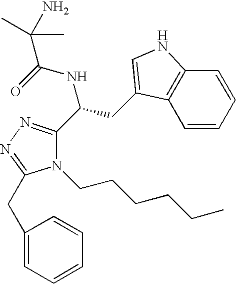

### compound 111 (R)-N-(1-(4-(2-(1H-indol-3-yl)ethyl)-5-benzyl-4H-1,2,4-triazol-3-yl)-2-(1H-indol-3-yl)ethyl)-2-amino-2-methylpropanamide,

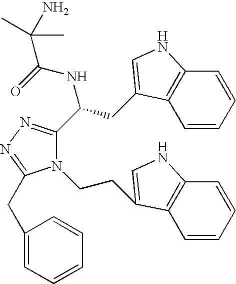

### compound 112 (S)-N-(1-(4-(2,4-dimethoxybenzyl)-5-benzyl-4H-1,2,4-triazol-3-yl)-2-(1H-indol-3-yl)ethyl)-2-amino-2-methylpropanamide,

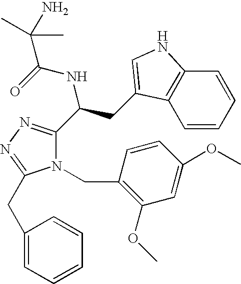

### compound 113 (R)-N-(1-(4-(3,5-dimethoxybenzyl)-5-phenethyl-4H-1,2,4-triazol-3-yl)-2-(1H-indol-3-yl)ethyl)-2-amino-2-methylpropanamide,

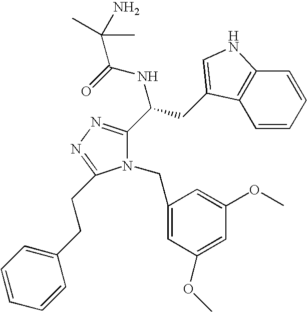

### compound 114 (R)-N-(1-(4-(4-bromobenzyl)-5-benzyl-4H-1,2,4-triazol-3-yl)-2-(1H-indol-3-yl)ethyl)-2-amino-2-methylpropanamide,

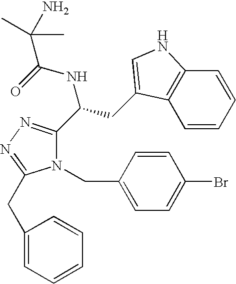

### compound 115 (R)-N-(1-(4-(2-methoxybenzyl)-5-benzyl-4H-1,2,4-triazol-3-yl)-2-(1H-indol-3-yl)ethyl)-2-amino-2-methylpropanamide,

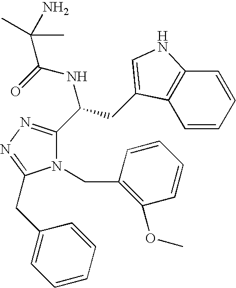

### compound 116 (S)-N-(1-(4-(2,4-dimethoxybenzyl)-5-phenethyl-4H-1,2,4-triazol-3-yl)-2-(1H-indol-3-yl)ethyl)-2-amino-2-methylpropanamide,

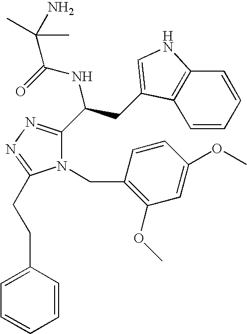

### compound 117 (R)-N-(1-(4,5-diphenethyl-4H-1,2,4-triazol-3-yl)-2-(1H-indol-3-yl)ethyl)-2-amino-2-methylpropanamide,

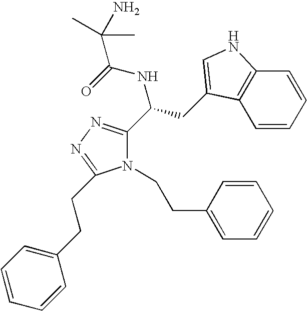

### compound 118 (R)-N-(1-(4-(3,4-dichlorobenzyl)-5-benzyl-4H-1,2,4-triazol-3-yl)-2-(1H-indol-3-yl)ethyl)-2-amino-2-methylpropanamide,

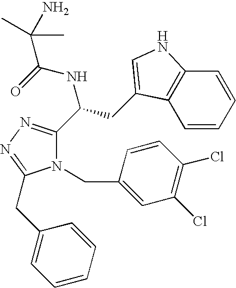

### compound 119 (R)-1-(5-(2-(1H-indol-3-yl)ethyl)-4-(2,4-dimethoxybenzyl)-4H-1,2,4-triazol-3-yl)-2-(1H-indol-3-yl)ethanamine,

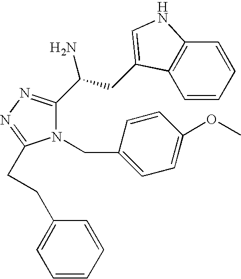

### compound 120 (R)-N-(1-(4-(4-methoxybenzyl)-5-benzyl-4H-1,2,4-triazol-3-yl)-2-phenylethyl)-2-amino-2-methylpropanamide,

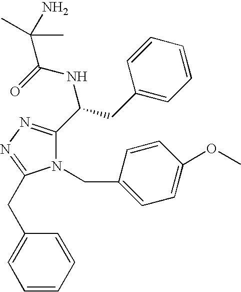

### compound 121 (R)-N-(1-(4-(4-fluorobenzyl)-5-phenethyl-4H-1,2,4-triazol-3-yl)-2-(1H-indol-3-yl)ethyl)-2-amino-2-methylpropanamide,

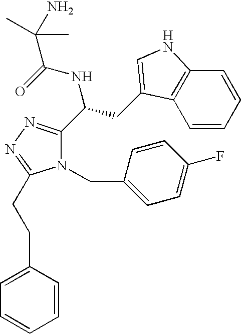

### compound 122 (R)-N-(1-(4-(3,4-dichlorobenzyl)-5-phenethyl-4H-1,2,4-triazol-3-yl)-2-(1H-indol-3-yl)ethyl)-2-amino-2-methylpropanamide,

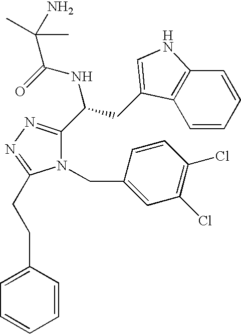

### compound 124 (R)-N-(1-(4-(4-methylbenzyl)-5-benzyl-4H-1,2,4-triazol-3-yl)-2-(1H-indol-3-yl)ethyl)-2-amino-2-methylpropanamide,

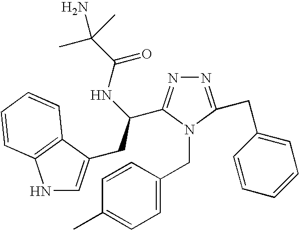

### compound 125 (S)-N-(1-(4-(4-methoxybenzyl)-5-(3-phenylpropyl)-4H-1,2,4-triazol-3-yl)-2-(1H-indol-3-yl)ethyl)-2-amino-2-methylpropanamide,

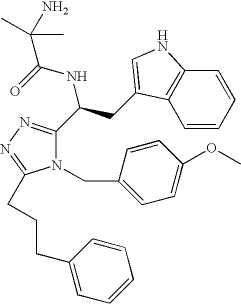

### compound 126 (S)-N-(1-(4-(4-methoxybenzyl)-5-benzyl-4H-1,2,4-triazol-3-yl)-2-(1H-indol-3-yl)ethyl)-2-amino-2-methylpropanamide,

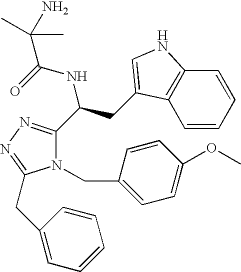

### compound 128 N-((R)-1-(4-(4-nitrobenzyl)-5-phenethyl-4H-1,2,4-triazol-3-yl)-2-(1H-indol-3-yl)ethyl)-2-amino-2-methylpropanamide,

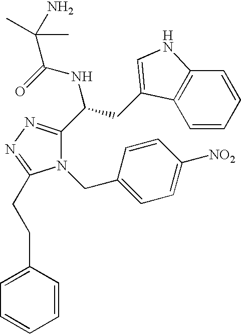

### compound 129 (S)-N-(1-(4-(4-methoxybenzyl)-5-phenethyl-4H-1,2,4-triazol-3-yl)-2-(1H-indol-3-yl)ethyl)-2-amino-2-methylpropanamide,

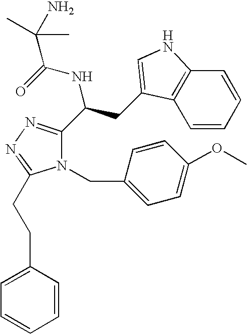

### compound 130 (R)-N-(1-(4-(4-methoxyphenethyl)-5-phenethyl-4H-1,2,4-triazol-3-yl)-2-(1H-indol-3-yl)ethyl)-2-amino-2-methylpropanamide,

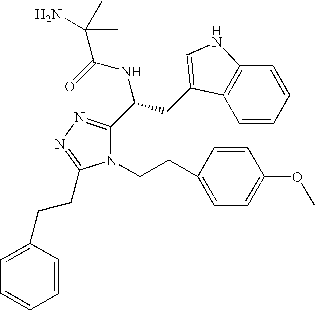

### compound 131 (R)-N-(2-(1H-indol-3-yl)-1-(5-phenethyl-4-(thiophen-2-ylmethyl)-4H-1,2,4-triazol-3-yl)ethyl)-2-amino-2-methylpropanamide,

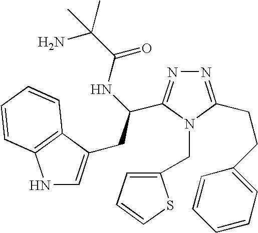

### compound 132 (R)-N-(2-(1H-indol-3-yl)-1-(5-phenethyl-4-(pyridin-2-ylmethyl)-4H-1,2,4-triazol-3-yl)ethyl)-2-amino-2-methylpropanamide,

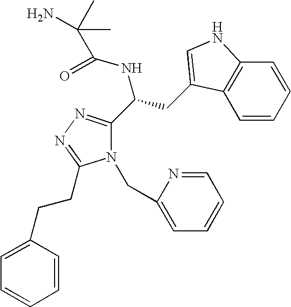

### compound 133 (R)-N-(2-(1H-indol-3-yl)-1-(5-phenethyl-4-(pyridin-2-ylmethyl)-4H-1,2,4-triazol-3-yl)ethyl)piperidine-3-carboxamide,

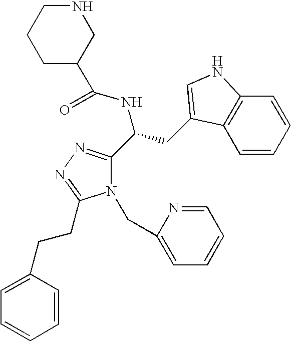

### compound 134 (S)-N-((R)-1-(4-(4-ethylbenzyl)-5-phenethyl-4H-1,2,4-triazol-3-yl)-2-(1H-indol-3-yl)ethyl)pyrrolidine-2-carboxamide,

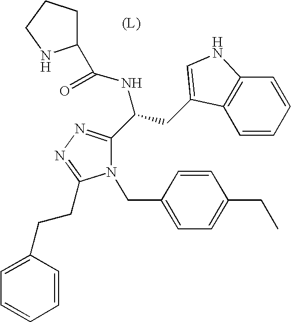

### compound 135 N-((R)-1-(4-(4-ethylbenzyl)-5-phenethyl-4H-1,2,4-triazol-3-yl)-2-(1H-indol-3-yl)ethyl)-2-aminoacetamide,

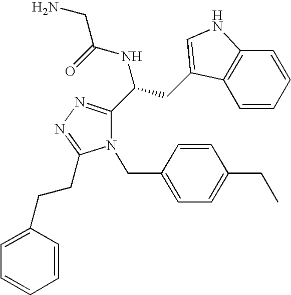

### compound 136 N-((R)-1-(5-(2-(1H-indol-3-yl)ethyl)-4-(4-methoxybenzyl)-4H-1,2,4-triazol-3-yl)-2-(1H-indol-3-yl)ethyl)-2-(pyridin-4-yl)acetamide,

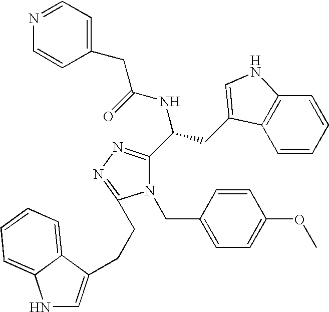

### compound 137 (2R)-N-((R)-1-(4-(4-ethylbenzyl)-5-phenethyl-4H-1,2,4-triazol-3-yl)-2-(1H-indol-3-yl)ethyl)piperidine-2-carboxamide,

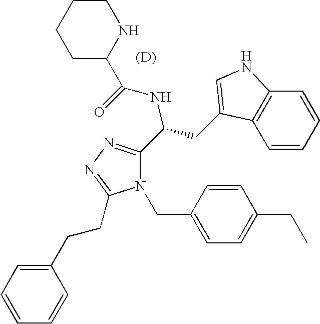

### compound 138 N-((R)-1-(4-(4-ethylbenzyl)-5-phenethyl-4H-1,2,4-triazol-3-yl)-2-(1H-indol-3-yl)ethyl)picolinamide,

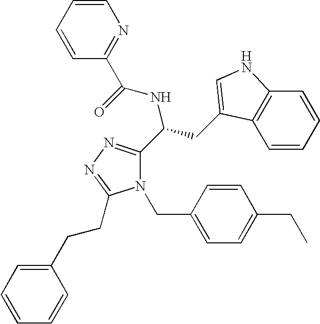

### compound 139 N-((R)-1-(4-(4-ethylbenzyl)-5-phenethyl-4H-1,2,4-triazol-3-yl)-2-(1H-indol-3-yl)ethyl)-2-aminopyridine-3-carboxamide,

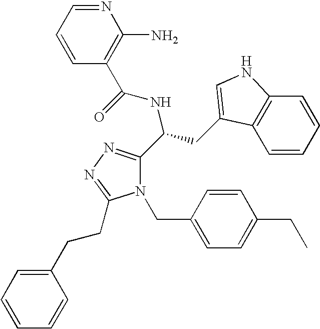

### compound 140 (2S)-N-((R)-1-(4-(4-ethylbenzyl)-5-phenethyl-4H-1,2,4-triazol-3-yl)-2-(1H-indol-3-yl)ethyl)-2-aminopropanamide,

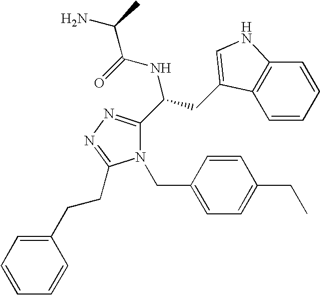

### compound 141 N-((R)-1-(4-(4-ethylbenzyl)-5-phenethyl-4H-1,2,4-triazol-3-yl)-2-(1H-indol-3-yl)ethyl)isonicotinamide,

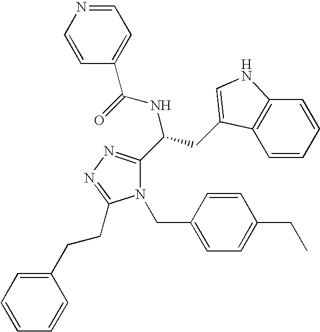

### compound 142 N-((R)-2-(1H-indol-3-yl)-1-(5-phenethyl-4-phenyl-4H-1,2,4-triazol-3-yl)ethyl)piperidine-4-carboxamide,

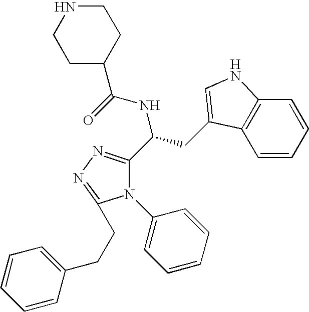

### compound 143 (2S)-N-((R)-2-(1H-indol-3-yl)-1-(5-phenethyl-4-phenyl-4H-1,2,4-triazol-3-yl)ethyl)pyrrolidine-2-carboxamide,

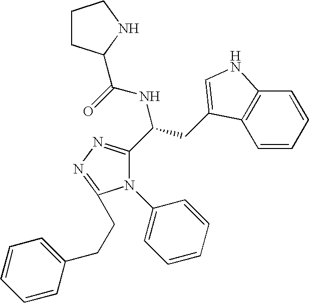

### compound 144 N-((R)-2-(1H-indol-3-yl)-1-(5-phenethyl-4-phenyl-4H-1,2,4-triazol-3-yl)ethyl)-2-aminoacetamide,

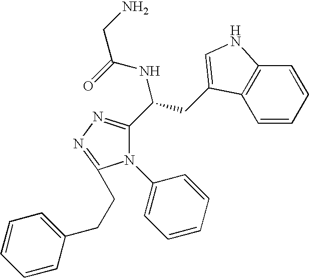

### compound 145 N-((R)-2-(1H-indol-3-yl)-1-(5-phenethyl-4-phenyl-4H-1,2,4-triazol-3-yl)ethyl)-2-(pyridin-2-yl)acetamide,

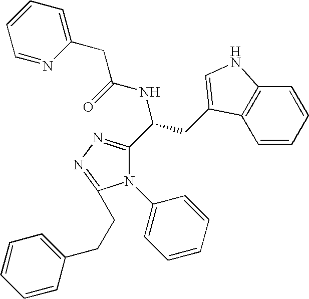

### compound 146 N-((R)-2-(1H-indol-3-yl)-1-(5-phenethyl-4-phenyl-4H-1,2,4-triazol-3-yl)ethyl)picolinamide,

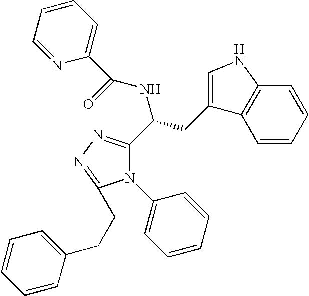

### compound 147 N-((R)-1-(5-(2-(1H-indol-3-yl)ethyl)-4-(4-ethylphenyl)-4H-1,2,4-triazol-3-yl)-2-(1H-indol-3-yl)ethyl)picolinamide,

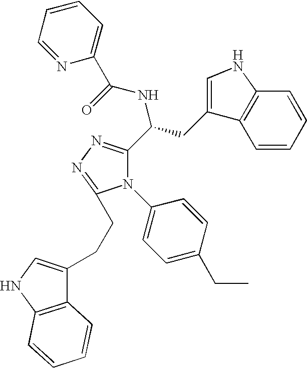

### compound 148 N-((R)-1-(5-(2-(1H-indol-3-yl)ethyl)-4-(4-ethylphenyl)-4H-1,2,4-triazol-3-yl)-2-(1H-indol-3-yl)ethyl)-2-(pyridin-2-yl)acetamide,

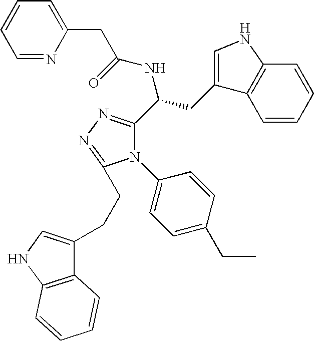

### compound 149 N-((R)-1-(5-(2-(1H-indol-3-yl)ethyl)-4-(4-ethylphenyl)-4H-1,2,4-triazol-3-yl)-2-(1H-indol-3-yl)ethyl)-2-aminoacetamide,

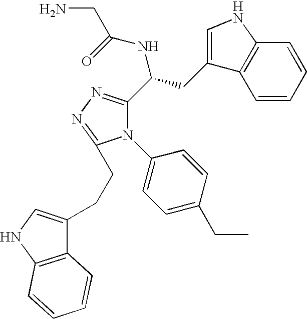

### compound 150 (2S)-N-((R)-1-(5-(2-(1H-indol-3-yl)ethyl)-4-(4-ethylphenyl)-4H-1,2,4-triazol-3-yl)-2-(1H-indol-3-yl)ethyl)pyrrolidine-2-carboxamide,

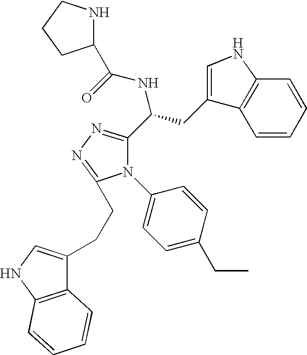

### compound 152 N-((R)-1-(5-(2-(1H-indol-3-yl)ethyl)-4-(4-methoxybenzyl)-4H-1,2,4-triazol-3-yl)-2-(1H-indol-3-yl)ethyl)-2-aminoacetamide,

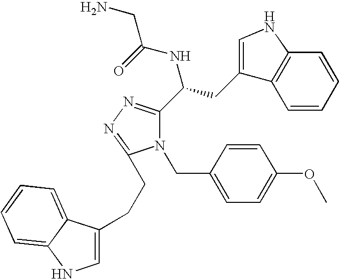

### compound 153 N-((R)-1-(4-(4-methoxybenzyl)-5-phenethyl-4H-1,2,4-triazol-3-yl)-2-(1H-indol-3-yl)ethyl)-2-trans-aminocyclohexanecarboxamide,

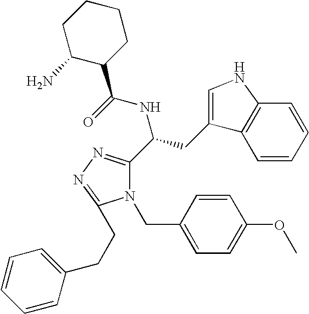

### compound 154 N-((R)-1-(4-(4-ethylbenzyl)-5-phenethyl-4H-1,2,4-triazol-3-yl)-2-(1H-indol-3-yl)ethyl)-2-(pyridin-3-yl)acetamide,

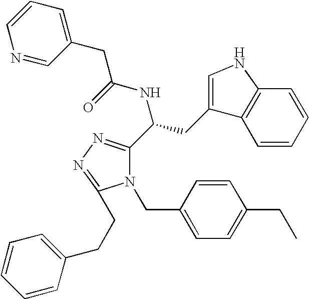

### compound 155 (3S)-N-((R)-1-(4-(4-ethylbenzyl)-5-phenethyl-4H-1,2,4-triazol-3-yl)-2-(1H-indol-3-yl)ethyl)piperidine-3-carboxamide,

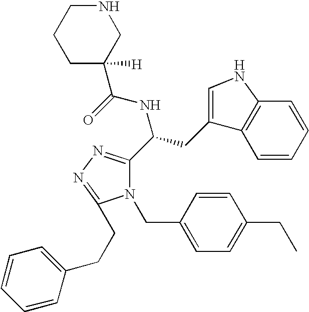

### compound 156 N-((R)-1-(4-(4-ethylbenzyl)-5-phenethyl-4H-1,2,4-triazol-3-yl)-2-(1H-indol-3-yl)ethyl)-2-aminobenzamide,

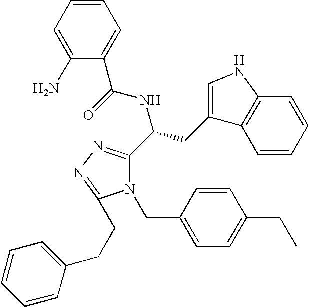

### compound 157 N-((R)-1-(5-(2-(1H-indol-3-yl)ethyl)-4-phenyl-4H-1,2,4-triazol-3-yl)-2-(1H-indol-3-yl)ethyl)picolinamide,

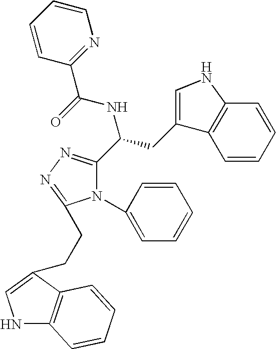

### compound 158 N-((R)-1-(5-(2-(1H-indol-3-yl)ethyl)-4-phenyl-4H-1,2,4-triazol-3-yl)-2-(1H-indol-3-yl)ethyl)piperidine-4-carboxamide,

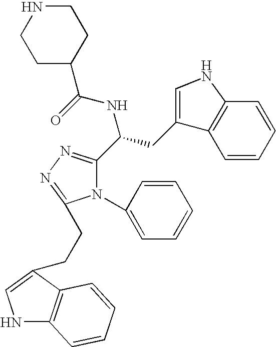

### compound 159 N-((R)-2-(1H-indol-3-yl)-1-(4-(2,4-dimethoxyphenyl)-5-phenethyl-4H-1,2,4-triazol-3-yl)ethyl)picolinamide,

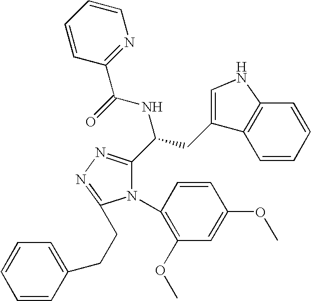

### compound 160 N-((R)-2-(1H-indol-3-yl)-1-(4-(2,4-dimethoxyphenyl)-5-phenethyl-4H-1,2,4-triazol-3-yl)ethyl)-2-(pyridin-2-yl)acetamide,

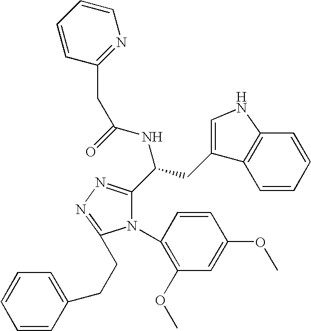

### compound 161 N-((R)-2-(1H-indol-3-yl)-1-(4-(2,4-dimethoxyphenyl)-5-phenethyl-4H-1,2,4-triazol-3-yl)ethyl)pyrazine-2-carboxamide,

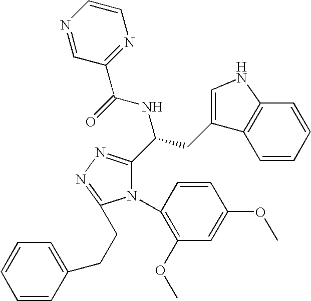

### compound 162 N-((R)-2-(1H-indol-3-yl)-1-(4-(2,4-dimethoxyphenyl)-5-phenethyl-4H-1,2,4-triazol-3-yl)ethyl)-2-aminoacetamide,

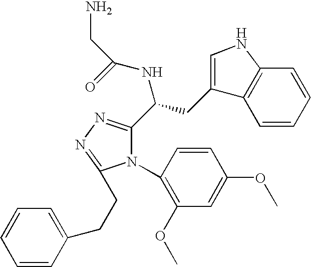

### compound 163 N-((R)-2-(1H-indol-3-yl)-1-(4-(2,4-dimethoxyphenyl)-5-phenethyl-4H-1,2,4-triazol-3-yl)ethyl)piperidine-4-carboxamide,

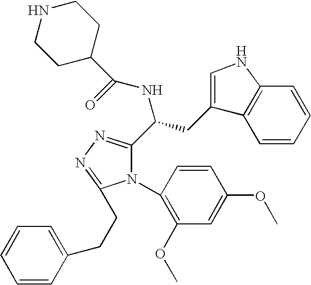

### compound 164 N-((R)-1-(5-benzyl-4-((pyridin-2-yl)methyl)-4H-1,2,4-triazol-3-yl)-2-(1H-indol-3-yl)ethyl)picolinamide,

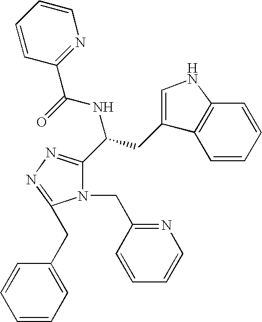

### compound 165 N-((R)-1-(5-benzyl-4-((pyridin-2-yl)methyl)-4H-1,2,4-triazol-3-yl)-2-(1H-indol-3-yl)ethyl)-2-amino-acetamide,

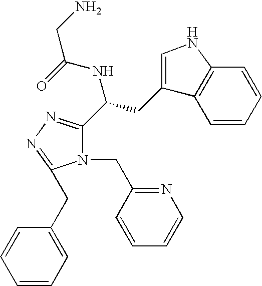

### compound 166 N-((R)-1-(5-benzyl-4-((pyridin-2-yl)methyl)-4H-1,2,4-triazol-3-yl)-2-(1H-indol-3-yl)ethyl)piperidine-4-carboxamide,

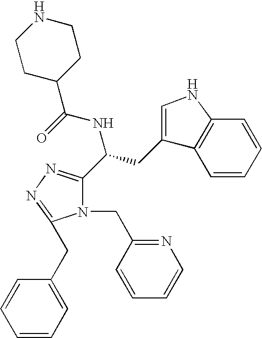

### compound 167 N-((R)-1-(5-benzyl-4-((pyridin-4-yl)methyl)-4H-1,2,4-triazol-3-yl)-2-(1H-indol-3-yl)ethyl)-2-amino-2-methylpropanamide,

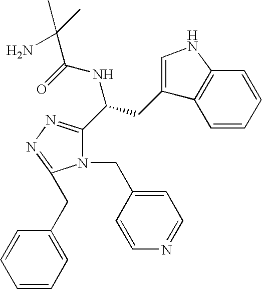

### compound 168 N-((R)-1-(5-(4-methoxybenzyl)-4-phenethyl-4H-1,2,4-triazol-3-yl)-2-(1H-indol-3-yl)ethyl)-2-amino-2-methylpropanamide,

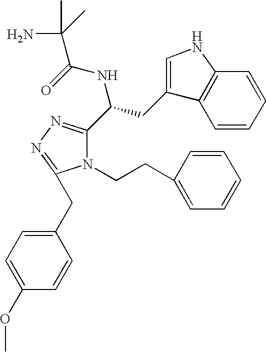

### compound 169 N-((R)-1-(5-benzyl-4-((pyridin-4-yl)methyl)-4H-1,2,4-triazol-3-yl)-2-(1H-indol-3-yl)ethyl)picolinamide,

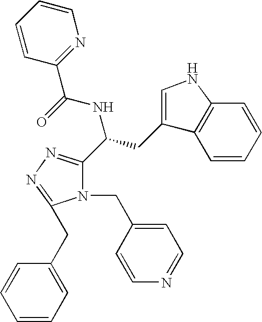

### compound 170 N-((R)-1-(5-benzyl-4-((pyridin-4-yl)methyl)-4H-1,2,4-triazol-3-yl)-2-(1H-indol-3-yl)ethyl)2-amino-acetamide,

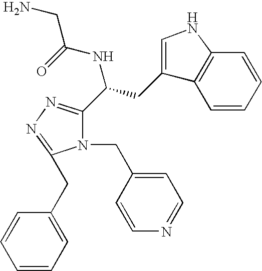

### compound 171 (R)-benzyl-3-(2-aminoisobutyramido)-3-(5-(2-(1H-indol-3-yl)ethyl)-4-(4-methoxybenzyl)-4H-1,2,4-triazol-3-yl)-propanoate,

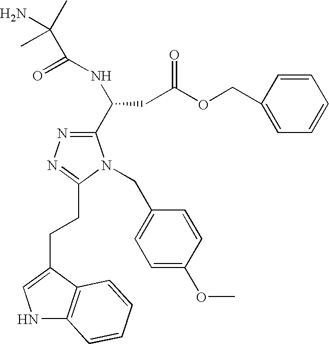

### compound 172 N-((R)-1-(5-benzyl-4-((pyridin-3-yl)methyl)-4H-1,2,4-triazol-3-yl)-2-(1H-indol-3-yl)ethyl)-2-amino-2-methylpropanamide,

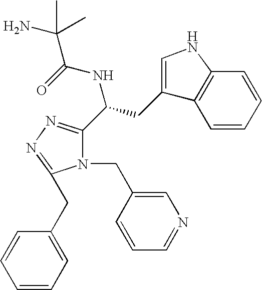

### compound 173 N-((R)-1-(4-benzyl-5-phenethyl-4H-1,2,4-triazol-3-yl)-2-(1H-indol-3-yl)ethyl)-2-amino-2-methylpropanamide,

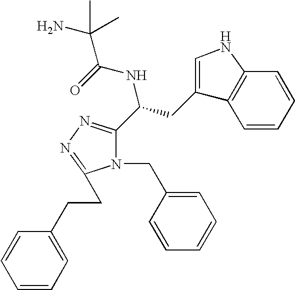

### compound 174 N-((R)-2-(1H-indol-3-yl)-1-(4-methyl-5-phenethyl-4H-1,2,4-triazol-3-yl)ethyl)picolinamide,

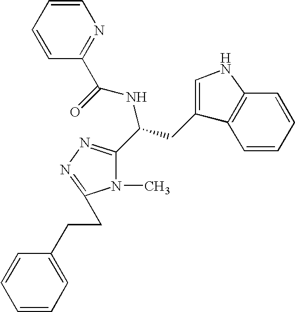

### compound 175 N-((R)-1-(5-(2-(1H-indol-3-yl)ethyl)-4-phenyl-4H-1,2,4-triazol-3-yl)-2-(1H-indol-3-yl)ethyl)-2-amino-2-methylpropanamide,

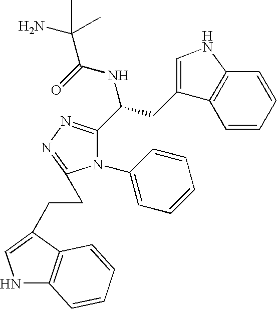

### compound 176 N-((R)-1-(4-(4-methoxybenzyl)-5-phenethyl-4H-1,2,4-triazol-3-yl)-2-(1H-indol-3-yl)ethyl)benzamide,

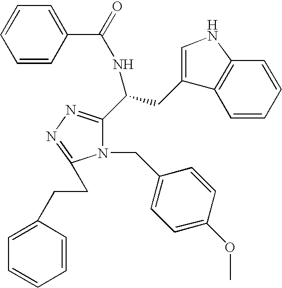

### compound 177 (R)-1-(4-(2,4-dimethoxybenzyl)-5-phenethyl-4H-1,2,4-triazol-3-yl)-2-(1H-indol-3-yl)-N-phenylmethanesulfonylamine,

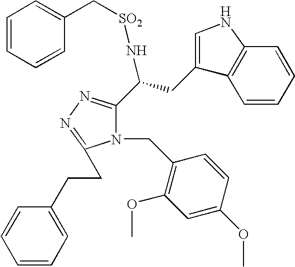

### compound 178 (R)-1-(4-(2,4-dimethoxybenzyl)-5-phenethyl-4H-1,2,4-triazol-3-yl)-2-(1H-indol-3-yl)-N-tosylethanamine,

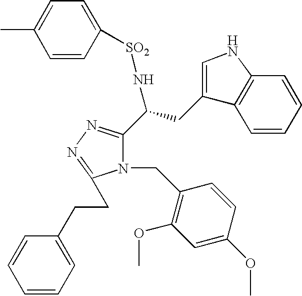

### compound 179 N-((R)-1-(4-(2,4-dimethoxybenzyl)-5-phenethyl-4H-1,2,4-triazol-3-yl)-2-(1H-indol-3-yl)ethyl)-2-amino-2-methylpropanamide,

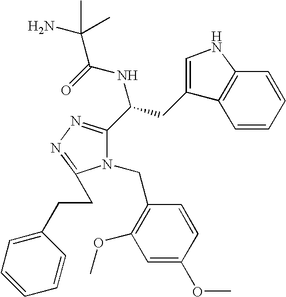

### compound 180 N-1-((R)-1-(4-(2,4-dimethoxybenzyl)-5-phenethyl-4H-1,2,4-triazol-3-yl)-2-(1H-indol-3-yl)ethyl)ethane-1,2-diamine,

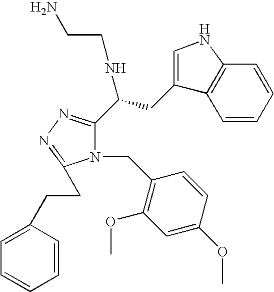

### compound 181 N-((R)-1-(4-((furan-2-yl)methyl)-5-phenethyl-4H-1,2,4-triazol-3-yl)-2-(1H-indol-3-yl)ethyl)-2-amino-2-methylpropanamide,

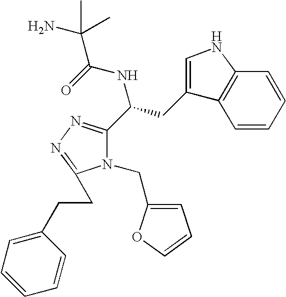

### compound 182 N-((R)-1-(4-((furan-2-yl)methyl)-5-phenethyl-4H-1,2,4-triazol-3-yl)-2-(1H-indol-3-yl)ethyl)picolinamide,

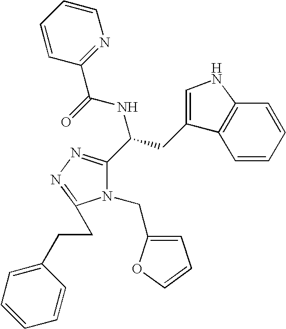

### compound 183 N-((R)-1-(4-((furan-2-yl)methyl)-5-phenethyl-4H-1,2,4-triazol-3-yl)-2-(1H-indol-3-yl)ethyl)piperidine-4-carboxamide,

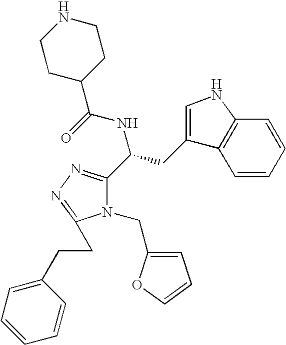

### compound 184 N-((R)-1-(4-(4-methoxybenzyl)-5-phenethyl-4H-1,2,4-triazol-3-yl)-2-(1H-indol-3-yl)ethyl)-tetrahydro-2H-pyran-4-carboxamide,

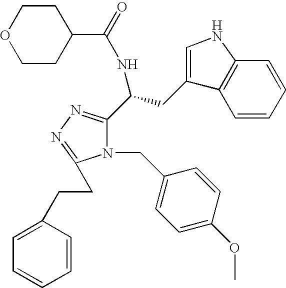

### compound 185 N-((R)-1-(5-((1H-indol-3-yl)methyl)-4-(3-methoxybenzyl)-4H-1,2,4-triazol-3-yl)-2-(1H-indol-3-yl)ethyl)-2-amino-2-methylpropanamide,

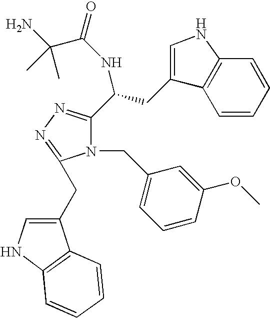

### compound 186 (2S)-N-((R)-1-(4-(4-methoxybenzyl)-5-phenethyl-4H-1,2,4-triazol-3-yl)-2-(1H-indol-3-yl)ethyl)-2-amino-3-phenylpropanamide,

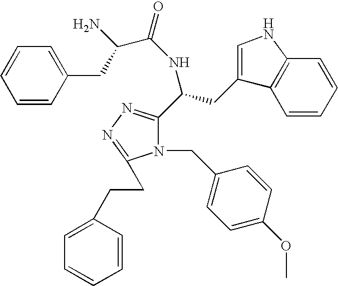

### compound 187 (R)-1-(5-(2-(1H-indol-3-yl)ethyl)-4-(2,4-dimethoxybenzyl)-4H-1,2,4-triazol-3-yl)-2-(1H-indol-3-yl)-N-tosylethanamine,

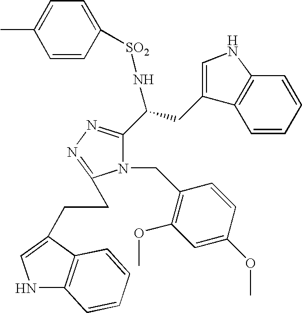

### compound 188 N-((R)-1-(4-(2,4-dimethoxybenzyl)-5-phenethyl-4H-1,2,4-triazol-3-yl)-2-(1H-indol-3-yl)ethyl)-4-azidobenzamide,

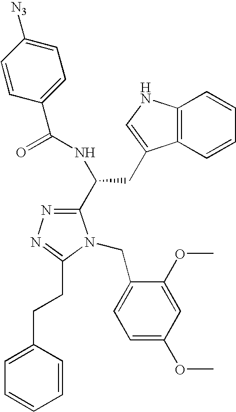

### compound 189 N-benzyl-(R)-1-(4-(2,4-dimethoxybenzyl)-5-phenethyl-4H-1,2,4-triazol-3-yl)-2-(1H-indol-3-yl)ethanamine,

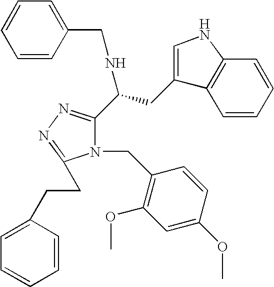

### compound 190 (2S)-N-((R)-1-(5-(2-(1H-indol-3-yl)ethyl)-4-(4-methoxybenzyl)-4H-1,2,4-triazol-3-yl)-2-(1H-indol-3-yl)ethyl)-2,5-dihydro-1H-pyrrole-2-carboxamide,

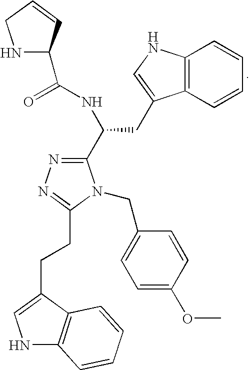

For the avoidance of doubt, if chemical name and chemical structure of the above illustrated compounds do not correspond by mistake, the chemical structure is regarded to unambigously define the compound.

In a preferred embodiment these compounds can be used for the manufacture of a medicament for the treatment or prophylaxis of physiological and/or pathophysiological conditions in mammals, where the physiological and/or pathophysiological conditions are mediated by at least one G-protein coupled receptor (GPCR); with the proviso that the following G-protein coupled receptors (GPCR) are excluded: “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

All compounds of formula (I) as shown above, the preferred embodiments and the triazole compounds 1 to 190 mentioned explicitly are referred to collectively hereinafter as “compounds of the invention”.

The terms indicated for explanation of the above compounds of formula (I) and preferred embodiments always, unless indicated otherwise in the description or in the claims, have the following meanings:

The term “substituted” means that the corresponding radical or group has one or more substituents. Where a radical has a plurality of substituents, and a selection of various substituents is specified, the substituents are selected independently of one another and need not be identical. The term “unsubstituted” means that the corresponding group has no substituent. The term “optionally substituted” means that the corresponding group is either unsubstituted or substituted by one or more substituents. The term “substituted by up to 3 substituents” means that the corresponding radical or group is substituted either by one or by two or three substituents.

The term “alkyl” includes for the purposes of this invention acyclic saturated hydrocarbons having C1-C12 carbon atoms, which may be straight-chain or branched. The term “alkyl” preferably stands for alkyl chains of 1 to 8, particularly preferably 1 to 6, carbon atoms. Examples of suitable alkyl radicals are methyl, ethyl, n-propyl, isopropyl, n-butyl, isobutyl. sec-butyl, tert-butyl, n-pentyl, tert-pentyl, iso-Pentyl, neo-Pentyl, n-hexyl, isohexyl, 2-Hexyl, n-heptyl, n-octyl, n-nonyl, n-decyl, n-undecyl, n-dodecyl.

The term “alkenyl” includes for the purposes of this invention acyclic unsaturated or partially unsaturated hydrocarbons having C2-C12 carbon atoms, i.e. C₂-12-alkenyls, which may be straight-chain or branched and contain one or more C—C double bonds. The term “alkenyl” preferably stands for alkenyl chains of 2 to 8, particularly preferably 2 to 6, carbon atoms. Examples are ethylenyl (vinyl), propenyl (—CH₂CH═CH₂; —CH═CH—CH₃, —C(═CH₂)—CH₃), butenyl, pentenyl, hexenyl, heptenyl, octenyl, octadienyl.

The term “alkynyl” refers to acyclic unsaturated or partially unsaturated hydrocarbons having C2-C12 carbon atoms, i.e. C₂-₈-alkynyls, which may be straight-chain or branched and contain one or more C—C triple bonds. Alkynyls may additionally also contain at least one C—C double bond. The term “alkynyl” preferably stands for alkynyl chains of 2 to 8, particularly preferably 2 to 6, carbon atoms. Examples are ethynyl, propynyl (—CH₂—C≡CH, —C≡C—CH₃), butynyl, pentynyl, hexynyl, heptynyl, octynyl.

The term “cycloalkyl” stands for a saturated or partially unsaturated non-aromatic cyclic hydrocarbon group/radical, containing 1 to 3 rings, including monocyclic alkyl, bicyclic alkyl and tricyclic alkyl, and containing a total of 3 to 20 carbon atoms forming the rings, preferably 3 to 12, most preferably (C3-C8)-cycloalkyl. The cycloalkyl radical may also be part of a bi- or polycyclic system, where, for example, the cycloalkyl radical is fused to an aryl, heteroaryl or heterocyclyl radical as defined herein by any possible and desired ring member(s). The bonding to the compounds of formula (I) can be effected via any possible ring member of the cycloalkyl radical. Examples of suitable cycloalkyl radicals are cyclopropyl, cyclobutyl, cyclopentyl, cyclohexyl, cycloheptyl, cyclooctyl, cyclodecyl, cyclohexenyl, cyclopentenyl, cyclooctadienyl.

The term “cycloalkylalkyl” refers to a radical in which the cycloalkyl group is linked via an alkyl, alkenyl or alkynyl group, where the alkyl, alkenyl, alkynyl and cycloalkyl groups have the meanings defined herein, preferably a (C3-C8)-cycloalkyl-(C1-C4)-alkyl radical. Examples thereof are cyclopropylmethyl, cyclohexylmethyl, cyclopentylethyl, cyclohexenylethyl.

The term “heterocyclyl” refers to a 3- to 14-membered, preferably 3-, 4-, 5-, 6-, 7- or 8-membered, cyclic organic radical which contains at least 1 heteroatom, optionally 2, 3, 4 or 5 heteroatoms, especially nitrogen, oxygen and/or sulphur, the heteroatoms being the same or different and the cyclic radical being saturated or unsaturated, but not aromatic. The heterocyclyl radical may also be part of a bi- or polycyclic system, where, for example, the heterocyclyl radical is fused to an aryl, heteroaryl or cycloalkyl radical as defined herein by any possible and desired ring member(s). The bonding to the compounds of formula (I) can be effected via any possible ring member of the heterocyclyl radical. Examples are tetrahydrofuryl, pyrrolidinyl, imidazolidinyl, thiazolidinyl, tetrahydropyranyl, piperidinyl, piperazinyl, morpholinyl, thiapyrrolidinyl, oxapiperazinyl, oxapiperidinyl and oxadiazolyl.

The term “heterocyclylalkyl” refers to radicals in which the heterocyclyl group is linked via an alkyl, alkenyl or alkynyl group, where the alkyl, alkenyl, alkynyl and heterocyclyl groups have the meanings defined herein.

The term “aryl” refers to aromatic hydrocarbon systems having 3 to 14, preferably 5 to 14, carbon atoms. The aryl radical may also be part of a bi- or polycyclic system, where, for example, the aryl radical is fused to a heterocyclyl, heteroaryl or cycloalkyl radical as defined herein by any possible and desired ring member. The bonding to the compounds of formula (I) can be effected via any possible ring member of the aryl radical. Preferred aryl radicals are phenyl, biphenyl, naphthyl and anthracenyl, but likewise indanyl, indenyl or 1,2,3,4-tetrahydronaphthyl.

The term “heteroaryl” refers to a 5-, 6- or 7-membered cyclic aromatic radical which comprises at least 1, where appropriate also 2, 3, 4 or 5 heteroatoms, preferably nitrogen, oxygen and/or sulfur, where the heteroatoms are identical or different. The number of nitrogen atoms is preferably between 0 and 3, and that of the oxygen and sulfur atoms is between 0 and 1. The heteroaryl radical may also be part of a bi- or polycyclic system, where, for example, the heteroaryl radical is fused to a heterocyclyl, aryl or cycloalkyl radical as defined herein by any possible and desired ring member. The bonding to the compounds of formula (I) can be effected via any possible ring member of the heteroaryl radical. Examples are pyrrolyl, furyl, thienyl, thiazolyl, isothiazolyl, oxazolyl, oxadiazolyl, isoxazolyl, pyrazolyl, imidazolyl, triazole, tetrazole, pyridinyl, pyrimidinyl, pyridazinyl, pyrazinyl, triazinyl, phthalazinyl, indolyl, indazolyl, indolizinyl, quinolinyl, isoquinolinyl, quinoxalinyl, quinazolinyl, pteridinyl, carbazolyl, phenazinyl, phenoxazinyl, phenothiazinyl, and acridinyl.

The terms “arylalkyl” and “heteroarylalkyl” refer to radicals in which the aryl or heteroaryl radical is linked via an alkyl, alkenyl or alkynyl group, where the alkyl, alkenyl, alkynyl, aryl and heteroaryl groups have the meanings defined herein. Preferred “arylalkyl” groups are phenyl-(C₁-C₄)-alkyl radicals, preferably benzyl or phenylethyl radicals. Preferred “heteroarylalkyl” groups are indolyl-(C₁-C₄)-alkyl radicals, preferably 1H-indole-3-yl-methyl or 2(1H-indole-3-yl)-ethyl.

The terms “alkylsulfonyl”, “arylsulfonyl” and “arylalkylsulfonyl” refer to radicals in which the alkyl, aryl or arylalkyl group is linked via a —SO₂— group, where the alkyl, aryl and arylalkyl groups have the meanings defined herein. Examples are methylsulfonyl and phenylsulfonyl.

The term “halogen”, “halogen atom” or “halogen substituent” (Hal-) refers to one, where appropriate, a plurality of fluorine (F, fluoro), bromine (Br, bromo), chlorine (Cl, chloro), or iodine (I, iodo) atoms. The designations “dihalogen”, “trihalogen” and “perhalogen” refer respectively to two, three and four substituents, where each substituent can be selected independently from the group consisting of fluorine, chlorine, bromine and iodine. “Halogen” preferably means a fluorine, chlorine or bromine atom.

The term “natural alpha-amino acid side chain” for the purpose of the present invention refers to all side chains of the known 20 proteinogenic alpha-amino acids as well as to side chains of naturally occurring (i.e. in any biological systems) alpha-amino acids, such as for instance selenocystein, pyrrolysine, citrulline, ornithine, homocysteine, N-methylariginine, N-acetyllysine, gamma-carboxyglutamate, 5-hydroxylysine, 3-methylhistidine and/or N,N,N,-trimethyllysine. In this connection “side chain” refers to the residue that is attached to the alpha-carbon atom, e.g. methyl in case of an Ala side chain or benzyl in case of a Phe side chain.

The term “unnatural alpha amino acid side chain” for the purpose of the present invention refers to all side chains of known alpha-amino acids that are not proteinogenic nor are known to occur naturally (i.e. in any biological systems). Examples are norleucine, cyclohexylglycine, 2-naphthylalanine, substituted alpha-amino acids (e.g. halogen substituted Tyr or Phe) as well as protected alpha-amino acid side chains, where a protection group such as Fmoc, Boc, Z, CBZ, Aloc, trityl, acetyl and/or benzyl is directly attached/reacted to a functionalization (e.g. amino, hydroxy and/or carboxy residue). In this connection “side chain” is referred to as for “natural alpha amino acid side chains”.

Above embodiments of radicals R1 to R10 that possess functionalization (e.g. amino, hydroxy and/or carboxy residues), such as alkyl-CO—NH₂, -alkyl-CO—OH, -alkyl-NH₂, -alkyl-NH—C(NH)—NH₂, —CO—C*(R9R10)NH₂, —CO—CH₂—C*(R9R10)—NH₂, —CO—C*(R9R10)—CH₂—NH₂ and/or 2-amino-2-carbonyl-propane (2-amino-isobutyric acid/Aib residue), may be protected with protection groups as mentioned above. Such protection group carrying embodiments are regarded as belonging to/ within the scope and spirit of the invention.

All stereoisomers of the compounds of the invention are contemplated, either in a mixture or in pure or substantially pure form. The compounds of the present invention can have asymmetric centers at any of the carbon atoms including any one of the R radicals. Consequently, compounds of the invention can exist in the form of their racemates, in the form of the pure enantiomers and/or diastereomers or in the form of mixtures of these enantiomers and/or diastereomers: The mixtures may have any desired mixing ratio of the stereoisomers. All these different stereochemical forms and mixtures are within the scope of the present invention.

Thus, for example, the compounds of the invention which have one or more centers of chirality and which occur as racemates or as diastereomer mixtures can be fractionated by methods known per se into their optical pure isomers, i.e. enantiomers or diastereomers. The separation of the compounds of the invention can take place by column separation on chiral or nonchiral phases or by recrystallization from an optionally optically active solvent or with use of an optically active acid or base or by derivatization with an optically active reagent such as, for example, an optically active alcohol, and subsequent elimination of the radical.

The compounds of the invention may be present in the form of their double bond isomers as “pure” E or Z isomers, or in the form of mixtures of these double bond isomers.

Where possible, the compounds of the invention may be in the form of the tautomers.

It is likewise possible for the compounds of the invention to be in the form of any desired prodrugs such as, for example, esters, carbonates, carbamates, ureas, amides or phosphates, in which cases the actually biologically active form is released only through metabolism. Any compound that can be converted in vivo to provide the bioactive agent (i.e. compounds of the invention) is a prodrug within the scope and spirit of the invention.

Various forms of prodrugs are well known in the art and are described for instance in:
(i) Wermuth C G et al., Chapter 31: 671-696, The Practice of Medicinal Chemistry, Academic Press 1996;
(ii) Bundgaard H, Design of Prodrugs, Elsevier 1985; and
(iii) Bundgaard H, Chapter 5: 131-191, A Textbook of Drug Design and Development, Harwood Academic Publishers 1991.

Said references are incorporated herein by reference.

It is further known that chemical substances are converted in the body into metabolites which may where appropriate likewise elicit the desired biological effect—in some circumstances even in more pronounced form.

Any biologically active compound that was converted in vivo by metabolism from any of the compounds of the invention is a metabolite within the scope and spirit of the invention.

The compounds of the invention can, if they have a sufficiently basic group such as, for example, a primary, secondary or tertiary amine, be converted with inorganic and organic acids into salts. The pharmaceutically acceptable salts of the compounds of the invention are preferably formed with hydrochloric acid, hydrobromic acid, iodic acid, sulfuric acid, phosphoric acid, methanesulfonic acid, p-toluenesulfonic acid, carbonic acid, formic acid, acetic acid, sulfoacetic acid, trifluoroacetic acid, oxalic acid, malonic acid, maleic acid, succinic acid, tartaric acid, racemic acid, malic acid, embonic acid, mandelic acid, fumaric acid, lactic acid, citric acid, taurocholic acid, glutaric acid, stearic acid, glutamic acid or aspartic acid. The salts which are formed are, inter alia, hydrochlorides, chlorided, hydrobromides, bromides, iodides, sulfates, phosphates, methanesulfonates, tosylates, carbonates, bicarbonates, formates, acetates, sulfoacetates, triflates, oxalates, malonates, maleates, succinates, tartrates, malates, embonates, mandelates, fumarates, lactates, citrates, glutarate, stearate, aspartates and glutamates. The stoichiometry of the salts formed from the compounds of the invention may moreover be an integral or non-integral multiple of one.

The compounds of the invention can, if they contain a sufficiently acidic group such as, for example, the carboxy, sulfonic acid, phosphoric acid or a phenolic group, be converted with inorganic and organic bases into their physiologically tolerated salts. Examples of suitable inorganic bases are ammonium, sodium hydroxide, potassium hydroxide, calcium hydroxide, and of organic bases are ethanolamine, diethanolamine, triethanolamine, ethylenediamine, t-butylamine, t-octylamine, dehydroabietylamine, cyclohexylamine, dibenzylethylene-diamine and lysine. The stoichiometry of the salts formed from the compounds of the invention can moreover be an integral or nonintegral multiple of one.

It is likewise possible for the compounds of the invention to be in the form of their solvates and, in particular, hydrates which can be obtained for example by crystallization from a solvent or from aqueous solution. It is moreover possible for one, two, three or any number of solvate or water molecules to combine with the compounds of the invention to give solvates and hydrates.

It is known that chemical substances form solids which exist in different order states which are referred to as polymorphic forms or modifications. The various modifications of a polymorphic substance may differ greatly in their physical properties. The compounds of the invention can exist in various polymorphic forms, and certain modifications may moreover be metastable. All these polymorphic forms of the compounds of the invention are to be regarded as belonging to the invention.

In a further preferred embodiment, the compounds of the invention can be used for the manufacture of a medicament for the treatment or prophylaxis of physiological and/or pathophysiological conditions in mammals.

In yet a further preferred embodiment, the compounds of the invention can be used for the manufacture of a medicament for the treatment or prophylaxis of physiological and/or pathophysiological conditions in mammals, where the treatment is achieved by modulation of one or more G-protein coupled receptors (GPCR) as described and defined herein.

In another preferred embodiment, in the course of the herein described treatments the compounds of the invention are G-protein coupled receptor (GPCR) antagonists.

In another preferred embodiment, in the course of the herein described treatments the compounds of the invention are G-protein coupled receptor (GPCR) agonists.

The triazole derivatives (compounds of the invention) as illustrated herein are ligands of GPCRs as described and defined herein. Thus, the aforementioned compounds of the invention are suitable for studying physiological and/or pathophysiological conditions associated with such GPCR receptors and to study biological activity of the GPCR receptors. Further, the compounds of the invention are suitable for the treatment or prophylaxis of physiological and/or pathophysiological conditions mediated by GPCR receptors as described and defined herein and/or physiological and/or pathophysiological conditions which can be influenced by modulation of these receptors, and thus prevented, treated and/or alleviated.

For the purpose of the present invention, the term “treatment” is also intended to include prophylactic treatment or alleviation.

The term “ligand” is intended to refer for the purposes of the present invention to every compound which binds in any way to a receptor and induces either activation, inhibition and/or another conceivable effect (e.g. allosteric effects) at this receptor. The term “ligand” thus includes agonists, antagonists, partial agonists/antagonists, inverse agonists and other ligands which cause an effect at the receptor which is similar to the effect of agonists, antagonists, partial agonists/antagonists or inverse agonist.

For the purpose of the present invention, the term “G-protein coupled receptor (GPCR) antagonist” or “antagonist of GPCRs” refers to compounds of the invention that bind to G-protein coupled receptors (GPCR) as described and defined herein but do not elicit a proper activation of the receptors as assessed by recording an increase of intracellular calcium which is characteristic for activation of G-protein coupled receptors (GPCRs).

The ability to properly activate the receptors is assessed for any compound of the invention by comparing the degree of activation (increase of intracellular calcium) of a GPCR, for instance GPR103, by the compound to be tested (at 10⁻⁶ M concentration) to the degree of activation (increase of intracellular calcium) of the GPCR, for instance GPR103, by 10⁻⁶ M of its cognate ligand (100%), for instance QRFP(26)[26RFa] as ligand of GPR103, and to the basal level (0%). Such assessment can be readily performed by the skilled artisan due to his expert knowledge. The output is a percentage value for each compound to be tested.

Any compound of the invention that does not show a degree of activation (increase of intracellular calcium) of a respective GPCR as described and defined herein of at least 20% as assessed in accordance with above specification is regarded as not eliciting a proper activation and therefore as GPCR receptor antagonist. Preferably such compounds do show an antagonizing effect (counteraction/decrease) on the intracellular calcium increase stimulated by the cognate ligand of the respective GPCR, for instance QRFP(26)[26RFa] as ligand of GPR103, and/or other activating ligand, prevent such stimulation or even act as inverse agonists (an inverse agonists is an ligand which binds to the same receptor binding-site as an agonist or antagonist but causes an inhibition of the basal/constitutive activity of the receptor, in principle an agonists with a negative intrinsic activity). Such compounds may furthermore exhibit an inhibitory activity on other physiological or pathophysiological conditions or effects, such as food intake or lipogenesis. Their effects may be dissociated. Thus, they may have no inihibitory impact at all on intracellular calcium increase while inhibiting other physiological effects.

For the purpose of the present invention, the term “G-protein coupled receptor (GPCR) agonist” or “agonist of GPCR” refers to compounds of the invention that bind to G-protein coupled receptors (GPCR) as described and defined herein and elicit a proper activation of the receptor as assessed by recording an increase of intracellular calcium which is characteristic for activation of G-protein coupled receptors.

Any compound of the invention that shows a degree of activation (increase of intracellular calcium) of a respective GPCR as described and defined herein of at least 20% as assessed in accordance with above specification is regarded as eliciting a proper activation and therefore as a GPCR receptor agonist. Such compounds may mimic the effects of the cognate GPCR receptor ligangs, such as QRFP(26)[26RFa] as ligand of GPR103, and other stimulating ligands on intracellular calcium increase and for instance food intake or lipogenesis. Like for antagonists, the effects of agonist compounds may be dissociated from the intracellular calcium increase effect.

As illustrated above the following receptors are excluded from the genus of G-protein coupled receptors (GPCR) in the course of the present invention. These receptors are herein referred to as “other receptor(s)” and consist of “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

With regard to the “other receptor(s)” and the terms “ligand”, “agonist”, “antagonist”, “partial agonist/antagonist”, “inverse agonist” and “other ligands which cause an effect at the receptor which is similar to the effect of agonists, antagonists, partial agonists/antagonists or inverse agonists” the same above definitions apply as for the genus of G-protein coupled receptors (GPCR) of the present invention as described and defined herein.

The term “GHS receptor” or “GHS-R” is intended to comprise for the purposes of the present invention receptors that bind at least one known peptidyl and/or nonpeptidyl GHS and/or ghrelin. The term “GHS receptor” or “GHS-R” is also intended to comprise different GHS binding sites in the various tissues and/or organs as illustrated herein, that bind at least one known peptidyl and/or non-peptidyl GHS and/or ghrelin and which are probably not yet characterized GHS-R subtypes.

Binding of a given known peptidyl and/or non-peptidyl GHS and/or ghrelin can be easily verified by the skilled artisan on the basis of his expert knowledge, e.g. by appropriate binding assays which represent only routine experimentation.

Such GHS receptors may be stimulated/activated by ghrelin (ghrelin responsive) or may not be stimulated/activated by ghrelin (ghrelin non-responsive)—with regard to both acylated and non-acylated ghrelin, respectively. Stimulation/activation of such receptors may cause but does not compulsorily have to elicit GH production and/or GH secretion and/or increase GH plasma levels.

As illustrated supra, the compounds of the invention are ligands of GPCR as described and defined herein. They can be administered to various mammalian species, including human, for the treatment or prophylaxis of physiological and/or pathophysiological condition in such mammals.

For the purpose of the present invention, all mammalian species are regarded as being comprised. Preferably, such mammals are selected from the group consisting of “human, domestic animals, cattle, livestock, pets, cow, sheep, pig, goat, horse, pony, donkey, hinny, mule, hare, rabbit, cat, dog, guinea pig, hamster, rat, mouse”. More preferably, such mammals are human.

As illustrated supra, the compounds of the invention are ligands of at least one GPCR as described and defined herein. In a preferred embodiment, the compounds of the invention can be used for the manufacture of a medicament for the herein described treatments, where the at least one G-protein coupled receptor (GPCR) is selected from the group consisting of: “5-hydroxytryptamine (serotonin) receptor, 5-hydroxytryptamine (serotonin) receptor 1A [5-HT1A, synonyms: 5HT1a; ADRBRL1, ADRB2RL1, GenBank accession number: NM_{—}000524 (human 5-HT1A)], 5-hydroxytryptamine (serotonin) receptor 1B [5-HT1B, synonyms: S12; HTR1D2; HTR1DB; 5-HT1DB, GenBank accession number: NM_{—}000863 (human 5-HT1B)], 5-hydroxytryptamine (serotonin) receptor 1D [5-HT1D, synonyms: HTRL; RDC4; HT1DA; HTR1DA, GenBank accession number: NP_{—}000855 (human 5-HT1D)], 5-hydroxytryptamine (serotonin) receptor 1E [5-HT1E, GenBank accession number: NM_{—}000865 (human 5-HT1E)], 5-hydroxytryptamine (serotonin) receptor 1F [5-HT1F, synonyms: MR77; HTR1EL, GenBank accession number: NM_{—}000866 (human 5-HT1F)], 5-hydroxytryptamine (serotonin) receptor 2A [5-HT2A, synonyms: HTR2; GenBank accession number: NM_{—}000621 (human 5-HT2A)], 5-hydroxytryptamine (serotonin) receptor 2B [5-HT2B, synonyms: 5-HT(2B), GenBank accession number: NM_{—}000867 (human 5-HT2B)], 5-hydroxytryptamine (serotonin) receptor 2C [5-HT2C, synonyms: HTR1C; GenBank accession number: NM_{—}000868 (human 5-HT2C)], 5-hydroxytryptamine (serotonin) receptor 4 [5-HT4, synonyms: 5-HT4R, GenBank accession number: NM_{—}000870 (human 5-HT4, isoform b)], 5-hydroxytryptamine (serotonin) receptor 5A [5-HT5A, synonyms: MGC138226, GenBank accession number: NM_{—}024012 (human 5-HT5A)], 5-hydroxytryptamine (serotonin) receptor 6 [5-HT6, GenBank accession number: NM_{—}000871 (human 5-HT6)], 5-hydroxytryptamine (serotonin) receptor 7 [5-HT7, synonyms: GenBank accession number: NM_{—}000872 (human 5-HT7, transcript variant a)], muscarinic cholinergic receptor, muscarinic cholinergic receptor 1, [M1, synonyms: HM1; MGC30125, GenBank accession number: NM_{—}000738 (human M1)], muscarinic cholinergic receptor 2, [M2, synonyms: HM2; FLJ43243; MGC120006; MGC120007, GenBank accession number: NM_{—}001006630 (human M2, transcript variant 1)], muscarinic cholinergic receptor 3, [M3, synonyms: HM3, GenBank accession number: NM_{—}000740 (human M3)], muscarinic cholinergic receptor 4, [M4, synonyms: HM4, GenBank accession number: NM_{—}000741 (human M4)], muscarinic cholinergic receptor 5, [M5, synonyms: HM5; MGC41838, GenBank accession number: NM_{—}012125 (human M5)], Alpha-adrenergic receptor, Alpha-1-adrenergic receptor, Alpha-1A-adrenergic receptor, [alpha-1A, synonyms: ADRA1A, ADRA1C; ADRA1L1; ALPHA1MR, GenBank accession number: NM_{—}000680 (human alpha-1A transcript variant 1)], Alpha-1B-adrenergic receptor, [alpha-1B, synonyms: ADRA1; ALPHA1BAR, GenBank accession number: NM_{—}000679 (human alpha-1B)], Alpha-1D-adrenergic receptor, [alpha-1D, synonyms: ADRA1D, DAR; ADRA1; ADRA1A; ADRA1R; ALPHA1; dJ779E11.2, GenBank accession number: NM_{—}000678 (human alpha-1D)], Alpha-2-adrenergic receptor, Alpha-2A-adrenergic receptor, [alpha-2A, synonyms: ADRA2A, ADRA2; ADRAR; ZNF32; ADRA2R; ALPHA2AAR, GenBank accession number: NM_{—}000681 (human alpha-2A)], Alpha-2B-adrenergic receptor, [alpha-2B, synonyms: ADRA2B, ADRA2L1; ADRARL1; ADRA2RL1; ALPHA2BAR, GenBank accession number: NM_{—}000682 (human alpha-2B)], Alpha-2C-adrenergic receptor, [alpha-2C, synonyms: ADRA2C, ADRA2L2; ADRARL2; ADRA2RL2; AL-PHA2CAR, GenBank accession number: NM_{—}000683 (human alpha-2C)], Beta-adrenergic receptor, Beta-1-adrenergic receptor, [beta-1, synonyms: ADRB1, RHR; B1AR; ADRB1R; BETA1AR, GenBank accession number: NM_{—}000684 (human beta-1)], Beta-2-adrenergic receptor, [beta-2, synonyms: ADRB2, BAR; B2AR; ADRBR; ADRB2R; BETA2AR, GenBank accession number: NM_{—}000024 (human beta-2)], Beta-3-adrenergic receptor, [beta-3, synonyms: ADRB3, BETA3AR, GenBank accession number: NM_{—}000025 (human beta-3)], angiotensin II receptor, angiotensin II receptor type 1, [AT1, synonyms: AGTR1, AG2S; AT1B; AT2R1; HAT1R; AGTR1A; AGTR1B; AT2R1A; AT2R1B, GenBank accession number: NM_{—}031850 (human AT1, transcript variant 4)], angiotensin II receptor type 2, [AT2, synonyms: AGTR2, ATGR2; MRX88, GenBank accession number: NM_{—}000686 (human AT2)], neuromedin B receptor, [NMBR, synonyms: BB1, GenBank accession number: NM_{—}002511 (human NMBR)], gastrin-releasing peptide receptor, [GRPR, synonyms: BB2, GenBank accession number: NM_{—}005314 (human GRPR)], bombesin-like receptor 3, [BRS3, synonyms: BB3, GenBank accession number: NM_{—}001727 (human BRS3)], calcitonin receptor, [CALCR, synonyms: CT, CRT; CTR; CTR1, GenBank accession number: NM_{—}001742 (human CALCR)], calcitonin receptor-like, [CALCRL, synonyms: CRLR; CGRPR, GenBank accession number: NM_{—}005795 (human CALCRL)], cannabinoid receptor, cannabinoid receptor 1, [CB1, synonyms: CNR1, CNR; CB-R; CB1A; CANN6; CB1K5, GenBank accession number: NM_{—}016083 (human CB1, transcript variant 1)], cannabinoid receptor 2, [CB2, synonyms: CNR2, CX5, GenBank accession number: NM_{—}001841 (human CB2)], cholecystokinin receptor, cholecystokinin A receptor, [CCKAR, synonyms: CCK-A; CCKRA; CCK1-R, GenBank accession number: NM_{—}000730 (human CCKAR)], cholecystokinin B receptor, [CCKBR, synonyms: GASR; CCK-B, GenBank accession number: NM_{—}176875 (human CCKBR)], corticotropin releasing hormone receptor, corticotropin releasing hormone receptor 1, [CRF1, synonyms: CRHR1, CRHR; CRF-R; CRFR1; CRHR1f; CRH-R1h, GenBank accession number: NM_{—}004382 (human CRF1)], corticotropin releasing hormone receptor 2, [CRF2, synonyms: CRHR2, GenBank accession number: NM_{—}001883 (human CRF2)], dopamine receptor, dopamine receptor D1, [D1, synonyms: DRD1, DADR; DRD1A, GenBank accession number: NM_{—}000794 (human D1)], dopamine receptor D2, [D2, synonyms: DRD2, D2R; D2DR, GenBank accession number: NM_{—}000795 (human D2, transcript variant 1)], dopamine receptor D3, [D3,.synonyms: DRD3, D3DR; ETM1; FET1; MGC149204; MGC149205, GenBank accession number: NM_{—}000796 (human D3, transcript variant a)], dopamine receptor D4, [D4, synonyms: DRD4, D4DR, GenBank accession number: NM_{—}000797 (human D4)], dopamine receptor D5, [D5, synonyms: DRD5, DBDR; DRD1B; DRD1L2; MGC10601, GenBank accession number: NM_{—}000798 (human D5)], glucagon-like peptide receptor, glucagon-like peptide 1 receptor, [GLP-1, synonyms: GLP1R, MGC138331; GenBank accession number: NM_{—}002062 (human GLP-1)], glucagon-like peptide 2 receptor, [GLP-2, GenBank accession number: NM_{—}004246 (human GLP-2)], follicle stimulating hormone receptor, [FSHR, synonyms: LGR1; ODG1; FSHRO; MGC141667; MGC141668, GenBank accession number: NM_{—}004246 (human FSHR)], histamine receptor, histamine receptor H1, [H1, synonyms: HRH1, H1-R; hisH1, GenBank accession number: NM_{—}000861 (human H1)], histamine receptor H2, [H2, synonyms: HRH2, H2R, GenBank accession number: NM_{—}022304 (human H2)], histamine receptor H3, [H3, synonyms: HRH3, HH3R; GPCR97, GenBank accession number: NM_{—}007232 (human H3)], histamine receptor H4, [H4, synonyms: HRH4, H4; H4R; BG26; HH4R; AXOR35; GPRv53; GPCR105; MGC133027, GenBank accession number: NM_{—}021624 (human H4)], endothelial differentiation lysophosphatidic acid G-protein-coupled receptor, endothelial differentiation lysophosphatidic acid G-protein-coupled receptor 2, [EDG2, synonyms: LPA1, LPAR1; edg-2; vzg-1; Gpcr26; Mrec1.3; rec.1.3, GenBank accession number: NM_{—}001401 (human EDG2, transcript variant 1)], endothelial differentiation lysophosphatidic acid G-protein-coupled receptor 4, [EDG4, synonyms: LPA2, EDG-4; LPAR2, GenBank accession number: NM_{—}004720 (human EDG4)], endothelial differentiation lysophosphatidic acid G-protein-coupled receptor 7, [EDG7, synonyms: LPA3, GPCR; Edg-7; LP-A3; LPAR3; HOFNH30; RP4-67813, GenBank accession number: NM_{—}012152 (human EDG7)], melanin-concentrating hormone receptor, melanin-concentrating hormone receptor 1, [MCH1, synonyms: MCHR1, SLC1; GPR24; MCH1R; MGC32129, GenBank accession number: NM_{—}005297 (human MCH1)], melanin-concentrating hormone receptor 2, [MCH2, synonyms: MCHR2, SLT; MCH2R; GPR145, GenBank accession number: NM_{—}001040179 (human MCH2, transcript variant 1)], melanocortin receptor, melanocortin 1 receptor (alpha melanocyte stimulating hormone receptor), [MC1, synonyms: MCHR1, SLC1; GPR24; MCH1R; MGC32129, GenBank accession number: NM_{—}005297 (human MC1)], melanocortin 2 receptor (adrenocorticotropic hormone receptor), [MC2, synonyms: MCHR2, SLT; MCH2; MCH2R; GPR145, GenBank accession number: NM_{—}001040179 (human MC2, transcript variant 1)], melanocortin 3 receptor, [MC3, synonyms: MC3R, GenBank accession number: NM_{—}019888 (human MC3)], melanocortin 4 receptor, [MC4, synonyms: MC4R, MGC126851; MGC138197, GenBank accession number: NM_{—}005912 (human MC4)], melanocortin 5 receptor, [MC5, synonyms: MC5R, GenBank accession number: NM_{—}005913 (human MC5)], neuropeptide FF receptor, neuropeptide FF receptor 1, [NPFFR1, synonyms: NPFF1; GPR147; NPFF1R1; OT7T022; FLJ10751, GenBank accession number: NM_{—}022146 (human NPFFR1)], neuropeptide FF receptor 2, [NPFFR2, synonyms: GPR74; NPFF2; NPGPR, GenBank accession number: NM_{—}004885 (human NPFFR2)], neuropeptides B/W receptor, neuropeptides B/W receptor 1, [NPBWR1, synonyms: GPR7; MGC129755, GenBank accession number: NM_{—}005285 (human NPBWR1)], neuropeptides B/W receptor 2, [NPBWR2, synonyms: GPR8, GenBank accession number: NM_{—}005286 (human NPBWR2)], neuropeptide Y receptor, neuropeptide Y receptor Y1, [NPY1R, synonyms: NPYR, GenBank accession number: NM_{—}000909 (human NPY1R)], neuropeptide Y receptor Y2, [NPY2R, GenBank accession number: NM_{—}000910 (human NPY2R)], neuropeptide Y receptor Y4 (pancreatic polypeptide receptor 1), [NPY4R, synonyms: PPYR1, Y4; PP1; NPY4-R; MGC116897, GenBank accession number: NM_{—}005972 (human NPY4R)], neuropeptide Y receptor Y5, [NPY5R, GenBank accession number: NM_{—}006174 (human NPY5R)], opioid receptor, opioid receptor delta 1, [OPRD1, synonyms: OPRD, GenBank accession number: NM_{—}000911 (human OPRD1)], opioid receptor kappa 1, [OPRK1, synonyms: KOR; OPRK, GenBank accession number: NM_{—}000912 (human OPRK1)], opioid receptor mu 1, [OPRM1, synonyms: MOR1; OPRM; KIAA0403, GenBank accession number: NM_{—}000914 (human OPRM1, transcript variant MOR-1)], opiate receptor-like 1, [OPRL1, synonyms: OOR; ORL1; KOR-3; NOCIR; MGC34578, GenBank accession number: NM_{—}182647 (human OPRL1, transcript variant 1)], hypocretin (orexin) receptor, hypocretin (orexin) receptor 1, [HCRTR1, synonyms: OX1R, GenBank accession number: NM_{—}001525 (human HCRTR1)], hypocretin (orexin) receptor 2, [HCRTR2, synonyms: OX2R, GenBank accession number: NM_{—}001526 (human HCRTR2)], G protein-coupled receptor 103, [GPR103, synonyms: AQ27; SP9155; MGC149217, GenBank accession number: NM_{—}198179 (human GPR103)], G protein-coupled receptor 103A, [GPR103A, GenBank accession number: NM_{—}198192 (mouse GPR103A)], G protein-coupled receptor 103B, [GPR103B, GenBank accession number: AX665940 (mouse GPR103B)], prolactin releasing hormone receptor, [PRLHR, synonyms: GR3; GPR10; PrRPR; MGC126539; MGC126541, GenBank accession number: NM_{—}004248 (human PRLHR)], somatostatin receptor, somatostatin receptor 1, [SSTR1, synonyms: SRIF-2, GenBank accession number: NM_{—}001049 (human SSTR1)], somatostatin receptor 2, [SSTR2, GenBank accession number: NM_{—}001050 (human SSTR2)], somatostatin receptor 3, [SSTR3, GenBank accession number: NM_{—}001051 (human SSTR3)], somatostatin receptor 4, [SSTR4, synonyms: SRIF-2, GenBank accession number: NM_{—}001052 (human SSTR4)], somatostatin receptor 5, [SSTR5, GenBank accession number: NM_{—}001053 (human SSTR5)], galanin receptor, galanin receptor 1, [GALR1, synonyms: GALNR; GALNR1, GenBank accession number: NM_{—}001480 (human GALR1)], galanin receptor 2, [GALR2, synonyms: GALNR2; MGC125983; MGC125984, GenBank accession number: NM_{—}003857 (human GALR2)], galanin receptor 3, [GALR3, GenBank accession number: NM_{—}003614 (human GALR3)], cortistatin receptor, cortistatin receptor MAS-related GPR member X1, [MRGX1, synonyms: MRGPRX1, GPCR; SNSR4, GenBank accession number: NM_{—}147199 (human MRGX1)], cortistatin receptor MAS-related GPR member X2, [MRGX2, synonyms: MRGPRX2, GenBank accession number: NM_{—}054030 (human MRGX2)], cortistatin receptor MAS-related GPR member X3, [MRGX3, synonyms: MRGPRX3, GPCR; SNSR1, GenBank accession number: NM_{—}054031 (human MRGX3)], and/or cortistatin receptor MAS-related GPR member X4, [MRGX4, synonyms: MRGPRX4, GPCR; SNSR6; MGC129753; MGC129754, GenBank accession number: NM_{—}054032 (human MRGX4)]”.

Preferably, the at least one G-protein coupled receptor (GPCR) is selected from the group consisting of: “5-hydroxytryptamine (serotonin) receptor 2C [5-HT2C, synonyms: HTR1C; GenBank accession number: NM_{—}000868 (human 5-HT2C)], muscarinic cholinergic receptor 3, [M3, synonyms: HM3, GenBank accession number: NM_{—}000740 (human M3)], Beta-3-adrenergic receptor, [beta-3, synonyms: ADRB3, BETA3AR, GenBank accession number: NM_{—}000025 (human beta-3)], angiotensin II receptor type 1, [AT1, synonyms: AGTR1, AG2S; AT1B; AT2R1; HAT1R; AGTR1A; AGTR1B; AT2R1A; AT2R1B, GenBank accession number: NM_{—}031850 (human AT1, transcript variant 4)], angiotensin II receptor type 2, [AT2, synonyms: AGTR2, ATGR2; MRX88, GenBank accession number: NM_{—}000686 (human AT2)], gastrin-releasing peptide receptor, [GRPR, synonyms: BB2, GenBank accession number: NM_{—}005314 (human GRPR)], bombesin-like receptor 3, [BRS3, synonyms: BB3, GenBank accession number: NM_{—}001727 (human BRS3)], calcitonin receptor, [CALCR, synonyms: CT, CRT; CTR; CTR1, GenBank accession number: NM_{—}001742 (human CALCR)], calcitonin receptor-like, [CALCRL, synonyms: CRLR; CGRPR, GenBank accession number: NM_{—}005795 (human CALCRL)], cannabinoid receptor 1, [CB1, synonyms: CNR1, CNR; CB-R; CB1A; CANN6; CB1K5, GenBank accession number: NM_{—}016083 (human CB1, transcript variant 1)], cannabinoid receptor 2, [CB2, synonyms: CNR2, CX5, GenBank accession number: NM_{—}001841 (human CB2)], cholecystokinin A receptor, [CCKAR, synonyms: CCK-A; CCKRA; CCK1-R, GenBank accession number: NM_{—}000730 (human CCKAR)], cholecystokinin B receptor, [CCKBR, synonyms: GASR; CCK-B, GenBank accession number: NM_{—}176875 (human CCKBR)], corticotropin releasing hormone receptor 1, [CRF1, synonyms: CRHR1, CRHR; CRF-R; CRFR1; CRHR1f; CRH-R1h, GenBank accession number: NM_{—}004382 (human CRF1)], corticotropin releasing hormone receptor 2, [CRF2, synonyms: CRHR2, GenBank accession number: NM_{—}001883 (human CRF2)], dopamine receptor D1, [D1, synonyms: DRD1, DADR; DRD1A, GenBank accession number: NM_{—}000794 (human D1)], dopamine receptor D2, [D2, synonyms: DRD2, D2R; D2DR, GenBank accession number: NM_{—}000795 (human D2, transcript variant 1)], dopamine receptor D3, [D3, synonyms: DRD3, D3DR; ETM1; FET1; MGC149204; MGC149205, GenBank accession number: NM_{—}000796 (human D3, transcript variant a)], dopamine receptor D4, [D4, synonyms: DRD4, D4DR, GenBank accession number: NM_{—}000797 (human D4)], dopamine receptor D5, [D5, synonyms: DRD5, DBDR; DRD1B; DRD1L2; MGC10601, GenBank accession number: NM_{—}000798 (human D5)], glucagon-like peptide 1 receptor, [GLP-1, synonyms: GLP1R, MGC138331, GenBank accession number: NM_{—}002062 (human GLP-1)], follicle stimulating hormone receptor, [FSHR, synonyms: LGR1; ODG1; FSHRO; MGC141667; MGC141668, GenBank accession number: NM_{—}004246 (human FSHR)], histamine receptor H1, [H1, synonyms: HRH1, H1-R; hisH1, GenBank accession number: NM_{—}000861 (human H1)], histamine receptor H3, [H3, synonyms: HRH3, HH3R; GPCR97, GenBank accession number: NM_{—}007232 (human H3)], endothelial differentiation lysophosphatidic acid G-protein-coupled receptor 2, [EDG2, synonyms: LPA1, LPAR1; edg-2; vzg-1; Gpcr26; Mrec1.3; rec.1.3, GenBank accession number: NM_{—}001401 (human EDG2, transcript variant 1)], melanin-concentrating hormone receptor 1, [MCH1, synonyms: MCHR1, SLC1; GPR24; MCH1R; MGC32129, GenBank accession number: NM_{—}005297 (human MCH1)], melanocortin 1 receptor (alpha melanocyte stimulating hormone receptor), [MC1, synonyms: MCHR1, SLC1; GPR24; MCH1R; MGC32129, GenBank accession number: NM_{—}005297 (human MC1)], melanocortin 3 receptor, [MC3, synonyms: MC3R, GenBank accession number: NM_{—}019888 (human MC3)], melanocortin 4 receptor, [MC4, synonyms: MC4R, MGC126851; MGC138197, GenBank accession number: NM_{—}005912 (human MC4)], neuropeptide FF receptor 1, [NPFFR1, synonyms: NPFF1; GPR147; NPFF1R1; OT7T022; FLJ10751, GenBank accession number: NM_{—}022146 (human NPFFR1)], neuropeptide FF receptor 2, [NPFFR2, synonyms: GPR74; NPFF2; NPGPR, GenBank accession number: NM_{—}004885 (human NPFFR2)], neuropeptides B/W receptor 1, [NPBWR1, synonyms: GPR7; MGC129755, GenBank accession number: NM_{—}005285 (human NPBWR1)], neuropeptides B/W receptor 2, [NPBWR2, synonyms: GPR8, GenBank accession number: NM_{—}005286 (human NPBWR2)], neuropeptide Y receptor Y1, [NPY1R, synonyms: NPYR, GenBank accession number: NM_{—}000909 (human NPY1R)], neuropeptide Y receptor Y2, [NPY2R, GenBank accession number: NM_{—}000910 (human NPY2R)], neuropeptide Y receptor Y5, [NPY5R, GenBank accession number: NM_{—}006174 (human NPY5R)], opioid receptor kappa 1, [OPRK1, synonyms: KOR; OPRK, GenBank accession number: NM_{—}000912 (human OPRK1)], hypocretin (orexin) receptor 1, [HCRTR1, synonyms: OX1R, GenBank accession number: NM_{—}001525 (human HCRTR1)], hypocretin (orexin) receptor 2, [HCRTR2, synonyms: OX2R, GenBank accession number: NM_{—}001526 (human HCRTR2)], G protein-coupled receptor 103, [GPR103, synonyms: AQ27; SP9155; MGC149217, GenBank accession number: NM_{—}198179 (human GPR103)], G protein-coupled receptor 103A, [GPR103A, GenBank accession number: NM_{—}198192 (mouse GPR103A)], G protein-coupled receptor 103B, [GPR103B, GenBank accession number: AX665940 (mouse GPR103B)], prolactin releasing hormone receptor, [PRLHR, synonyms: GR3; GPR10; PrRPR; MGC126539; MGC126541, GenBank accession number: NM_{—}004248 (human PRLHR)], galanin receptor 1, [GALR1, synonyms: GALNR; GALNR1, GenBank accession number: NM_{—}001480 (human GALR1)] and/or cortistatin receptor MAS-related GPR member X2, [MRGX2, synonyms: MRGPRX2, GenBank accession number: NM_{—}054030 (human MRGX2)]”.

Most preferably, the at least one G-protein coupled receptor (GPCR) is selected from the group consisting of: “cholecystokinin A receptor, [CCKAR, synonyms: CCK-A; CCKRA; CCK1-R, GenBank accession number: NM_{—}000730 (human CCKAR)], chole-cystokinin B receptor, [CCKBR, synonyms: GASR; CCK-B, GenBank accession number: NM_{—}176875 (human CCKBR)], neuropeptide FF receptor 1, [NPFFR1, synonyms: NPFF1; GPR147; NPFF1R1; OT7T022; FLJ10751, GenBank accession number: NM_{—}022146 (human NPFFR1)], neuropeptide FF receptor 2, [NPFFR2, synonyms: GPR74; NPFF2; NPGPR, GenBank accession number: NM_{—}004885 (human NPFFR2)], neuropeptide Y receptor Y1, [NPY1R, synonyms: NPYR, GenBank accession number: NM_{—}000909 (human NPY1R)], neuropeptide Y receptor Y2, [NPY2R, GenBank accession number: NM_{—}000910 (human NPY2R)], neuropeptide Y receptor Y5, [NPY5R, GenBank accession number: NM_{—}006174 (human NPY5R)], hypocretin (orexin) receptor 1, [HCRTR1, synonyms: OX1R, GenBank accession number: NM_{—}001525 (human HCRTR1)], hypocretin (orexin) receptor 2, [HCRTR2, synonyms: OX2R, GenBank accession number: NM_{—}001526 (human HCRTR2)], G protein-coupled receptor 103, [GPR103, synonyms: AQ27; SP9155; MGC149217, GenBank accession number: NM_{—}198179 (human GPR103)], G protein-coupled receptor 103A, [GPR103A, GenBank accession number: NM_{—}198192 (mouse GPR103A)], G protein-coupled receptor 103B, [GPR103B, GenBank accession number: AX665940 (mouse GPR103B)], galanin receptor 1, [GALR1, synonyms: GALNR; GALNR1, GenBank accession number: NM_{—}001480 (human GALR1)] and/or cortistatin receptor MAS-related GPR member X2, [MRGX2, synonyms: MRGPRX2, GenBank accession number: NM_{—}054030 (human MRGX2)]”.

Preferably, the compounds of the invention are antagonists and/or agonists of one or more of the above mentioned preferred, more preferred and/or most preferred GPCR, most preferably of G protein-coupled receptor 103, [GPR103, synonyms: AQ27; SP9155; MGC149217, GenBank accession number: NM_{—}198179 (human GPR103)], G protein-coupled receptor 103A, [GPR103A, GenBank accession number: NM_{—}198192 (mouse GPR103A)], G protein-coupled receptor 103B, [GPR103B, GenBank accession number: AX665940 (mouse GPR103B)].

The compounds of the invention being non-peptidic ligands of GPCR as described and defined herein are surprisingly characterized by a strong binding affinity to such receptors. Such compounds for instance may preferably exhibit an IC₅₀ value of less than 1000 nM for binding to a GPCR, for instance GPR103. More preferably, such compounds may exhibit an IC₅₀ value of less than 500 nM, even more preferably of less than 300 nM and most preferably of less than 100 nM for binding to a GPCR, such as GPR103.

Due to their surprisingly strong receptor binding, the compounds of the invention can be advantageously administered at lower doses compared to other less potent binders while still achieving equivalent or even superior desired biological effects. In addition, such a dose reduction may advantageously lead to less or even no medicinal adverse effects. Further, the high binding specificity of the compounds of the invention may translate into a decrease of undesired side effects on its own regardless of the dose applied.

Furthermore, the compounds of the invention, being of non-peptidic nature, are resistant to degradation by enzymes of the gastro-intestinal tract. Hence, they offer the advantage to be given by oral route. They surprisingly display an improved metabolic stability and/or an improved bioavailability. Hence, again an advantageous dose reduction may be achievable which may cause less or even no side effects.

In another aspect, the object of the invention has been surprisingly solved by providing the compounds of the invention according to the herein disclosed uses and treatments, where the physiological and/or pathophysiological conditions are mediated by at least one G-protein coupled receptor (GPCR) as described and defined herein and at least one other receptor (as defined herein) selected from the group consisting of: “GHS receptors, GHS type 1 receptor, GHS-R 1a, GHS-R 1b, motilin receptor, motilin receptor 1a, neurotensin receptor, TRH receptor, GPR38 (FM1), GPR39 (FM2), FM3, GHS-R subtype, GHS binding site, cardiac GHS-R, mammary GHS-R”.

In a preferred embodiment, the treatment is achieved by modulation of one or more of the G-protein coupled receptors (GPCR) as described and defined herein and/or one or more of the other receptors (as defined herein).

In another preferred embodiment, the at least one G-protein coupled receptor (GPCR) is selected from the group consisting of: “cholecystokinin A receptor, [CCKAR, synonyms: CCK-A; CCKRA; CCK1-R, GenBank accession number: NM_{—}000730 (human CCKAR)], cholecystokinin B receptor, [CCKBR, synonyms: GASR; CCK-B, GenBank accession number: NM_{—}176875 (human CCKBR)], neuropeptide FF receptor 1, [NPFFR1, synonyms: NPFF1; GPR147; NPFF1R1; OT7T022; FLJ10751, GenBank accession number: NM_{—}022146 (human NPFFR1)], neuropeptide FF receptor 2, [NPFFR2, synonyms: GPR74; NPFF2; NPGPR, GenBank accession number: NM_{—}004885 (human NPFFR2)], neuropeptide Y receptor Y1, [NPY1R, synonyms: NPYR, GenBank accession number: NM_{—}000909 (human NPY1R)], neuropeptide Y receptor Y2, [NPY2R, GenBank accession number: NM_{—}000910 (human NPY2R)], neuropeptide Y receptor Y5, [NPY5R, GenBank accession number: NM_{—}006174 (human NPY5R)], hypocretin (orexin) receptor 1, [HCRTR1, synonyms: OX1R, GenBank accession number: NM_{—}001525 (human HCRTR1)], hypocretin (orexin) receptor 2, [HCRTR2, synonyms: OX2R, GenBank accession number: NM_{—}001526 (human HCRTR2)], G protein-coupled receptor 103, [GPR103, synonyms: AQ27; SP9155; MGC149217, GenBank accession number: NM_{—}198179 (human GPR103)], G protein-coupled receptor 103A, [GPR103A, GenBank accession number: NM_{—}198192 (mouse GPR103A)], G protein-coupled receptor 103B, [GPR103B, GenBank accession number: AX665940 (mouse GPR103B)], and/or galanin receptor 1, [GALR1, synonyms: GALNR; GALNR1, GenBank accession number: NM_{—}001480 (human GALR1)]” and the at least one other receptor is selected from the group consisting of: “GHS type 1 receptor, GHS-R 1a [GenBank accession number: NM_{—}198407 (human GHS-R 1a)], GHS-R 1b [GenBank accession number: NM_{—}004122 (human GHS-R 1b)]”. More preferably, the at least one G-protein coupled receptor (GPCR) is selected from the group consisting of: “G protein-coupled receptor 103, [GPR103, synonyms: AQ27; SP9155; MGC149217, GenBank accession number: NM_{—}198179 (human GPR103)], G protein-coupled receptor 103A, [GPR103A, GenBank accession number: NM_{—}198192 (mouse GPR103A)] and/or G protein-coupled receptor 103B, [GPR103B, GenBank accession number: AX665940 (mouse GPR103B)]” and the at least one other receptor is selected from the group consisting of: “GHS type 1 receptor, GHS-R 1a [GenBank accession number: NM_{—}198407 (human GHS-R 1a)], GHS-R 1b [GenBank accession number: NM_{—}004122 (human GHS-R 1b)]”.

In yet a further preferred embodiment, the compound of the invention is an antagonist of one or more of the G-protein coupled receptors (GPCR) as described and defined herein and/or one or more of the other receptors (as defined herein).

In yet a further preferred embodiment, the compound of the invention is an agonist of one or more of the G-protein coupled receptors (GPCR) as described and defined herein and/or one or more of the other receptors (as defined herein).

In yet a further preferred embodiment, the compound of the invention is an antagonist of one or more of the G-protein coupled receptors (GPCR) as described and defined herein and an agonist of one or more of the other receptors (as defined herein).

In yet a further preferred embodiment, the compound of the invention is an agonist of one or more of the G-protein coupled receptors (GPCR) as described and defined herein and an antagonist of one or more of the other receptors (as defined herein).

The terms “at least one GPCR”, “one or more GPCRs”, “at least one other receptor” and “one or more other receptors” in the course of the present invention mean that the treatment or prophylaxis as disclosed herein is achieved by modulation of one receptor or simultaneous modulation of two or more receptors as defined herein by at least one compound of the invention.

The compounds of the invention can either be antagonists or agonists of GPCR as described and defined herein and, optionally, in addition antagonists or agonists of the other receptors as described and defined herein.

Thus, the compounds of the invention are suitable for the treatment or prophylaxis of various physiological and/or pathophysiological conditions.

For the purpose of the present invention, all physiological and/or pathophysiological conditions are intended to be comprised that are known to be mediated by the GPCR as described and defined herein and, optionally, in addition the other receptors as described and defined herein.

Preferably, these physiological and/or pathophysiological conditions are selected from the group consisting of: “metabolic disorders, lipid metabolism, disorders of lipid metabolism, adipogenesis, adiposity, anorexia, body weight gain, body weight reduction, bulimia, cachexia, diabetes, diabetes type I, diabetes type II, energy balance, energy homeostasis, food intake, hyperlipidemia, hyperphagia, insulin resistance in the heart, insulin resistance in type 2 diabetic patients, insulin resistance including NIDDM, metabolic homeostasis maintenance, obesity, overweight, short-, medium- and/or long-term regulation of energy balance, short-, medium- and/or long-term regulation (stimulation and/or inhibition) of food intake, as well as diseases for which above mentioned physiological and/or pathophysiological conditions are risk factors and which are selected from the group consisting of: “premature death, diabetes, type 2 diabetes, heart disease, heart attack, coronary heart disease, congestive heart failure, stroke, hypertension (high blood pressure), gallbladder disease, osteoarthritis, sleep apnea, asthma, breathing problems, endometrial cancer, colon cancer, kidney cancer, gallbladder cancer, postmenopausal breast cancer, hypercholesterolemia (high blood cholesterol), insulin resistance, complications of pregnancy, menstrual irregularities, hirsutism, stress incontinence, increased surgical risk, psychological disorders such as depression (for the risk factors diseases, please refer to publication: “Statistics related to Overweight and Obesity, US Department of Health and Human Services, NIDDK 2006, http://win.niddk.nih.gov/publications/PDFs/stat904z.pdf).

More preferably, these physiological and/or pathophysiological conditions are selected from the group consisting of: “adipogenesis, adiposity, cachexia, diabetes, diabetes type I, diabetes type II, energy balance, energy homeostasis, food intake, hyperlipidemia, hyperphagia, obesity, short-, medium- and/or long-term regulation of energy balance, short-, medium- and/or long-term regulation (stimulation and/or inhibition) of food intake”.

In a further aspect of the present invention, the compounds of the invention may be used in combination with at least one additional pharmacologically active substance.

Such additional pharmacologically active substance may be other compounds of the invention and/or other “suitable therapeutic agents” useful in the treatment or prophylaxis of the aforementioned physiological and/or pathophysiological conditions.

The additional pharmacologically active substance may be an antagonist or an agonist of GPCR as described and defined herein and, optionally, in addition an antagonist or an agonist of other receptors as described and defined herein, depending on the purpose of the combined use. Selection and combination of the additional pharmacologically active substance(s) can be easily performed by the skilled artisan on the basis of his expert knowledge and depending on the purpose of the combined use and physiological and/or pathophysiological conditions targeted.

In a preferred embodiment, the compounds of the invention are used for the treatment or prophylaxis of the aforementioned physiological and/or pathophysiological conditions in the form of a medicament, where such medicament comprises at least one additional pharmacologically active substance.

In another preferred embodiment, the compounds of the invention are used for the treatment or prophylaxis of the aforementioned physiological and/or pathophysiological conditions in the form of a medicament, where the medicament is applied before and/or during and/or after treatment with at least one additional pharmacologically active substance.

The above mentioned “suitable therapeutic agents” include: “GHS, anti-diabetic agents; anti-obesity agents; anti-hypertensive agents; cholesterol/lipid lowering agents; anabolic agents; testosterone and preferably, a “suitable therapeutic agents” is selected of the group consisting of this agents.

Examples of suitable GHS for use in combination with the compounds of the present invention include GHRP-6, GHRP-1 as described in U.S. Pat. No. 4,411,890; and publications WO 89/07110 and WO 89/07111 and B-HT920 or growth hormone releasing factor and its analogs or growth hormone and its analogs or somatomedins including IGF-1 and IGF-2 as well as GHS described in WO 01/96300.

Examples of suitable anti-diabetic agents for use in combination with the compounds of the present invention include biguanides (e.g. metformin), glucosidase inhibitors (e.g. acarbose), insulins (including insulin secretagogues or insulin sensitizers), meglitinides (e.g. repaglinide), sulfonylureas (e.g., glimepiride, glyburide and glipizide), biguanide/glyburide combinations (e.g., glucovance), thiozolidinediones (e.g. troglitazone, rosiglitazone and pioglitazone), PPAR-alpha agonists, PPAR-gamma agonists, PPAR alpha/gamma dual agonists, SGLT2 inhibitors, inhibitors of fatty acid binding protein (aP2) such as those disclosed in U.S. Pat. No. 6,548,529, glucagon-like peptide-1 (GLP-1), and dipeptidyl peptidase IV (DP4) inhibitors.

Examples of suitable anti-obesity agents for use in combination with the compounds of the present invention include endocannabinoid receptor antagonists, e.g. CB1 receptor antagonists such as rimonabant (1H-Pyrazole-3-carboxamide, 5-(4-chlorophenyl)-1-(2,4-dichlorophenyl)-4-methyl-N-1-piperidinyl-, monohydrochloride; CAS Registry Number: 158681-13-1; SR-14171;U.S. Pat. No. 5,624,941), aP2 inhibitors such as those disclosed in U.S. Pat. No. 6,548,529, PPAR gamma antagonists, PPAR delta agonists, orlistat and neurotransmitter reuptake inhibitors, such as sibutramine [1-(4-chlorophenyl)-N,N-dimethyl-alpha-(2-methylpropyl)-cyclobutane-methanamine; CAS Registry Number: 106650-56-0] and sibutramide hydrochloride monohydrate, which block the reuptake of the neurotransmitters dopamine, norepinephrine and serotonin.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradii), diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265, Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable cholesterol/lipid lowering agents for use in combination with the compounds of the present invention include HMG-CoA reductase inhibitors [e.g., pravastatin lovastatin, atorvastatin, simvastatin, NK-104 (a.k.a. itavastatin, or nisvastatin or nisbastatin] and ZD-4522 (a.k.a. rosuvastatin, or atavastatin or visastatin)), squalene synthetase inhibitors, fibrates, bile acid sequestrants, ACAT inhibitors, MTP inhibitors, lipooxygenase inhibitors, choesterol absorption inhibitors, and cholesterol ester transfer protein inhibitors (e.g., CP-529414).

Examples of suitable anabolic agents for use in combination with the compounds of the present invention include testosterone and SARMs.

The above other therapeutic agents, when employed in combination with the compounds of the present invention, may be used, for example, in those amounts indicated in the Physicians' Desk Reference (PDR) or as otherwise determined by one of ordinary skill in the art.

In a preferred embodiment, the compounds of the invention are used for the treatment or prophylaxis of the aforementioned physiological and/or pathophysiological conditions in the form of a medicament, where such medicament comprises at least one additional pharmacologically active substance selected from the group consisting of: “GHS receptor agonist and/or antagonist, GHS type 1 receptor agonist and/or antagonist, GHS-R 1a agonist and/or antagonist, GHS-R 1b agonist and/or antagonist, motilin receptor agonist and/or antagonist, motilin receptor 1a agonist and/or antagonist, neurotensin receptor agonist and/or antagonist, TRH receptor agonist and/or antagonist, GPR38 (FM1) agonist and/or antagonist, GPR39 (FM2) agonist and/or antagonist, FM3 agonist and/or antagonist, GHS-R subtype agonist and/or antagonist, GHS binding site agonist and/or antagonist, cardiac GHS-R agonist and/or antagonist, mammary GHS-R agonist and/or antagonist, endocannabinoid receptor agonist and/or antagonist, CB1 receptor agonist and/or antagonist, rimonabant [1H-Pyrazole-3-carboxamide, 5-(4-chlorophenyl)-1-(2,4-dichlorophenyl)-4-methyl-N-1-piperidinyl-, monohydrochloride], sibutramine [1-(4-chlorophenyl)-N,N-dimethyl-alpha-(2-methylpropyl)-cyclobutane-methanamine; CAS Registry Number: 106650-56-0], sibutramide hydrochloride monohydrate”.

In another preferred embodiment, the compounds of the invention are used for the treatment or prophylaxis of the aforementioned physiological and/or pathophysiological conditions in the form of a medicament, where the medicament is applied before and/or during and/or after treatment with at least one additional pharmacologically active substance selected from the group consisting of: “GHS receptor agonist and/or antagonist, GHS type 1 receptor agonist and/or antagonist, GHS-R 1a agonist and/or antagonist, GHS-R 1b agonist and/or antagonist, motilin receptor agonist and/or antagonist, motilin receptor 1a agonist and/or antagonist, neurotensin receptor agonist and/or antagonist, TRH receptor agonist and/or antagonist, GPR38 (FM1) agonist and/or antagonist, GPR39 (FM2) agonist and/or antagonist, FM3 agonist and/or antagonist, GHS-R sub-type agonist and/or antagonist, GHS binding site agonist and/or antagonist, cardiac GHS-R agonist and/or antagonist, mammary GHS-R agonist and/or antagonist, endocannabinoid receptor agonist and/or antagonist, CB1 receptor agonist and/or antagonist, rimonabant [1H-Pyrazole-3-carboxamide, 5-(4-chlorophenyl)-1-(2,4-dichlorophenyl)-4-methyl-N-1-piperidinyl-, monohydrochloride], sibutramine [1-(4-chlorophenyl)-N,N-dimethyl-alpha-(2-methylpropyl)-cyclobutane-methanamine; CAS Registry Number: 106650-56-0], sibutramide hydrochloride monohydrate”.

The compounds of the present invention and/or, where appropriate, the additional pharmacologically active substances can be administered in a known manner. The route of administration may thereby be any route which effectively transports the active compound to the appropriate or desired site of action, for example orally or non-orally, in particular topically, transdermally, pulmonary, rectally, intravaginally, nasally or parenteral or by implantation. Oral administration is preferred.

The compounds of the invention and/or where appropriate the additional pharmacologically active substances are converted into a form which can be administered and are mixed where appropriate with pharmaceutically acceptable carriers or diluents. Suitable excipients and carriers are described for example in Zanowiak P, Ullmann's Encyclopedia of Industrial Chemistry 2005, Pharmaceutical Dosage Forms, 1-33; Spiegel A J et al., Journal of Pharmaceutical Sciences 1963, 52: 917-927; Czetsch-Lindenwald H, Pharm. Ind. 1961, 2: 72-74; Fiedler H P, Lexikon der Hilfsstoffe für Pharmazie, Kosmetik and angrenzende Gebiete 2002, Editio Cantor Verlag, p 65-68.

Oral administration can take place for example in solid form as tablet, capsule, gel capsule, coated tablet, granulation or powder, but also in the form of a drinkable solution. The compounds of the invention can for oral administration be combined with known and ordinarily used, physiologically tolerated excipients and carriers such as, for example, gum arabic, talc, starch, sugars such as, for example, mannitol, methylcellulose, lactose, gelatin, surface-active agents, magnesium stearate, cyclodextrins, aqueous or nonaqueous carriers, diluents, dispersants, emulsifiers, lubricants, preservatives and flavorings (e.g. essential oils). The compounds of the invention can also be dispersed in a microparticulate, e.g. nanoparticulate, composition.

Non-oral administration can take place for example by intravenous, subcutaneous, intramuscular injection of sterile aqueous or oily solutions, suspensions or emulsions, by means of implants or by ointments, creams or suppositories. Administration as sustained release form is also possible where appropriate. Implants may comprise inert materials, e.g. biodegradable polymers or synthetic silicones such as, for example, silicone rubber. Intravaginal administration is possible for example by means of vaginal rings. Intrauterine administration is possible for example by means of diaphragms or other suitable intrauterine devices. Transdermal administration is additionally provided, in particular by means of a formulation suitable for this purpose and/or suitable means such as, for example, patches.

The dosage may vary within a wide range depending on type and/or severity of the physiological and/or pathophysiological condition, the mode of administration, the age, gender, bodyweight and sensitivity of the subject to be treated. It is within the ability of a skilled worker to determine a “pharmacologically effective amount” of a compound of the invention and/or additional pharmacologically active substance. Administration can take place in a single dose or a plurality of separate dosages.

A suitable unit dose is, for example, from 0.001 mg to 100 mg of the active ingredient, i.e. at least one compound of the invention and, where appropriate, at least one additional pharmacologically active substance, per kg of a patient's bodyweight.

### General Syntheses Schemes

The compounds of the present invention may be prepared according to the following general synthetic schemes, as well as relevant published literature procedures that are known to the one skilled in the art (e.g. WO 00/54729 and cited references therein).

Exemplary reagents and procedures for these reactions appear hereinafter and in the working examples. Unless otherwise specified, the various substituents (radicals) of the compounds have the meanings as defined for formula (I) herein.

Amide bond formation (peptide coupling) is conducted under standard peptide coupling procedures known in the prior art. Optimally, the reaction is conducted in a solvent such as dichloromethane (DCM) at room temperature using benzotriazol-1-yl-oxytris(dimethylamino)phosphonium-hexafluoorophosphate (BOP) (Castro B et al., Tetrahedron Lett. 1975, 14:1219-1222) and a base, for example N-methyl-morpholine or diisopropylethylamine.

Thionation of the formed amide was performed using Lawesson's reagent (Pons J F et al., Tetrahedron Lett. 2000, 41: 4965-4968).

Cyclisation: the obtained thioamide was then submitted to the conditions reported by Hitosuyanagi et al. (Hitotsuyanagi Y. et al., J. Org. Chem. 2002, 67: 3266-3271) which were slightly modified (5 eq. of hydrazide and 1.1 eq. of mercury (II) acetate in acetonitrile). Cyclisation into triazoles was generally achieved within three hours. When the hydrazide was not commercially available, it was prepared by known methods from its acid or methyl ester precursors.

Deprotection of the tert-butyloxycarbonyl group (Boc) was performed at room temperature in acidic medium as usually described.

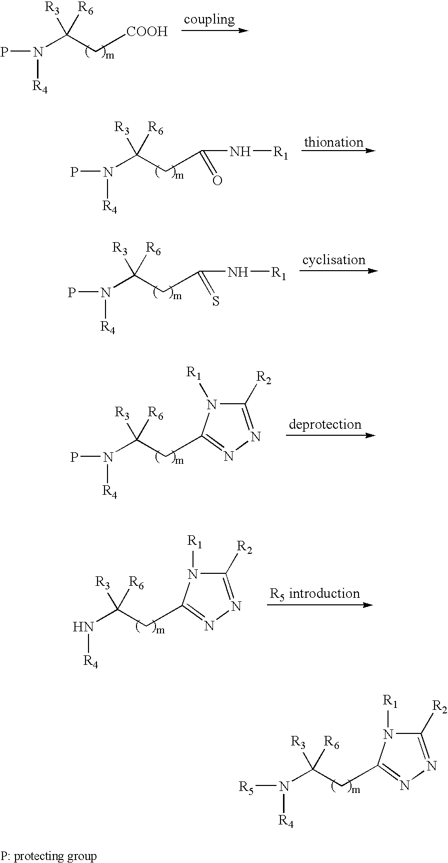

For R5=—CO-alkyl, —CO-cycloalkyl, —CO-cycloalkylalkyl, —CO-aryl, —CO-arylalkyl, —CO-heteroaryl, —CO-heteroarylalkyl, —CO-heterocyclyl, —CO-heterocyclylalkyl, —CO—C*(R9R10)NH₂, —CO—CH₂—C*(R9R10)NH₂, —CO—C*(R9R10)CH₂—NH₂(R):

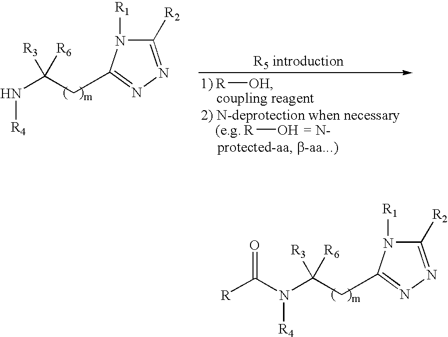

For R5=alkyl, cycloalkyl, cycloalkylalkyl, aryl, heteroaryl, arylalkyl, heteroarylalkyl, heterocyclyl, heterocyclylalkyl (R):

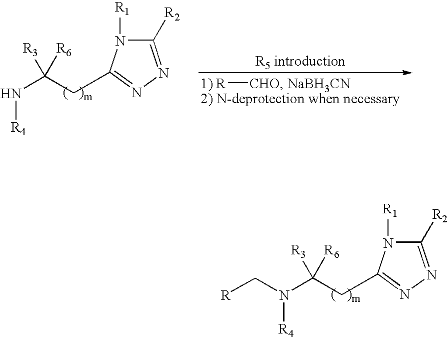

For R5=alkyl-SO₂—, aryl-SO₂—, arylalkyl-SO₂— (R=alkyl, aryl, arylalkyl):

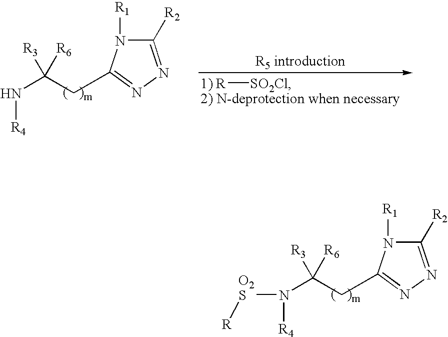

The compounds of the invention, especially compounds 1 to 190 were named from the drawn formula using the Chem Draw Ultra 8 software (CambridgeSoft Corporation, Cambridge, USA).

The contents of all cited references and patents are hereby incorporated by reference in their entirety. The invention is explained in more detail by means of the following examples without, however, being restricted thereto.

### EXAMPLES

### I) Synthesis of Compounds of the Invention

### Example 1

### (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide (Compound 1)

Compound 1 was obtained from Boc-(D)-Trp (10 mmoles), (2,4-dimethoxyphenyl)-methanamine, 3-(1H-indol-3-yl)propane hydrazide and Boc-2-amino-2-methylpropanoic acid according to the general synthetic schemes with a total yield after purification by HPLC of 35%.

¹H NMR (400 MHz, 300° K, DMSO-d⁶):

δ 1.32 (3H, s, CH₃ Aib), 1.36 (3H, s, CH₃ Aib), 2.93 (2H, m, CH₂—CH₂-indole), 2.97 (2H, m, CH₂—CH₂-indole), 3.31 (1H, dd, J=14.5, J=6.1, 1H CH₂ βTrp), 3.38 (1H, dd, J=14.5, J=9.1, 1H CH₂ βTrp), 3.66 (3H, s, o-OCH₃), 3.72 (3H, s, p-OCH₃), 4.93 (1H, d, J=16.9, 1H CH₂ o,p-dimethoxybenzyl), 5.10 (1H, d, J=16.9, 1H CH₂ o,p-dimethoxybenzyl), 5.23 (1H, m, CαH Trp), 6.31 (1H, dd, J=8.5, J=1.7, H₅ o,p-dimethoxybenzyl), 6.45 (1H, d, J=8.5, H₆ o,p-dimethoxybenzyl), 6.59 (1H, d, J=1.7, H₃ o,p-dimethoxybenzyl), 6.88 (1H, t, J=7.5, H₅ Trp), 6.94 (1H, t, J=7.5, H₅ indole), 7.04 (1H, t, H₆ Trp), 7.06 (1H, t, H₆ indole), 7.08 (1H, s, H₂ indole), 7.11 (1H, s, H₂ Trp), 7.18 (1H, d, J=7.9, H₄ Trp), 7.33 (3H, H₄, H₇ indole, H₇ Trp), 8.05 (2H, s, NH₂ Aib), 8.95 (1H, d, J=7.9, NH Trp), 10.80 (1H, s, NH indole), 10.82 (1H, s, NH indole Trp).

¹³C NMR (400 MHz, DMSO-d⁶):

δ 22.4 (CH₂—CH₂ indole), 23.2 (CH₃ Aib), 23.3 (CH₃ Aib), 25.4 (CH₂—CH₂ indole), 28.7 (Cβ Trp), 41.3 (CH₂— o,p-dimethoxybenzyl), 45.3 (Cα Trp), 55.2 (p-OCH₃), 55.4 (o-OCH₃), 56.3 (Cq Aib), 98.6 (C₃ o,p-dimethoxybenzyl), 104.7 (C₅ o,p-dimethoxybenzyl), 109.5 (C₃ Trp), 111.3 (C₇ Trp, C₇ indole), 112.9 (C₃ indole), 115.2 (C₁ o,p-dimethoxybenzyl), 117.8 (C₄ indole), 117.9 (C₄ Trp), 118.2 (C₅ Trp, C₅ indole), 120.9 (C₆ Trp, C₆indole), 122.4 (C₂ indole), 124.3 (C₂ Trp), 126.8 (C₉ Indole), 126.9 (C₉ Trp), 127.5 (C₆ o,p-dimethoxybenzyl), 136.0 (C₈ Trp), 136.2 (C₈ indole), 154.6 (2Cq triazole), 157.3 (C₂ o,p-dimethoxybenzyl), 160.4 (C₄ o,p-dimethoxybenzyl), 171.3 (CO Aib).

ESI-MS: found: m/z 606.3 [M+H]⁺/calculated: 604.3 g/mol

### Example 2

### (R)-N-(1-(5-benzyl-4-(naphthalen-1-ylmethyl)-4H-1,2,4-triazol-3-yl)-2-(1H-indol-3-yl)ethyl)-2-amino-2-methylpropanamide (Compound 4)

Compound 4 was obtained from Boc-(D)-Trp (10 mmoles), naphthalen-1-yl-methanamine, 2-phenylacetohydrazide and Boc-2-amino-2-methylpropanoic acid according to the general synthetic schemes with a total yield after purification by HPLC of 42%.

¹H NMR (300 MHz, DMSO-d⁶, 300° K):

δ(ppm) 1.18 (3H, s, CH₃ Aib), 1.24 (3H, s, CH₃ Aib), 3.17 (1H, dd, J=14 Hz and 5 Hz, CH₂ βTrp), 3.36 (1H, dd, J=14 and 9 Hz, CH₂ βTrp), 4.05 (2H, m, CH₂-benzyl), 4.90 (1H, m, CH αTrp), 5.65 (1H, d, J=18 Hz, CH₂-naphtyl), 5.81 (1H, d, J=18 Hz, CH₂-naphtyl), 6.12 (1H, d, Jₒ=7 Hz, H₂ naphtyl), 6.38 (1H, t, Jₒ=7 Hz, H₅ Trp), 6.47 (1H, d, Jₒ=8 Hz, H₄ Trp), 6.85 (1H, t, Jₒ=8 Hz, H₆ Trp), 7.03 (1H, d, Jₘ=2 Hz, H₂ Trp), 7.05-7.12 (5H, m, CHar benzyl), 7.15 (1H, d, Jₒ=8 Hz, H₇ Trp), 7.19 (1H, d, Jₒ=8 Hz, H₃ naphtyl), 7.58 (2H, m, H₆ and H₇ naphtyl), 7.81 (1H, d, Jₒ=8 Hz, H₄ naphtyl), 7.89-8.01 (5H, m, NH₂ Aib, H₅ and H₈ naphtyl), 8.92 (1H, d, J=8 Hz, NH amide), 10.73 (1H, s, NH indole Trp).

¹³C NMR (75 MHz, DMSO-d⁶, 300° K):

δ(ppm) 23.5 (CH₃ Aib), 23.6 (CH₃ Aib), 29.2 (CH₂ βTrp), 30.5 (CH₂-benzyl), 44.0 (CH₂-naphtyl), 45.6 (CH αTrp), 56.6 (Cq Aib), 109.7 (C₃ Trp), 111.7 (C₇ Trp), 117.9 (C₄ Trp), 118.4 (C₅ Trp), 121.1 (C₆ Trp), 122.1 (C₂ naphtyl), 122.8 (C₈ naphtyl), 124.9 (C₂ Trp), 125.7 (C₃ naphtyl), 126.7 (C₆ naphtyl), 126.9 (C₉ Trp), 127.0 (C₇ naphtyl), 128.2 (C₄ benzyl), 128.7-129.1 (C₂, C₃, C₅ and C₆ benzyl, C₄ and C₅ naphtyl), 129.9 (C₉ naphtyl), 131.5 (C₁ naphtyl), 133.5 (C₁₀ naphtyl), 136.2 (C₁ benzyl), 136.4 (C₈ Trp), 154.2 (Cq triazole), 155.7 (Cq triazole), 171.9 (CO Aib).

ESI-MS: found: m/z 543.4 [M+H]⁺/calculated: 542.2 g/mol

### Example 3

### (R)-N-(1-(5-(2-(1H-indol-3-yl)ethyl)-4-(naphthalen-1-ylmethyl)-4H-1,2,4-triazol-3-yl)-2-(1H-indol-3-yl)ethyl)-2-amino-2-methylpropanamide (Compound 5)

Compound 5 was obtained from Boc-(D)-Trp (10 mmoles), naphthalen-1-yl-methanamine, 3-(1H-indol-3-yl)propane hydrazide and Boc-2-amino-2-methylpropanoic acid according to the general synthetic schemes with a total yield after purification by HPLC of 33%.

¹H NMR (400 MHz, DMSO-d⁶, 300° K):

δ(ppm) 1.25 (3H, s, CH₃ Aib), 1.28 (3H, s, CH₃ Aib), 2.93 (2H, m, CH₂—CH₂-indole), 3.01 (2H, m, CH₂—CH₂-indole), 3.30 (1H, dd, ³J=14.3 and 5.8 Hz, CH₂ βTrp), 3.40 (1H, dd, ³J=14.3 and 8.8 Hz, CH₂ βTrp), 5.03 (1H, m, CH αTrp), 5.62 (1H, d, J=18.0 Hz, CH₂-naphtyl), 5.76 (1H, J=18.0 Hz, CH₂-naphtyl), 3.36 (1H, d, Jₒ=7.2 Hz, H₂ naphtyl), 6.51 (1H, t, Jₒ=7.4 Hz, H₅ Trp), 6.72 (1H, d, Jₒ=7.9 Hz, H₄ Trp), 6.76 (1H, t, Jₒ=7.5 Hz, H₅ indole), 6.92 (1H, t, Jₒ=7.5 Hz, H₆ Trp), 7.0 (1H, t, Jₒ=7.5 Hz, H₆ indole), 7.02 (1H, d, J=2.0 Hz, H₂ indole), 7.09 (1H, d, J=2.0 Hz, H₂ Trp), 7.13 (1H, d, Jₒ=7.9 Hz, H₄ indole), 7.26 (1H, Jₒ=7.9 Hz, H₇ Trp), 7.27 (1H, t, Jₒ=8.2 Hz, H₃ naphtyl), 7.29 (1H, d, H₇ indole), 7.58-7.64 (2, m, H₆ and H₇ naphtyl), 7.88 (1H, d, Jₒ=8.2 Hz, H₄ naphtyl), 7.93 (1H, d, Jₒ=7.9 Hz, H₈ naphtyl), 7.98 (3H, brs, NH₂ Aib), 8.03 (1H, d, Jₒ=8.2 Hz, H₅ naphtyl), 8.96 (1H, d, Jₒ=7.9 Hz, NH Trp), 10.75 (1H, brs, NH indole), 10.77 (1H, brs, NH indole Trp).

¹³C NMR (100 MHz, DMSO-d⁶, 300° K):

δ(ppm) 22.6 (CH₂—CH₂-indole), 23.1 (CH₃ Aib), 23.2 (CH₃ Aib), 25.3 (CH₂—CH₂-indole), 28.8 (CH₂ βTrp), 43.3 (CH₂-naphtyl), 45.3 (CH αTrp), 56.2 (Cq Aib), 109.4 (C₃ Trp), 111.2 (C₇ indole and C₇ Trp), 112.9 (C₃ indole), 117.5 (C₄ Trp), 117.8 (C₄ indole), 118.0 (C₅ Trp), 118.1 (C₅ indole), 120.7 (C₆ Trp), 120.8 (C₆ indole), 121.6 (C₂ naphtyl), 122.5 (C₂ indole and C₈ naphtyl), 124.4 (C₂ Trp), 125.4 (C₃ naphtyl), 126.3 (C₆ naphtyl), 126.6 (C₉ indole, C₉ Trp and C₇ naphtyl), 127.9 (C₄ naphtyl), 128.6 (C₅ naphtyl), 129.5 (C₉ naphtyl), 131.4 (C₁ naphtyl), 133.1 (C₁₀ naphtyl), 135.9 (C₈ Trp), 136.1 (C₈ indole), 154.7 (2 Cq triazole), 171.4 (CO Aib).

ESI-MS: found: m/z 596.4 [M+H]⁺/calculated: 595.3 g/mol

### Example 4

### (R)-N-(1-(5-(2-(1H-indol-3-yl)ethyl)-4-(3-methoxybenzyl)-4H-1,2,4-triazol-3-yl)-2-(1H-indol-3-yl)ethyl)-2-amino-2-methylpropanamide (Compound 6)

Compound 6 was obtained from Boc-(D)-Trp (10 mmoles), (3-methoxyphenyl)-methanamine, 3-(1H-indol-3-yl)propane hydrazide and Boc-2-amino-2-methylpropanoic acid according to the general synthetic schemes with a total yield after purification by HPLC of 25%.

¹H NMR (400 MHz, DMSO-d⁶):

δ(ppm) 1.28 (3H, s, CH₃ Aib), 1.30 (3H, s, CH₃ Aib), 2.92 (2H, m, CH₂—CH₂-indole), 2.98 (2H, m, CH₂—CH₂-indole), 3.33 (1H, dd, J=14.5, J=6.2, 1H CH₂ βTrp), 3.40 (1H, dd, J=14.5, J=8.8, 1H CH₂ βTrp), 3.66 (3H, s, OCH₃), 5.09 (2H, m, CH₂ m-methoxybenzyl), 5.22 (1H, m, CαH Trp), 6.38 (1H, d, J=7.5, H₆ m-methoxybenzyl), 6.59 (1H, s, H₂ m-methoxybenzyl), 6.86 (1H, t, H₅ Trp), 6.87 (1H, d, H₄ m-methoxybenzyl), 6.92 (1H, t, J=7.5, H₅ indole), 7.03 (1H, t, J=7.9, H₆ Trp), 7.05 (1H, t, H₆ indole), 7.07 (1H, s, H₂ indole), 7.11 (1H, s, H₂ Trp), 7.18 (1H, t, H₅ m-methoxybenzyl), 7.19 (1H, d, H₄ Trp), 7.31 (1H, H₄ indole), 7.32 (2H, H₇ Trp, H₇ indole), 8.00 (2H, s, NH₂ Aib), 8.96 (1H, d, J=8.1, NH Trp), 10.78 (1H, s, NH indole), 10.80 (1H, s, NH indole Trp).

¹³C NMR (400 MHz, DMSO-d⁶):

δ(ppm) 22.4 (CH₂—CH₂ indole), 23.1 (CH₃ Aib), 23.3 (CH₃ Aib), 25.4 (CH₂—CH₂ indole), 28.7 (Cβ Trp), 45.3 (CH₂ m-methoxybenzyl), 45.4 (Cα Trp), 55.0 (OCH₃), 56.3 (Cq Aib), 109.5 (C₃ Trp), 111.3 (C₇ Trp, C₇ indole), 112.0 (C₂ m-methoxybenzyl), 113.0 (C₄ m-methoxybenzyl, C₃ indole), 117.8 (C₄ Trp, C₆ m-methoxybenzyl), 118.0 (C₄ indole), 118.2 (C₅ indole), 118.3 (C₅ Trp), 120.8 (C₆ indole), 120.9 (C₆ Trp), 122.4 (C₂ indole), 124.3 (C₂ Trp), 126.7 (C₉ indole), 126.9 (C₉ Trp), 130.0 (C₅ m-methoxybenzyl), 136.0 (C₈ indole), 136.1 (C₈ Trp), 137.2 (C₁ m-methoxybenzyl), 154.3 (2Cq triazole), 159.6 (C₃ m-methoxybenzyl), 171.4 (CO Aib).

ESI-MS: found: m/z 576.6 [M+H]⁺/calculated: 575.3 g/mol

### Example 5

### (R)-N-(1-(4-(3-methoxybenzyl)-5-benzyl-4H-1,2,4-triazol-3-yl)-2-(1H-indol-3-yl)ethyl)-2-amino-2-methylpropanamide (Compound 7)

Compound 7 was obtained from Boc-(D)-Trp (10 mmoles), (3-methoxyphenyl)-methanamine, 2-phenylacetohydrazide and Boc-2-amino-2-methylpropanoic acid according to the general synthetic schemes with a total yield after purification by HPLC of 30%.

¹H NMR (400 MHz, DMSO-d⁶):

δ(ppm) 1.25 (3H, s, CH₃ Aib), 1.29 (3H, s, CH₃ Aib), 3.24 (1H, dd, J=14.3, J=5.8, 1H CH₂ βTrp), 3.38 (1H, dd, J=14.3, J=9.1, 1H CH₂ βTrp), 3.61 (3H, s, m-OCH₃), 4.04 (2H, m, CH₂ benzyl), 5.07 (1H, d, J=17.4, 1H CH₂ m-methoxybenzyl), 5.13 (1H, d, J=17.4, 1H CH₂ m-methoxybenzyl), 5.14 (1H, m, CαH Trp), 6.32 (1H, d, J=7.8, H₆ m-methoxybenzyl), 6.40 (1H, m, H₂ m-methoxybenzyl), 6.82 (1H, t, H₅ Trp), 6.83 (1H, d, J=7.8, H₄ m-methoxybenzyl), 7.01 (1H, t, J=8.2, H₆ Trp), 7.04 (1H, d, J=8.2, H₄ Trp), 7.06 (1H, d, J=2.0, H₂ Trp), 7.12 (2H, m, H₂, H₆ Benzyl), 7.13 (1H, t, J=7.9, H₅ m-methoxybenzyl), 7.20 (1H, m, H₄ benzyl), 7.24 (2H, m, H₃, H₅ benzyl), 7.29 (1H, d, J=8.2, H₇ Trp), 7.99 (2H, s, NH₂ Aib), 8.92 (1H, d, J=8.2, NH Trp), 10.77 (1H, s, NH indole Trp).

¹³C NMR (400 MHz, DMSO-d⁶):

δ(ppm) 23.0 (CH₃ Aib), 23.3 (CH₃ Aib), 28.6 (Cp Trp), 30.1 (CH₂ benzyl), 45.2 (Ca Trp), 45.6 (CH₂— m-methoxybenzyl), 54.9 (m-OCH₃), 56.2 (Cq Aib), 109.4 (C₃ Trp), 111.2 (C₇ Trp), 111.7 (C₂ m-methoxybenzyl), 113.1 (C₄ m-methoxybenzyl), 117.9 (C₄ Trp, C₆ m-methoxybenzyl), 118.2 (C₅ Trp), 120.8 (C₆ Trp), 124.3 (C₂ Trp), 126.6 (C₄ benzyl), 126.8 (C₉ Trp), 128.3 (C₃, C₅ benzyl), 128.4 (C₂, C₆ benzyl), 129.9 (C₅ m-methoxybenzyl), 135.9 (C₁ benzyl, C₈ Trp), 137.0 (C₁ m-methoxybenzyl ), 153.5 (Cq triazole), 154.8 (Cq triazole), 159.5 (C₃ m-methoxybenzyl), 171.3 (CO Aib).

ESI-MS: found: m/z 523.3 [M+H]⁺/calculated: 522.3 g/mol

### Example 6

### (R)-N-(1-(5-(2-(1H-indol-3-yl)ethyl)-4-benzyl-4H-1,2,4-triazol-3-yl)-2-(1H-indol-3-yl)ethyl)-2-amino-2-methylpropanamide (Compound 8)

Compound 8 was obtained from Boc-(D)-Trp (10 mmoles), phenylmethanamine, 3-(1H-indol-3-yl)propane hydrazide and Boc-2-amino-2-methylpropanoic acid according to the general synthetic schemes with a total yield after purification by HPLC of 45%.

¹H NMR (400 MHz, DMSO-d⁶):

δ(ppm) 1.29 (3H, s, CH₃ Aib), 1.30 (3H, s, CH₃ Aib), 2.88 (2H, m, CH₂—CH₂-indole), 2.97 (2H, m, CH₂—CH₂-indole), 3.37 (2H, m, CH₂ βTrp), 5.11 (2H, s, CH₂ benzyl), 5.21 (1H, m, CαH Trp), 6.86 (1H, t, J=7.4, H₅ Trp), 6.88 (2H, H₂, H₆ benzyl), 6.92 (1H, t, J=7.6, H₅ indole), 7.03 (1H, t, J=7.6, H₆ Trp), 7.05 (2H, H₆ indole, H₂ indole), 7.09 (1H, d, J=1.8, H₂ Trp), 7.17 (1H, d, J=7.9, H₄ Trp), 7.26 (2H, H₃, H₅ benzyl), 7.27 (1H, H₄ benzyl), 7.30 (1H, H₄ indole), 7.32 (2H, H₇ Trp, H₇ indole), 8.03 (2H, brs, NH₂ Aib), 8.95 (1H, d, J=8.1, NH Trp), 10.77 (1H, s, NH indole), 10.81 (1H, s, NH indole Trp).

¹³C NMR (400 MHz, DMSO-d⁶):

δ(ppm) 22.4 (CH₂—CH₂ indole), 23.1 (CH₃ Aib), 23.3 (CH₃ Aib), 25.4 (CH₂—CH₂ indole), 28.7 (Cβ Trp), 45.3 (Cα Trp, CH₂-benzyl), 56.3 (Cq Aib), 109.5 (C₃ Trp), 111.3 (C₇ Trp, C₇ indole), 113.0 (C₃ indole), 117.8 (C₄ Trp), 118.0 (C₄ indole), 118.2 (C₅ indole), 118.3 (C₅ Trp), 120.9 (C₆ Trp, C₆ indole), 122.4 (C₂ indole), 124.3 (C₂ Trp), 125.9 (C₂, C₆ benzyl), 126.7 (C₉ Indole), 126.9 (C₉ Trp), 127.6 (C₄ benzyl), 128.8 (C₃, C₅ benzyl), 135.7 (C₁ benzyl), 136.0 (C₈ Trp), 136.1 (C₈ indole), 154.3 (Cq triazole), 154.5 (Cq triazole), 171.4 (CO Aib).

ESI-MS: found: m/z 546.3 [M+H]⁺/calculated: 545.3 g/mol

### Example 7

### (R)-N-(1-(5-(3-(1H-indol-3-yl)propyl)-4-benzyl-4H-1,2,4-triazol-3-yl)-2-(1H-indol-3-yl)ethyl)-2-amino-2-methylpropanamide (Compound 9)

Compound 9 was obtained from Boc-(D)-Trp (10 mmoles), phenylmethanamine, 4-(1H-indol-3-yl)butanehydrazide and Boc-2-amino-2-methylpropanoic acid according to the general synthetic schemes with a total yield after purification by HPLC of 38%.

¹H NMR (400 MHz, DMSO-d⁶):

δ(ppm) 1.29 (3H, s, CH₃ Aib), 1.31 (3H, s, CH₃ Aib), 1.90 (2H, m, CH₂—CH₂—CH₂-indole), 2.61 (2H, m, CH₂—CH₂—CH₂-indole), 2.69 (2H, m, CH₂—CH₂—CH₂-indole), 3.37 (2H, m, CH₂ βTrp), 5.09 (2H, s, CH₂ benzyl), 5.20 (1H, m, CαH Trp), 6.85 (3H, m, H₂, H₆ benzyl, H₅ Trp), 6.94 (1H, t, J=7.5, H₅ indole), 7.01 (1H, s, H₂ indole), 7.02 (1H, t, J=7.8, H₆ Trp), 7.05 (1H, t, J=8, H₆ indole), 7.08 (1H, d, J=2.0, H₂ Trp), 7.14 (1H, d, J=8.0, H₄ Trp), 7.25 (3H, m, H₃, H₄, H₅ benzyl), 7.31 (1H, d, J=8.0, H₇ Trp), 7.32 (1H, d, J=8.0, H₇ indole), 7.42 (1H, d, J=7.8, H₄ indole), 8.03 (2H, s, NH₂ Aib), 8.95 (1H, d, J=8.1, NH Trp), 10.73 (1H, s, NH indole), 10.80 (1H, d, J=2.0, NH indole Trp).

¹³C NMR (400 MHz, DMSO-d⁶):

δ(ppm) 23.1 (CH₃ Aib), 23.3 (CH₃ Aib), 23.8 (CH₂—CH₂—CH₂-indole), 24.1 (CH₂—CH₂—CH₂-indole), 27.2 (CH₂—CH₂—CH₂-indole), 28.7 (Cβ Trp), 45.4 (Cα Trp), 45.5 (CH₂ benzyl), 56.3 (Cq Aib), 109.4 (C₃ Trp), 111.3 (C₇ Trp, C₇ indole), 113.6 (C₃ indole), 117.8 (C₄ Trp), 118.0 (C₅ indole), 118.2 (C₄ indole), 118.3 (C₅ Trp), 120.8 (C₆ indole, C₆ Trp), 122.2 (C₂ indole), 124.3 (C₂ Trp), 125.9 (C₂, C₆ benzyl), 126.8 (C₉ Trp), 127.0 (C₉ indole), 127.7 (C₄ benzyl), 128.7 (C₃, C₅ benzyl), 135.5 (C₁ benzyl), 136.0 (C₈ Trp), 136.2 (C₈ indole), 154.3 (Cq triazole), 154.7 (Cq triazole), 171.4 (CO Aib).

ESI-MS: found: m/z 560.4 [M+H]⁺/calculated: 559.3 g/mol

### Example 8

### (R)-N-(1-(5-(3-(1H-indol-3-yl)propyl)-4-(3-methoxybenzyl)-4H-1,2,4-triazol-3-yl)-2-(1H-indol-3-yl)ethyl)-2-amino-2-methylpropanamide (Compound 10)

Compound 10 was obtained from Boc-(D)-Trp (10 mmoles), (3-methoxyphenyl)-methanamine, 4-(1H-indol-3-yl)butanehydrazide and Boc-2-amino-2-methylpropanoic acid according to the general synthetic schemes with a total yield after purification by HPLC of 25%.

¹H NMR (400 MHz, DMSO-d⁶):

δ(ppm) 1.27 (3H, s, CH₃ Aib), 1.30 (3H, s, CH₃ Aib), 1.92 (2H, m, CH₂—CH₂—CH₂-indole), 2.62 (2H, m, CH₂—CH₂—CH₂-indole), 2.68 (2H, m, CH₂—CH₂—CH₂-indole), 3.24 (1H, dd, J=14.5, J=5.8, 1H CH₂ βTrp), 3.39 (1H, dd, J=14.5, J=9.0, 1H CH₂ βTrp), 3.66 (3H, s, m-OCH₃), 5.07 (2H, s, CH₂ m-methoxybenzyl), 5.18 (1H, m, CαH Trp), 6.35 (1H, d, J=7.5, H₆ m-methoxybenzyl), 6.54 (1H, bs, H₂ m-methoxybenzyl), 6.84 (1H, t, J=7.5, H₅ Trp), 6.87 (1H, dd, J=8.0, J=2.1, H₄ m-methoxybenzyl), 6.94 (1H, t, J=7.3, H₅ indole), 7.02 (1H, t, H₆ Trp), 7.02 (1H, s, H₂ indole), 7.05 (1H, t, J=7.8, H₆ indole), 7.08 (1H, d, J=2.1, H₂Trp), 7.13 (1H, d, J=8.1, H₄Trp), 7.17 (1H, t, J=8.1, H₅ m-methoxybenzyl), 7.30 (1H, d, H₇ Trp), 7.32 (1H, d, J=8, H₇ indole), 7.42 (1H, d, J=7.6, H₄ indole), 7.98 (2H, s, NH₂ Aib), 8.93 (1H, d, J=8.2, NH Trp), 10.71 (1H, s, NH indole), 10.77 (1H, s, NH indole Trp).

¹³C NMR (400 MHz, DMSO-d⁶):

δ(ppm) 23.1 (CH₃ Aib), 23.3 (CH₃ Aib), 23.9 (CH₂—CH₂—CH₂-indole), 24.3 (CH₂—CH₂—CH₂-indole), 27.4 (CH₂—CH₂—CH₂ indole), 28.8 (Cβ Trp), 45.2 (CH₂— m-methoxybenzyl), 45.4 (Cα Trp), 55.1 (m-OCH₃), 56.3 (Cq Aib), 109.5 (C₃ Trp), 111.3 (C₇ Trp, C₇ indole), 111.8 (C₂ m-methoxybenzyl), 113.0 (C₄ m-methoxybenzyl), 113.8 (C₃ indole), 117.8 (C₆ m-methoxybenzyl), 117.9 (C₄ Trp), 118.1 (C₅ indole), 118.2 (C₅ Trp, C₄ indole), 120.8 (C₆ Trp), 120.9 (C₆ indole), 122.2 (C₂ indole), 124.3 (C₂ Trp), 126.8 (C₉ Trp), 127.0 (C₉ Indole), 130.0 (C₅ m-methoxybenzyl), 136.0 (C₈ Trp), 136.2 (C₈ indole), 137.4 (C₁ m-methoxybenzyl), 154.3 (Cq triazole), 154.6 (Cq triazole), 159.7 (C₃ m-methoxybenzyl), 171.4 (CO Aib).

ESI-MS: found: m/z 590.3 [M+H]⁺/calculated: 589.3 g/mol

### Example 9

### (R)-N-(1-(5-(3-(1H-indol-3-yl)propyl)-4-(naphthalen-1-ylmethyl)-4H-1,2,4-triazol-3-yl)-2-(1H-indol-3-yl)ethyl)-2-amino-2-methylpropanamide (Compound 11)

Compound 11 was obtained from Boc-(D)-Trp (10 mmoles), naphthalen-1-ylmethanamine, 4-(1H-indol-3-yl)butanehydrazide and Boc-2-amino-2-methylpropanoic acid according to the general synthetic schemes with a total yield after purification by HPLC of 22%.

¹H NMR (300 MHz, DMSO-d⁶, 300° K):

δ(ppm) 1.20 (3H, s, CH₃ Aib), 1.25 (3H, s, CH₃ Aib), 1.93 (2H, m, CH₂—CH₂—CH₂-indole), 2.66 (4H, m, CH₂—CH₂—CH₂-indole), 3.25 (1H, dd, J=14 Hz and 5 Hz, CH₂ βTrp), 3.40 (1H, dd, J=14 Hz and 9 Hz, CH₂ βTrp), 4.95 (1H, m, CH αTrp), 5.66 (1H, d, J=18 Hz, CH₂-naphtyl), 5.81 (1H, d, J=18 Hz, CH₂-naphtyl), 6.37 (1H, d, Jₒ=7 Hz, H₂ naphtyl), 6.43 (1H, t, Jₒ=7 Hz, H₅ Trp), 6.59 (1H, d, Jₒ=8 Hz, H₄ Trp), 6.86 (3H, m, H₅ and H₆ indole, H₆ Trp), 6.95 (1H, d, J=2 Hz, H₂ indole), 7.00 (1H, d, Jₒ=8 Hz, H₄ indole), 7.06 (1H, d, J=2 Hz, H₂ Trp), 7.20-7.33 (4H, m, H₄ and H₇ indole, H₇ Trp, H₃ naphtyl), 7.60 (2H, m, H₆ and H₇ naphtyl), 7.87 (1H, d, Jₒ=8 Hz, H₄ naphtyl), 7.99 (5H, m, NH₂ Aib, H₅ and H₈ naphtyl), 8.95 (1H, d, J=8 Hz, NH amide), 10.70 (1H, s, NH indole), 10.77 (1H, s, NH indole Trp).

¹³C NMR (75 MHz, DMSO-d⁶, 300° K):

δ(ppm) 23.5 (CH₃ Aib), 23.6 (CH₃ Aib), 24.1 (CH₂—CH₂—CH₂-indole), 24.5 (CH₂—CH₂—CH₂-indole), 27.6 (CH₂—CH₂—CH₂-indole), 29.1 (CH₂ βTrp), 44.1 (CH₂-naphtyl), 45.7 (CH α Trp), 56.7 (Cq Aib), 109.7 (C₃ Trp), 111.7 (C₇ indole and C₇ Trp), 113.9 (C₃ indole), 117.9 (C₄ Trp), 118.5 (C₄ indole, C₅ Trp), 118.6 (C₅ indole), 121.1 (C₆ Trp), 121.2 (C₆ indole), 122.1 (C₂ naphtyl), 122.7 (C₂ indole), 122.9 (C₈ naphtyl), 125.0 (C₂ Trp), 125.9 (C₃ naphtyl), 126.8 (C₆ naphtyl), 127.0 (C₉ indole), 127.1 (C₇ naphtyl), 127.4 (C₉ Trp), 128.5 (C₄ naphtyl), 129.2 (C₅ naphtyl), 129.9 (C₉ naphtyl), 131.6 (C₁ naphtyl), 133.6 (C₁₀ naphtyl), 136.4 (C₈ Trp), 136.7 (C₈ indole), 155.4 (Cqs triazole), 171.9 (CO Aib).

ESI-MS: found: m/z 610.3 [M+H]⁺/calculated: 609.3 g/mol

### Example 10

### (R)-N-(1-(5-(2-(1H-indol-3-yl)ethyl)-4-(4-methoxybenzyl)-4H-1,2,4-triazol-3-yl)-2-(1H-indol-3-yl)ethyl)-2-amino-2-methylpropanamide (Compound 12)

Compound 12 was obtained from Boc-(D)-Trp (10 mmoles), (4-methoxyphenyl)-methanamine, 3-(1H-indol-3-yl)propane hydrazide and Boc-2-amino-2-methylpropanoic acid according to the general synthetic schemes with a total yield after purification by HPLC of 28%.

¹H NMR (400 MHz, DMSO-d⁶):

δ(ppm) 1.30 (3H, s, CH₃ Aib), 1.33 (3H, s, CH₃ Aib), 2.91 (2H, m, CH₂—CH₂-indole), 2.97 (2H, m, CH₂—CH₂-indole), 3.37 (2H, d, CH₂ βTrp), 3.71(3H, s, OCH₃), 5.02 (2H, s, CH₂ p-methoxybenzyl), 5.23 (1H, m, CαH Trp), 6.78 (4H, m, CHar p-methoxybenzyl), 6.87 (1H, t, J=7.5, H₅ Trp), 6.93 (1H, t, J=7.5, H₅ indole), 7.03 (1H, t, H₆ Trp), 7.05 (1H, t, H₆ indole), 7.07 (1H, s, H₂ indole), 7.09 (1H, s, H₂ Trp), 7.21 (1H, d, J=8, H₄ Trp), 7.32 (3H, H₄ indole, H₇ Trp, H₇ indole), 8.02 (2H, s, NH₂ Aib), 8.97 (1H, d, J=8.1, NH Trp), 10.77 (1H, s, NH indole), 10.80 (1H, s, NH indole Trp).

¹³C NMR (400 MHz,DMSO-d⁶):

δ(ppm) 22.4 (CH₂—CH₂ indole), 23.1 (CH₃ Aib), 23.4 (CH₃ Aib), 25.5 (CH₂—CH₂ indole), 28.9 (Cβ Trp), 44.9 (CH₂ p-methoxybenzyl), 45.3 (Cα Trp), 55.0 (OCH₃), 56.3 (Cq Aib), 109.5 (C₃ Trp), 111.3 (C₇ Trp, C₇ indole), 113.0 (C₃ indole), 114.1 (C₃, C₅ p-methoxybenzyl), 117.9 (C₄ Trp), 118.0 (C₄ indole), 118.2 (C₅ indole), 118.3 (C₅ Trp), 120.9 (C₆ indole, C₆ Trp), 122.0 (C₂ indole), 124.4 (C₂ Trp), 126.7 (C₉ indole), 126.9 (C₉ Trp), 127.3 (C₂, C₆ p-methoxybenzyl), 127.4 (C₁ p-methoxybenzyl), 135.9 (C₈ Trp), 136.1 (C₈ indole), 154.2 (Cq triazole), 154.5 (Cq triazole), 158.4 C₄ p-methoxybenzyl), 171.4 (CO Aib).

ESI-MS: found: m/z 576.3 [M+H]⁺/calculated: 575.3 g/mol

### Example 11

### (R)-N-(1-(4-(4-methoxybenzyl)-5-benzyl-4H-1,2,4-triazol-3-yl)-2-(1H-indol-3-yl)ethyl)-2-amino-2-methylpropanamide (Compound 13)

Compound 13 was obtained from Boc-(D)-Trp (10 mmoles), (4-methoxyphenyl)-methanamine, 2-phenylacetohydrazide and Boc-2-amino-2-methylpropanoic acid according to the general synthetic schemes with a total yield after purification by HPLC of 37%.

¹H NMR (300 MHz, DMSO-d⁶, 300° K):

δ(ppm) 1.24 (3H, s, CH₃ Aib), 1.28 (3H, s, CH₃ Aib), 3.26 (1H, dd, ³J=14 Hz and 6 Hz, CH₂ βTrp), 3.31 (1H, dd, ³J=14 Hz and 9 Hz, CH₂ βTrp), 3.67 (3H, s, OCH₃), 3.99 (2H, s, CH₂-benzyl), 4.99 (2H, s, CH₂-p-methoxybenzyl), 5.12 (1H, m, CH αTrp), 6.67 (4H, m, CHar p-methoxybenzyl), 6.80 (1H, t, Jₒ=8 Hz, H₅ Trp), 6.98 (1H, t, Jₒ=8 Hz, H₆ Trp), 7.02-7.06 (4H, m, H₂ and H₆ benzyl, H₂ and H₄ Trp), 7.12-7.25 (3H, m, H₃, H₄ and H₅ benzyl), 7.26 (1H, d, Jₒ=8 Hz, H₇ Trp), 8.01 (3H, brs, NH₂ Aib), 8.92 (1H, d, J=8 Hz, NH Trp), 10.77 (1H, s, NH indole Trp).

¹³C NMR (75 MHz, DMSO-d⁶, 300° K):

δ(ppm) 23.5 (CH₃ Aib), 23.7 (CH₃ Aib), 29.1 (CH₂ βTrp), 30.6 (CH₂-benzyl), 45.7 (CH₂-p-methoxybenzyl), 45.7 (CH αTrp), 55.5 (OCH₃), 56.7 (Cq Aib), 109.8 (C₃ Trp), 111.7 (C₇ Trp), 114.5 (C₃ and C₅ p-methoxybenzyl), 118.3 (C₄ Trp), 118.7 (C₅ Trp), 121.3 (C₆ Trp), 124.8 (C₂ Trp), 127.1 (C₂ and C₆ benzyl), 127.3 (C₉ Trp), 127.6 (C₁ p-methoxybenzyl), 127.8 (C₂ and C₆ p-methoxybenzyl), 128.8 (C₃, C₄ and C₅ p-methoxybenzyl), 136.3 (C₁ benzyl), 136.4 (C₈ Trp), 153.8 (Cq triazole), 155.2 (Cq triazole), 159.1 (C₄ p-methoxybenzyl), 171.9 (CO Aib).

ESI-MS: found: m/z 524.1 [M+H]⁺/calculated: 522.3 g/mol

### Example 12

### (R)-N-(1-(5-(2-(1H-indol-3-yl)ethyl)-4-hexyl-4H-1,2,4-triazol-3-yl)-2-(1H-indol-3-yl)ethyl)-2-amino-2-methylpropanamide (Compound 15)

Compound 15 was obtained from Boc-(D)-Trp (10 mmoles), hexan-1-amine, 3-(1H-indol-3-yl)propane hydrazide and Boc-2-amino-2-methylpropanoic acid according to the general synthetic schemes with a total yield after purification by HPLC of 28%.

¹H NMR (400 MHz, DMSO-d⁶):

δ(ppm) 0.77 (3H, t, J=7.2 (CH₂)₅—CH₃), 1.01 (4H, m, 2CH₂), 1.11 (2H, m, CH₂—CH₃), 1.14 (1H, m, 1H N—CH₂—CH₂), 1.33 (1H, m, 1H N—CH₂—CH₂), 1.40 (3H, s, CH₃ Aib), 1.42 (3H, s, CH₃ Aib), 3.05 (2H, m, CH₂—CH₂-indole), 3.10 (2H, m, CH₂—CH₂-indole), 3.37 (1H, dd, J=14.2, J=7.6, 1H CH₂ βTrp), 3.44 (1H, dd, J=14.2, J=7.6, 1H CH₂ βTrp), 3.58 (1H, m, 1H N—CH₂), 3.71 (1H, m, 1H N—CH₂), 5.21 (1H, m, CαH Trp), 6.96 (1H, H₅ Trp), 6.97 (1H, H₅ indole), 7.06 (2H, H₆ Trp, H₆ indole), 7.09 (1H, s, H₂ Trp), 7.13 (1H, s, H₂ indole), 7.34 (2H, H₇ Trp, H₇ indole),7.48 (1H, d, H₄ indole), 7.50 (1H, H₄ Trp), 8.14 (2H, s, NH₂ Aib), 9.08 (1H, d, J=7.8, NH Trp), 10.84 (1H, s, NH indole), 10.88 (1H, s, NH indole Trp).

¹³C NMR (400 MHz, DMSO-d⁶):

δ(ppm) 13.7 (CH₂)₅—CH₃), 21.7 (CH₂—CH₃), 22.4 (CH₂—CH_{□}indole), 23.1 (CH₃ Aib), 23.3 (CH₃ Aib), 25.1 (CH₂—CH_{□}indole), 25.5 (CH₃—CH₂—CH₂—CH₂), 29.1 (Cβ Trp), 29.3 (N—CH₂—CH₂), 30.4 (CH₃—CH₂—CH₂), 42.6 (N—CH₂—CH₂), 45.6 (Cα Trp), 56.3 (Cq Aib), 109.2 (C₃ Trp), 111.4-111.5 (C₇ Trp, C₇ indole), 112.8 (C₃ indole), 117.7 (C₄ Trp), 118.0 (C₅ indole), 118.2 (C₄ indole), 118.4 (C₅ Trp), 120.9 (C₆ indole, C₆ Trp), 122.6 (C₂ indole), 124.3 (C₂ Trp), 126.8 (C₉ Trp), 126.9 (C₉ indole), 136.0 (C₈ Trp), 136.2 (C₈ indole), 154.0 (Cq triazole), 154.1 (Cq triazole), 171.4 (CO Aib).ESI-MS: found: m/z 540.3 [M+H]⁺/calculated: 539.3 g/mol

### Example 13

### (S)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide (Compound 18)

Compound 18 was obtained from Boc-(L)-Trp (10 mmoles), (2,4-dimethoxyphenyl)-methanamine, 3-(1H-indol-3-yl)propane hydrazide and Boc-2-amino-2-methylpropanoic acid according to the general synthetic schemes with a total yield after purification by HPLC of 30%.

¹H NMR (300 MHz, DMSO-d⁶, 300° K):

δ(ppm) 1.27 (3H, s, CH₃ Aib), 1.31 (3H, s, CH₃ Aib), 2.89 (2H, m, CH₂CH₂-indole), 2.93 (2H, m, CH₂CH₂-indole), 3.27 (2H, m, CH₂ βTrp), 3.62 (3H, s, o-OCH₃), 3.68 (3H, s, p-OCH₃), 4.89 (1H, d, ³J=17 Hz, CH₂-o,p-dimethoxybenzyl), 5.06 (1H, d, ³J=17 Hz, CH₂-o,p-dimethoxybenzyl), 5.18 (1H, m, CH αTrp), 6.27 (1H, dd, Jₒ=8 Hz and Jₚ=2 Hz, H₅ o,p-dimethoxybenzyl), 6.40 (1H, d, 8 Hz, H₆ o,p-dimethoxybenzyl), 6.56 (1H, d, Jₚ=2 Hz, H₃ o,p-dimethoxybenzyl), 6.83 (1H, t, Jₒ=7 Hz, H₅ Trp), 6.90 (1H, t, Jₒ=7 Hz, H₅ indole), 7.02 (1H, t, H₆ Trp), 7.04 (1H, t, H₆ indole), 7.07 (1H s, H₂ indole), 7.08 (1H, s, H₂ Trp), 7.12 (1H, d, Jₒ=8 Hz, H₄ Trp), 7.29 (3H, H₄ and H₇ indole, H₇ Trp), 8.00 (3H, brs, NH₂ Aib), 8.93 (1H, d, J=8 Hz, NH amide), 10.76 (1H, s, NH indole), 10.79 (1H, s, NH indole Trp).

¹³C NMR (75 MHz, DMSO-d⁶, 300° K):

δ(ppm) 22.9 (CH₂CH₂-indole), 23.6 (CH₃ Aib), 23.7 (CH₃ Aib), 25.8 (CH₂CH₂-indole), 29.2 (CH₂ βTrp), 41.4 (CH₂-o,p-dimethoxybenzyl), 45.7 (CαTrp), 55.7 (p-OCH₃), 55.9 (o-OCH₃), 56.7 (Cq Aib), 99.0 (C₃ o,p-dimethoxybenzyl), 105.1 (C₅ o,p-dimethoxybenzyl), 109.9 (C₃ Trp), 111.8 (C₇ Trp, C₇ indole), 113.4 (C₃ indole), 115.6 (C₁ o,p-dimethoxybenzyl), 118.3 (C₄ indole), 118.4 (C₄ Trp), 118.6 (C₅ Trp, C₅ indole), 121.3 (C₆ Trp, C₆ indole), 122.6 (C₂ indole), 124.4 (C₂ Trp), 127.2 (C₉ indole), 127.3 (C₉ Trp), 128.0 (C₆ o,p-dimethoxybenzyl), 136.4 (C₈ Trp), 136.6 (C₈ indole), 155.0 (2 Cq triazole), 157.7 (C₂ o,p-dimethoxybenzyl), 160.9 (C₄ o,p-dimethoxybenzyl), 171.6 (CO Aib).

ESI-MS: found: m/z 606.2 [M+H]⁺/calculated: 605.3 g/mol

### Example 14

### (R)-N-(1-(5-(3-(1H-indol-3-yl)propyl)-4-(4-methoxybenzyl)-4H-1,2,4-triazol-3-yl)-2-(1H-indol-3-yl)ethyl)-2-amino-2-methylpropanamide (Compound 23)

Compound 23 was obtained from Boc-(D)-Trp (10 mmoles), (4-methoxyphenyl)-methanamine, 4-(1H-indol-3-yl)butanehydrazide and Boc-2-amino-2-methylpropanoic acid according to the general synthetic schemes with a total yield after purification by HPLC of 25%.

¹H NMR (300 MHz, DMSO-d⁶, 300° K):

δ(ppm) 1.27 (3H, s, CH₃ Aib), 1.30 (3H, s, CH₃ Aib), 1.84 (2H, m, CH₂CH₂CH₂-indole), 2.58 (2H, m, CH₂CH₂CH₂-indole), 2.65 (2H, m, CH₂CH₂CH₂-indole), 3.34 (2H, d, ³J=7 Hz, CH₂ βTrp), 3.67 (3H, s, OCH₃), 4.96 (2H, s, CH₂-p-methoxybenzyl), 5.19 (1H, m, CH αTrp), 6.71 (4H, s, CH ar p-methoxybenzyl), 6.89 (1H, t, Jₒ=7 Hz, H₅ Trp), 6.92 (1H, t, Jₒ=7 Hz, H₅ indole), 7.02 (1H, s, H₂ indole), 7.05 (1H, s, H₂ Trp), 7.14 (1H, d, Jₒ=8 Hz, H₄ Trp), 7.33 (3H, H₄ indole, H₇ Trp, H₇ indole), 8.02 (3H, brs, NH₂ Aib), 7.90 (1H, d, J=8 Hz, NH amide), 10.73 (1H, s, NH indole), 10.79 (1H, s, NH indole Trp).

¹³C NMR (75 MHz, DMSO-d⁶, 300° K):

δ(ppm) 23.6 (CH₃ Aib), 23.8 (CH₃ Aib), 24.3 (CH₂CH₂CH₂-indole), 24.8 (CH₂CH₂CH₂-indole), 27.7 (CH₂CH₂CH₂-indole), 29.1 (Cβ Trp), 45.5 (N—CH₂-p-methoxybenzyl), 45.8 (Cα Trp), 55.5 (OCH₃), 56.8 (Cq Aib), 109.8 (C₃ Trp), 111.7 (C₇ Trp, C₇ indole), 114.0 (C₃ indole), 114.5 (C₃, C₅ p-methoxybenzyl), 118.3 (C₄ indole, C₄ Trp), 118.5 (C₅ indole), 118.8 (C₅ Trp), 121.3 (C₆ indole, C₆ Trp), 127.3 (C₉ indole), 127.4 (C₉ Trp), 127.6 (C₁ p-methoxybenzyl), 127.9 (C₂, C₆ p-methoxybenzyl, C₂ Trp, C₂ indole), 136.1 (C₈ indole), 136.4 (C₈ Trp), 154.7 (Cq triazole), 155.1 (Cq triazole), 159.2 (C₄ p-methoxybenzyl), 171.9 (CO Aib).

ESI-MS: found: m/z 590.0 [M+H]⁺/calculated: 589.3 g/mol

### Example 15

### (R)-N-(1-(5-(2-(1H-indol-3-yl)ethyl)-4-(2-methoxy)benzyl)-4H-1,2,4-triazol-3-yl)-2-(1H-indol-3-yl)ethyl)-2-amino-2-methylpropanamide (Compound 25)

Compound 25 was obtained from Boc-(D)-Trp (10 mmoles), (2-methoxyphenyl)-methanamine, 3-(1H-indol-3-yl)propane hydrazide and Boc-2-amino-2-methylpropanoic acid according to the general synthetic schemes with a total yield after purification by HPLC of 28%.

¹H NMR (300 MHz, DMSO-d⁶, 300° K):

δ(ppm) 1.27 (3H, s, CH₃ Aib), 1.29 (3H, s, CH₃ Aib), 2.90 (2H, m, CH₂—CH₂-indole), 2.96 (2H, m, CH₂—CH₂-indole), 3.29 (2H, m, CH₂ βTrp), 3.65 (3H, s, OCH₃), 5.09 (3H, m, CH₂-o-methoxybenzyl and CH αTrp), 6.49 (1H, d, Jₒ=8 Hz, H₃ o-methoxybenzyl), 6.76 (1H, t, Jₒ=8 Hz, H₅ Trp), 6.81 (1H, t, Jₒ=8 Hz, H₅ indole), 6.89 (1H, t, Jₒ=7 Hz, H₆ Trp), 6.96 (1H, t, Jₒ=8 Hz, H₆ indole), 6.98 (1H, s, H₂ indole), 7.02 (3H, m, H₄, H₅ and H₆ o-methoxybenzyl), 7.07 (1H, d, Jₒ=6 Hz, H₄ Trp), 7.18 (1H, m, H₄ indole), 7.29 (2H, m, H₇ indole and H₇ Trp), 8.07 (3H, brs, NH₂ Aib), 8.97 (1H, d, J=8 Hz, NH amide), 10.80 (1H, s, NH indole), 10.82 (1H, s, NH indole Trp).

¹³C NMR (75 MHz, DMSO-d⁶, 300° K):

δ(ppm) 22.8 (CH₂—CH₂-indole), 23.6 (CH₃ Aib), 23.7 (CH₃ Aib), 25.8 (CH₂—CH₂-indole), 29.1 (CH₂ βTrp), 42.3 (CH₂-o-methoxybenzyl), 45.7 (CH α Trp), 55.8 (OCH₃), 56.7 (Cq Aib), 109.8 (C₃ Trp), 111.5 (C₃ o-methoxybenzyl), 111.8 (C₇ indole and C₇ Trp), 113.2 (C₃ indole), 118.2 (C₄ Trp), 118.4 (C₄ indole), 118.7 (C₅ indole and C₅ Trp), 121.0 (C₆ indole), 121.3 (C₆ Trp), 121.4 (C₅ o-methoxybenzyl), 123.0 (C₂ indole and C₂ Trp), 123.3 (C₁ o-methoxybenzyl), 127.0 (C₄ o-methoxybenzyl), 127.1 (C₉ indole), 127.3 (C₉ Trp), 129.8 (C₆ o-methoxybenzyl), 136.4 (C₈ indole), 136.6 (C₈ Trp), 155.2 (Cq triazole), 171.9 (CO Aib).

ESI-MS: found: m/z 576.1 [M+H]⁺/calculated: 575.3 g/mol

Data on further exemplary embodiments that were synthesized according to the general synthesis schemes are compiled below (please refer also to Table 1):

(R)-N-(1-(5-(3-(1H-indol-3-yl)propyl)-4-phenethyl-4H-1,2,4-triazol-3-yl)-2-(1H-indol-3-yl)ethyl)-2-amino-2-methylpropanamide (Compound 3):

¹H NMR (300 MHz, DMSO-d⁶, 300° K):

δ(ppm) 1.32 (s, 3H, CH₃ Aib), 1.37 (s, 3H, CH₃ Aib), 1.86 (2H, m, CH₂—CH₂—CH₂-indole), 2.38 (2H, m, CH₂—CH₂—CH₂-indole), 2.65 (4H, m, CH₂—CH₂—CH₂-indole and CH₂—CH₂-phenyl), 3.38 (2H, m, CH₂—CH₂-phenyl), 3.74 (1H, m, CH₂ βTrp), 3.92 (1H, m, CH₂ βTrp), 5.23 (1H, m, CH αTrp), 6.78 (2H, m, H₅ indole and H₅ Trp), 6.93 (1H, t, Jₒ=8 Hz, H₆ Trp), 7.01 (3H, m, H₆ indole, H₂ and H₆ phenyl), 7.05 (1H, d, J=2 Hz, H₂ Trp), 7.08 (1H, d, J=2 Hz, H₂ indole), 7.15 (3H, m, H₃, H₄ and H₅ phenyl), 7.29 (1H, d, Jₒ=8 Hz, H₄ Trp), 7.31 (1H, d, Jₒ=8 Hz, H₇ Trp), 7.44 (1H, d, Jₒ=8 Hz, H₇ indole), 7.46 (1H, d, Jₒ=8 Hz, H₄ indole), 8.06 (3H, brs, NH₂ Aib), 9.05 (1H, d, 8 Hz, NH amide), 10.76 (1H, s, NH indole), 10.85 (1H, d, J=2 Hz, NH indole Trp).

¹³C NMR (75 MHz, DMSO-d⁶, 300° K):

δ(ppm) 23.5 (CH₃ Aib), 23.6 (CH₂—CH₂—CH₂-indole), 23.9 (CH₃ Aib), 24.5 (CH₂—CH₂—CH₂-indole), 27.3 (CH₂—CH₂—CH₂-indole), 29.4 (CH₂ βTrp), 35.7 (CH₂—CH₂-phenyl), 44.5 (CH₂—CH₂-phenyl), 46.1 (CH αTrp), 56.8 (Cq Aib), 109.6 (C₃ Trp), 111.8 (C₇ indole), 111.9 (C₇ indole), 113.9 (C₃ indole), 118.3 (C₄ Trp), 118.6 (C₅ indole), 118.7 (C₄ indole), 118.9 (C₅ Trp), 121.3 (C₆ Trp), 121.4 (C₆ indole), 122.8 (C₂ indole and C₂ Trp), 127.1 (C₄ phenyl), 127.3 (C₉ Trp), 127.5 (C₉ indole), 128.8 (C₂ and C₆ phenyl), 129.1 (C₃ and C₅ phenyl), 136.5 (C₁ phenyl), 136.8 (C₈ Trp), 137.2 (C₈ indole), 154.7 (Cq triazole), 172.0 (CO Aib).

### (R)-N-(1-(5-(3-(1H-indol-3-yl)propyl)-4-hexyl-4H-1,2,4-triazol-3-yl)-2-(1H-indol-3-yl)ethyl)-2-amino-2-methylpropanamide (Compound 16):

¹H NMR (300 MHz, DMSO-d⁶, 300° K):

δ(ppm) 0.74 (3H, t, J=6 Hz, CH₃—CH₂—CH₂—CH₂—CH₂—CH₂), 0.95 (4H, brs, CH₃—CH₂—CH₂—CH₂—CH₂—CH₂), 1.06 (3H, m, CH₃—CH₂—CH₂—CH₂—CH₂—CH₂ and 1H N—CH₂—CH₂), 1.38 (7H, s, CH₃ Aib and 1H N—CH₂—CH₂), 1.97 (2H, m, CH₂—CH₂—CH₂-indole), 2.71 (4H, m, CH₂—CH₂—CH₂-indole), 3.37 (2H, m, CH₂ βTrp), 3.56 (2H, m, N—CH₂), 5.15 (1H, m, CH αTrp), 6.91 (2H, m, H₅ indole and H₅ Trp), 7.00 (2H, m, H₆ indole and H₆ Trp), 7.07 (2H, s, H₂ indole and H₂ Trp), 7.29 (2H, d, Jₒ=8 Hz, H₇ indole and H₇ Trp), 7.45 (2H, d, Jo=7 Hz, H₄ indole and H₄ Trp), 8.15 (3H, brs, NH₂ Aib), 9.10 (1H, d, J=6 Hz, NH amide), 10.77 (1H, s, NH indole), 10.85 (1H, s, NH indole Trp).

¹³C NMR (75 MHz, DMSO-d⁶, 300° K):

δ(ppm) 14.2 (CH₃—CH₂—CH₂—CH₂—CH₂—CH₂), 22.2 (CH₃—CH₂—CH₂—CH₂—CH₂—CH₂ and CH₂—CH₂—CH₂-indole), 23.6 (CH₃ Aib), 23.7 (CH₃ Aib), 24.5 (CH₂—CH₂—CH₂-indole), 25.9 (CH₃—CH₂—CH₂—CH₂—CH₂—CH₂), 27.5 (CH₂ βTrp and CH₂—CH₂—CH₂-indole), 29.7 (N—CH₂—CH₂), 30.8 (CH₃—CH₂—CH₂—CH₂—CH₂—CH₂), 43.2 (N—CH₂), 46.1 (CH αTrp), 56.8 (Cq Aib), 109.5 (C₃ Trp), 111.8 (C₇ Trp), 111.9 (C₇ indole), 113.9 (C₃ indole), 118.1 (C₄ Trp), 118.5 (C₅ indole), 118.6 (C₄ indole), 118.9 (C₅ Trp), 121.3 (C₆ indole and C₆ Trp), 122.8 (C₂ indole and C₂ Trp), 127.3 (C₉ Trp), 127.4 (C₉ indole), 136.5 (C₈ Trp), 136.8 (C₈ indole), 154.7 (Cq triazole), 172.0 (CO Aib).

### (R)-N-(1-(4,5-bis(2-(1H-indol-3-yl)ethyl)-4H-1,2,4-triazol-3-yl)-2-(1H-indol-3-yl)ethyl)-2-amino-2-methylpropanamide (Compound 17):

¹H NMR (300 MHz, DMSO-d⁶, 300° K):

δ(ppm) 1.30 (3H, s, CH₃ Aib), 1.37 (3H, s, CH₃ Aib), 2.50 (2H, m, N—CH₂—CH₂-indole), 2.68 (2H, t, Jₒ=8 Hz, C—CH₂—CH₂-indole), 2.91 (2H, t, Jₒ=8 Hz, C—CH₂—CH₂-indole), 3.34 (2H, m, N—CH₂—CH₂-indole), 3.93 (2H, m, CH₂ βTrp), 5.25 (1H, m, CH αTrp), 6.72-6.94 (4H, m, H₅ and H₆ Trp, H₅ indole from C—CH₂—CH₂-indole and H₅ indole from N—CH₂—CH₂-indole), 6.98-7.04 (4H, m, H₂ Trp, H₆ indole from C—CH₂—CH₂-indole, H₂ and H₆ indole from N—CH₂—CH₂-indole), 7.11 (1H, s, H₂ indole from C—CH₂—CH₂-indole), 7.19 (1H, d, Jₒ=8 Hz, H₄ indole from N—CH₂—CH₂-indole), 7.28 (3H, m, H₄ and H₇ Trp, H₇ indole from N—CH₂—CH₂-indole), 7.40 (1H, d, Jₒ=8 Hz, H₇ indole from C—CH₂—CH₂-indole), 7.44 (1H, d, Jₒ=8 Hz, H₄ indole from C—CH₂—CH₂-indole), 8.04 (3H, brs, NH₂ Aib), 9.69 (1H, d, J=8 Hz, NH amide), 10.73 (1H, s, NH indole from C—CH₂—CH₂-indole), 10.82 (1H, d, J=2 Hz, NH indole Trp), 10.84 (1H, s, NH indole from N—CH₂—CH₂-indole).

¹³C NMR (75 MHz, DMSO-d⁶, 300° K):

δ(ppm) 22.7 (C—CH₂—CH₂-indole), 23.6 (CH₃ Aib), 23.8 (CH₃ Aib), 25.4 (C—CH₂—CH₂-indole), 26.0 (N—CH₂—CH₂-indole), 29.6 (CH₂ βTrp), 43.9 (N—CH₂—CH₂-indole), 46.0 (CH αTrp), 56.8 (Cq Aib), 109.5 (C₃ indole from N—CH₂—CH₂-indole), 109.9 (C₃ Trp), 111.7 (C₇ Trp), 111.9 (C₇ indole from N—CH₂—CH₂-indole and C₇ indole from C—CH₂—CH₂-indole), 113.5 (C₃ indole from C—CH₂—CH₂-indole), 118.3 (C₄ indole from N—CH₂—CH₂-indole), 118.4 (C₄ Trp), 118.5 (C₅ indole from C—CH₂—CH₂-indole), 118.7 (C₄ indole from C—CH₂—CH₂-indole), 118.9 (C₅ Trp), 119.0 (C₅ indole from N—CH₂—CH₂-indole), 121.3 (C₆ Trp), 121.5 (C₆ indole from C—CH₂—CH₂-indole and C₆ indole from N—CH₂—CH₂-indole), 122.8 (C₂ Trp, C₂ indole from C—CH₂—CH₂-indole and indole from N—CH₂—CH₂-indole), 127.1 (C₉ Trp), 127.2 (C₉ indole from C—CH₂—CH₂-indole), 127.4 (C₉ indole from N—CH₂—CH₂-indole), 136.5 (C₈ Trp and C₈ indole from C—CH₂—CH₂-indole), 136.6 (C₈ indole from N—CH₂—CH₂-indole), 154.5 (Cq triazole), 154.8 (Cq triazole), 171.8 (CO amide).

### (R)-N-(1-(4-(3-methoxybenzyl)-5-phenethyl-4H-1,2,4-triazol-3-yl)-2-(1H-indol-3-yl)ethyl)-2-amino-2-methylpropanamide (Compound 19):

¹H NMR (300 MHz, DMSO-d⁶, 300° K):

δ(ppm) 1.24 (3H, s, CH₃ Aib), 1.27 (3H, s, CH₃ Aib), 2.82 (4H, m, CH₂—CH₂-phenyl), 3.32 (2H, m, CH₂ βTrp), 3.63 (3H, s, OCH₃), 5.08 (2H, m, CH2-m-methoxybenzyl), 5.18 (1H, m, CH αTrp), 6.35 (1H, d, Jₒ=8 Hz, H₆ m-methoxybenzyl), 6.57 (1H, s, H₂ m-methoxybenzyl), 6.82 (1H, t, Jₒ=8 Hz, H₅ Trp), 6.84 (1H, d, Jₒ=8 Hz, H₄ m-methoxybenzyl), 6.99 (1H, t, Jₒ=8 Hz, H₆Trp), 7.08 (1H, m, H₄phenyl), 7.11-7.16 (5H, m, H₂ and H₄ Trp, H₂ and H₆ phenyl, H₅ m-methoxybenzyl), 7.20 (2H, m, H₃ and H₅ phenyl), 7.27 (1H, d, Jₒ=8 Hz, H₇ Trp), 8.01 (3H, brs, NH₂ Aib), 8.96 (1H, d, J=8 Hz, NH amide), 10.81 (1H, d, J=2 Hz, NH indole).

¹³C NMR (75 MHz, DMSO-d⁶, 300° K):

δ(ppm) 23.5 (CH₃ Aib), 23.8 (CH₃ Aib), 26.4 (CH₂—CH₂-phenyl), 29.1 (CH₂ βTrp), 32.7 (CH₂—CH₂-phenyl), 45.7 (CH αTrp), 45.8 (CH₂-m-methoxybenzyl), 55.5 (OCH₃), 56.7 (Cq Aib), 109.8 (C₃ Trp), 111.8 (C₇ Trp), 112.5 (C₂ m-methoxybenzyl), 113.5 (C₄ m-methoxybenzyl), 118.2 (C₄ Trp), 118.4 (C₆ m-methoxybenzyl), 118.7 (C₅ Trp), 121.3 (C₆ Trp), 124.8 (C₂ Trp), 126.5 (C₄ phenyl), 127.3 (C₉ Trp), 128.7 (C₂, C₃, C₅ and C₆ phenyl), 130.5 (C₅ m-methoxybenzyl), 136.4 (C₈ Trp), 137.7 (C₁ m-methoxybenzyl), 170.9 (C₁ phenyl), 154.6 (Cq triazole), 154.9 (Cq triazole), 160.1 (C₃ m-methoxybenzyl), 171.9 (CO amide).

### (R)-N-(1-(4-(4-methoxybenzyl)-5-phenethyl-4H-1,2,4-triazol-3-yl)-2-(1H-indol-3-yl)ethyl)-2-amino-2-methylpropanamide (Compound 20):

¹H NMR (300 MHz, DMSO-d⁶, 300° K):

δ(ppm) 1.28 (3H, s, CH₃ Aib), 1.32 (3H, s, CH₃ Aib), 2.46 (2H, m, CH₂—CH₂-phenyl), 2.82 (2H, m, CH₂—CH₂-phenyl), 3.35 (2H, d, J=7 Hz, CH₂ βTrp), 3.68 (3H, s, OCH₃), 5.02 (2H, s, CH₂-p-methoxybenzyl), 5.22 (1H, m, CH αTrp), 6.73-6.81 (4H, m, CHar p-methoxybenzyl), 6.84 (1H, t, Jₒ=7 Hz, H₅ Trp), 7.00 (1H, t, Jₒ=7 Hz, H₆ Trp), 7.05-7.11 (4H, m, H₂ and H₆ phenyl, H₂ and H₄ Trp), 7.14-7.22 (3H, m, H₃, H₄ and H₅ phenyl), 7.29 (1H, d, Jₒ=8 Hz, H₇ Trp), 8.09 (3H, brs, NH₂ Aib), 8.99 (1H, d, J=8 Hz, NH amide), 10.83 (1H, s, NH indole Trp).

¹³C NMR (75 MHz, DMSO-d⁶, 300° K):

δ(ppm) 23.5 (CH₃ Aib), 23.8 (CH₃ Aib), 26.5 (CH₂—CH₂-phenyl), 29.1 (CH₂ βTrp), 32.6 (CH₂—CH₂-phenyl), 45.5 (CH₂-p-methoxybenzyl), 45.7 (CH αTrp), 55.5 (OCH₃), 56.8 (Cq Aib), 109.7 (C₃ Trp), 111.8 (C₇ Trp), 114.6 (C₃ and C₅ p-methoxybenzyl), 118.3 (C₄ Trp), 118.7 (C₅ Trp), 121.3 (C₆ Trp), 124.9 (C₂ Trp), 126.6 (C₂ and C₆ phenyl), 127.3 (C₉ Trp), 127.6 (C₁ p-methoxybenzyl), 128.0 (C₂ and C₆ p-methoxybenzyl), 128.7 (C₃, C₄ and C₅ phenyl), 136.4 (C₈ Trp), 140.8 (C₁ phenyl), 154.5 (Cq triazole), 154.8 (Cq triazole), 159.2 (C₄ p-methoxybenzyl), 172.0 (CO Aib).

### (R)-N-(1-(4-(4-methoxybenzyl)-5-(3-phenylpropyl)-4H-1,2,4-triazol-3-yl)-2-(1H-indol-3-yl)ethyl)-2-amino-2-methylpropanamide (Compound 22):

¹H NMR (300 MHz, DMSO-d⁶, 300° K):

δ(ppm) 1.27 (3H, s, CH₃ Aib), 1.31 (3H, s, CH₃ Aib), 1.73 (2H, m, CH₂—CH₂—CH₂-phenyl), 2.47 (2H, m, CH₂—CH₂—CH₂-phenyl), 2.52 (2H, t, ³J=7 Hz, CH₂—CH₂—CH₂-phenyl), 3.35 (2H, d, J=7 Hz, CH₂ βTrp), 3.68 (3H, s, OCH₃), 4.98 (2H, s, CH₂-p-methoxybenzyl), 5.20 (1H, m, CH αTrp), 6.75 (4H, m, CHar p-methoxybenzyl), 6.82 (1H, t, Jₒ=7 Hz, H₅ Trp), 6.99 (1H, t, Jₒ=7 Hz, H₆ Trp), 7.04-7.07 (4H, m, H₂ and H₆ phenyl, H₂ and H₄ Trp), 7.13-7.24 (3H, m, H₃, H₄ and H₅ phenyl), 7.29 (1H, d, Jₒ=8 Hz, H₇ Trp), 8.03 (3H, brs, NH₂ Aib), 8.96 (1H, d, J=8 Hz, NH amide), 10.80 (1H, d, J=2 Hz, NH indole Trp).

¹³C NMR (75 MHz, DMSO-d⁶, 300° K):

δ(ppm) 23.6 (CH₃ Aib), 23.6 (CH₃ Aib), 24.06 (CH₂—CH₂—CH₂-phenyl), 28.5 (CH₂—CH₂—CH₂-phenyl),29.2 (CH₂ βTrp), 34.7 (CH₂—CH₂—CH₂-phenyl), 45.5 (CH₂-p-methoxybenzyl), 45.8 (CH αTrp), 55.5 (OCH₃), 56.8 (Cq Aib), 109.8 (C₃ Trp), 111.8 (C₇ Trp), 114.6 (C₃ and C₅ p-methoxybenzyl), 118.3 (C₄ Trp), 118.7 (C₅ Trp), 121.3 (C₆ Trp), 124.9 (C₂ Trp), 126.2 (C₂ and C₆ phenyl), 127.3 (C₉ Trp), 127.8 (C₁ p-methoxybenzyl), 127.9 (C₂ and C₆ p-methoxybenzyl), 128.7 (C₃, C₄ and C₅ phenyl), 136.4 (C₈ Trp), 141.7 (C₁ phenyl), 154.8 (Cq triazole), 159.2 (C₄ p-methoxybenzyl), 171.9 (CO Aib).

### (R)-N-(1-(4-(2-(1H-indol-3-yl)ethyl)-5-(3-(1H-indol-3-yl)propyl)-4H-1,2,4-triazol-3-yl)-2-(1H-indol-3-yl)ethyl)-2-amino-2-methylpropanamide (Compound 24):

¹H NMR (300 MHz, DMSO-d⁶, 300° K):

δ(ppm) 1.29 (3H, s, CH₃ Aib), 1.35 (3H, s, CH₃ Aib), 1.78 (2H, m, CH₂—CH₂—CH₂-indole), 2.34 (2H, m, CH₂—CH₂—CH₂-indole), 2.48 (2H, m, N—CH₂—CH₂-indole), 2.80 (2H, m, CH₂—CH₂—CH₂-indole), 3.34 (2H, m, N—CH₂—CH₂-indole), 3.94 (2H, m, CH₂ βTrp), 5.27 (1H, m, CH αTrp), 6.73-6.94 (4H, m, H₅ and H₆ Trp, H₅ indole from N—CH₂—CH₂-indole and H₅ indole from CH₂—CH₂—CH₂-indole), 6.99-7.04 (5H, m, H₂ Trp, H₂ and H₆ indole from N—CH₂—CH₂-indole, H₂ and H₆ indole from CH₂—CH₂—CH₂-indole), 7.20 (1H, d, Jₒ=8 Hz, H₄ indole from N—CH₂—CH₂-indole), 7.29 (3H, m, H₄ and H₇ Trp, H₇ indole from N—CH₂—CH₂-indole), 7.40 (1H, d, Jₒ=8 Hz, H₇ indole from CH₂—CH₂—CH₂-indole), 7.44 (1H, d, Jₒ=8 Hz, H₄ indole from CH₂—CH₂—CH₂-indole), 8.05 (3H, brs, NH₂ Aib), 9.07 (1H, d, J=8 Hz, NH amide), 10.75 (1H, s, NH indole from CH₂—CH₂—CH₂-indole), 10.86 (1H, s, NH indole Trp), 10.90 (1H, s, NH indole from N—CH₂—CH₂-indole).

¹³C NMR (75 MHz, DMSO-d⁶, 300° K):

δ(ppm) 23.6 (CH₃ Aib), 23.8 (CH₃ Aib), 24.5 (CH₂—CH₂—CH₂-indole), 25.8 (CH₂—CH₂—CH₂-indole), 27.2 (CH₂—CH₂—CH₂-indole), 29.4 (CH₂ βTrp), 44.1 (N—CH₂—CH₂-indole), 46.0 (CH αTrp), 52.9 (N—CH₂—CH₂-indole), 56.8 (Cq Aib), 109.7 (C₃ Trp and C₃ indole from N—CH₂—CH₂-indole), 111.8 (C₇ Trp), 111.9 (C₇ indole from N—CH₂—CH₂-indole and C₇ indole from CH₂—CH₂—CH₂-indole), 114.0 (C₃ indole from CH₂—CH₂—CH₂-indole), 118.2 (C₄ indole from N—CH₂—CH₂-indole), 118.3 (C₄ Trp), 118.5 (C₅ indole from CH₂—CH₂—CH₂-indole), 118.6 (C₄ indole from CH₂—CH₂—CH₂-indole), 118.9 (C₅ Trp), 119.0 (C₅ indole from N—CH₂—CH₂-indole), 121.3 (C₆ Trp), 121.4 (C₆ indole from CH₂—CH₂—CH₂-indole), 121.6 (C₆ indole from N—CH₂—CH₂-indole), 122.7 (C₂ Trp, C₂ indole from N—CH₂—CH₂-indole and C₂ indole from CH₂—CH₂—CH₂-indole), 127.1 (C₉ Trp), 127.4 (C₉ indole from N—CH₂—CH₂-indole and C₉ indole from CH₂—CH₂—CH₂-indole), 136.4 (C₈ Trp), 136.5 (C₈ indole from CH₂—CH₂—CH₂-indole), 136.7 (C₈ indole from N—CH₂—CH₂-indole), 154.7 (2 Cq triazole), 171.9 (CO amide).

### (R)-N-(1-(4-(2-methoxybenzyl)-5-phenethyl-4H-1,2,4-triazol-3-yl)-2-(1H-indol-3-yl)ethyl)-2-amino-2-methylpropanamide (Compound 26):

¹H NMR (300 MHz, DMSO-d⁶, 300° K):

δ(ppm) 1.26 (3H, s, CH₃ Aib), 1.29 (3H, s, CH₃ Aib), 2.78-2.92 (4H, m, CH₂—CH₂-phenyl), 3.29 (2H, m, CH₂ βTrp), 3.65 (3H, s, OCH₃), 4.97-5.21 (3H, m, CH αTrp and CH₂-o-methoxybenzyl), 6.52 (1H, d, Jₒ=7 Hz, H₃ o-methoxybenzyl), 6.78 (1H, t, Jₒ=7 Hz, H₅ Trp), 6.82 (1H, t, Jₒ=8 Hz, H₆ Trp), 6.84-7.04 (3H, m, H₄, H₅ and H₆ o-methoxybenzyl), 7.15 (1H, d, Jₒ=7 Hz, H₄ Trp), 7.19-7.29 (4H, m, H₃, H₄ and H₅ phenyl, H₇ Trp), 8.03 (3H, brs, NH₂ Aib), 8.94 (1H, d, J=8 Hz, NH amide), 10.82 (1H, s, NH indole Trp).

¹³C NMR (75 MHz, DMSO-d⁶, 300° K):

δ(ppm) 23.6 (CH₃ Aib), 23.7 (CH₃ Aib), 26.3 (CH₂—CH₂-phenyl), 29.0 (CH₂ βTrp), 32.5 (CH₂—CH₂-phenyl), 42.3 (CH₂-o-methoxybenzyl), 45.7 (CH αTrp), 55.8 (OCH₃), 56.7 (Cq Aib), 109.7 (C₃ Trp), 111.5 (C₇ Trp), 111.8 (C₃ o-methoxybenzyl), 118.2 (C₄ Trp), 118.7 (C₅ Trp), 121.0 (C₆ Trp), 121.3 (C₅ o-methoxybenzyl), 123.2 (C₁ o-methoxybenzyl), 124.9 (C₂ Trp), 126.6 (C₂ and C₆ phenyl), 127.2 (C₉ Trp and C₄ o-methoxybenzyl), 128.7 (C₃, C₄ and C₅ phenyl), 129.9 (C₆ o-methoxybenzyl), 136.4 (C₈ Trp), 140.6 (C₁ phenyl), 154.8 (Cq triazole), 155.2 (Cq triazole), 156.7 (C₂ o-methoxybenzyl), 171.9 (CO Aib).

### (R)-N-(2-(1H-indol-3-yl)-1-(4-(naphthalen-1-ylmethyl)-5-phenethyl-4H-1,2,4-triazol-3-yl)ethyl)-2-amino-2-methylpropanamide (Compound 27):

¹H NMR (300 MHz, DMSO-d⁶, 300° K):

δ(ppm) 1.21 (3H, s, CH₃ Aib), 1.25 (3H, s, CH₃ Aib), 2.46 (2H, m, CH₂—CH₂-phenyl), 2.88 (2H, m, CH₂—CH₂-phenyl), 3.26 (2H, dd, ³J=14 Hz and 6 Hz, CH₂ βTrp), 3.36 (2H, dd, ³J=14 Hz and 9 Hz, CH₂ βTrp), 4.99 (1H, m, CH αTrp), 5.65 (1H, d, ³J=18 Hz, CH₂-naphtyl), 5.78 (1H, d, ³J=18 Hz, CH₂-naphtyl), 6.29 (1H, d, Jₒ=7 Hz, H₂ naphtyl), 6.45 (1H, t, Jₒ=7 Hz, H₅ Trp), 6.62 (1H, d, Jₒ=8 Hz, H₄ Trp), 6.88 (1H, t, Jₒ=8 Hz, H₆ Trp), 7.04-7.06 (4H, m, H₂ and H₇ Trp, H₂ and H₆ phenyl), 7.07-7.25 (H₃ naphtyl, H₃, H₄ and H₅ phenyl), 7.57-7.60 (2H, m, H₆ and H₇ naphtyl), 7.86 (1H, d, Jₒ=8 Hz, H₄ naphtyl), 7.98-8.00 (4H, m, H₅ and H₈ naphtyl, NH₂ Aib), 8.96 (1H, d, J=8 Hz, NH amide), 10.77 (1H, s, NH indole Trp).

¹³C NMR (75 MHz, DMSO-d⁶, 300° K):

δ(ppm) 23.5 (CH₃ Aib), 23.6 (CH₃ Aib), 26.3 (CH₂CH₂-phenyl), 29.2 (CH₂ βTrp), 32.6 (CH₂—CH₂-phenyl), 43.8 (CH₂-naphtyl), 45.6 (CH αTrp), 56.7 (Cq Aib), 109.7 (C₃ Trp), 111.7 (C₇ Trp), 117.9 (C₄ Trp), 118.4 (C₅ Trp), 121.1 (C₆ Trp), 122.1 (C₂ naphtyl), 123.0 (C₈ naphtyl), 124.9 (C₂ Trp), 125.9 (C₃ naphtyl), 126.5 (C₆ naphtyl), 126.9 (C₂ and C₆ phenyl), 127.0 (C₉ Trp and C₇ naphtyl), 127.1 (C₄ naphtyl), 128.4 (C₅ naphtyl), 128.7 (C₃, C₄ and C₅ phenyl), 130.0 (C₉ naphtyl), 131.7 (C₁ naphtyl), 133.6 (C₁₀ naphtyl), 136.4 (C₈ Trp), 140.8 (C₁ phenyl), 154.8 (Cq triazole), 155.3 (Cq triazole), 171.9 (CO Aib).

(R)-N-(1-(5-(2-(1H-indol-3-yl)ethyl)-4-(3,4-dichlorobenzyl)-4H-1,2,4-triazol-3-yl)-2-(1H-indol-3-yl)ethyl)-2-amino-2-methylpropanamide (Compound 28):

¹H NMR (300 MHz, DMSO-d⁶, 300° K):

δ(ppm) 1.26 (6H, s, CH₃ Aib), 2.87 (2H, m, CH₂—CH₂-indole), 2.96 (2H, m, CH₂—CH₂-indole), 3.32 (2H, m, CH₂ βTrp), 5.13 (3H, m, CH αTrp and CH₂-m,p-dichlorobenzyl), 6.58 (1H, d, Jₒ=8 Hz, H₆ m,p-dichlorobenzyl), 6.85 (1H, t, Jₒ=7 Hz, H₅ Trp), 6.96 (1H, t, Jₒ=7 Hz, H₅ indole), 7.01 (2H, m, H₆ indole and H₆ Trp), 7.04 (1H, s, H₂ Trp), 7.08 (1H, s, H₂ indole), 7.13 (1H, d, Jₒ=8 Hz, H₅ m,p-dichlorobenzyl), 7.20-7.30 (4H, m, H₄ and H₇ indole, H₇ Trp and H₂ m,p-dichlorobenzyl), 7.36 (1H, d, Jₒ=8 Hz, H₄ Trp), 8.08 (3H, brs, NH₂ Aib), 8.98 (1H, d, J=8 Hz, NH amide), 10.80 (1H, s, NH indole), 10.82 (1H, s, NH indole Trp).

¹³C NMR (75 MHz, DMSO-d⁶, 300° K):

δ(ppm) 22.8 (CH₂—CH₂-indole), 23.4 (CH₃ Aib), 23.8 (CH₃ Aib), 25.8 (CH₂—CH₂-indole), 29.0 (CH₂ βTrp), 44.8 (CH₂-m,p-dichlorobenzyl), 45.6 (CH αTrp), 56.8 (Cq Aib), 109.7 (C₃ indole), 111.8 (C₇ indole and C₇ Trp), 118.1 (C₄ Trp), 118.4 (C₅ indole), 118.6 (C₄ indole and C₅ Trp), 121.3 (C₆ indole and C₆ Trp), 123.0 (C₂ indole and C₂ Trp), 126.4 (C₆ m,p-dichlorobenzyl), 127.1 (C₉ Trp), 127.3 (C₉ indole), 128.6 (C₂ m,p-dichlorobenzyl), 130.9 (C₄ m,p-dichlorobenzyl), 131.3 (C₅ m,p-dichlorobenzyl), 132.0 (C₃ m,p-dichlorobenzyl), 136.4 (C₈ Trp), 136.6 (C₈ indole), 137.2 (C₁ m,p-dichlorobenzyl), 154.7 (Cq triazole), 155.1 (Cq triazole), 172.0 (CO Aib).

### (R)-N-(1-(4-(4-fluorobenzyl)-5-benzyl-4H-1,2,4-triazol-3-yl)-2-(1H-indol-3-yl)ethyl)-2-amino-2-methylpropanamide (Compound 30):

¹H NMR (300 MHz, DMSO-d⁶, 300° K):

δ(ppm) 1.27 (3H, s, CH₃ Aib), 1.29 (3H, s, CH₃ Aib), 3.33 (2H, m, CH₂ βTrp), 4.02 (2H, s, CH₂-benzyl), 5.10 (3H, m, CH₂-p-fluorobenzyl and CH αTrp), 6.71 (2H, m, H₃ and H₅ p-fluorobenzyl), 6.80 (1H, t, Jₒ=8 Hz, H₅ Trp), 6.90 (2H, d, Jₒ=8 Hz, H₂ and H₆ p-fluorobenzyl), 6.94 (1H, t, Jₒ=8 Hz, H₆ Trp), 6.99-7.10 (4H, m, H₂ and H₄ Trp, H₂ and H₆ benzyl), 7.20 (3H, m, H₃, H₄ and H₅ benzyl), 7.27 (1H, d, Jₒ=8 Hz, H₇ Trp), 8.09 (3H, brs, NH₂ Aib), 8.97 (1H, d, J=8 Hz, NH amide), 10.79 (1H, s, NH indole Trp).

¹³C NMR (75 MHz, DMSO-d⁶, 300° K):

δ(ppm) 23.5 (CH₃ Aib), 23.8 (CH₃ Aib), 29.0 (CH₂ βTrp), 31.1 (CH₂-benzyl), 45.7 (CH₂-p-fluorobenzyl), 45.8 (CH αTrp), 56.8 (Cq Aib), 109.6 (C₃ Trp), 111.8 (C₇ Trp), 115.6 and 115.9 (C₃ and C₅ p-fluorobenzyl), 118.2 (C₄ Trp), 118.7 (C₅ Trp), 121.3 (C₆ Trp), 124.8 (C₂ Trp), 127.1 (C₄ benzyl), 127.2 (C₉ Trp), 128.8 and 128.9 (C₂ and C₆ p-fluorobenzyl), 129.4 (C₂, C₃, C₅ and C₆ p-fluorobenzyl), 131.6 (C₁ p-fluorobenzyl), 135.9 (C₁ benzyl), 136.4 (C₈ Trp), 154.0 (C₄ p-fluorobenzyl), 155.3 (Cq triazole), 172.0 (CO amide).

### (R)-N-(1-(4-(4-methylbenzyl)-5-(3-phenylpropyl)-4H-1,2,4-triazol-3-yl)-2-(1H-indol-3-yl)ethyl)-2-amino-2-methylpropanamide (Compound 33):

¹H NMR (300 MHz, DMSO-d⁶, 300° K):

δ(ppm) 1.25 (3H, s, CH₃ Aib), 1.28 (3H, s, CH₃ Aib), 1.73 (2H, m, CH₂—CH₂—CH₂-phenyl), 2.23 (3H, s, CH₃ p-methylbenzyl), 2.49-2.54 (4H, m, CH₂—CH₂—CH₂-phenyl), 3.33 (2H, m, CH₂ βTrp), 5.04 (2H, s, CH₂-p-methylbenzyl), 5.16 (1H, m, CH αTrp), 6.74 (2H, d, Jₒ=8 Hz, H₃ and H₅ p-methylbenzyl), 6.80 (1H, t, Jₒ=7 Hz, H₅ Trp), 6.98 (1H, t, Jₒ=7 Hz, H₆ Trp), 7.03 (1H, d, J=2 Hz, H₂ Trp), 7.06 (5H, m, CHar phenyl), 7.14 (1H, d, Jₒ=7 Hz, H₄ Trp), 7.20 (2H, d, Jₒ=7 Hz, H₂ and H₆ p-methylbenzyl), 7.27 (1H, d, Jₒ=8 Hz, H₇ Trp), 8.01 (3H, brs, NH₂ Aib), 8.95 (1H, d, J=8 Hz, NH amide), 10.80 (1H, d, J=2 Hz, NH indole Trp).

¹³C NMR (75 MHz, DMSO-d⁶, 300° K):

δ(ppm) 21.0 (CH₃ p-methylbenzyl), 23.5 (CH₃ Aib), 23.8 (CH₃ Aib), 24.0 (CH₂—CH₂—CH₂-phenyl), 28.5 (CH₂—CH₂—CH₂-phenyl), 29.1 (CH₂ βTrp), 34.7 (CH₂—CH₂—CH₂-phenyl), 45.7 (CH αTrp), 45.8 (CH₂-p-methylbenzyl), 56.8 (Cq Aib), 109.8 (C₃ Trp), 111.8 (C₇ Trp), 118.3 (C₄ Trp), 118.7 (C₅ Trp), 121.3 (C₆ Trp), 124.9 (C₂ Trp), 126.2 (C₄ phenyl), 126.4 (C₃ and C₅ p-methylbenzyl), 127.3 (C₉ Trp), 128.7 (C₂, C₃, C₅ and C₆ phenyl), 129.8 (C₂ and C₆ p-methylbenzyl), 133.0 (C₁ p-methylbenzyl), 136.4 (C₈ Trp), 137.5 (C₄ p-methylbenzyl), 141.7 (C₁ phenyl), 154.8 (Cq triazole), 171.9 (CO Aib).

### (R)-N-(1-(5-(2-(1H-indol-3-yl)ethyl)-4-(4-methylbenzyl)-4H-1,2,4-triazol-3-yl)-2-(1H-indol-3-yl)ethyl)-2-amino-2-methylpropanamide (Compound 34):

¹H NMR (300 MHz, DMSO-d⁶, 300° K):

δ(ppm) 1.25 (3H, s, CH₃ Aib), 1.28 (3H, s, CH₃ Aib), 2.23 (3H, s, CH₃-p-methylbenzyl), 2.84-2.97 (4H, m, CH₂—CH₂-indole), 3.32 (2H, m, CH₂ βTrp), 5.04 (2H, s, CH₂— p-methylbenzyl), 5.16 (1H, m, CH αTrp), 6.79-6.86 (4H, m, CH ar p-methylbenzyl), 6.99-7.05 (4H, m, H₅ and H₆ indole, H₅ and H₆ Trp), 7.08 (3H, m, H₂ indole, H₂ and H₄ Trp), 7.25-7.30 (3H, m, H₄ and H₇ indole, H₇ Trp), 8.00 (3H, brs, NH₂ Aib), 8.94 (1H, d, J=8 Hz, NH amide), 10.76 (1H, s, NH indole), 10.78 (1H, s, NH indole Trp).

¹³C NMR (75 MHz, DMSO-d⁶, 300° K):

δ(ppm) 21.0 (CH₃— p-methylbenzyl), 22.8 (CH₂—CH₂-indole), 23.8 (CH₃ Aib), 23.9 (CH₃ Aib), 25.9 (CH₂—CH₂-indole), 28.5 (CH₂ βTrp), 45.7 (CH₂— p-methylbenzyl and CH αTrp), 56.7 (Cq Aib), 109.9 (C₃ Trp), 111.8 (C₇ indole and C₇ Trp), 113.4 (C₃ indole), 118.1 (C₄ Trp), 118.3 (C₄ indole), 118.5 (C₅ indole), 118.7 (C₅ Trp), 120.9 (C₆ indole and C₆ Trp), 121.3 (C₂ indole and C₂ Trp), 126.3 (C₃ and C₅ p-methylbenzyl), 127.2 (C₉ indole), 127.3 (C₉ Trp), 129.8 (C₂ and C₆ p-methylbenzyl), 133.1 (C₁ p-methylbenzyl), 135.8 (C₈ indole, C₈ Trp), 136.4 (C₄ p-methylbenzyl), 154.8 (Cq triazole), 155.0 (Cq triazole), 171.9 (CO Aib).

### (R)-N-(1-(4-(4-methylbenzyl)-5-phenethyl-4H-1,2,4-triazol-3-yl)-2-(1H-indol-3-yl)ethyl)-2-amino-2-methylpropanamide (Compound 37):

¹H NMR (300 MHz, DMSO-d⁶, 300° K):

δ(ppm) 1.25 (3H, s, CH₃ Aib), 1.28 (3H, s, CH₃ Aib), 2.23 (3H, s, CH₃ p-methylbenzyl), 2.83 (4H, m, CH₂—CH₂-phenyl), 3.32 (2H, m, CH₂ βTrp), 5.05 (2H, s, CH₂-p-methylbenzyl), 5.18 (1H, m, CH αTrp), 6.75 (2H, d, Jₒ=8 Hz, H₃ and H₅ p-methylbenzyl), 6.82 (1H, t, Jₒ=8 Hz, H₅ Trp), 6.99 (1H, t, Jₒ=8 Hz, H₆ Trp), 7.02-7.11 (6H, m, H₂ Trp and CHar phenyl), 7.15 (1H, d, Jₒ=7 Hz, H₄ Trp), 7.20 (2H, d, Jₒ=7 Hz, H₂ and H₆ p-methylbenzyl), 7.28 (1H, d, Jₒ=8 Hz, H₇ Trp), 8.01 (3H, brs, NH₂ Aib), 8.93 (1H, d, J=8 Hz, NH amide), 10.77 (1H, s, NH indole Trp).

¹³C NMR (75 MHz, DMSO-d⁶, 300° K):

δ(ppm) 21.0 (CH₃ p-methylbenzyl), 23.6 (CH₃ Aib), 23.8 (CH₃ Aib), 26.5 (CH₂—CH₂-phenyl), 29.1 (CH₂ βTrp), 32.7 (CH₂—CH₂-phenyl), 45.7 (CH αTrp and CH₂-p-methylbenzyl), 56.8 (Cq Aib), 109.8 (C₃ Trp), 111.8 (C₇ Trp), 118.3 (C₄ Trp), 118.7 (C₅ Trp), 121.3 (C₆ Trp), 124.8 (C₂ Trp), 126.4 (C₃ and C₅ p-methylbenzyl), 126.6 (C₄ phenyl), 127.3 (C₉ Trp), 128.7 (C₂, C₃, C₅ and C₆ phenyl), 129.8 (C₂ and C₆ p-methylbenzyl), 133.0 (C₁ p-methylbenzyl), 136.4 (C₈ Trp), 137.5 (C₄ p-methylbenzyl), 140.9 (C₁ phenyl), 154.5 (Cq triazole), 154.9 (Cq triazole), 171.9 (CO amide).

### (R)-N-(1-(5-benzyl-4-(pyridin-2-ylmethyl)-4H-1,2,4-triazol-3-yl)-2-(1H-indol-3-yl)ethyl)-2-amino-2-methylpropanamide (Compound 39):

¹H NMR (300 MHz, DMSO-d⁶, 300° K):

δ(ppm) 1.23 (3H, s, CH₃ Aib), 1.27 (3H, s, CH₃ Aib), 3.34 (1H, dd, J=14 Hz and 6 Hz, CH₂ βTrp), 3.43 (1H, dd, J=14 Hz and 9 Hz, CH₂ βTrp), 4.13 (2H, s, CH₂-benzyl), 5.22 (1H, s, CH αTrp), 5.35 (2H, s, CH₂-o-pyridyl), 6.80 (1H, t, Jₒ=8 Hz, H₅ Trp), 6.92 (1H, t, Jₒ=8 Hz, H₅ pyridyl), 6.97 (1H, t, Jₒ=8 Hz, H₆ Trp), 7.04 (1H, d, Jₒ=8 Hz, H₄ Trp), 7.07 (1H, d, J=2 Hz, H₂ Trp), 7.10-7.16 (5H, m, CHar benzyl), 7.19 (1H, s, H₃ o-pyridyl), 7.26 (1H, d, Jₒ=8 Hz, H₇Trp), 7.57 (1H, t, Jₒ=9 Hz, H₄ o-pyridyl), 8.16 (3H, brs, NH₂ Aib), 8.36 (1H, d, J_{αβ}=5 Hz, H₆ o-pyridyl), 9.01 (1H, d, J=8 Hz, NH amide), 10.85 (1H, s, NH indole Trp).

¹³C NMR (75 MHz, DMSO-d⁶, 300° K):

δ(ppm) 23.4 (CH₃ Aib), 23.7 (CH₃ Aib), 28.6 (CH₂ βTrp), 30.4 (CH₂-benzyl), 45.7 (CH αTrp), 47.7 (CH₂— o-pyridyl), 56.7 (Cq Aib), 109.8 (C₃ Trp), 111.8 (C₇ Trp), 118.3 (C₄ Trp), 118.6 (C₅ Trp), 121.2 (C₆ Trp), 121.7 (C₃ o-pyridyl), 123.3 (C₅ o-pyridyl), 124.8 (C₂ Trp), 127.1 (C₄ benzyl), 127.3 (C₉ Trp), 128.8 (C₂ and C₆ benzyl), 129.0 (C₃ and C₅ benzyl), 135.6 (C₁ benzyl), 136.4 (C₈ Trp), 137.5 (C₄ o-pyridyl), 149.5 (C₆ o-pyridyl), 154.1 (Cq triazole), 154.2 (Cq triazole), 155.7 (C₂ o-pyridyl), 172.0 (CO amide).

### (R)-N-(1-(4-(4-ethylbenzyl)-5-phenethyl-4H-1,2,4-triazol-3-yl)-2-(1H-indol-3-yl)ethyl)-2-amino-2-methylpropanamide (Compound 43):

¹H NMR (300 MHz, DMSO-d⁶, 300° K):

δ(ppm) 1.10 (3H, t, J=8 Hz, CH₃—CH₂ p- ethylbenzyl), 1.25 (3H, s, CH₃ Aib), 1.28 (3H, s, CH₃ Aib), 2.53 (2H, q, J=8 Hz, CH₃—CH₂ p-ethylbenzyl), 2.83 (4H, m, CH₂—CH₂-phenyl), 3.34 (2H, m, CH₂ βTrp), 5.07 (2H, s, CH₂-p-ethylbenzyl), 5.19 (1H, m, CH αTrp), 6.77 (2H, d, Jₒ=8 Hz, H₃ and H₅ p-ethylbenzyl), 6.81 (1H, t, Jₒ=7 Hz, H₅ Trp), 7.13 (1H, d, J=2 Hz, H₂ Trp), 7.20 (1H, d, Jₒ=7 Hz, H₄ Trp), 7.28 (1H, d, Jₒ=8 Hz, H₇ Trp), 8.03 (3H, brs, NH₂ Aib), 8.94 (1H, d, J=8 Hz, NH amide), 10.79 (1H, s NH indole Trp).

¹³C NMR (75 MHz, DMSO-d⁶, 300° K):

δ(ppm) 15.9 (CH₃—CH₂ p-ethylbenzyl), 23.5 (CH₃ Aib), 23.8 (CH₃ Aib), 26.5 (CH₂—CH₂-phenyl), 28.1 (CH₃—CH₂ p-ethylbenzyl), 29.1 (CH₂ βTrp), 32.7 (CH₂—CH₂-phenyl), 45.7 (CH αTrp), 45.8 (CH₂-p-ethylbenzyl), 56.8 (Cq Aib), 109.8 (C₃ Trp), 111.8 (C₇ Trp), 118.3 (C₄ Trp), 118.7 (C₅ Trp), 121.3 (C₆ Trp), 124.9 (C₂ Trp), 126.5 (C₃ and C₅ p-ethylbenzyl), 126.6 (C₄ phenyl), 127.3 (C₉ Trp), 128.6 (C₂ and C₆ p-ethylbenzyl, C₂, C₃, C₅ and C₆ phenyl), 133.1 (C₁ p-ethylbenzyl), 136.5 (C₈ Trp), 140.8 (C₁ phenyl), 143.8 (C₄ p-ethylbenzyl), 154.6 (Cq triazole), 154.9 (Cq triazole), 171.9 (CO amide).

### (R)-N-(1-(4-(4-methoxybenzyl)-5-phenethyl-4H-1,2,4-triazol-3-yl)-2-(1H-indol-3-yl)ethyl)piperidine-4-carboxamide (Compound 44):

¹H NMR (300 MHz, DMSO d⁶, 300° K):

δ(ppm) 1.42 (m, 2H, H₃ and H₅ piperidyl ), 1.55 (m, 1H, H₅ piperidyl ), 2.23 (m, 1H, H₄ piperidyl ), 2.75 (m, 5H, H₂ piperidyl and CH₂—CH₂-phenyl), 3.04 (m, 1H, H₆ piperidyl), 3.13 (m, 1H, H₂ piperidyl ), 3.32 (m, 2H, CH₂ βTrp), 3.66 (s, 3H, OCH₃), 4.97 (m, 2H, CH₂-p-methoxybenzyl), 5.23 (m, 1H, CH αTrp), 6.70 (s, 4H, CHar p-methoxybenzyl), 6.87 (t, 1H, Jₒ=8 Hz, H₅ Trp), 7.00 (m, 2H, H₂ and H₆ Trp), 7.07 (d, 2H, Jₒ=8 Hz, H₂ and H₆ phenyl), 7.14 (d, 1H, Jₒ=7 Hz, H₄ Trp), 7.18-7.30 (m, 4H, H₇ Trp, H₃, H₄ and H₅ phenyl), 8.16 and 8.46 (2 m, 2H, NH piperidyl TFA salt), 8.66 (d, 1H, J=8 Hz, NH amide), 10.75 (1H, s, NH indole Trp).

¹³C NMR (75 MHz, DMSO d⁶, 300° K):

δ(ppm) 24.9 (C₃ piperidyl ), 25.4 (C₅ piperidyl ), 26.5 (CH₂—CH₂-phenyl), 29.2 (CH₂ βTrp), 32.7 (CH₂—CH₂-phenyl), 38.7 (C₄ piperidyl ), 42.7 (C₂ and C₆ piperidyl ), 44.7 (CH Trp), 45.3 (CH₂-p-methoxybenzyl), 55.5 (OCH₃), 110.2 (C₃ Trp), 111.7 (C₇ Trp), 114.4 (C₃ and C₅ p-methoxybenzyl), 118.5 (C₄ Trp), 118.7 (C₅ Trp), 121.3 (C₆ Trp), 124.4 (C₂ Trp), 126.5 (C₂ and C₆ phenyl), 127.5 (C₉ Trp), 127.8 (C₁, C₂ and C₆ p-methoxybenzyl), 128.7 (C₃, C₄ and C₅ phenyl), 136.4 (C₈ Trp), 140.8 (C₁ phenyl), 155.3 (Cq triazole), 155.4 (Cq triazole), 159.1 (C₄ p-methoxybenzyl), 173.1 (CO amide).

### (R)-N-(1-(5-(2-(1H-indol-3-yl)ethyl)-4-(4-methoxybenzyl)-4H-1,2,4-triazol-3-yl)-2-indol-3-yl)ethyl)piperidine-4-carboxamide (Compound 45):

¹H NMR (300 MHz, DMSO d⁶, 300° K):

δ(ppm) 1.41 (m, 2H, H₃ and H₅ piperidyl ), 1.54 (dd, 1H, J=13 Hz and 2 Hz, H₅ piperidyl ), 2.23 (m, 1H, H₄ piperidyl ), 2.72 (m, 2H, H₂ and H₆ piperidyl ), 2.77-2.93 (m, 4H, CH₂—CH₂-indole), 3.06 (m, 2H, H₂ and H₆ piperidyl ), 3.32 (m, 2H, CH₂ βTrp), 3.65 (s, 3H, OCH₃), 4.94 (s, 2H, CH₂-p-methoxybenzyl), 5.22 (m, 1H, CH αTrp), 6.68 (s, 4H, CHar p-methoxybenzyl), 6.87 (m, 3H, H₅ and H₆ Trp, H₅ indole), 6.98 (m, 4H, H₂ and H₆ indole, H₂ and H₄ Trp), 7.20-7.33 (m, 3H, H₄ and H₇ indole, H₇ Trp), 8.15 and 8.46 (2 m, 2H, NH piperidyl TFA salt), 8.64 (d, 1H, J=8 Hz, NH amide), 10.74 (s, 2H, NH indole and NH indole Trp).

¹³C NMR (75 MHz, DMSO d⁶, 300° K):

δ(ppm) 22.9 (CH₂—CH₂-indole), 24.9 (C₃ piperidyl), 25.4 (C₅ piperidyl), 26.0 (CH₂—CH₂-indole), 29.3 (CH₂ βTrp), 39.1 (C₄ piperidyl), 42.7 (C₂ and C₆ piperidyl ), 44.7 (CH αTrp), 45.3 (CH₂-p-methoxybenzyl), 55.5 (OCH₃), 109.5 (C₃ Trp), 111.7 (C₇ indole and C₇ Trp), 113.5 (C₃ indole), 114.4 (C₃ and C₅ p-methoxybenzyl), 118.5 (C₄ indole and C₄ Trp), 118.6 (C₅ indole and C₅ Trp), 121.2 (C₆ indole), 121.3 (C₆ Trp), 122.9 (C₂ indole and C₂ Trp), 127.2 (C₉ indole), 127.6 (C₉ Trp, C₂ and C₆ p-methoxybenzyl), 127.9 (C₁ p-methoxybenzyl), 136.4 (C₈ Trp), 136.6 (C₈ indole), 154.9 (Cq triazole), 155.2 (Cq triazole), 159.0 (C₄ p-methoxybenzyl), 173.0 (CO amide).

### (R)-N-(1-(4-(4-methoxybenzyl)-5-phenethyl-4H-1,2,4-triazol-3-yl)-2-(1H-indol-3-yl)ethyl)-2-aminoacetamide (Compound 50):

¹H NMR (300 MHz, DMSO d⁶, 300° K):

δ(ppm) 2.78 (m, 4H, CH₂—CH₂-phenyl), 3.26 (1H, dd, J=14 Hz and 7 Hz, CH₂ βTrp), 3.39 (m, 3H, CH₂ βTrp and CH₂—NH₂), 3.65 (s, 3H, OCH₃), 4.95 (m, 2H, CH₂-p-methoxybenzyl), 5.20 (m, 1H, CH αTrp), 6.63 (s, 4H, CHar p-methoxybenzyl), 6.86 (t, 1H, Jₒ=7 Hz, H₅ Trp), 6.99 (s, 1H, H₂ Trp), 7.02 (t, 1H, Jₒ=7 Hz, H₆ Trp), 7.10 (m, 2H, H₂ and H₆ phenyl), 7.15 (d, 1H, Jₒ=7 Hz, H₄ Trp), 7.23 (m, 3H, H₃, H₄ and H₅ Trp), 7.31 (d, 1H, Jₒ=8 Hz, H₇ Trp), 7.95 (brs, 3H, NH₂ Gly, TFA salt), 9.20 (d, 1H, J=8 Hz, NH amide), 10.82 (s, 1H, NH indole Trp).

¹³C NMR (75 MHz, DMSO d⁶, 300° K):

δ(ppm) 26.5 (CH₂—CH₂-phenyl), 29.8 (CH₂ βTrp), 32.7 (CH₂—CH₂-phenyl), 39.0 (CH₂—NH₂), 45.3 (CH₂— p-methoxybenzyl), 45.4 (CH αTrp), 55.4 (OCH₃), 109.7 (C₃ Trp), 111.8 (C₇ Trp), 114.5 (C₃ and C₅ p-methoxybenzyl), 118.3 (C₄ Trp), 118.9 (C₅ Trp), 121.4 (C₆ Trp), 124.6 (C₂ Trp), 126.5 (C₂ and C₆ phenyl), 127.3 (C₉ Trp), 127.7 (C₁ p-methoxybenzyl), 127.8 (C₂ and C₆ p-methoxybenzyl), 128.7 (C₃, C₄ and C₅ phenyl), 136.4 (C₈ Trp), 140.9 (C₁ phenyl), 154.3 (Cq triazole), 154.8 (Cq triazole), 159.0 (C₄ p-methoxybenzyl), 166.1 (CO amide).

### (R)-N-(1-(4-(4-methoxybenzyl)-5-phenethyl-4H-1,2,4-triazol-3-yl)-2-(1H-indol-3-yl)ethyl)-2-(pyridin-2-yl)acetamide (Compound 51):

¹H NMR (300 MHz, DMSO d⁶, 300° K):

δ(ppm) 2.77-2.88 (m, 4H, CH₂—CH₂-phenyl), 3.37 (m, 2H, CH₂ βTrp), 3.64 (s, 3H, OCH₃), 3.74 (m, 2H, CH₂-o-pyridyl), 5.03 (m, 2H, CH₂-p-methoxybenzyl), 5.24 (m, 1H, CH αTrp), 6.65 (s, 4H, CHar p-methoxybenzyl), 6.85 (t, 1H, Jₒ=7 Hz, H₅ Trp), 7.01 (m, 2H, H₂ and H₆ Trp), 7.08 (d, 2H, Jₒ=7 Hz, H₂ and H₆ phenyl), 7.15 (d, 1H, Jₒ=7 Hz, H₄ Trp), 7.21 (m, 3H, H₃, H₄ and H₅ phenyl), 7.27-7.36 (m, 2H, H₇ Trp and H₃ o-pyridyl), 7.58 (t, 1H, J=6 Hz, H₅ o-pyridyl), 8.04 (t, 1H, Jₒ=8 Hz, H₄ o-pyridyl), 8.62 (d, 1H, J_{αβ}=5 Hz, H₆ o-pyridyl), 9.17 (d, 1H, J=8 Hz, NH amide), 10.81 (s, 1H, NH indole Trp).

¹³C NMR (75 MHz, DMSO d⁶, 300° K):

δ(ppm) 26.4 (CH₂—CH₂-phenyl), 29.2 (CH₂ βTrp), 32.4 (CH₂—CH₂-phenyl), 41.7 (CH₂-o-pyridyl), 45.3 (CH αTrp), 45.7 (CH₂-p-methoxybenzyl), 55.5 (OCH₃), 109.7 (C₃ Trp), 111.8 (C₇ Trp), 114.4 (C₃ and C₅ p-methoxybenzyl), 118.4 (C₄ Trp), 118.8 (C₅ Trp), 121.4 (C₆ Trp), 124.1 (C₃ o-pyridyl), 124.6 (C₂ Trp), 126.4 (C₅ o-pyridyl), 126.6 (C₂ and C₆ phenyl), 127.2 (C₉ Trp), 127.4 (C₁ p-methoxybenzyl), 127.9 (C₂ and C₆ p-methoxybenzyl), 128.7 (C₃, C₄ and C₅ phenyl), 136.4 (C₈ Trp), 140.5 (C₁ phenyl), 142.1 (C₄ o-pyridyl), 145.3 (C₆ o-pyridyl), 153.0 (C₂ o-pyridyl), 154.5 (Cq triazole), 155.3 (Cq triazole), 159.1 (C₄ p-methoxybenzyl), 167.9 (CO amide).

### (R)-N-(1-(4-(2,4-dimethoxybenzyl)-5-phenethyl-4H-1,2,4-triazol-3-yl)-2-(1H-indol-3-yl)ethyl)picolinamide (Compound 64):

¹H NMR (300 MHz, DMSO d⁶, 300° K):

δ(ppm) 2.83 (m, 2H, CH2—CH₂-phenyl), 2.90 (m, 2H, CH₂—CH₂-phenyl), 3.48 (m, 2H, CH₂ βTrp), 3.57 (s, 3H, OCH₃), 3.61 (s, 3H, OCH₃), 4.97 (d, 1H, J=17 Hz, CH₂-o,p-dimethoxybenzyl), 5.09 (d, 1H, J=17 Hz, CH₂-o,p-dimethoxybenzyl), 5.56 (m, 1H, CH αTrp), 6.18 (dd, 1H, Jₒ=8 Hz and Jₘ=2 Hz, H₅ o,p-dimethoxybenzyl), 6.41 (d, 1H, Jₘ=2 Hz, H₃ o,p-dimethoxybenzyl), 6.55 (d, 1H, Jₒ=8 Hz, H₆ o,p-dimethoxybenzyl), 6.87 (t, 1H, Jₒ=8 Hz, H₅ Trp), 7.01 (t, 1H, Jₒ=8 Hz, H₆ Trp), 7.08 (m, 3H, H₂ Trp, H₂ and H₆ phenyl), 7.14 (d, 1H, Jₒ=7 Hz, H₄ Trp), 7.19-7.31 (m, 4H, H₇ Trp, H₃, H₄ and H₅ phenyl), 7.56 (t, 1H, J=8 Hz, NH amide), 7.91 (m, 2H, H₄ and H₅ o-pyridyl), 8.57 (d, 1H, J_{αβ}=5 Hz, H₆ o-pyridyl), 9.16 (d, 1H, Jₒ=8 Hz, H₃ o-pyridyl), 10.80 (s, 1H, NH indole Trp).

¹³C NMR (75 MHz, DMSO d⁶, 300° K):

δ(ppm) 26.2 (CH₂—CH₂-phenyl), 28.8 (CH₂ βTrp), 32.1 (CH₂—CH₂-phenyl), 43.0 (CH₂-o,p-dimethoxybenzyl), 45.5 (CH αTrp), 55.6 (OCH₃), 55.8 (OCH₃), 98.9 (C₃ o,p-dimethoxybenzyl), 105.0 (C₅ o,p-dimethoxybenzyl), 109.5 (C₃ Trp), 111.8 (C₇ Trp), 114.4 (C₁ o,p-dimethoxybenzyl), 118.4 (C₄ Trp), 118.8 (C₅ Trp), 121.4 (C₆ Trp), 122.4 (C₃ o-pyridyle), 124.4 (C₂ Trp), 126.7 (C₆ o,p-dimethoxybenzyl), 127.2 (C₅ o-pyridyle), 127.5 (C₉ Trp), 128.6-128.8 (C₂, C₃, C₄, C₅ and C₆ phenyl), 136.4 (C₈ Trp), 138.1 (C₄ o-pyridyle), 140.2 (C₁ phenyl), 148.8 (C₆ o-pyridyle), 149.3 (C₂ o-pyridyle), 155.2 (Cq triazole), 155.4 (Cq triazole), 157.9 (C₂ o,p-dimethoxybenzyl), 161.0 (C₄ o,p-dimethoxybenzyl), 163.9 (CO amide).

### (R)-N-(1-(4-(2,4-dimethoxybenzyl)-5-phenethyl-4H-1,2,4-triazol-3-yl)-2-(1H-indol-3-yl)ethyl)pyrazine-2-carboxamide (Compound 66):

¹H NMR (300 MHz, DMSO d⁶, 300° K):

δ(ppm) 2.87 (m, 4H, CH₂—CH₂-phenyl), 3.51 (m, 2H, CH₂ βTrp), 3.58 (s, 3H, OCH₃), 3.59 (s, 3H, OCH₃), 4.97 (d, 1H, J=17 Hz, CH₂-o,p-dimethoxybenzyl), 5.08 (d, 1H, J=17 Hz, CH₂-o,p-dimethoxybenzyl), 5.56 (s, 1H, CH αTrp), 6.10 (dd, 1H, Jₒ=8 Hz and Jₘ₌2 Hz, H₅ o,p-dimethoxybenzyl), 6.36 (d, 1H, Jm=2 Hz, H₃ o,p-dimethoxybenzyl), 6.40 (d, 1H, Jₒ=8 Hz, H₆ o,p-dimethoxybenzyl), 6.87 (t, 1H, Jₒ=8 Hz, H₅ Trp), 7.00 (t, 1H, Jₒ=7 Hz, H₆ Trp), 7.09 (m, 3H, H₂ Trp, H₂ and H₆ phenyl), 7.15 (d, 1H, Jₒ=7 Hz, Trp), 7.19-7.28 (m, 3H, H₃, H₄ and H₅ phenyl), 7.36 (d, 1H, Jₒ=8 Hz, H₇ Trp), 8.61 (t, 1H, J=2 Hz, H₃ o-pyrazinyl), 8.78 (d, 1H, J=2 Hz, H₅ o-pyrazinyl), 8.94 (d, 1H, J=1 Hz, H₆ o-pyrazinyl), 9.26 (d, 1H, J=8 Hz, NH amide), 10.78 (s, 1H, NH indole Trp).

¹³C NMR (75 MHz, DMSO d⁶, 300° K):

δ(ppm) 26.1 (CH₂—CH₂-phenyl), 28.4 (CH₂ βTrp), 32.1 (CH₂—CH₂-phenyl), 42.9 (CH₂-o,p-dimethoxybenzyl), 45.5 (CH αTrp), 55.5 (OCH₃), 55.8 (OCH₃), 98.7 (C₃ o,p-dimethoxybenzyl), 104.9 (C₅ o,p-dimethoxybenzyl), 109.7 (C₃ o,p-dimethoxybenzyl), 111.8 (C₇ Trp), 114.5 (C₁ o,p-dimethoxybenzyl), 118.5 (C₄ Trp), 118.8 (C₅ Trp), 121.4 (C₆ Trp), 124.4 (C₂ Trp), 126.7 (C₆ o,p-dimethoxybenzyl), 127.5 (C₉ Trp), 128.1 (C₄ phenyl), 128.7-128.8 (C₂, C₃, C₅ and C₆ phenyl), 136.4 (C₈ Trp), 140.3 (C₁ phenyl), 141.2 (C₆ o,p-dimethoxybenzyl), 144.2 (C₂ o-pyrazinyl), 144.3 (C₃ o-pyrazinyl), 146.8 (C₅ o-pyrazinyl), 155.2 (Cqs triazole), 157.7 (C₂ o,p-dimethoxybenzyl), 160.7 (CO amide), 162.9 (C₄ o,p-dimethoxybenzyl).

### (S)-N-((R)-1-(5-(2-(1H-indol-3-yl)ethyl)-4-(2,4-dimethoxybenzyl)-4H-1,2,4-triazol-3-yl)-2-(1H-indol-3-yl)ethyl)pyrrolidine-2-carboxamide (Compound 70):

¹H NMR (300 MHz, DMSO d⁶, 300° K):

δ(ppm) 1.40 (m, 1H, H₃ Pro ), 1.53 (m, 1H, H₄ Pro ), 1.71 (m, 1H, H₄ Pro ), 2.09 (m, 1H, H₃ Pro ), 2.90 (m, 4H, CH₂—CH₂-indole), 3.06 (t, 2H, J=6 Hz, H₅ Pro), 3.28 (m, 2H, CH₂ βTrp), 3.40(s, 3H, OCH₃), 3.80(s, 3H, OCH₃), 3.80(m, 1H, CH αPro), 4.80(d, 1H, J=17 Hz, CH₂-o,p-dimethoxybenzyl), 5.4 (d, 1H, J=17 Hz, CH₂-o,p-dimethoxybenzyl), 5.1 (m, 1H, CH αTrp), 6.26 (dd, 1H, Jₒ=8 Hz and Jₘ=2 Hz, H₅ o,p-dimethoxybenzyl), 6.38 (d, 1H, Jₒ=8 Hz, H₆ o,p-dimethoxybenzyl), 6.53 (d, 1H, Jₘ=2 Hz, H₃ o,p-dimethoxybenzyl), 6.84 (t, 1H, H₅ indole), 6.91 (t, 1H, Jₒ=8 Hz, H₅ Trp), 6.93-7.07 (m, 4H, H₂ and H₆ indole, H₂ and H₆ Trp), 7.16 (d, 1H, Jₒ=8 Hz, H₄ Trp), 7.29 (m, 3H, H₄ and H₇ indole, H₇ Trp), 8.39 and 9.10 (2 m, 2H, NH Pro TFA salt), 9.22 (d, 1H, J=8 Hz, NH amide), 10.76 (s, 1H, NH indole), 10.80 (s, 1H, NH indole Trp).

¹³C NMR (75 MHz, DMSO d⁶, 300° K):

δ(ppm) 22.9 (CH₂—CH₂-indole), 23.5 (C₄ Pro), 25.8 (CH₂—CH₂-indole), 29.6 (CH₂ βTrp), 29.8 (C₃ Pro), 41.9 (CH₂-o,p-dimethoxybenzyl), 45.2 (CH αTrp), 46.0 (C₅ Pro ), 55.7 (OCH₃), 55.9 (OCH₃), 59.3 (CH αPro), 99.0 (C₃ o,p-dimethoxybenzyl), 105.1 (C₅ o,p-dimethoxybenzyl), 109.7 (C₃ Trp), 111.8 (C₇ indole and C₇ Trp), 113.4 (C₃ indole), 115.6 (C₁ o,p-dimethoxybenzyl), 118.4 (C₄ indole and C₄ Trp), 118.7 (C₅ indole and C₅ Trp), 121.4 (C₆ indole and C₆ Trp), 123.0 (C₂ indole and C₂ Trp), 127.2 (C₉ indole), 127.3 (C₉ Trp), 128.1 (C₆ o,p-dimethoxybenzyl), 136.5 (C₈ Trp), 136.6 (C₈ indole), 155.0 (Cq triazole), 157.8 (C₂ o,p-dimethoxybenzyl), 160.9 (C₄ o,p-dimethoxybenzyl), 168.1 (CO amide).

### (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)pyrazine-2-carboxamide (Compound 71):

¹H NMR (300 MHz, DMSO d⁶, 300° K):

δ(ppm) 3.01 (m, 2H, CH₂—CH₂-indole), 3.10 (m, 2H, CH₂—CH₂-indole), 3.51 (m, 2H, CH₂ βTrp), 3.55 (s, 3H, OCH₃), 3.57 (s, 3H, OCH₃), 5.15 (d, 2H, J=7 Hz, CH₂-o,p-dimethoxybenzyl), 5.63 (m, 1H, CH αTrp), 6.08 (dd, 1H, Jₒ=8 Hz and Jₘ=2 Hz, H₅ o,p-dimethoxybenzyl), 6.35 (d, 1H, Jₘ=2 Hz, H₃ o,p-dimethoxybenzyl), 6.53 (d, 1H, Jₒ=8 Hz, H₆ o,p-dimethoxybenzyl), 6.89 (m, 2H, H₅ indole and H₅ Trp), 6.99 (m, 2H, H₆ indole and H₆ Trp), 7.08 (m, 2H, H₂ indole and H₂ Trp), 7.29 (m, 3H, H₄ Trp, H₄ and H₇ indole), 7.41 (d, 1H, Jₒ=8 Hz, H₇ Trp), 8.61 (t, 1H, J=2 Hz, H₃ o-pyrazine), 8.79 (d, 1H, J=2 Hz, H₅ o-pyrazine), 8.94 (d, 1H, J=1 Hz, H₆ o-pyrazine), 9.43 (d, 1H, J=8 Hz, NH amide), 10.84 (s, 2H, NH indole and NH indole Trp).

¹³C NMR (75 MHz, DMSO d⁶, 300° K):

δ(ppm) 22.0 (CH₂—CH₂-indole), 25.3 (CH₂—CH₂-indole), 27.9 (CH₂ βTrp), 44.1 (CH₂-o,p-dimethoxybenzyl), 45.5 (CH αTrp), 55.5 (OCH₃), 55.8 (OCH₃), 98.8 (C₃ o,p-dimethoxybenzyl), 104.9 (C₅ o,p-dimethoxybenzyl), 109.3 (C₃ Trp), 111.8 (C₇ indole and C₇ Trp), 112.1 (C₃ indole), 113.4 (C₁ o,p-dimethoxybenzyl), 118.4 (C₄ indole), 118.5 (C₄ Trp), 118.8 (C₅ indole and C₅ Trp), 121.5 (C₆ indole and C₆ Trp), 127.0 (C₉ indole), 127.4 (C₉ Trp), 136.4 (C₈ Trp), 136.6 (C₈ indole), 141.2 (C₆ o-pyrazine), 144.1 (C₂ o-pyrazine), 144.3 (C₃ o-pyrazine), 146.8 (C₅ o-pyrazine), 155.4 (Cq triazole), 156.0 (Cq triazole), 157.8 (C₂ o,p-dimethoxybenzyl), 161.0 (CO amide), 163.2 (C₄ o,p-dimethoxybenzyl).

### (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)picolinamide (Compound 73):

¹H NMR (300 MHz, DMSO d⁶, 300° K):

δ(ppm) 2.94 (m, 4H, CH₂—CH₂-indole), 3.47 (m, 2H, CH₂ βTrp), 3.57 (s, 3H, OCH₃), 3.60 (s, 3H, OCH₃), 5.05 (m, 2H, CH₂-o,p-dimethoxybenzyl), 5.56 (m, 1H, CH αTrp), 6.14 (dd, 1H, Jₒ=8 Hz and Jₘ=2 Hz, H₅ o,p-dimethoxybenzyl), 6.41 (d, 1H, Jₘ=2 Hz, H₃ o,p-dimethoxybenzyl), 6.52 (d, 1H, Jₒ=8 Hz, H₆ o,p-dimethoxybenzyl), 6.88 (t, 2H, Jₒ=7 Hz, H₅ indole and H₅ Trp), 6.99 (t, 1H, Jₒ=8 Hz, H₆ Trp), 7.01 (t, 1H, Jₒ=8 Hz, H₆ indole), 7.04 (d, 1H, J=2 Hz, H₂ Trp), 7.07 (d, 1H, J=2 Hz, H₂ indole), 7.27-7.33 (m, 4H, H₄ and H₇ Trp, H₄ and H₇ indole), 7.55 (m, 1H, NH amide), 7.90 (m, 2H, H₄ and H₅ o-pyridyl), 8.57 (d, 1H, J_{αβ}=4 Hz, H₆ o-pyridyl), 9.15 (d, 1H, J=8 Hz, H₃ o-pyridyl), 10.78 (brs, 2H NH indole and NH indole Trp).

¹³C NMR (75 MHz, DMSO d⁶, 300° K):

δ(ppm) 22.3 (CH₂—CH₂-indole), 25.6 (CH₂—CH₂-indole), 28.9 (CH₂ βTrp), 43.1 (CH₂-o,p-dimethoxybenzyl), 45.5 (CH αTrp), 55.5 (OCH₃), 55.8 (OCH₃), 98.9 (C₃ o,p-dimethoxybenzyl), 105.0 (C₅ o,p-dimethoxybenzyl), 109.5 (C₃ Trp), 111.8 (C₇ indole and C₇ Trp), 112.8 (C₃ indole), 114.4 (C₁ o,p-dimethoxybenzyl), 118.4 (C₄ indole and C₄ Trp), 118.7 (C₅ indole), 118.8 (C₅ Trp), 121.4 (C₆ indole and C₆ Trp), 122.5 (C₂ indole and C₃ o-pyridyl), 123.1 (C₂ Trp), 127.1 (C₅ o-pyridyl), 127.2 (C₉ indole), 127.5 (C₉ Trp), 128.6 (C₆ o,p-dimethoxybenzyl), 136.4 (C₈ Trp), 136.6 (C₈ indole), 139.1 (C₄ o-pyridyl), 146.6 (C₆ o-pyridyl), 150.6 (C₂ o-pyridyl), 155.5 (Cq triazole), 155.6 (Cq triazole), 157.8 (C₂ o,p-dimethoxybenzyl), 161.0 (C₄ o,p-dimethoxybenzyl), 163.9 (CO amide).

### (R)-1-(5-(2-(1H-indol-3-yl)ethyl)-4-(2,4-dimethoxybenzyl)-4H-1,2,4-triazol-3-yl)-2-(1H-indol-3-yl)ethanamine (Compound 74):

¹H NMR (300 MHz, DMSO d⁶, 300° K):

δ(ppm) 2.79 (m, 2H, CH₂—CH₂-indole), 2.86 (m, 2H, CH₂—CH₂-indole), 3.30 (dd, 1H, ³J=14 Hz and 5 Hz, CH₂ βTrp), 3.38 (dd, 1H, ³J=14 Hz and 6 Hz, CH₂ βTrp), 3.62 (s, 3H, OCH₃), 3.63 (s, 3H, OCH₃), 4.47 (d, 1H, ³J=17 Hz, CH₂-o,p-dimethoxybenzyl), 4.59 (d, 1H, ³J=17 Hz, CH₂-o,p-dimethoxybenzyl), 6.11 (dd, 1H, Jₒ=8 Hz and Jₘ=2 Hz, H₅ o,p-dimethoxybenzyl), 6.20 (d, 1H, Jₒ=8 Hz, H₆ o,p-dimethoxybenzyl), 6.45 (d, 1H, Jₘ=2 Hz, H₃ o,p-dimethoxybenzyl), 6.87 (t, 1H, Jₒ=8 Hz, H₅ Trp), 6.91 (t, 1H, Jₒ=8 Hz, H₅ indole), 7.00-7.04 (m, 2H, H₆ indole and H₆ Trp), 7.07 (s, 1H, H₂ indole), 7.09 (s, 1H, H₂ Trp), 7.17-7.35 (m, 4H, H₄ and H₇ indole, H₄ and H₇ Trp), 8.75 (brs, 3H, NH₂ TFA salt), 10.78 (s, 1H, NH indole), 11.00 (s, 1H, NH indole Trp).

¹³C NMR (75 MHz, DMSO d⁶, 300° K):

δ(ppm) 22.9 (CH₂—CH₂-indole), 25.7 (CH₂—CH₂-indole), 29.9 (CH₂ βTrp), 41.5 (CH₂-o,p-dimethoxybenzyl), 46.5 (CH αTrp), 55.6 (OCH₃), 55.9 (OCH₃), 98.8 (C₃ o,p-dimethoxybenzyl), 105.0 (C₅ o,p-dimethoxybenzyl), 107.4 (C₃ Trp), 111.8 (C₇ indole and C₇ Trp), 113.4 (C₃ indole), 115.1 (C₁ o,p-dimethoxybenzyl), 118.0 (C₄ indole), 118.4 (C₄ Trp), 118.6 (C₅ Trp), 119.0 (C₅ indole), 121.3 (C₆ Trp), 121.6 (C₆ indole), 122.9 (C₂ indole), 125.4 (C₂ Trp), 127.1 (C₉ indole and C₉ Trp), 128.5 (C₆ o,p-dimethoxybenzyl), 136.5 (C₈ Trp), 136.6 (C₈ indole), 152.3 (Cq triazole), 155.6 (Cq triazole), 157.6 (C₂ o,p-dimethoxybenzyl), 160.8 (C₄ o,p-dimethoxybenzyl).

### (R)-N-(1-(4-(4-methoxybenzyl)-5-phenethyl-4H-1,2,4-triazol-3-yl)-2-(1H-indol-3-yl)ethyl)picolinamide (Compound 79):

¹H NMR (300 MHz, DMSO d⁶, 300° K):

δ(ppm) 2.85 (m, 4H, CH₂—CH₂-phenyl), 3.51 (m, 2H, CH₂ βTrp), 3.59 (s, 3H, OCH₃), 5.11 (d, 1H, J=17 Hz, CH₂-p-methoxybenzyl), 5.23 (d,1H, J=17 Hz, CH₂— p-methoxybenzyl), 5.51 (m, 1H, CH αTrp), 6.59 (d, 2H, Jₒ=8 Hz, H₃ and H₅ p-methoxybenzyl), 6.73 (d, 2H, Jₒ=8 Hz, H₂ and H₆ p-methoxybenzyl), 6.87 (t, 1H, Jₒ=8 Hz, H₅ Trp), 7.01 (t, 1H, Jₒ=8 Hz, H₆ Trp), 7.06 (m, 2H, H₂ and H₆ phenyl), 7.10 (d, 1H, J=2 Hz, H₂ Trp), 7.14 (d, 1H, Jₒ=7 Hz, H₄ Trp), 7.24 (m, 3H, H₃, H₄ and H₅ phenyl), 7.34 (d, 1H, Jₒ=8 Hz, H₇ Trp), 7.55 (m, 1H, NH amide), 7.88 (m, 2H, H₄ and H₅ o-pyridyl), 8.56 (d, 1H, J_{αβ}=4 Hz, H6 o-pyridyl), 9.20 (d, 1H, Jₒ=8 Hz, H₃ o-pyridyl), 10.80 (s, 1H, NH indole Trp).

¹³C NMR (75 MHz, DMSO d⁶, 300° K):

δ(ppm) 26.2 (CH₂—CH₂-phenyl), 28.6 (CH₂ βTrp), 32.1 (CH₂—CH₂-phenyl), 45.5 (CH αTrp), 46.2 (CH₂-p-methoxybenzyl), 55.4 (OCH₃), 109.6 (C₃ Trp), 111.8 (C₇ Trp), 114.3 (C₃ and C₅ p-methoxybenzyl), 118.5 (C₄ Trp), 118.8 (C₅ Trp), 121.4 (C₆ Trp), 122.5 (C₃ o-pyridyl), 124.5 (C₂ Trp), 127.2 (C₂ and C₆ phenyl), 127.4 (C₉ Trp and C₁ p-methoxybenzyl), 127.8 (C₅ o-pyridyl), 128.7 (C₂ and C₆ p-methoxybenzyl), 128.8 (C₃, C₄ and C₅ phenyl), 136.4 (C₈ Trp), 138.1 (C₄ o-pyridyl), 140.3 (C₁ phenyl), 148.7 (C₆ o-pyridyl), 149.3 (C₂ o-pyridyl), 155.0 (Cq triazole), 155.3 (Cq triazole), 159.0 (C₄ p-methoxybenzyl), 164.1 (CO amide).

### (R)-N-(1-(5-(2-(1H-indol-3-yl)ethyl)-4-(4-methoxybenzyl)-4H-1,2,4-triazol-3-yl)-2-(1H-indol-3-yl)ethyl)picolinamide (Compound 80):

¹H NMR (300 MHz, DMSO d⁶, 300° K):

δ(ppm) 2.95 (m, 4H, CH₂—CH₂-indole), 3.48 (m, 2H, CH₂ βTrp), 3.58 (s, 3H, OCH₃), 5.16 (m, 2H, CH₂-p-methoxybenzyl), 5.50 (m, 1H, CH αTrp), 6.57 (d, 2H, Jₒ=8 Hz, H₃ and H₅ p-methoxybenzyl), 6.72 (d, 2H, Jₒ=8 Hz, H₂ and H₆ p-methoxybenzyl), 6.87 (t, 2H, Jₒ=8 Hz, H₅ Trp and H₅ indole), 6.96-7.07 (m, 5H, H₂ and H₆ indole, H₂, H₄ and H₆ Trp), 7.27-7.34 (m, 3H, H₄ and H₇ indole, H₇ Trp), 7.55 (m, 1H, NH amide), 7.88 (m, 2H, H₄ and H₅ o-pyridyl), 8.56 (d, 1H, J_{αβ}=4 Hz, H₆ o-pyridyl), 9.18 (d, 1H, Jₒ=8 Hz, H₃ o-pyridyl), 10.77 (brs, 2H, NH indole Trp and NH indole).

¹³C NMR (75 MHz, DMSO d⁶, 300° K):

δ(ppm) 22.4 (CH₂—CH₂-indole), 25.7 (CH₂—CH₂-indole), 28.9 (CH₂ βTrp), 45.5 (CH αTrp), 46.2 (CH₂-p-methoxybenzyl), 55.4 (OCH₃), 109.7 (C₃ Trp), 111.8 (C₇ Trp and C₇ indole), 112.6 (C₃ indole), 114.3 (C₃ and C₅ p-methoxybenzyl), 118.4 (C₄ Trp and C₄ indole), 118.7 (C₅ indole), 118.8 (C₅ Trp), 121.4 (C₆ Trp and C₆ indole), 122.4 (C₃ o-pyridyl and C₂ indole), 124.5 (C₂ Trp), 126.7 (C₉ indole), 127.1 (C₉ Trp), 127.2 (C₅ o-pyridyl), 127.5 (C₁ p-methoxybenzyl), 127.7 (C₂ and C₆ p-methoxybenzyl), 136.4 (C₈ Trp), 136.6 (C₈ indole), 138.1 (C₄ o-pyridyl), 148.7 (C₆ o-pyridyl), 149.3 (C₂ o-pyridyl), 155.3 (Cq triazole), 159.0 (C₄ p-methoxybenzyl), 164.0 (CO amide).

### (R)-N-1-(5-(2-(1H-indol-3-yl)ethyl)-4-(4-methoxybenzyl)-4H-1,2,4-triazol-3-yl)-2-(1H-indol-3-yl)ethyl)piperazine-2-carboxamide (Compound 81):

¹H NMR (300 MHz, DMSO d⁶, 300° K):

δ(ppm) 2.18 (m, 2H, NH piperazine), 2.96 (m, 6H, H₂, H₅ and H₆ piperazine), 3.34 (d, 2H, J=7 Hz, CH₂ βTrp), 3.57 (m, 4H, CH₂—CH₂-indole), 3.61 (s, 3H, OMe), 3.64 (m, 1H, H₃ piperazine), 4.82 (m, 2H, CH₂-p-methoxybenzyl), 5.40 (m, 1H, CH αTrp), 6.45 (d, 2H, Jₒ=8 Hz, H₃ and H₅ p-methoxybenzyl), 6.51 (d, 2H, Jₒ=8 Hz, H₂ and H₆ p-methoxybenzyl), 6.65-7.47 (m, 10H, CHar, indole and indole Trp), 8.95 (m, 1H, NH amide), 10.88 (d, 1H, J=2 Hz, NH indole), 10.91 (s, 1H, NH indole Trp).

¹³C NMR (75 MHz, DMSO d⁶, 300° K):

δ(ppm) 22.5 (CH₂—CH₂-indole), 25.6 (CH₂—CH₂-indole), 31.3 (CH₂ βTrp), 41.9 (CH₂-p-methoxybenzyl), 47.7 (CH αTrp, C₅ and C₆ piperazine), 55.5 (OCH₃ and C₂ piperazine), 61.1 (C₃ piperazine), 109.3 (C₃ Trp), 111.7 (C₇ indole and C₇ Trp), 114.0 (C₃ indole), 114.3 (C₃ and C₅ p-methoxybenzyl), 118.6 (C₄ indole), 118.7 (C₄ Trp), 118.9 (C₅ indole and C₅ Trp), 121.4 (C₆ indole), 121.5 (C₆ Trp), 123.9 (C₂ indole and C₂ Trp), 127.0 (C₉ indole), 127.2 (C₉ Trp), 127.7 (C₁ p-methoxybenzyl), 128.1 (C₂ and C₆ p-methoxybenzyl), 136.3 (C₈ Trp), 136.5 (C₈ indole), 155.5 (Cq triazole), 162.2 (C₄p-methoxybenzyl), 171.1 (CO amide).

### (S)-N-((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide (Compound 89):

¹H NMR (300 MHz, DMSO d⁶, 300° K):

δ(ppm) 1.42 (m, 1H, H₃ Pro ), 1.52 (m, 1H, H₄ Pro), 1.72 (m, 1H, H₄ Pro), 2.07 (m, 1H, H₃ Pro), 2.94 (m, 4H, CH₂—CH₂-indole), 3.05 (t, 2H, J=6 Hz, H₅ Pro), 3.30 (m, 2H, CH₂ βTrp), 3.67 (s, 3H, OCH₃), 3.96 (m, 1H, CH αPro), 5.02 (s, 2H, CH₂-p-methoxybenzyl), 5.19 (m, 1H, CH αTrp), 6.73 (s, 4H, CHar p-methoxybenzyl), 6.84 (t, 1H, Jₒ=8 Hz, H₅ indole), 6.90 (t, 1H, Jₒ=8 Hz, H₅ Trp), 6.93-7.06 (m, 4H, H₂ and H₆ indole, H₂ and H₆ Trp), 7.17 (d, 1H, Jₒ=8 Hz, H₄ Trp), 7.29 (d, 3H, Jₒ=8 Hz, H₄ and H₇ indole, H₇ Trp), 8.39 and 9.10 (2 m, 2H, NH Pro TFA salt), 9.25 (d, 1H, J=8 Hz, NH amide), 10.76 (s, 1H, NH indole), 10.80 (d, 1H, J=2 Hz, NH indole Trp).

¹³C NMR (75 MHz, DMSO d⁶, 300° K):

δ(ppm) 22.8 (CH₂—CH₂-indole), 23.5 (C₄ Pro), 25.8 (CH₂—CH₂-indole), 29.4 (CH₂ βTrp), 29.8 (C₃ Pro), 45.2 (CH αTrp), 45.5 (CH₂-p-methoxybenzyl), 46.0 (C₅ Pro), 55.5 (OCH₃), 59.3 (CH αPro), 109.6 (C₃ Trp), 111.8 (C₇ indole and C₇ Trp), 113.4 (C₃ indole), 114.6 (C₃ and C₅ p-methoxybenzyl), 118.4 (C₄ indole), 118.5 (C₄ Trp), 118.7 (C₅ indole and C₅ Trp), 121.4 (C₆ indole and C₆ Trp), 123.0 (C₂ Trp), 124.7 (C₂ indole), 127.2 (C₉ indole), 127.3 (C₉ Trp), 127.7 (C₁ p-methoxybenzyl), 127.8 (C₂ and C₆ p-methoxybenzyl), 126.5 (C₈ Trp), 136.6 (C₈ indole), 154.8 (Cq triazole), 154.9 (Cq triazole), 159.2 (C₄ p-methoxybenzyl), 168.2 (CO amide).

### (R)-N-((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide (Compound 90):

¹H NMR (300 MHz, DMSO d⁶, 300° K):

δ(ppm) 1.23 (m, 1H, H₃ Pro), 1.52 (m, 1H, H₄ Pro), 1.74 (m, 1H, H₄ Pro), 2.08 (m, 1H, H₃ Pro ), 2.81 (m, 2H, H₅ Pro), 2.90-3.14 (m, 4H, CH₂—CH₂-indole), 3.31 (dd, 1H, J=14 Hz and 7 Hz, CH₂ βTrp), 3.41 (dd, 1H, J=14 Hz and 8 Hz, CH₂ βTrp), 3.63 (s, 3H, OCH₃), 4.05 (m, 1H, CH αPro), 4.86 (s, 2H, CH₂-p-methoxybenzyl), 5.21 (m, 1H, CH αTrp), 6.63 (s, 4H, CHar p-methoxybenzyl), 6.88 (t, 2H, Jₒ=7 Hz, H₅ indole and H₅ Trp), 7.02 (m, 4H, H₂ and H₆ indole, H₂ and H₆ Trp), 7.26-7.34 (m, 4H, H₄ and H₇ indole, H₄ and H₇ Trp), 8.51 and 9.18 (2 m, 2H, NH Pro, TFA salt), 9.27 (d, 1H, J=8 Hz, NH amide), 10.73 (s, 1H, NH indole), 10.80 (s, 1H, NH indole Trp).

¹³C NMR (75 MHz, DMSO d⁶, 300° K):

δ(ppm) 22.8 (CH₂—CH₂-indole), 23.8 (C₄ Pro), 25.9 (CH₂—CH₂-indole), 29.7 (CH₂ βTrp and C₃ Pro), 45.4 (CH₂-p-methoxybenzyl), 45.8 (CH αTrp), 46.1 (C₅ Pro), 55.5 (OCH₃), 59.2 (CH αPro), 109.7 (C₃ Trp), 111.8 (C₇ Trp), 111.9 (C₇ indole), 113.4 (C₃ indole), 114.4 (C₃ and C₅ p-methoxybenzyl), 118.3 (C₄ indole), 118.5 (C₄ Trp), 118.6 (C₅ indole), 118.9 (C₅ Trp), 121.4 (C₆ indole and C₆ Trp), 122.9 (C₂ indole and C₂ Trp), 127.2 (C₉ indole), 127.4 (C₉ Trp), 127.6 (C₁, C₂ and C₆ p-methoxybenzyl), 136.5 (C₈ Trp), 136.6 (C₈ indole), 154.4 (Cq triazole), 155.0 (Cq triazole), 159.1 (C₄ p-methoxybenzyl), 168.3 (CO amide).

### (R)-N-(1-(5-(2-(1H-indol-3-yl)ethyl)-4-(4-bromobenzyl)-4H-1,2,4-triazol-3-yl)-2-(1H-indol-3-yl)ethyl)-2-amino-2-methylpropanamide (Compound 92):

¹H NMR (400 MHz, DMSO-d⁶):

δ(ppm) 1.28 (3H, s, CH₃ Aib), 1.30 (3H, s, CH₃ Aib), 2.90 (2H, m, CH₂—CH₂-indole), 3.00 (2H, m, CH₂—CH₂-indole), 3.37 (2H, m, CH₂ βTrp), 5.10 (2H, s, CH₂ 4-bromobenzyl), 5.13 (1H, m, CαH Trp), 6.75 (2H, d, J=8.1, H₂, H₆ 4-bromobenzyl), 6.88 (1H, t, J=7.3, H₅ Trp), 6.93 (1H, t, J=7.5, H₅ indole), 7.03 (1H, t, J=7.0, H₆ Trp), 7.05 (1H, H₆ indole), 7.07 (1H, d, J=1.7, H₂ indole), 7.09 (1H, d, J=1.8, H₂ Trp), 7.12 (1H, d, J=8.2, H₄ Trp), 7.28 (1H, d, J=7.9, H₄ indole), 7.32 (2H, d, J=8.2, H₇ Trp, H₇ indole), 7.41 (2H, d, J=8.1, H₃, H₅ 4-bromobenzyl), 8.01 (2H, s, NH₂ Aib), 8.95 (1H, d, J=7.9, NH Trp), 10.77 (1H), brs, NH indole), 10.80 (1H, brs, NH indole Trp).

¹³C NMR (400 MHz,DMSO-d⁶):

δ(ppm) 22.4 (CH₂—CH_{□}indole), 23.1 (CH₃ Aib), 23.4 (CH₃ Aib), 25.4 (CH₂—CH_{□}indole), 28.7 (Cβ Trp), 44.8 (CH₂ 4-bromobenzyl), 45.2 (Cα Trp,), 56.3 (Cq Aib), 109.4 (C₃ Trp), 111.3 (C₇ Trp, C₇ indole), 113.0 (C₃ indole), 117.8 (C₄ Trp), 118.0 (C₄ indole), 118.2 (C₅ indole), 118.3 (C₅ Trp), 120.8 (C₄ 4-bromobenzyl), 120.9 (C₆ Trp, C₆ indole), 122.5 (C₂ indole), 124.4 (C₂ Trp), 126.7 (C₉ Indole), 126.8 (C₉ Trp), 128.0 (C₂, C₆ 4-bromobenzyl), 131.6 (C₃, C₅ 4-bromobenzyl), 135.1 (C₁ 4-bromobenzyl), 136.1 (C₈ Trp, C₈ indole), 154.2 (Cq triazole), 154.5 (Cq triazole), 171.4 (CO Aib).

### (R)-N-(1-(5-(2-(1H-indol-3-yl)ethyl)-4-(4-methoxybenzyl)-4H-1,2,4-triazol-3-yl)-2-phenylethyl)-2-amino-2-methylpropanamide (Compound 93):

¹H NMR (300 MHz, DMSO-d⁶, 300° K):

δ(ppm) 1.18 (3H, s, CH₃ Aib), 1.27 (3H, s, CH₃ Aib), 2.91 (4H, m, CH₂—CH₂-indole), 3.19 (2H, m, CH₂ βPhe), 3.69 (3H, s, OCH₃), 5.05 (2H, m, CH₂-p-methoxybenzyl), 5.20 (1H, m, CH αPhe), 6.82 (2H, d, Jₒ=8 Hz, H₃ and H₅ p-methoxybenzyl), 6.88 (2H, d, Jₒ=8 Hz, H₂ and H₆ p-methoxybenzyl), 6.92 (1H, t, Jₒ=8 Hz, H₅ indole), 7.02 (1H, t, Jₒ=7 Hz, Jₒ=8 Hz, H₄ and H₇ indole), 7.99 (3H, brs, NH₂ Aib), 8.93 (1H, d, J=8 Hz, NH amide) 10.77 (1H, s, NH indole).

¹³C NMR (75 MHz, DMSO-d⁶, 300° K):

δ(ppm) 22.8 (CH₂—CH₂-indole), 23.5 (CH₃ Aib), 23.9 (CH₃ Aib), 25.9 (CH₂—CH₂-indole), 38.7 (CH₂ βPhe), 45.7 (CH₂-p-methoxybenzyl), 46.4 (CH αPhe), 55.6 (OCH₃), 56.8 (Cq Aib), 111.8 (C₇ indole), 113.4 (C₃ indole), 114.7 (C₃ and C₅ p-methoxybenzyl), 118.5 (C₄ Trp), 118.6 (C₅ Trp), 121.4 (C₆ Trp), 123.0 (C₂ Trp), 127.0 (C₄ phenyl), 127.2 (C₉ indole), 127.9 (C₁ p-methoxybenzyl), 128.1 (C₂ and C₆ phenyl), 128.5 (C₃ and C₅ phenyl), 129.8 (C₂ and C₆ p-methoxybenzyl), 136.6 (C₈ indole), 137.7 (C₁ phenyl), 155.2 (Cq triazole), 154.4 (Cq triazole), 159.3 (C₄ p-methoxybenzyl), 171.7 (CO Aib).

### (R)-N-(1-(4-(2-(1H-indol-3-yl)ethyl)-4H-1,2,4-triazol-3-yl)-2-(1-H-indol-3-yl)ethyl)-2-amino-2-methylpropanamide (Compound 95):

¹H NMR (400 MHz, DMSO-d⁶):

δ(ppm) 1.29 (3H, s, CH₃ Aib), 1.35 (3H, s, CH₃ Aib), 2.85 (1H, m, 1H N—CH₂—CH₂—In), 2.89 (1H, m, 1H, —N—CH₂—CH₂—In), 3.28 (1H, dd, J=14.2, J=6.8, 1H CH₂β Trp), 3.40 (1H, dd, J=14.2, J=8.4, 1H CH₂β Trp), 4.10 (2H, m, —N—CH₂—CH₂—In), 5.25 (1H, m, CHα Trp), 6.85 (1H, d, J=2.0, H₂ indole), 6.90-6.98 (2H, m, H₅ indole), 7.01 (1H, d, J=2.0, H₂ indole), 7.02-7.12 (2H, m, H₆ indole), 7.30 (1H, d, J=8.2, H₇ indole), 7.33 (1H, d, J=8.3, H₇ indole), 7.40 (1H, d, J=7.9, H₄ indole), 7.47 (1H, d, J=7.8, H₄ indole), 8.04 (2H, brs, NH₂ Aib), 8.42 (1H, s, H triazole), 9.01 (1H, d, J=8.0, NH Trp), 10.81 (1H, s, NH indole), 10.90 (1H, s, NH indole).

### (R)-N-(1-(5-((1H-indol-3-yl)methyl)-4-methyl-4H-1,2,4-triazol-3-yl)-2-(1H-indol-3-yl)ethyl)-2-amino-2-methylpropanamide (Compound 96):

¹H NMR (400 MHz, DMSO-d⁶):

δ(ppm) 1.20 (3H, s, CH₃ Aib), 1.28 (3H, s, CH₃ Aib), 3.32 (3H, d, N—CH₃), 3.30-3.45 (2H, m, CH₂β Trp), 4.22 (2H, s, —CH₂—In), 5.30 (1H, m, CHα Trp), 6.91 (1H, t, J=7.5, H₅ indole), 6.94 (1H, d, J=7.5, H₅ indole), 7.02 (1H, t, J=7.9, H₆ indole), 7.05 (1H, t, J=7.9, H₆ indole), 7.08 (1H, d, J=1.9, H₂ indole), 7.12 (1H, d, J=1.9, H₂ indole), 7.29 (1H, d, J=8.1, H₇ indole), 7.33 (1H, d, J=8.2, H₇ indole), 7.48 (1H, d, J=7.9, H₄ indole), 7.57 (1H, d, J=7.9 H₄ indole), 8.00 (2H, brs, NH₂ Aib), 8.85 (1H, d, J=8.2, NH Trp), 10.82 (1H, s, NH indole), 10.98 (1H, s, NH indole).

### (R)-N-(1-(5-(2-(1H-indol-3-yl)ethyl)-4-methyl-4H-1,2,4-triazol-3-yl)-2-(1H-indol-3-yl)ethyl)-2-amino-2-methylpropanamide (Compound 97):

¹H NMR (400 MHz, DMSO-d⁶):

δ(ppm) 1.30 (3H, s, CH₃ Aib), 1.40 (3H, s, CH₃ Aib), 3.00-3.20 (4H, m, —CH₂—CH₂—In), 3.36 (3H, s, N—CH₃), 3.45-3.50 (2H, m, CH₂β Trp), 5.30 (1H, m, CHα Trp), 6.95-7.04 (2H, t, H₅ indole), 7.06-7.13 (2H, m, H₆ indole), 7.18 (2H, brs, H₂ indole), 7.34 (1H, d, J=8.0, H₇ indole), 7.36 (1H, d, J=8.0, H₇ indole), 7.48 (1H, d, J=7.8, H₄ indole), 7.58 (1H, d, J=7.8, H₄ indole), 8.10 (2H, brs, NH₂ Aib), 8.95 (1H, d, J=8.1, NH Trp), 10.95 (1H, s, NH indole), 10.96 (1H, s, NH indole).

¹³C NMR (100 MHz,DMSO-d⁶):

δ(ppm) 22.9-25.9 (—CH₂—CH₂-indole), 24.1 (CH₃ Aib), 24.3 (CH₃ Aib), 28.8 (CH₂β Trp), 30.7 (—NCH₃), 46.2 (CHα Trp), 57.2 (Cq Aib), 110.2 (C₃ indole), 112.3 (2C₇ indole), 113.5 (C₃ indole), 118.9-119.2 (2C₅, 2C₄ indole), 121.9 (2C₆ indole), 123.6 (C₂ indole), 125.3 (C₂ indole), 127.7 (C₉ indole), 128.0 (C₉ indole), 136.9 (C₈ indole), 137.1 (C₈ indole), 155.3 (Cq triazole), 155.8 (Cq triazole), 172.2 (CO Aib).

### (R)-N-(1-(5-((1H-indol-3-yl)methyl)-4H-1,2,4-triazol-3-yl)-2-(1H-indol-3-yl)ethyl)-2-amino-2-methylpropanamide (Compound 98):

¹H NMR (400 MHz, DMSO-d⁶):

δ(ppm) 1.31 (3H, s, CH₃ Aib), 1.42 (3H, s, CH₃ Aib), 3.19 (1H, dd, J=14.5, J=9.6, 1H CH₂ βTrp), 3.35 (1H, dd, J=14.5, J=5.3, 1H CH₂ βTrp), 4.15 (2H, s, CH₂ indole), 5.26 (1H, m, CαH Trp), 6.95 (1H, t, H₅ Trp), 6.96 (1H, t, H₅ indole), 7.05 (1H, t, H₆ Trp), 7.06 (1H, s, H₂ Trp), 7.07 (1H, t, H₆ indole), 7.21 (1H, s, H₂ indole), 7.32 (1H, d, H₇ Trp), 7.37 (1H, d, H₇ indole), 7.51 (1H, d, J=7.8, H₄ indole), 7.58 (1H, d, J=7.8, H₄ Trp), 8.00 (2H, s, NH₂ Aib), 8.64 (1H, d, J=8.7, NH Trp), 10.77 (1H, s, NH indole Trp), 10.92 (1H, s, NH indole).

¹³C NMR (400 MHz,DMSO-d⁶):

δ(ppm) 22.9 (CH₂ indole), 23.2 (CH₃ Aib), 23.3 (CH₃ Aib), 29.4 (CP Trp), 48.3 (Cα Trp), 56.3 (Cq Aib), 109.7 (C₃ indole), 110.3 (C₃ Trp), 111.2 (C₇ Trp), 111.3 (C₇ indole), 118.1 (C₄ Trp, C₅ Trp), 118.3 (C₄ indole), 118.4 (C₅ indole), 120.7 (C₆ Trp), 121.0 (C₆ indole), 123.4 (C₂ indole), 123.6 (C₂ Trp), 126.8 (C₉ indole), 127.1 (C₉ Trp), 136.0 (C₈ Trp), 136.2 (C₈ indole), 157.5 (Cq triazole), 161.7 (Cq triazole), 170.8 (CO Aib).

### (R)-N-(1-(5-((1H-indol-3-yl)methyl)-4-(2,4-dimethoxybenzyl)-4H-1,2,4-triazol-3-yl)-2-(1H-indol-3-yl)ethyl)-2-amino-2-methylpropanamide (Compound 99):

¹H NMR (400 MHz, DMSO-d⁶):

δ(ppm) 1.27 (3H, s, CH₃ Aib), 1.29 (3H, s, CH₃ Aib), 3.25 (1H, dd, J=14.3, J=5.6, 1H CH₂ βTrp), 3.38 (1H, dd, J=14.3, J=9.1, 1H CH₂ βTrp), 3.68 (3H, s, OCH₃), 3.72 (3H, s, OCH₃), 4.10 (1H, d, J=16.5, 1H CH₂ indole), 4.16 (1H, d, J=16.5, 1H CH₂ indole), 4.96 (1H, d, J=16.8, 1H CH₂ o,p-dimethoxybenzyl), 5.12 (1H, d, J=16.8, 1H CH₂ o,p-dimethoxybenzyl), 5.16 (1H, m, CαH Trp), 6.21 (1H, dd, J=8.5, J=2.1, H₅ o,p-dimethoxybenzyl), 6.27 (1H, d, J=8.5, H₆ o,p-dimethoxybenzyl), 6.57 (1H, d, J=2.1, H₃ o,p-dimethoxybenzyl), 6.83 (1H, t, H₅ Trp), 6.94 (1H, t, H₅ indole), 7.02 (1H, t, H₆ Trp), 7.05 (1H, t, H₂ indole),7.06 (1H, t, H₆ indole), 7.07 (1H, s, H₂ Trp), 7.07 (1H, t, H₄ Trp), 7.31 (1H, d, H₇ Trp), 7.33 (1H, d, H₇indole), 7.36 (1H, d, J=7.8, H₄ indole), 8.00 (2H, br s, NH₂ Aib), 8.92 (1H, d, J=8.2, NH Trp), 10.79 (1H, s, NH indole Trp), 10.89 (1H, s, NH indole).

¹³C NMR (400 MHz,DMSO-d⁶):

δ(ppm) 21.2 (CH₂indole), 23.1 (CH₃ Aib), 23.2 (CH₃ Aib), 28.6 (Cβ Trp), 41.4 (N—CH₂ o,p-dimethoxybenzyl), 45.1 (Cα Trp), 55.2 (OCH₃), 55.4 (OCH₃), 56.2 (Cq Aib), 98.5 (C₃ o,p-dimethoxybenzyl), 104.6 (C₅ o,p-dimethoxybenzyl), 107.9 (C₃ indole), 109.5 (C₃ Trp), 111.2 (C₇ Trp), 111.3 (C₇ indole), 115.1 (C₁ o,p-dimethoxybenzyl), 117.8 (C₄ Trp), 118.1 (C₅ Trp), 118.3 (C₄ indole), 118.4 (C₅ indole), 120.8 (C₆ Trp), 121.1 (C₆ indole), 123.5 (C₂ indole), 124.3 (C₂ Trp), 126.6 (C₉ indole), 126.8 (C₉ Trp), 127.2 (C₆ o,p-dimethoxybenzyl), 136.0 (C₈ Trp), 136.2 (C₈ indole), 157.5 (Cq triazole), 154.9 (Cq triazole), 157.2 (C₂ o,p-dimethoxybenzyl), 160.3 (C₄ o,p-dimethoxybenzyl), 171.2 (CO Aib).

### (R)-N-(1-(5-((1H-indol-3-yl)methyl)-4-(4-methoxybenzyl)-4H-1,2,4-triazol-3-yl)-2-(1H-indol-3-yl)ethyl)-2-amino-2-methylpropanamide (Compound 101):

¹H NMR (300 MHz, DMSO-d⁶, 300° K):

δ(ppm) 1.22 (3H, s, CH₃ Aib), 1.25 (3H, s, CH₃ Aib), 3.22 (1H, dd, J=14 Hz and 6 Hz, CH₂ βTrp), 3.34 (1H, dd, J=14 Hz and 9 Hz, CH₂ βTrp), 3.68 (3H, s, OCH₃), 4.11 (2H, m, CH₂-indole), 5.09 (3H, m, CH αTrp and CH₂-p-methoxybenzyl), 6.70 (4H, s, CHar p-methoxybenzyl), 6.78 (2H, m, H₅ indole and H₅ Trp), 6.93 (2H, m, H₆ indole and H₆ Trp), 7.01-7.06 (3H, m, H₂ indole, H₂ and H₄ Trp), 7.31 (3H, m, H₄ and H₇ indolo, H₇ Trp), 7.98 (3H, brs, NH₂ Aib), 8.92 (1H, d, J=8 Hz, NH amide), 10.77 (1H, s, NH indole), 10.89 (1H, s, NH indole Trp).

¹³C NMR (75 MHz, DMSO-d⁶, 300° K):

δ(ppm) 21.7 (CH₂-indole), 23.5 (CH₃ Aib), 23.7 (CH₃ Aib), 28.9 (CH₂ βTrp), 45.6 (CH αTrp), 45.8 (CH₂— p-methoxybenzyl), 55.5 (OCH₃), 56.7 (Cq Aib), 108.1 (C₃ indole), 109.7 (C₃ Trp), 111.7 (C₇ Trp), 111.9 (C₇ indole), 114.5 (C₃ and C₅ p-methoxybenzyl), 118.3 (C₄ Trp), 118.7 (C₄ indole), 118.8 (C₅ indole), 118.9 (C₅ Trp), 121.3 (C₆ indole), 121.6 (C₆ Trp), 124.2 (C₂ indole), 125.3 (C₂ Trp), 127.1 (C₉ indole), 127.2 (C₉ Trp), 127.6 (C₁ p-methoxybenzyl), 127.8 (C₂ and C₆ p-methoxybenzyl), 136.4 (C₈ Trp), 136.7 (C₈ indole), 154.2 (Cq triazole), 155.2 (Cq triazole), 159.2 (C₄ p-methoxybenzyl), 171.9 (CO Aib).

### (R)-N-(1-(4-(2,4-dimethoxybenzyl)-5-benzyl-4H-1,2,4-triazol-3-yl)-2-(1H-indol-3-yl)ethyl)-2-amino-2-methylpropanamide (Compound 102):

¹H NMR (400 MHz, DMSO-d⁶):

δ(ppm) 1.30 (3H, s, CH₃ Aib), 1.33 (3H, s, CH₃ Aib), 3.26 (1H, dd, J=14.2, J=5.9, 1H CH₂ βTrp), 3.38 (1H, dd, J=14.2, J=8.7, 1H CH₂ βTrp), 3.69 (3H, s, OCH₃), 3.72 (3H, s, OCH₃), 4.02 (2H, s, CH₂ benzyl), 4.87 (1H, d, J=16.7, 1H CH₂ o,p-dimethoxybenzyl), 5.08 (1H, d, J=16.7, 1H CH₂ o,p-dimethoxybenzyl), 5.17 (1H, m, CαH Trp), 6.24 (1H, dd, J=8.4, J=1.7, H₅ o,p-dimethoxybenzyl), 6.28 (1H, d, J=8.4, H₆ o,p-dimethoxybenzyl), 6.56 (1H, d, J=1.7, H₃ o,p-dimethoxybenzyl), 6.85 (1H, t, J=7.5, H₅ Trp), 7.02 (1H, t, H₆ Trp), 7.07 (2H, m, H₂, H₆ benzyl), 7.08 (1H, s, H₂ Trp), 7.09 (1H, d, H₄ Trp), 7.16-7.29 (3H, m, H₃, H₄, H₅ benzyl), 7.31 (1H, d, J=8.2, H₇ Trp), 8.01 (2H, s, NH₂ Aib), 8.92 (1H, d, J=7.9, NH Trp), 11.79 (1H, s, NH indole Trp).

¹³C NMR (400 MHz,DMSO-d⁶):

δ(ppm) 23.2 (2CH₃ Aib), 28.7 (Cβ Trp), 30.2 (CH₂-benzyl), 41.3 (CH₂— o,p-dimethoxybenzyl), 45.2 (Cα Trp), 55.2 (OCH₃), 55.4 (OCH₃), 56.2 (Cq Aib), 98.5 (C₃ o,p-dimethoxybenzyl), 104.7 (C₅ o,p-dimethoxybenzyl), 109.5 (C₃ Trp), 111.3 (C₇ Trp), 115.1 (C₁ o,p-dimethoxybenzyl), 117.8 (C₄ Trp), 118.2 (C₅ Trp), 120.8 (C₆ Trp), 124.3 (C₂ Trp), 126.5 (C₂, C₆ benzyl), 126.8 (C₉ Trp), 127.3 (C₆ o,p-dimethoxybenzyl), 128.3 (C₃, C₄, C₅ Benzyl), 135.8 (C₁ benzyl), 136.0 (C₈ Trp), 153.4 (Cq triazole), 155.0 (Cq triazole), 157.2 (C₂ o,p-dimethoxybenzyl), 160.3 (C₄ o,p-dimethoxybenzyl), 171.3 (CO Aib).

### (R)-N-(1-(5-((1H-indol-3-yl)methyl)-4-phenethyl-4H-1,2,4-triazol-3-yl)-2-(1H-indol-3-yl)ethyl)-2-amino-2-methylpropanamide (Compound 104):

¹H NMR (300 MHz, DMSO-d⁶, 300° K):

δ(ppm) 1.27 (3H, s, CH₃ Aib), 1.29 (3H, s, CH₃ Aib), 2.39-2.53 (4H, m, CH₂—CH2-phenyl), 3.74 (1H, m, CH₂ βTrp), 3.92 (1H, m, CH₂ βTrp), 3.99 (2H, s, CH₂-indole), 5.21 (1H, m, CH αTrp), 6.74 (2H, m, H₅ indole and H₅ Trp), 6.90 (1H, t, Jₒ=8 Hz, H₆ Trp), 6.92 (1H, t, Jₒ=8 Hz, H₆ indole), 7.01-7.06 (4H, m, H₂ and H₆ phenyl, H₂ indole and H₂ Trp), 7.16 (3H, m, H₃, H₄ and H₅ phenyl), 7.27 (1H, d, Jₒ=8 Hz, H₄ Trp), 7.32 (1H, d, Jₒ=8 Hz, H₇ Trp), 7.36 (1H, d, Jₒ=8 Hz, H₇ indole), 7.50 (1H, d, Jₒ=8 Hz, H₄ indole), 7.99 (3H, brs, NH₂ Aib), 9.02 (1H, s, J=8 Hz, NH amide), 10.79 (1H, s, NH indole Trp), 10.94 (1H, s, NH indole).

¹³C NMR (75 MHz, DMSO-d⁶, 300° K):

δ(ppm) 21.4 (CH₂-indole), 23.5 (CH₃ Aib), 23.8 (CH₃ Aib), 29.5 (CH₂ βTrp), 35.8 (CH₂—CH₂-phenyl), 44.5 (CH₂—CH₂-phenyl), 45.8 (CH αTrp), 56.7 (Cq Aib), 108.5 (C₃ indole), 109.9 (C₃ Trp), 114.0 (C₇ indole and C₇ Trp), 118.4 (C₄ Trp), 118.8 (C₄ indole and C₅ Trp), 119.0 (C₅ indole), 121.4 (C₆ Trp), 121.7 (C₆ indole), 124.0 (C₂ indole and C₂ Trp), 127.1 (C₄ phenyl), 127.7 (C₉ indole and C₉ Trp), 128.8 (C₂ and C₆ phenyl), 129.1 (C₃ and C₅ phenyl), 136.5 (C₈ Trp), 136.6 (C₈ indole), 137.5 (C₁ phenyl), 153.6 (Cq triazole), 155.0 (Cq triazole), 171.8 (CO Aib).

### (R)-N-(1-(5-benzyl-4-(2,2-diphenylethyl)-4H-1,2,4-triazol-3-yl)-2-(1H-indol-3-yl)ethyl)-2-amino-2-methylpropanamide (Compound 106):

¹H NMR (300 MHz, DMSO-d⁶, 300° K):

δ(ppm) 1.29 (3H, s, CH₃ Aib), 1.34 (3H, s, CH₃ Aib), 3.37 (4H, m, CH₂ βTrp and CH₂-benzyl), 3.74 (1H, t, J=7 Hz, CH₂—CH(Phe)₂), 4.21 (1H, dd, J=14 Hz and 8 Hz, CH₂—CH(Phe)₂), 4.51 (1H, dd, J=14 Hz and 8 Hz, CH₂—CH(Phe)₂), 5.08 (1H, m, CH αTrp), 6.72 (2H, m, H₂ and H₆ benzyl), 6.86-6.93 (5H, m, H₃, H₄ and H₅ benzyl, H₅ and H₆ Trp), 7.03 (1H, s, H₂ Trp), 7.06-7.25 (CHar phenyl from CH(Phe)₂), 7.33 (1H, d, Jₒ=8 Hz, H₄ Trp), 7.47 (1H, d, Jₒ=8 Hz, H₇ indole), 8.10 (3H, brs, NH₂ Aib), 8.98 (1H, d, J=8 Hz, NH amide), 10.94 (1H, s, NH indole Trp).

¹³C NMR (75 MHz, DMSO-d⁶, 300° K):

δ(ppm) 23.5 (CH₃ Aib), 23.7 (CH₃ Aib), 29.4 (CH₂ βTrp), 30.1 (CH₂-benzyl), 46.0 (CH αTrp), 47.7 (CH₂—CH(Phe)₂), 51.3 (CH(Phe)₂), 56.8 (Cq Aib), 109.8 (C₃ Trp), 112.0 (C₇ Trp), 118.5 (C₄ Trp), 119.0 (C₅ Trp), 121.5 (C₆ Trp), 124.8 (C₂ Trp), 127.1 (C₄ phenyl from CH(Phe)₂), 127.4 (C₉ Trp, C₂ and C₆ benzyl), 128.3 (C₂ and C₆ phenyl from CH(Phe)₂), 128.8-129.1 (C₃ and C₅ phenyl from CH(Phe)₂, C₃, C₄ and C₅ benzyl), 136.2 (C₁ benzyl), 136.5 (C₈ Trp), 141.0 (C₁ phenyl from CH(Phe)₂), 153.5 (Cq triazole), 155.1 (Cq triazole), 172.0 (CO Aib)

### (R)-N-(1-(5-(2-(1H-indol-3-yl)ethyl)-4-(2,2-diphenylethyl)-4H-1,2,4-triazol-3-yl)-2-(1H-indol-3-yl)ethyl)-2-amino-2-methylpropanamide (Compound 107):

¹H NMR (300 MHz, DMSO-d⁶, 300° K):

δ(ppm) 1.34 (3H, s, CH₃ Aib), 1.38 (3H, s, CH₃ Aib), 2.06 (1H, m, CH₂—CH₂-indole), 2.30 (1H, m, CH₂—CH₂-indole), 2.78 (2H, m, CH₂—CH₂-indole), 3.35 (1H, dd, J=14 Hz and 7 Hz, CH₂ βTrp), 3.46 (1H, dd, J=14 Hz and 9 Hz, CH₂ βTrp), 3.58 (1H, t, J=7 Hz, CH₂—CH(Phe)₂), 4.14 (1H, dd, J=14 Hz and 8 Hz, CH₂—CH(Phe)₂), 4.39 (1H, dd, J=14 Hz and 7 Hz, CH₂—CH(Phe)₂), 5.12 (1H, m, CH αTrp), 6.50 (2H, m, H₅ indole and H₅ Trp), 6.76 (2H, m, H₆ indole and H₆ Trp), 6.87 (2H, m, H₂ indole and H₂ Trp), 6.89-6.96 (2H, m, H₄ phenyl), 7.03-7,15 (8H, m, H₂, H₃, H₅ and H₆ phenyl), 7.33 (3H, m, H₄ indole, H₄ and H₇ Trp), 7.47 (1H, d, J=8 Hz, H₇ indole), 8.11 (3H, brs, NH₂ Aib), 9.04 (1H, d, J=8 Hz, NH amide), 10.76 (1H, s, NH indole), 10.96 (1H, s, NH indole Trp).

¹³C NMR (75 MHz, DMSO-d⁶, 300° K):

δ(ppm) 22.4 (CH₂—CH₂-indole), 23.6 (CH₃ Aib), 23.8 (CH₃ Aib), 24.9 (CH₂—CH₂-indole), 29.6 (CH₂ βTrp), 46.1 (CH αTrp), 47.5 (CH₂—CH(Phe)₂), 51.5 (CH₂—CH(Phe)₂), 56.8 (Cq Aib), 109.8 (C₃ Trp), 111.8 (C₇ Trp), 112.1 (C₇ indole), 113.5 (C₃ indole), 118.4 (C₄ Trp), 118.7 (C₄ and C₅ indole), 119.0 (C₅ Trp), 121.4 (C₆ indole and C₆ Trp), 122.8 (C₂ indole), 125.0 (C₂ Trp), 127.2 (C₉ indole and C₉ Trp), 127.3 (C₄ phenyl), 128.2 (C₂ and C₆ phenyl), 128.7 (C₃ and C₅ phenyl), 136.6 (C₈ indole and C₈ Trp), 141.0 (C₁ phenyl), 154.6 (2 Cq triazole), 172.0 (CO Aib).

### (R)-N-(1-(4,5-dibenzyl-4H-1,2,4-triazol-3-yl)-2-(1H-indol-3-yl)ethyl)-2-amino-2-methylpropanamide (Compound 109):

¹H NMR (300 MHz, DMSO-d⁶, 300° K):

δ(ppm) 1.23 (3H, s, CH₃ Aib), 1.26 (3H, s, CH₃ Aib), 3.23 (1H, dd, J=14 Hz and 6 Hz, CH₂ βTrp), 3.35 (1H, dd, J=14 Hz and 9 Hz, CH₂ βTrp), 3.99 (2H, s, C—CH₂-phenyl), 5.10 (3H, m, N—CH₂-phenyl and CH αTrp), 6.77 (3H, m, H₅ Trp, H₂ and H₆ phenyl from N—CH₂-phenyl), 6.99 (2H, m, H₂ and H₆ Trp), 7.01-7.07 (3H, m, H₄ Trp, H₂ and H₆ from C—CH₂-phenyl), 7.15-7.23 (6H, m, H₃, H₄ and H₅ phenyl from N—CH₂-phenyl and from C—CH₂-phenyl), 7.25 (1H, d, J=8 Hz, H₇ Trp), 8.01 (3H, brs, NH₂ Aib), 8.91 (1H, d, J=8 Hz, NH amide), 10.78 (1H, s, NH indole Trp).

¹³C NMR (75 MHz, DMSO-d⁶, 300° K):

δ(ppm) 23.5 (CH₃ Aib), 23.7 (CH₃ Aib), 29.0 (CH₂ βTrp), 30.6 (C—CH₂-phenyl), 45.7 (CH αTrp), 46.1 (N—CH₂-phenyl), 56.7 (Cq Aib), 109.8 (C₃ Trp), 111.7 (C₇ Trp), 118.3 (C₄ Trp), 118.7 (C₅ Trp), 121.2 (C₆ Trp), 124.8 (C₂ Trp), 126.3 (C₂ and C₆ phenyl from N—CH₂-phenyl), 127.0 (C₂ and C₆ phenyl from C—CH₂-phenyl), 127.2 (C₉ Trp), 128.0 (C₄ phenyl from N—CH₂-phenyl), 128.8 (C₃, C₄ and C₅ phenyl from C—CH₂-phenyl), 128.9 (C₃ and C₅ phenyl from N—CH₂-phenyl), 135.8 (C₁ phenyl from N—CH₂-phenyl), 136.2 (C₁ phenyl from C—CH₂-phenyl), 136.4 (C₈ Trp), 153.9 (Cq triazole), 155.3 (Cq triazole), 171.9 (CO Aib).

### (R)-N-(1-(5-benzyl-4-hexyl-4H-1,2,4-triazol-3-yl)-2-(1H-indol-3-yl)ethyl)-2-amino-2-methylpropanamide (Compound 110):

¹H NMR (300 MHz, DMSO-d⁶, 300° K):

δ(ppm) 0.71 (3H, t, ³J=7 Hz, (CH₂)₅—CH₃), 0.87 (4H, m, 2 CH₂), 0.95 (2H, m, CH₂—CH₃), 1.00 (2H, m, N—CH₂—CH₂), 1.36 (6H, s, CH₃ Aib), 3.36 (1H, dd, ³J=14 Hz and 7 Hz, CH₂ δTrp), 3.41 (1H, dd, ³J=14 Hz and 7 Hz, CH₂ βTrp), 3.50 (1H, m, N—CH₂), 3.65 (1H, m, N—CH₂), 4.11 (2H, s, CH₂-benzyl), 5.14 (1H, m, CH αTrp), 6.90 (1H, t, Jₒ=7 Hz, H₅ Trp), 7.01 (1H, t, Jₒ=7 Hz, H₆ Trp), 7.04 (1H, s, H₂ Trp), 7.09 (2H, m, H₂ and H₆ benzyl), 7.17-7.29 (4H, m, H₄ Trp, H₃, H₄ and H₅ benzyl), 7.47 (1H, d, Jₒ=8 Hz, H₇ Trp), 8.10 (3H, brs, NH₂ Aib), 9.05 (1H, d, J=7 Hz, NH amide), 10.84 (1H, s, NH indole Trp).

¹³C NMR (75 MHz, DMSO-d⁶, 300° K):

δ(ppm) 14.1 ((CH₂)₅—CH₃), 22.1 (CH₂—CH₃), 23.8 (CH₃ Aib), 23.5 (CH₃ Aib), 25.8 (CH₃—CH₂—CH₂—CH₂), 29.5 (CH₂ βTrp), 30.3 (N—CH₂—CH₂), 30.8 (CH₂-benzyl and CH₃—CH₂—CH₂), 43.3 (N—CH₂—CH₂), 46.1 (CH αTrp), 56.8 (Cq Aib), 109.6 (C₃ Trp), 111.9 (C₇ Trp), 118.2 (C₄ Trp), 118.8 (C₅ Trp), 121.4 (C₆ Trp), 124.7 (C₂ Trp), 127.2 (C₂ and C₆ benzyl), 127.3 (C₉ Trp), 128.8 (C₃, C₄ and C₅ benzyl), 136.2 (C₁ benzyl), 136.5 (C₈ Trp), 153.1 (Cq triazole), 155.1 (Cq triazole), 171.9 (CO Aib).

### (R)-N-(1-(4-(2-(1H-indol-3-yl)ethyl)-5-benzyl-4H-1,2,4-triazol-3-yl)-2-(1H-indol-3-yl)ethyl)-2-amino-2-methylpropanamide (Compound 111):

¹H NMR (400 MHz, DMSO-d₆, 300° K):

δ(ppm) 1.31 (3H, s, CH₃ Aib), 1.35 (3H, s, CH₃ Aib), 2.51 (2H, m, CH₂—CH2-indole), 3.37 (2H, m, CH₂ βTrp), 3.76-3.90 (4H, m, CH₂-benzyl and CH₂—CH₂-indole), 5.25 (1H, m, CH αTrp), 6.88 (2H, t, Jₒ=7 Hz, H₅ indole and H₅ Trp), 6.95 (2H, t, Jₒ=7 Hz, H₆ indole and H₆ Trp), 7.03 (4H, m, H₂ Trp, H₂ indole, H₂ and H₆ benzyl), 7.16 (2H, d, Jₒ=8 Hz, H₄ indole and H₄ Trp), 7.20-7.30 (4H, 3, H₇ indole, H₃, H₄ and H₅ benzyl), 7.47 (1H, d, Jₒ=8 Hz, H₇ Trp), 8.05 (3H, brs, NH₂ Aib), 9.05 (1H, d, J=8 Hz, NH amide), 10.83 (1H, s, NH indole), 10.88 (1H, s, NH indole Trp).

¹³C NMR (100 MHz, DMSO-d⁶, 300° K):

δ(ppm) 23.5 (CH₃ Aib), 23.7 (CH₃ Aib), 29.6 (CH₂.βTrp), 30.3 (CH₂-benzyl and CH₂—CH₂-indole), 44.1 (CH₂—CH₂-indole), 46.1 (CH αTrp), 56.8 (Cq Aib), 109.8 (C₃ Trp), 109.9 (C₃ indole), 111.9 (C₇ indole and C₇ Trp), 118.3 (C₄ Trp), 118.4 (C₄ indole), 118.9 (C₅ indole and C₅ Trp), 121.3 (C₆ Trp), 121.5 (C₆ indole), 123.7 (C₂ indole), 124.7 (C₂ Trp), 127.0 (C₉ indole), 127.2 (C₂ and C₆ benzyl), 127.4 (C₉ Trp), 128.8 (C₃ and C₅ benzyl), 129.0 (C₄ benzyl), 136.3 (C₁ benzyl), 136.4 (C₈ indole and C₈ Trp), 153.1 (Cq triazole), 155.3 (Cq triazole), 171.8 (CO Aib).

### (S)-N-(1-(4-(2,4-dimethoxybenzyl)-5-benzyl-4H-1,2,4-triazol-3-yl)-2-(1H-indol-3-yl)ethyl)-2-amino-2-methylpropanamide (Compound 112):

¹H NMR (300 MHz, DMSO-d⁶, 300° K):

δ(ppm) 1.26 (3H, s, CH₃ Aib), 1.29 (3H, s, CH₃ Aib), 3.24 (1H, dd, J=14 Hz and 6 Hz, CH₂ βTrp), 3.33 (1H, dd, J=14 Hz and 9 Hz, CH₂ βTrp), 3.64 (3H, s, OCH₃), 3.68 (3H, s, OCH₃), 3.99 (2H, s, CH₂ phenyl), 4.84 (1H, d, J=17 Hz, CH₂-o,p-dimethoxybenzyl), 5.05 (1H, d, J=17 Hz, CH₂-o,p-dimethoxybenzyl), 5.13 (1H, m, CH αTrp), 6.24 (2H, m, H₅ and H₆ o,p-dimethoxybenzyl), 6.52 (1H, d, Jₘ=2 Hz, H₃ o,p-dimethoxybenzyl), 6.83 (1H, t, Jₒ=7 Hz, H₅ Trp), 7.01 (1H, t, Jₒ=8 Hz, H₆ Trp), 7.04 (1H, s, H₂ Trp), 7.05-7.23 (6H, m, H4 Trp and CHar phenyl), 7.27 (1H, d, Jₒ=8 Hz, H₇ Trp), 8.01 (3H, brs, NH₂ Aib), 8.90 (1H, d, J=8 Hz, NH amide), 10.77 (1H, d, J=2 Hz, NH indole Trp).

¹³C NMR (75 MHz, DMSO-d⁶, 300° K):

δ(ppm) 23.6 (CH₃ Aib), 29.1 (CH₂ βTrp), 30.6 (CH₂-phenyl), 41.9 (CH₂-o,p-dimethoxybenzyl), 45.7 (CH αTrp), 55.7 (OCH₃), 55.9 (OCH₃), 56.7 (Cq Aib), 99.9 (C₃ o,p-dimethoxybenzyl), 105.1 (C₅ o,p-dimethoxybenzyl), 109.9 (C₃ Trp), 111.7 (C₇ Trp), 115.5 (C₁ o,p-dimethoxybenzyl), 118.3 (C₄ Trp), 118.6 (C₅ Trp), 121.3 (C₆ Trp), 124.2 (C₂ Trp), 127.0 (C₆ o,p-dimethoxybenzyl), 127.3 (C₉ Trp), 127.8 (C₄ phenyl), 128.8 (C₂, C₃, C₅ and C₆ phenyl), 136.2 (C₈ Trp), 136.4 (C₁ phenyl), 153.9 (Cq triazole), 155.5 (Cq triazole), 157.6 (C₂ o,p-dimethoxybenzyl), 160.8 (C₄ o,p-dimethoxybenzyl), 171.8 (CO amide).

### (R)-N-(1-(4-(3,5-dimethoxybenzyl)-5-phenethyl-4H-1,2,4-triazol-3-yl)-2-(1H-indol-3-yl)ethyl)-2-amino-2-methylpropanamide (Compound 113):

¹H NMR (300 MHz, DMSO-d⁶, 300° K):

δ(ppm) 1.24 (3H, s, CH₃ Aib), 1.27 (3H, s, CH₃ Aib), 2.83 (4H, s, CH₂—CH₂-phenyl), 3.32 (2H, m, CH₂ βTrp), 3.61 (6H, s, OCH₃), 5.02 (2H, m, CH₂-m-dimethoxybenzyl), 5.18 (1H, m, CH αTrp), 6.07 (2H, d, Jₘ=2 Hz, H₂ and H₆ m-dimethoxybenzyl), 6.42 (1H, brs, H₄ m-dimethoxybenzyl), 6.83 (1H, t, Jₒ=7 Hz, H₅ Trp), 6.99 (1H, t, Jₒ=8 Hz, H₆ Trp), 7.08 (1H, d, J=2 Hz, H₂ Trp), 7.13 (3H, t, Jₒ=8 Hz, H₃, H₄ and H₅ phenyl), 7.20 (3H, d, Jₒ=7 Hz, H₂ and H₆ phenyl, H₄ Trp), 7.28 (1H, d, Jₒ=8 Hz, H₇ Trp), 7.99 (3H, brs, NH₂ Aib), 8.92 (1H, d, J=8 Hz, NH amide), 10.77 (1H, s, NH indole).

¹³C NMR (75 MHz, DMSO-d⁶, 300° K):

δ(ppm) 23.5 (CH₃ Aib), 23.8 (CH₃ Aib), 26.5 (CH₂—CH₂-phenyl), 29.2 (CH₂ βTrp), 32.7 (CH₂—CH₂-phenyl), 45.6 (CH αTrp), 45.8 (CH₂— m-dimethoxybenzyl), 55.6 (OCH₃), 56.8 (Cq Aib), 99.6 (C₄ m-dimethoxybenzyl), 104.6 (C₂ and C₆ m-dimethoxybenzyl), 109.9 (C₃ Trp), 111.8 (C₇ Trp), 118.2 (C₄ Trp), 118.7 (C₅ Trp), 121.3 (C₆ Trp), 124.8 (C₂ Trp), 126.5 (C₄ phenyl), 127.3 (C₉ Trp), 128.7 (C₂, C₃, C₅ and C₆ phenyl), 136.4 (C₈ Trp), 138.6 (C₄ m-dimethoxybenzyl), 140.9 (C₁ phenyl), 154.6 (Cq triazole), 154.8 (Cq triazole), 161.4 (C₃ and C₅ m-dimethoxybenzyl), 171.8 (CO amide).

### (R)-N-(1-(4-(4-bromobenzyl)-5-benzyl-4H-1,2,4-triazol-3-yl)-2-(1H-indol-3-yl)ethyl)-2-amino-2-methylpropanamide (Compound 114):

¹H NMR (300 MHz, DMSO-d⁶, 300° K):

δ(ppm)1.23 (3H, s, CH₃ Aib), 1.25 (3H, s, CH₃ Aib), 3.26 (1H, dd, ³J=14 Hz and 6 Hz, CH₂ Trp), 3.34 (1H, dd, ³J=14 Hz and 9 Hz, CH₂ βTrp), 4.01 (2H, m, CH₂-benzyl), 5.01 (1H, m, CH αTrp), 5.08 (2H, s, CH₂-p-bromobenzyl), 6.59 (2H, d, Jₒ=8 Hz, H₂ and H₆ p-bromobenzyl), 6.81 (1H, t, Jₒ=7 Hz, H₅ Trp), 6.94 (1H, s, H₂ Trp), 6.98 (1H, t, Jₒ=7 Hz, H₆ Trp), 7.06 (2H, m, H₂ and H₆ benzyl), 7.12 (1H, d, Jₒ=7 Hz, H₄ Trp), 7.16-7.20 (3H, m, H₃, H₄ and H₅ benzyl), 7.26 (1H, d, Jₒ=8 Hz, H₇ Trp), 7.29 (2H, d, Jₒ=8 Hz, H₃ and H₅ benzyl), 8.00 (3H, brs, NH₂ Aib), 8.92 (1H, d, J=8 Hz, NH amide), 10.78 (1H, s, NH indole Trp).

¹³C NMR (75 MHz, DMSO-d⁶, 300° K):

δ(ppm) 23.5 (CH₃ Aib), 23.8 (CH₃ Aib), 29.0 (CH₂ βTrp), 30.5 (CH₂-benzyl), 45.6 (CH₂-p-bromobenzyl), 45.7 (CH α Trp), 56.7 (Cq Aib), 109.7 (C₃ Trp), 111.8 (C₇ Trp), 118.2 (C₄ tryptophae), 118.7 (C₅ Trp), 121.2 (C₄ p-bromobenzyl), 121.3 (C₆ Trp), 124.9 (C₂ typtophane), 127.0 (C₂ and C₆ benzyl), 127.2 (C₉ Trp), 128.4 (C₂ and C₆ p-bromobenzyl), 128.9 (C₃, C₄ and C₅ benzyl), 131.9 (C₃ and C₅ p-bromobenzyl), 135.2 (C₁ p-bromobenzyl), 136.2 (C₈ Trp), 136.4 (C₁ benzyl), 153.9 (Cq triazole), 155.3 (Cq triazole), 171.9 (CO Aib).

### (R)-N-(1-(4-(2-methoxybenzyl)-5-benzyl-4H-1,2,4-triazol-3-yl)-2-(1H-indol-3-yl)ethyl)-2-amino-2-methylpropanamide (Compound 115):

¹H NMR (300 MHz, DMSO-d⁶, 300° K):

δ(ppm) 1.24 (3H, s, CH₃ Aib), 1.27 (3H, s, CH₃ Aib), 3.20 (1H, dd, J=14 Hz and 5 Hz, CH₂ βTrp), 3.33 (1H, dd, J=14 Hz and 9 Hz, CH₂ βTrp), 3.68 (3H, s, OCH₃), 4.00 (2H, s, CH₂-phenyl), 4.95 (1H, d, J=17 Hz, CH₂-o-methoxybenzyl), 5.07 (1H, m, CH αTrp), 5.18 (1H, d, J=17 Hz, CH₂-o-methoxybenzyl), 6.27 (1H, d, Jₒ=8 Hz, H₃ o-methoxybenzyl), 6.67 (1H, t, Jₒ=7 Hz, H₅ Trp), 6.77 (1H, t, Jₒ=6 Hz, H₆ Trp), 6.92-7.05 (6H, m, H₂ Trp, H₂ and H₆ phenyl, H₄, H₅ and H₆ o-methoxybenzyl), 7.14-7.26 (5H, m, H₄ and H₇ Trp, H₃, H₄ and H₅ phenyl), 8.03 (3H, brs, NH₂ Aib), 8.91 (1H, d, J=8 Hz, NH amide), 10.78 (1H, s, NH indole Trp).

¹³C NMR (75 MHz, DMSO-d⁶, 300° K):

δ(ppm) 23.5 (CH₃ Aib), 23.6 (CH₃ Aib), 29.1 (CH₂ β Trp), 30.5 (CH₂-phenyl), 42.1 (CH₂-o-methoxybenzyl), 45.7 (CH α Trp), 55.9 (OCH₃), 56.7 (Cq Aib), 109.8 (C₃ Trp), 111.3 (C₃ o-methoxybenzyl), 111.7 (C₇ Trp), 118.2 (C₄ Trp), 118.7 (C₅ Trp), 120.9 (C₅ o-methoxybenzyl), 121.2 (C₆ Trp), 123.3 (C₁ o-methoxybenzyl), 124.8 (C₂ Trp), 126.6 (C₂ and C₆ phenyl), 127.1 (C₄ o-methoxybenzyl), 127.2 (C₉ Trp), 128.8 (C₃, C₄ and C₅ phenyl), 129.5 (C₆ o-methoxybenzyl), 136.0 (C₁ phenyl), 136.4 (C₈ Trp), 154.0 (Cq triazole), 155.6 (Cq triazole), 156.5 (C₂ o-methoxybenzyl), 171.8 (CO Aib).

### (S)-N-(1-(4-(2,4-dimethoxybenzyl)-5-phenethyl-4H-1,2,4-triazol-3-yl)-2-(1H-indol-3-yl)ethyl)-2-amino-2-methylpropanamide (Compound 116):

¹H NMR (300 MHz, DMSO-d⁶, 300° K):

δ(ppm) 1.28 (3H, s, CH₃ Aib), 1.32 (3H, s, CH₃ Aib), 2.81 (4H, m, CH₂—CH₂-phenyl), 3.30 (2H, t, CH₂ βTrp), 3.61 (3H, s, OCH₃), 3.69 (3H, s, OCH₃), 4.87 (1H, d, J=17 Hz, CH₂-o,p-dimethoxybenzyl), 5.03 (1H, d, J=17 Hz, CH₂-o,p-dimethoxybenzyl), 5.20 (1H, m, CH αTrp), 6.29 (1H, dd, Jₒ=8 Hz and Jₘ=2 Hz, H₅ o,p-dimethoxybenzyl), 6.43 (1H, d, Jₒ=8 Hz, H₆ o,p-dimethoxybenzyl), 6.55 (1H, d, Jₘ=2 Hz, H₃ o,p-dimethoxybenzyl), 6.83 (1H, t, Jₒ=8 Hz, H₅ Trp), 6.99 (1H, t, Jₒ=8 Hz, H₆ Trp), 7.06 (1H, d, J=2 Hz, H₂ Trp), 7.09-7.25 (6H, m, H₄ Trp and CHar phenyl), 7.28 (1H, d, Jₒ=8 Hz, H₇ Trp), 8.04 (3H, brs, NH₂ Aib), 8.92 (1H, d, J=8 Hz, NH amide), 10.79 (1H, s, NH indole Trp).

¹³C NMR (75 MHz, DMSO-d⁶, 300° K):

δ(ppm) 23.6 (CH₃ Aib), 23.7 (CH₃ Aib), 26.5 (CH₂—CH₂-phenyl), 29.1 (CH₂ βTrp), 32.7 (CH₂—CH₂-phenyl), 41.8 (CH₂-o,p-dimethoxybenzyl), 45.7 (CH αTrp), 55.7 (OCH₃), 55.9 (OCH₃), 56.8 (Cq Aib), 99.1 (C₃ o,p-dimethoxybenzyl), 105.2 (C₅ o,p-dimethoxybenzyl), 109.9 (C₃ Trp), 111.8 (C₇ Trp), 115.6 (C₁ o,p-dimethoxybenzyl), 118.3 (C₄ Trp), 118.7 (C₅ Trp), 121.3 (C₆ Trp), 124.4 (C₂ Trp), 126.6 (C₄ phenyl), 127.3 (C₉ Trp), 128.2 (C₆ o,p-dimethoxybenzyl), 128.7 (C₂, C₃, C₅ and C₆ phenyl), 136.4 (C₈ Trp), 140.9 (C₁ phenyl), 154.6 (Cq triazole), 155.0 (Cq triazole), 157.8 (C₂ o,p-dimethoxybenzyl), 160.9 (C₄ o,p-dimethoxybenzyl), 171.8 (CO Aib).

### (R)-N-(1-(4,5-diphenethyl-4H-1,2,4-triazol-3-yl)-2-(1H-indol-3-yl)ethyl)-2-amino-2-methylpropanamide (Compound 117):

¹H NMR (300 MHz, DMSO-d⁶, 300° K):

δ(ppm) 1.32 (3H, s, CH₃ Aib), 1.37 (3H, s, CH₃ Aib), 2.59 (4H, m, C—CH₂—CH₂-phenyl), 2.83 (2H, t, J=8 Hz, N—CH₂—CH₂-phenyl), 3.38 (2H, m, N—CH₂—CH₂-phenyl), 3.84 (1H, m, CH₂ βTrp), 3.94 (1H, m, CH₂ βTrp), 5.23 (1H, m, CH aTrp), 6.84 (2H, m, H₄ phenyl from C—CH₂—CH₂-phenyl and H₄ phenyl from N—CH₂—CH₂-phenyl), 6.93 (1H, t, Jₒ=8 Hz, H₅ Trp), 7.00 (1H, t, Jₒ=8 Hz, H₆ Trp), 7.07 (1H, d, J=2 Hz, H₂ Trp), 7.11-7.27 (9H, m, H₂, H₃, H₅ and H₆ phenyl from C—CH₂—CH₂-phenyl, H₂, H₃, H₅ and H₆ phenyl from N—CH₂—CH₂-phenyl and H₄ Trp), 7.50 (1H, d, Jₒ=8 Hz, H₇ Trp), 8.07 (3H, brs, NH₂ Aib), 9.04 (1H, d, J=8 Hz, NH amide), 10.85 (1H, s, NH indole Trp).

¹³C NMR (75 MHz, DMSO-d⁶, 300° K):

δ(ppm) 23.5 (CH₃ Aib), 23.8 (CH₃ Aib), 25.9 (C—CH₂—CH₂-phenyl), 29.5 (CH₂ βTrp), 32.4 (C—CH₂—CH₂-phenyl), 35.8 (N—CH₂—CH₂-phenyl), 44.2 (N—CH₂—CH₂-phenyl), 45.9 (CH αTrp), 56.8 (Cq Aib), 109.7 (C₃ Trp), 111.9 (C₇ Trp), 118.4 (C₄ Trp), 118.9 (C₅ Trp), 121.4 (C₆ Trp), 124.8 (C₂ Trp), 126.6 (C₄ phenyl from C—C₂—C₂-phenyl), 127.2 (C₄ phenyl from N—CH₂—CH₂-phenyl), 127.4 (C₉ Trp), 128.7 (C₂ and C₆ phenyl from C—CH₂—CH₂-phenyl, C₂ and C₆ phenyl from N—CH₂—CH₂-phenyl), 128.8 (C₃ and C₅ phenyl from C—CH₂—CH₂-phenyl, C₃ and C₅ phenyl from N—CH₂—CH₂-phenyl), 136.5 (C₁ phenyl from N—CH₂—CH₂-phenyl), 137.5 (C₁ phenyl from C—CH₂—CH₂-phenyl), 140.8 (C₈ Trp), 154.1 (Cq triazole), 154.7 (Cq triazole), 171.9 (CO Aib).

### (R)-N-(1-(4-(3,4-dichlorobenzyl)-5-benzyl-4H-1,2,4-triazol-3-yl)-2-(1H-indol-3-yl)ethyl)-2-amino-2-methylpropanamide (Compound 118):

¹H NMR (300 MHz, DMSO-d⁶, 300° K):

δ(ppm) 1.24 (3H, s, CH₃ Aib), 1.25 (3H, s, CH₃ Aib), 3.33 (2H, m, CH₂ βTrp), 4.04 (2H, s, CH₂-benzyl), 5.05 (1H, m, CH αTrp), 5.12 (2H, s, CH₂-m,p-dichlorobenzyl), 6.49 (1H, dd, Jₒ=8 Hz and Jₘ=2 Hz, H₆ m,p-dichlorobenzyl), 6.80 (1H, t, Jₒ=8 Hz, H₅ Trp), 6.87 (1H, d, Jₘ=2 Hz, H₂ Trp), 6.98 (1H, t, Jₒ=7 Hz, H₆ Trp), 7.02-7.10 (3H, m, H₂ and H₆ benzyl, H₅ m,p-dichlorobenzyl), 7.18 (3H, m, H₃, H₄ and H₅ benzyl), 7.26 (2H, m, H₄ and H₇ Trp), 8.04 (3H, brs, NH₂ Aib), 8.94 (1H, d, J=9 Hz, NH amide), 10.81 (1H, s, NH indole Trp).

¹³C NMR (75 MHz, DMSO-d⁶, 300° K):

δ(ppm) 23.4 (CH₃ Aib), 23.8 (CH₃ Aib), 29.0 (CH₂ βTrp), 30.4 (CH₂-benzyl), 45.1 (CH₂-m,p-dichlorobenzyl), 45.6 (CH αTrp), 56.7 (Cq Aib), 109.6 (C₃ Trp), 111.8 (C₇ Trp), 118.1 (C₄ Trp), 118.6 (C₅ Trp), 121.3 (C₆ Trp), 124.9 (C₂ Trp), 126.4 (C₆ m,p-dichlorobenzyl), 127.0 (C₂ m,p-dichlorobenzyl), 127.2 (C₉ Trp), 128.4 (C₂ and C₆ benzyl), 130.7 (C₄ and C₅ m,p-dichlorobenzyl), 131.8 (C₃ m,p-dichlorobenzyl), 136.0 (C₁ benzyl), 136.4 (C₈ Trp), 136.7 (C₁ m,p-dichlorobenzyl), 154.1 (Cq triazole), 155.2 (Cq triazole), 172.0 (CO Aib).

### (R)-N-(1-(4-(4-methoxybenzyl)-5-benzyl-4H-1,2,4-triazol-3-yl)-2-phenylethyl)-2-amino-2-methylpropanamide (Compound 120):

¹H NMR (300 MHz, DMSO-d⁶, 300° K):

δ(ppm) 1.18 (3H, s, CH₃ Aib), 1.26 (3H, s, CH₃ Aib), 3.09 (1H, dd, J=14 Hz and 6 Hz, CH₂ βPhe), 3.18 (1H, dd, J=14 Hz and 9 Hz, CH₂ βPhe), 3.99 (2H, d, J=4 Hz, CH₂-phenyl), 4.95 (1H, d, J=16 Hz, CH₂-p-methoxybenzyl), 5.06 (1H, d, J=16 Hz, CH₂-p-methoxybenzyl), 5.13 (1H, m, CH αPhe), 6.78 (4H, s, CHar p-methoxybenzyl), 7.02-7.08 (4H, m, H₂ and H₆ phenyl, H₂ and H₆ Phe), 7.12-7.25 (6H, m, H₃, H₄ and H₅ phenyl, H₃, H₄ and H₅ Phe), 7.99 (3H, brs, NH₂ Aib), 8.92 (1H, d, J=8 Hz, NH amide).

¹³C NMR (75 MHz, DMSO-d⁶, 300° K):

δ(ppm) 23.4 (CH₃ Aib), 23.8 (CH₃ Aib), 30.7 (CH₂-phenyl), 38.7 (CH₂ βPhe), 45.9 (CH₂-p-methoxybenzyl), 46.4 (CH αPhe), 55.6 (OCH₃), 56.7 (Cq Aib), 114.6 (C₃ and C₅ p-methoxybenzyl), 126.9 and 127.1 (C₄ phenyl and C₄ Phe), 127.7 (C₁ p-methoxybenzyl), 128.2 (C₂ and C₆ Phe), 128.5 (C₃ and C₅ Phe), 128.9 (C₂, C₃, C₅ and C₆ phenyl), 129.7 (C₂ and C₆ p-methoxybenzyl), 136.3 (C₁ phenyl), 137.6 (C₁ Phe), 154.0 (Cq triazole), 154.9 (Cq triazole), 159.2 (C₄ p-methoxybenzyl), 171.7 (CO amide).

### (R)-N-(1-(4-(4-fluorobenzyl)-5-phenethyl-4H-1,2,4-triazol-3-yl)-2-(1H-indol-3-yl)ethyl)-2-amino-2-methylpropanamide (Compound 121):

¹H NMR (300 MHz, DMSO-d⁶, 300° K):

δ(ppm) 1.27 (3H, s, CH₃ Aib), 1.28 (3H, s, CH₃ Aib), 2.82 (4H, m, CH₂—CH₂-phenyl), 3.34 (2H, m, CH₂ βTrp), 5.06 (2H, s, CH₂-p-fluorobenzyl), 5.16 (1H, m, CH αTrp), 6.85 (3H, m, H₅ Trp, H₃ and H₅ p-fluorobenzyl), 6.98-7.04 (4H, m, H₂ and H₆ Trp, H₂ and H₆ phenyl), 7.09-7.11 (2H, m, H₂ and H₆ p-fluorobenzyl), 7.15 (1H, d, Jₒ=6 Hz, H₄ Trp), 7.19 (3H, t, Jₒ=8 Hz, H₃, H₄ and H₅ phenyl), 7.29 (1H, d, Jₒ=8 Hz, H₇ Trp), 8.01 (3H, brs, NH₂ Aib), 8.94 (1H, d, J=8 Hz, NH amide), 10.78 (1H, s, NH indole Trp).

¹³C NMR (75 MHz, DMSO-d⁶, 300° K):

δ(ppm) 23.5 (CH₃ Aib), 23.8 (CH₃ Aib), 26.5 (CH₂—CH₂-phenyl), 29.2 (CH₂ βTrp), 32.7 (CH₂—CH₂-phenyl), 45.4 (CH₂-p-fluorobenzyl), 45.7 (CH αTrp), 56.8 (Cq Aib), 109.8 (C₃ Trp), 111.8 (C₇ Trp), 115.9 and 116.2 (C₃ and C₅ p-fluorobenzyl), 118.3 (C₄ Trp), 118.7 (C₅ Trp), 121.3 (C₆ Trp), 124.8 (C₂ Trp), 126.5 (C₄ phenyl), 127.3 (C₉ Trp), 128.5 (C₂ and C₆ p-fluorobenzyl), 128.7 (C₂, C₃, C₅ and C₆ phenyl), 132.2 (C₁ p-fluorobenzyl), 136.4 (C₈ Trp), 140.9 (C₁ phenyl), 154.5 (Cq triazole), 154.7 (Cq triazole), 160.3 (C₄ p-fluorobenzyl), 171.9 (CO amide).

NMR ¹⁹F (282 MHz, DMSO-d⁶, 300° K):

δ(ppm) −114.9 (1F, m, p-fluorobenzyl).

### (R)-N-(1-(4-(3,4-dichlorobenzyl)-5-phenethyl-4H-1,2,4-triazol-3-yl)-2-(1H-indol-3-yl)ethyl)-2-amino-2-methylpropanamide (Compound 122):

¹H NMR (300 MHz, DMSO-d⁶, 300° K):

δ(ppm) 1.25 (3H, s, CH₃ Aib), 1.26 (3H, s, CH₃ Aib), 2.84 (4H, m, CH₂—CH₂-phenyl), 3.34 (2H, d, J=7 Hz, CH₂ βTrp), 5.11 (3H, m, CH αTrp and CH₂-m,p-dichlorobenzyl), 6.63 (1H, dd, Jₒ=8 Hz and Jₘ=2 Hz, H₆ m,p-dichlorobenzyl), 6.83 (1H, t, Jₒ=8 Hz, H₅ Trp), 6.99 (1H, t, Jₒ=8 Hz, H₆ Trp), 7.05 (1H, d, J=2 Hz, H₂ Trp), 7.12 (5H, m, CHar phenyl), 7.17 (1H, d, Jₒ=8 Hz, H₄ Trp), 7.21 (1H, s, H₂ m,p-dichlorobenzyl), 7.27 (1H, d, Jₒ=8 Hz, H₅ m,p-dichlorobenzyl), 7.39 (1H, d, Jₒ=8 Hz, H₇ Trp), 8.02 (3H, brs, NH₂ Aib), 8.94 (1H, d, J=8 Hz, NH amide), 10.78 (1H, s, NH indole Trp).

¹³C NMR (75 MHz, DMSO-d⁶, 300° K):

δ(ppm) 23.5 (CH₃ Aib), 23.8 (CH₃ Aib), 26.4 (CH₂—CH₂-phenyl), 29.1 (CH₂ βTrp), 32.6 (CH₂—CH₂-phenyl), 44.7 (CH₂-m,p-dichlorobenzyl), 45.6 (CH α Trp), 56.7 (Cq Aib), 109.7 (C₃ Trp), 111.8 (C₇ Trp), 118.1 (C₄ Trp), 118.7 (C₅ Trp), 121.3 (C₆ Trp), 124.9 (C₂ Trp), 126.5 (C₄ phenyl and C₆ m,p-dichlorobenzyl), 127.2 (C₉ Trp), 128.7 (C₂, C₃, C₅ and C₆ phenyl, C₂ m,p-dichlorobenzyl), 130.9 (C₄ m,p-dichlorobenzyl), 131.4 (C₅ m,p-dichlorobenzyl), 132.0 (C₃ m,p-dichlorobenzyl), 136.4 (C₈ Trp), 137.2 (C₅ m,p-dichlorobenzyl), 140.8 (C₁ phenyl), 154.5 (Cq triazole), 154.7 (Cq triazole), 171.9 (CO amide).

### (R)-N-(1-(4-(4-methylbenzyl)-5-benzyl-4H-1,2,4-triazol-3-yl)-2-(1H-indol-3-yl)ethyl)-2-amino-2-methylpropanamide (Compound 124):

¹H NMR (300 MHz, DMSO-d⁶, 300° K):

δ(ppm) 1.22 (3H, s, CH₃ Aib), 1.27 (3H, s, CH₃ Aib), 2.22 (3H, s, CH₃ p-methylbenzyl), 3.22 (1H, dd, J=14 Hz and 6 Hz, CH₂ βTrp), 3.33 (1H, dd, J=14 Hz and 9 Hz, CH₂ βTrp), 3.99 (2H, s, CH₂-benzyl), 5.04 (2H, s, CH₂-p-methylbenzyl), 5.09 (1H, m, CH αTrp), 6.64 (2H, d, Jₒ=8 Hz, H₃ and H₅ p-methylbenzyl), 6.78 (1H, t, Jₒ=7 Hz, H₅ Trp), 6.98 (4H, t, Jₒ=7 Hz, H₆ Trp, H₃, H₄ and H₅ benzyl), 7.01 (1H, d, J=2 Hz, H₂ Trp), 7.07 (2H, d, Jₒ=7 Hz, H₂ and H₆ p-methylbenzyl), 7.20 (3H, m, H₄ Trp, H₂ and H₆ benzyl), 7.26 (1H, d, Jₒ=8 Hz, H₇ Trp), 7.98 (3H, brs, NH₂ Aib), 8.89 (1H, d, J=8 Hz, NH amide), 10.74 (1H, s, NH indole Trp).

¹³C NMR (75 MHz, DMSO-d⁶, 300° K):

δ(ppm) 21.0 (CH₃ p-methylbenzyl), 23.5 (CH₃ Aib), 23.7 (CH₃ Aib), 29.1 (CH₂ βTrp), 30.6 (CH₂-benzyl), 45.7 (CH αTrp), 46.0 (CH₂-p-methylbenzyl), 56.7 (Cq Aib), 109.8 (C₃ Trp), 111.7 (C₇ Trp), 118.3 (C₄ Trp), 118.6 (C₅ Trp), 121.2 (C₆ Trp), 124.8 (C₂ Trp), 126.3 (C₃ and C₅ p-methylbenzyl), 127.0 (C₄ benzyl), 127.2 (C₉ Trp), 128.9 (C₂, C₃, C₅ and C₆ benzyl), 129.7 (C₂ and C₆ p-methylbenzyl), 132.9 (C₁ p-methylbenzyl), 136.3 (C₄ p-methylbenzyl), 136.4 (C₈ Trp), 137.4 (C₁ benzyl), 153.9 (Cq triazole), 155.3 (Cq triazole), 171.8 (CO amide).

### (S)-N-(1-(4-(4-methoxybenzyl)-5-(3-phenylpropyl)-4H-1,2,4-triazol-3-yl)-2-(1H-indol-3-yl)ethyl)-2-amino-2-methylpropanamide (Compound 125):

¹H NMR (300 MHz, DMSO-d⁶, 300° K):

δ(ppm) 1.27 (3H, s, CH₃ Aib), 1.31 (3H, s, CH₃ Aib), 1.74 (2H, m, (CH₂—CH₂—CH₂-phenyl), 2.52 (4H, t, J=7 Hz, CH₂—CH₂—CH₂-phenyl), 3.35 (2H, d, J=7 Hz, CH₂ βTrp), 3.68 (3H, s, OCH₃), 4.98 (2H, s, CH₂-p-methoxybenzyl), 5.20 (1H, m, CH αTrp), 6.75 (4H, s, CHar p-methoxybenzyl), 6.83 (1H, t, Jₒ=8 Hz, H₅ Trp), 6.99 (1H, t, Jₒ=7 Hz, H₆ Trp), 7.06 (3H, m, H₂ and H₆ phenyl, H₂ Trp), 7.14 (1H, d, Jₒ=7 Hz, H₄ Trp), 7.19 (3H, t, Jₒ=8 Hz, H₃, H₄ and H₅ phenyl), 7.29 (1H, d, Jₒ=8 Hz, H₇ Trp), 8.01 (3H, brs, NH₂ Aib), 8.94 (1H, d, J=8 Hz, NH amide), 10.79 (1H, d, J=2 Hz, NH indole Trp).

¹³C NMR (75 MHz, DMSO-d⁶, 300° K):

δ(ppm) 23.6 (CH₃ Aib), 23.8 (CH₃ Aib), 24.1 (CH₂—CH₂—CH₂-phenyl), 28.5 (CH₂—CH₂—CH₂-phenyl), 29.2 (CH₂ βTrp), 34.7 (CH₂—CH₂—CH₂-phenyl), 45.5 (CH₂-p-methoxybenzyl), 45.8 (CH αTrp), 55.5 (OCH₃), 56.8 (Cq Aib), 109.8 (C₃ Trp), 111.8 (C₇ Trp), 114.6 (C₃ and C₅ p-methoxybenzyl), 118.3 (C₄ Trp), 118.7 (C₅ Trp), 121.3 (C₆ Trp), 124.9 (C₂ Trp), 126.2 (C₄ phenyl), 127.3 (C₉ Trp), 127.8 (C₁ p-methoxybenzyl), 127.9 (C₃, C₄ and C₅ phenyl), 128.7 (C₂ and C₆ p-methoxybenzyl), 136.4 (C₈ Trp), 141.7 (C₁ phenyl), 154.7 (Cq triazole), 154.8 (Cq triazole), 159.2 (C₁ p-methoxybenzyl), 171.9 (CO amide).

### (S)-N-(1-(4-(4-methoxybenzyl)-5-benzyl-4H-1,2,4-triazol-3-yl)-2-(1H-indol-3-yl)ethyl)-2-amino-2-methylpropanamide (Compound 126):

¹H NMR (300 MHz, DMSO-d⁶, 300° K):

δ(ppm) 1.25 (3H, s, CH₃ Aib), 1.28 (3H, s, CH₃ Aib), 3.26 (1H, dd, J=14 Hz and 6 Hz, CH₂ βTrp), 3.34 (1H, dd, J=14 Hz and 8 Hz, CH₂ βTrp), 3.99 (2H, s, CH₂-phenyl), 4.98 (2H, s, CH₂-p-methoxybenzyl), 5.13 (1H, m, CH αTrp), 6.68 (4H, s, CHar p-methoxybenzyl), 6.80 (1H, t, Jₒ=8 Hz, H₅ Trp), 6.99 (1H, t, Jₒ=8 Hz, H₆ Trp), 7.02-7.06 (4H, m, H₂ and H₄ Trp, H₂ and H₆ phenyl), 7.20 (3H, m, H₃, H₄ and H₅ phenyl), 7.27 (1H, d, Jₒ=8 Hz, H₇ Trp), 8.00 (3H, s, NH₂ Aib), 8.91 (1H, d, J=8 Hz, NH amide), 10.76 (1H, s, NH indole Trp).

¹³C NMR (75 MHz, DMSO-d⁶, 300° K):

δ(ppm) 23.5 (CH₃ Aib), 23.7 (CH₃ Aib), 29.1 (CH₂ βTrp), 30.6 (CH₂-phenyl), 45.7 (CH αTrp and CH₂-p-methoxybenzyl), 55.5 (OCH₃), 56.7 (Cq Aib), 109.8 (C₃ Trp), 111.7 (C₇ Trp), 114.5 (C₃ and C₅ p-methoxybenzyl), 118.3 (C₄ Trp), 118.7 (C₅ Trp), 121.3 (C₆ Trp), 124.8 (C₂ Trp), 127.1 (C₄ phenyl), 127.3 (C₉ Trp), 127.6 (C₁ p-methoxybenzyl), 127.8 (C₂ and C₆ p-methoxybenzyl), 128.8 (C₂ and C₆ phenyl), 128.9 (C₃ and C₅ phenyl), 136.3 (C₈ Trp), 136.4 (C₁ phenyl), 153.8 (Cq triazole), 155.2 (Cq triazole), 159.2 (C₄ p-methoxybenzyl), 171.9 (CO amide).

### N-((R)-1-(4-(4-nitrobenzyl)-5-phenethyl-4H-1,2,4-triazol-3-yl)-2-(1H-indol-3-yl)ethyl)-2-amino-2-methylpropanamide (Compound 128):

¹H NMR (300 MHz, DMSO-d⁶, 300° K):

δ(ppm) 1.28 (3H, s, CH₃ Aib), 1.29 (3H, s, CH₃ Aib), 2.77-2.94 (4H, m, CH₂—CH₂-phenyl), 3.28 (1H, dd, ³J=14 Hz and 8 Hz, CH₂ βTrp), 3.43 (1H, dd, ³J=14 Hz and 7 Hz, CH₂ βTrp), 5.05 (1H, m, CH αTrp), 5.25 (2H, d, J=7 Hz, CH₂-p-nitrobenzyl), 6.72 (1H, t, Jₒ=7 Hz, H₅ Trp), 6.89 (2H, d, Jₒ=9 Hz, H₂ and H₆ p-nitrobenzyl), 6.92 (1H, t, Jₒ=7 Hz, H₆ Trp), 7.00 (1H, d, Jₘ=2 Hz, H₂ Trp), 7.08-7.15 (4H, m, H₄ and H₇ Trp, H₂ and H₆ phenyl), 7.17 (2H, Jₒ=7 Hz, H₃ and H₅ phenyl), 7.24 (1H, t, Jₒ=8 Hz, H₄ phenyl), 7.92 (2H, d, Jₒ=9 Hz, H₃ and H₅ p-nitrobenzyl), 8.06 (3H, brs, NH₂ Aib), 8.98 (1H, d, J=8 Hz, NH amide), 10.79 (1H, s, NH indole Trp).

¹³C NMR (75 MHz, DMSO-d⁶, 300° K):

δ(ppm) 23.5 (CH₃ Aib), 23.8 (CH₃ Aib), 26.4 (CH₂—CH₂-phenyl), 29.1 (CH₂ βTrp), 32.6 (CH₂—CH₂-phenyl), 45.3 (CH₂-p-nitrobenzyl), 45.7 (CH αTrp), 56.8 (Cq Aib), 109.6 (C₃ Trp), 111.8 (C₇ Trp), 118.1 (C₄ Trp), 118.5 (C₅ Trp), 121.2 (C₆ Trp), 124.1 (C₂ and C₅ p-nitrobenzyl), 124.8 (C₂ Trp), 126.5 (C₂ and C₅ phenyl), 127.2 (C₉ Trp, C₁ and C₆ p-nitrobenzyl), 128.7 (C₃, C₄ and C₅ phenyl), 136.4 (C₈ Trp), 140.8 (C₁ phenyl), 143.5 (C₁ p-nitrobenzyl), 154.5 (Cq triazole), 154.8 (Cq triazole), 172.0 (CO Aib).

### (S)-N-(1-(4-(4-methoxybenzyl)-5-phenethyl-4H-1,2,4-triazol-3-yl)-2-(1H-indol-3-yl)ethyl)-2-amino-2-methylpropanamide (Compound 129):

¹H NMR (300 MHz, DMSO-d⁶, 300° K):

δ(ppm) 1.27 (3H, s, CH₃ Aib), 1.30 (3H, s, CH₃ Aib), 2.81 (4H, m, CH₂—CH₂-phenyl), 3.34 (2H, d, J=7 Hz, CH₂ βTrp), 3.68 (3H, s, OCH₃), 4.99 (2H, s, CH₂-p-methoxybenzyl), 5.20 (1H, m, CH αTrp), 6.75 (4H, m, CHar p-methoxybenzyl), 6.83 (1H, t, Jₒ=7 Hz, H₅ Trp), 7.03 (1H, t, Jₒ=8 Hz, H₆ Trp), 7.04 (1H, d, J=2 Hz, H₂ Trp), 7.10 (2H, d, Jₒ=7 Hz, H₂ and H₆ phenyl), 7.13 (1H, d, Jₒ=8 Hz, H₄ Trp), 7.19 (3H, t, Jₒ=7 Hz, H₃, H₄ and H₅ phenyl), 7.29 (1H, d, Jₒ=8 Hz, H₇ Trp), 8.01 (3H, brs, NH₂ Aib), 8.94 (1H, d, J=8 Hz, NH amide), 10.78 (1H, d, J=2 Hz, NH indole Trp).

¹³C NMR (75 MHz, DMSO-d⁶, 300° K):

δ(ppm) 23.6 (CH₃ Aib), 23.8 (CH₃ Aib), 26.6 (CH₂—CH₂-phenyl), 29.2 (CH₂ βTrp), 32.7 (CH₂—CH₂-phenyl), 45.3 (CH₂-p-methoxybenzyl), 45.7 (CH αTrp), 55.5 (OCH₃), 56.8 (Cq Aib), 109.9 (C₃ Trp), 111.8 (C₇ Trp), 114.6 (C₃ and C₅ p-methoxybenzyl), 118.3 (C₄ Trp), 118.7 (C₅ Trp), 121.3 (C₆ Trp), 124.8 (C₂ Trp), 126.5 (C₄ phenyl), 127.3 (C₉ Trp and C₁ p-methoxybenzyl), 127.9 (C₂ and C₆ p-methoxybenzyl), 128.7 (C₂, C₃, C₅ and C₆ p-methoxybenzyl), 136.5 (C₈ Trp), 140.9 (C₁ phenyl), 154.4 (Cq triazole), 154.7 (Cq triazole), 159.2 (C₄ p-methoxybenzyl), 171.9 (CO amide).

### (R)-N-(1-(4-(4-methoxyphenethyl)-5-phenethyl-4H-1,2,4-triazol-3-yl)-2-(1H-indol-3-yl)ethyl)-2-amino-2-methylpropanamide (Compound 130):

¹H NMR (300 MHz, DMSO-d⁶, 300° K):

δ(ppm) 1.30 (3H, s, CH₃ Aib), 1.35 (3H, s, CH₃ Aib), 2.55 (4H, m, CH₂—CH₂-phenyl and CH₂—CH₂-p-methoxybenzyl), 2.83 (2H, t, J=8 Hz, CH₂—CH₂-phenyl), 3.37 (2H, m, CH₂—CH₂-p-methoxybenzyl), 3.65 (3H, s, OCH₃), 3.77 (1H, m, CH₂ βTrp), 3.89 (1H, m, CH₂ βTrp), 5.20 (1H, m, CH αTrp), 6.72 (4H, s, CHar p-methoxybenzyl), 6.94 (1H, t, Jₒ=7 Hz, H₅ Trp), 7.02 (1H, t, Jₒ=8 Hz, H₆ Trp), 7.05 (1H, d, J=2 Hz, H₂ Trp), 7.11 (2H, d, Jₒ=7 Hz, H₂ and H₆ phenyl), 7.16 (1H, d, Jₒ=7 Hz, H₄ Trp), 7.25 (3H m, H₃, H₄, H₅ phenyl), 7.50 (1H, d, Jₒ=8 Hz, H₇ Trp), 8.05 (3H, brs, NH₂ Aib), 9.02 (1H, d, J=8 hz, NH amide), 10.83 (1H, d, J=2 Hz, NH indole Trp).

¹³C NMR (75 MHz, DMSO-d⁶, 300° K):

δ(ppm) 23.5 (CH₃ Aib), 23.8 (CH₃ Aib), 26.0 (CH₂—CH₂-phenyl), 29.5 (CH₂ βTrp), 32.5 (CH₂—CH₂-phenyl), 35.0 (CH₂—CH₂-p-methoxybenzyl), 44.4 (CH₂—CH₂-p-methoxybenzyl), 45.8 (CH αTrp), 55.4 (OCH₃), 56.8 (Cq Aib), 109.9 (C₃ Trp), 111.9 (C₇ Trp), 114.2 (C₃ and C₅ p-methoxybenzyl), 118.4 (C₄ Trp), 118.9 (C₅ Trp), 121.4 (C₆ Trp), 124.7 (C₂ Trp), 126.5 (C₄ phenyl), 127.4 (C₉ Trp), 128.7 (C₂, C₃, C₅ and C₆ phenyl), 129.4 (C₁ p-methoxybenzyl), 130.3 (C₂ and C₆ p-methoxybenzyl), 136.5 (C₈ Trp), 141.0 (C₁ phenyl), 154.0 (Cq triazole), 154.5 (Cq triazole), 158.6 (C₄ p-methoxybenzyl), 171.8 (CO amide).

### (R)-N-(2-(1H-indol-3-yl)-1-(5-phenethyl-4-(pyridin-2-ylmethyl)-4H-1,2,4-triazol-3-yl)ethyl)-2-amino-2-methylpropanamide (Compound 132):

¹H NMR (300 MHz, DMSO-d⁶, 300° K):

δ(ppm) 1.26 (3H, s, CH₃ Aib), 1.29 (3H, s, CH₃ Aib), 2.95 (4H, m, CH₂—CH₂-phenyl), 3.40 (2H, m, CH₂ βTrp), 5.26 (1H, m, CH αTrp), 5.37 (2H, s, CH₂-o-pyridyl), 6.83 (1H, t, Jₒ=7 Hz, H₅ Trp), 6.98 (1H, t, Jₒ=8 Hz, H₆ Trp), 7.11-7.30 (10H, m, H₂, H₄ and H₇ Trp, CHar phenyl, H₃ and H₅ o-pyridyl), 7.71 (1H, t, Jₒ=7 Hz, H₄ o-pyridyl), 8.22 (3H, brs, NH₂ Aib), 8.42 (1H, d, J_{αβ}32 4 Hz, H₆ o-pyridyl), 9.05 (1H, d, J=8 Hz, NH amide), 10.87 (1H, s, NH indole Trp).

¹³C NMR (75 MHz, DMSO-d⁶, 300° K):

δ(ppm) 23.4 (CH₃ Aib), 23.7 (CH₃ Aib), 26.4 (CH₂—CH₂-phenyl), 28.6 (CH₂ βTrp), 32.5 (CH₂—CH₂-phenyl), 45.7 (CH αTrp), 47.6 (CH₂ o-pyridyl), 56.8 (Cq Aib), 109.8 (C₃ Trp), 111.8 (C₇ Trp), 118.3 (C₄ Trp), 118.6 (C₅ Trp), 121.2 (C₆ Trp), 122.0 (C₃ o-pyridyl), 123.6 (C₅ o-pyridyl), 126.3 (C₂ Trp), 126.6 (C₄ phenyl), 127.3 (C₉ Trp), 128.7 (C₂, C₃, C₅ and C₆ phenyl), 136.4 (C₈ trypophane), 137.7 (C₄ o-pyridyl), 140.7 (C₁ phenyl), 150.1 (C₆ o-pyridyl), 154.9 (Cq triazole), 155.2 (Cq triazole), 158.7 (C₂ o-pyridyl), 172.0 (CO amide).

### N-((R)-1-(4-(2,4-dimethoxybenzyl)-5-phenethyl-4H-1,2,4-triazol-3-yl)-2-(1H-indol-3-yl)ethyl)-2-amino-2-methylpropanamide (Compound 179):

¹H NMR (300 MHz, DMSO d⁶, 300° K):

δ(ppm) 1.26 (s, 3H, CH₃ Aib), 1.30 (s, 3H, CH₃ Aib), 2.82 (m, 4H, CH₂—CH₂-phenyl), 3.29 (t, 2H, J=8 Hz, CH₂ βTrp), 3.61 (s, 3H, OCH₃), 3.68 (s, 3H, OCH₃), 4.85 (d, 1H, J=17 Hz, CH₂-o,p-dimethoxybenzyl), 5.02 (d, 1H, J=17 Hz, CH₂-o,p-dimethoxybenzyl), 5.18 (m, 1H, CH αTrp), 6.29 (dd, 1H, Jₒ=8 Hz and Jₘ=2 Hz, H₅ o,p-dimethoxybenzyl), 6.40 (d, 1H, Jₒ=8 Hz, H₆ o,p-dimethoxybenzyl), 6.55 (d, 1H, Jₘ=2 Hz, H₃ o,p-dimethoxybenzyl), 6.82 (t, 1H, Jₒ=8 Hz, H₅ Trp), 6.99 (t, 1H, Jₒ=8 Hz, H₆ Trp), 7.05 (s, 1H, H₂ Trp), 7.09-7.24 (m, 6H, H₄ Trp and CHar phenyl), 7.27 (d, 1H, Jₒ=8 Hz, H₇ Trp), 7.99 (s, 3H, large, NH₂ Aib TFA salt), 8.89 (d, 1H, J=8 Hz, NH amide), 10.77 (s, 1H, NH indole Trp).

¹³C NMR (75 MHz, DMSO d⁶, 300° K):

δ(ppm) 23.6 (CH₃ Aib), 23.7 (CH₃ Aib), 26.5 (CH₂—CH₂-phenyl), 29.2 (CH₂ βTrp), 32.7 (CH₂—CH₂-phenyl), 41.6 (CH₂-o,p-dimethoxybenzyl), 45.7 (CH αTrp), 55.7 (OCH₃), 55.9 (OCH₃), 56.7 (Cq Aib), 99.1 (C₃ o,p-dimethoxybenzyl), 105.2 (C₅ o,p-dimethoxybenzyl), 110.0 (C₃ Trp), 111.8 (C₇ Trp), 115.7 (C₁ o,p-dimethoxybenzyl), 118.3 (C₄ Trp), 118.7 (C₅ Trp), 121.3 (C₆ Trp), 126.5 (C₂ Trp and C₆ o,p-dimethoxybenzyl), 127.3 (C₉ Trp), 128.1 (C₄ phenyl), 128.7 (C₂, C₃, C₅ and C₆ phenyl), 136.4 (C₈ Trp), 140.9 (C₁ phenyl), 154.5 (Cq triazole), 155.0 (Cq triazole), 157.8 (C₂ o,p-dimethoxybenzyl), 160.9 (C₄ o,p-dimethoxybenzyl), 171.7 (CO amide).

### N-((R)-1-(4-(4-methoxybenzyl)-5-phenethyl-4H-1,2,4-triazol-3-yl)-2-(1H-indol-3-yl)ethyl)-tetrahydro-2H-pyran-4-carboxamide (Compound 184):

¹H NMR (300 MHz, DMSO d⁶, 300° K):

δ(ppm) 1.20 (m, 1H, H₅ tetrahydropyrane), 1.30 (m, 3H, H₃ and H₅ tetrahydropyrane), 2.17 (m, 1H, H₄ tetrahydropyrane), 2.82 (m, 4H, CH₂—CH₂-phenyl), 3.16 (m, 2H, H₂ and H₆ tetrahydropyrane), 3.31 (dd, 1H, J=14 Hz and 8 Hz, CH₂ βTrp), 3.35 (dd, 1H, J=14 Hz and 7 Hz, CH₂ βTrp), 3.66 (s, 3H, OCH₃), 3.72 (m, 2H, H₂ and H₆ tetrahydropyrane), 5.08 (m, 2H, CH₂-p-methoxybenzyl), 5.26 (m, 1H, CH αTrp), 6.73 (s, 4H, CHar p-methoxybenzyl), 6.87 (t, 1H, Jₒ=8 Hz, H₅ Trp), 7.02 (m, 2H, H₂ and H₆ Trp), 7.07 (d, 2H, Jₒ=7 Hz, H₂ and H₆ phenyl), 7.18 (m, 3H, H₃, H₄ and H₅ phenyl), 7.30 (m, 2H, H₄ and H₇ Trp), 8.52 (d, 1H, J=8 Hz, NH amide), 10.77 (s, 1H, NH indole Trp).

¹³C NMR (75 MHz, DMSO d⁶, 300° K):

δ(ppm) 26.3 (CH₂—CH₂-phenyl), 28.8 (C₃ and C₅ tetrahydropyrane), 29.2 (CH₂ βTrp), 32.2 (CH₂—CH₂-phenyl), 40.7 (C₄ tetrahydropyrane), 44.8 (CH αTrp), 45.9 (CH₂-p-methoxybenzyl), 55.5 (OCH₃), 66.6 (C₂ and C₆ tetrahydropyrane), 109.8 (C₃ Trp), 111.7 (C₇ Trp), 114.5 (C₃ and C₅ p-methoxybenzyl), 118.4 (C₄ Trp), 118.7 (C₅ Trp), 121.3 (C₆ Trp), 124.5 (C₂ Trp), 126.7 (C₄ phenyl), 127.2 (C₉ Trp), 127.5 (C₁ p-methoxybenzyl), 128.8 (C₂ and C₆ phenyl), 128.7 (C₃ and C₅ phenyl), 127.9 (C₂ and C₆ p-methoxybenzyl), 136.4 (C₈ Trp), 140.4 (C₁ phenyl), 154.7 (Cq triazole), 155.7 (Cq triazole), 159.2 (C₄ p-methoxybenzyl), 174.2 (CO amide).

### N-((R)-1-(5-((1H-indol-3-yl)methyl)-4-(3-methoxybenzyl)-4H-1,2,4-triazol-3-yl)-2-(1H-indol-3-yl)ethyl)-2-amino-2-methylpropanamide (Compound 185):

¹H NMR (300 MHz, DMSO d⁶, 300° K):

δ(ppm) 1.19 (s, 3H, CH₃ Aib), 1.23 (s, 3H, CH₃ Aib), 3.14 (dd, 1H, J=14 Hz and 5 Hz, CH₂ βTrp), 3.34 (dd, 1H, J=14 Hz and 9 Hz, CH₂ βTrp), 3.46 (s, 3H, OCH₃), 3.72 (m, 2H, CH₂-indole), 5.06 (m, 1H, CH αTrp), 5.14 (m, 2H, CH₂-m-methoxybenzyl), 6.31 (s, 1H, H₂ m-methoxybenzyl), 6.32 (d, 1H, Jₒ=7 Hz, H₆ m-methoxybenzyl), 6.77 (m, 3H, H₅ indole, H₅ Trp and H₄ m-methoxybenzyl), 6.92 (m, 2H, H₆ indole and H₆ Trp), 7.02 (m, 2H, H₂ indole and H₂ Trp), 7.26-7.30 (m, 3H, H₅ m-methoxybenzyl, H₄ and H₇ indole, H₄ Trp), 7.41 (d, 1H, Jₒ=8 Hz, H₇ Trp), 7.94 (brs, 3H, NH₂ Aib TFA salt), 8.87 (d, 1H, J=8 Hz, NH amide), 10.73 (d, 1H, J=2 Hz, NH indole), 10.85 (s, 1H, NH indole Trp).

¹³C NMR (75 MHz, DMSO d⁶, 300° K):

δ(ppm) 21.7 (CH₂-indole), 23.5 (CH₃ Aib), 23.7 (CH₃ Aib), 28.9 (CH₂ βTrp), 45.5 (CH αTrp), 46.2 (CH₂-m-methoxybenzyl), 55.2 (OCH₃), 56.7 (Cq Aib), 108.2 (C₃ indole), 109.8 (C₃ Trp), 111.7 (C₇ Trp), 111.9 (C₇ indole and C₂ m-methoxybenzyl), 113.7 (C₄ m-methoxybenzyl), 118.2 (C₆ m-methoxybenzyl), 118.4 (C₄ Trp), 118.6 (C₄ indole), 118.9 (C₅ indole and C₅ Trp), 121.2 (C₆ indole), 121.6 (C₆ Trp), 127.1 (C₉ indole), 127.2 (C₉ Trp), 136.4 (C₈ Trp), 136.7 (C₈ indole), 137.5 (C₁ m-methoxybenzyl), 154.2 (Cq triazole), 155.3 (Cq triazole), 160.0 (C₃ m-methoxybenzyl), 171.8 (CO amide).

TABLE 1
Further exemplary embodiments with synthetic sequence and MS data
(compounds no. 2, 3, 14, 16, 17, 19-22, 24, 26-35, 36-122, 124-126, 128-150,
152-190):

| | Chemical name | ESI-MS | ESI-MS |
|---|---|---|---|
| No. | (Chem Draw Ultra 8) | (calculated) | [found (M + H)⁺] |
| 2 | (R)—N-(1-(5-(2-(1H-indol-3-yl)ethyl)-4-phenethyl- | 559.3 | 560.4 |
| | 4H-1,2,4-triazol-3-yl)-2-(1H-indol-3-yl)ethyl)-2- | | |
| | amino-2-methylpropanamide | | |
| 3 | (R)—N-(1-(5-(3-(1H-indol-3-yl)propyl)-4-phenethyl- | 573.3 | 574.3 |
| | 4H-1,2,4-triazol-3-yl)-2-(1H-indol-3-yl)ethyl)-2- | | |
| | amino-2-methylpropanamide | | |
| 14 | (R)—N-(1-(5-(3-(1H-indol-3-yl)propyl)-4-(4- | 637.2 | 638.1 |
| | bromobenzyl)-4H-1,2,4-triazol-3-yl)-2-(1H-indol-3- | | |
| | yl)ethyl)-2-amino-2-methylpropanamide | | |
| 16 | (R)—N-(1-(5-(3-(1H-indol-3-yl)propyl)-4-hexyl-4H- | 553.3 | 554.4 |
| | 1,2,4-triazol-3-yl)-2-(1H-indol-3-yl)ethyl)-2-amino- | | |
| | 2-methylpropanamide | | |
| 17 | (R)—N-(1-(4,5-bis(2-(1H-indol-3-yl)ethyl)-4H-1,2,4- | 598.3 | 599.2 |
| | triazol-3-yl)-2-(1H-indol-3-yl)ethyl)-2-amino-2- | | |
| | methylpropanamide | | |
| 19 | (R)—N-(1-(4-(3-methoxybenzyl)-5-phenethyl-4H- | 536.3 | 537.1 |
| | 1,2,4-triazol-3-yl)-2-(1H-indol-3-yl)ethyl)-2-amino- | | |
| | 2-methylpropanamide | | |
| 20 | (R)—N-(1-(4-(4-methoxybenzyl)-5-phenethyl-4H- | 536.3 | 537.3 |
| | 1,2,4-triazol-3-yl)-2-(1H-indol-3-yl)ethyl)-2-amino- | | |
| | 2-methylpropanamide | | |
| 21 | (R)—N-(1-(5-(2-(1H-indol-3-yl)ethyl)-4-(3,5- | 605.3 | 606.4 |
| | dimethoxybenzyl)-4H-1,2,4-triazol-3-yl)-2-(1H- | | |
| | indol-3-yl)ethyl)-2-amino-2-methylpropanamide | | |
| 22 | (R)—N-(1-(4-(4-methoxybenzyl)-5-(3-phenylpropyl)- | 550.3 | 551.3 |
| | 4H-1,2,4-triazol-3-yl)-2-(1H-indol-3-yl)ethyl)-2- | | |
| | amino-2-methylpropanamide | | |
| 24 | (R)—N-(1-(4-(2-(1H-indol-3-yl)ethyl)-5-(3-(1H-indol- | 612.3 | 612.8 |
| | 3-yl)propyl)-4H-1,2,4-triazol-3-yl)-2-(1H-indol-3- | | |
| | yl)ethyl)-2-amino-2-methylpropanamide | | |
| 26 | (R)—N-(1-(4-(2-methoxybenzyl)-5-phenethyl-4H- | 536.3 | 537.1 |
| | 1,2,4-triazol-3-yl)-2-(1H-indol-3-yl)ethyl)-2-amino- | | |
| | 2-methylpropanamide | | |
| 27 | (R)—N-(2-(1H-indol-3-yl)-1-(4-(naphthalen-1- | 556.3 | 557.2 |
| | ylmethyl)-5-phenethyl-4H-1,2,4-triazol-3-yl)ethyl)- | | |
| | 2-amino-2-methylpropanamide | | |
| 28 | (R)—N-(1-(5-(2-(1H-indol-3-yl)ethyl)-4-(3,4- | 613.2 | 614.2 |
| | dichlorobenzyl)-4H-1,2,4-triazol-3-yl)-2-(1H-indol- | | |
| | 3-yl)ethyl)-2-amino-2-methylpropanamide | | |
| 29 | (R)—N-(1-(5-(2-(1H-indol-3-yl)ethyl)-4-(4- | 563.3 | 564.1 |
| | fluorobenzyl)-4H-1,2,4-triazol-3-yl)-2-(1H-indol-3- | | |
| | yl)ethyl)-2-amino-2-methylpropanamide | | |
| 30 | (R)—N-(1-(4-(4-fluorobenzyl)-5-benzyl-4H-1,2,4- | 510.3 | 511.0 |
| | triazol-3-yl)-2-(1H-indol-3-yl)ethyl)-2-amino-2- | | |
| | methylpropanamide | | |
| 31 | (R)—N-(1-(5-(2-(1H-indol-3-yl)ethyl)-4-(2,4- | 631.3 | 632.0 |
| | dimethoxybenzyl)-4H-1,2,4-triazol-3-yl)-2-(1H- | | |
| | indol-3-yl)ethyl)piperidine-4-carboxamide | | |
| 32 | (R)—N-(1-(5-(2-(1H-indol-3-yl)ethyl)-4-(2,4- | 631.3 | 631.8 |
| | dimethoxybenzyl)-4H-1,2,4-triazol-3-yl)-2-(1H- | | |
| | indol-3-yl)ethyl)piperidine-3-carboxamide | | |
| 33 | (R)—N-(1-(4-(4-methylbenzyl)-5-(3-phenylpropyl)- | 534.3 | 535.4 |
| | 4H-1,2,4-triazol-3-yl)-2-(1H-indol-3-yl)ethyl)-2- | | |
| | amino-2-methylpropanamide | | |
| 34 | (R)—N-(1-(5-(2-(1H-indol-3-yl)ethyl)-4-(4- | 559.7 | 559.9 |
| | methylbenzyl)-4H-1,2,4-triazol-3-yl)-2-(1H-indol-3- | | |
| | yl)ethyl)-2-amino-2-methylpropanamide | | |
| 36 | (R)—N-(1-(5-(2-(1H-indol-3-yl)ethyl)-4-(2,4- | 631.3 | 631.8 |
| | dimethoxybenzyl)-4H-1,2,4-triazol-3-yl)-2-(1H- | | |
| | indol-3-yl)ethyl)piperidine-2-carboxamide | | |
| 37 | (R)—N-(1-(4-(4-methylbenzyl)-5-phenethyl-4H- | 520.3 | 521.0 |
| | 1,2,4-triazol-3-yl)-2-(1H-indol-3-yl)ethyl)-2-amino- | | |
| | 2-methylpropanamide | | |
| 38 | (R)—N-(1-(5-(2-(1H-indol-3-yl)ethyl)-4-(2,4- | 639.3 | 639.8 |
| | dimethoxybenzyl)-4H-1,2,4-triazol-3-yl)-2-(1H- | | |
| | indol-3-yl)ethyl)-2-aminobenzamide | | |
| 39 | (R)—N-(1-(5-benzyl-4-($$yridine-2-ylmethyl)-4H- | 493.3 | 493.9 |
| | 1,2,4-triazol-3-yl)-2-(1H-indol-3-yl)ethyl)-2-amino- | | |
| | 2-methylpropanamide | | |
| 40 | (2S,4R)-N-((R)-1-(4-(4-methoxybenzyl)-5- | 564.3 | 565.0 |
| | phenethyl-4H-1,2,4-triazol-3-yl)-2-(1H-indol-3- | | |
| | yl)ethyl)-4-hydroxypyrrolidine-2-carboxamide | | |
| 41 | (S)—N-((R)-1-(4-(4-methoxybenzyl)-5-phenethyl- | 562.3 | 563.0 |
| | 4H-1,2,4-triazol-3-yl)-2-(1H-indol-3- | | |
| | yl)ethyl)piperidine-3-carboxamide | | |
| 42 | (R)—N-((R)-1-(4-(4-methoxybenzyl)-5-phenethyl- | 562.3 | 562.9 |
| | 4H-1,2,4-triazol-3-yl)-2-(1H-indol-3- | | |
| | yl)ethyl)piperidine-3-carboxamide | | |
| 43 | (R)—N-(1-(4-(4-ethylbenzyl)-5-phenethyl-4H-1,2,4- | 534.3 | 535.0 |
| | triazol-3-yl)-2-(1H-indol-3-yl)ethyl)-2-amino-2- | | |
| | methylpropanamide | | |
| 44 | (R)—N-(1-(4-(4-methoxybenzyl)-5-phenethyl-4H- | 562.3 | 563.0 |
| | 1,2,4-triazol-3-yl)-2-(1H-indol-3-yl)ethyl)piperidine- | | |
| | 4-carboxamide | | |
| 45 | (R)—N-(1-(5-(2-(1H-indol-3-yl)ethyl)-4-(4- | 601.3 | 602.0 |
| | methoxybenzyl)-4H-1,2,4-triazol-3-yl)-2-(1H-indol- | | |
| | 3-yl)ethyl)piperidine-4-carboxamide | | |
| 46 | (S)—N-((R)-1-(4-(4-methoxybenzyl)-5-phenethyl- | 548.3 | 548.9 |
| | 4H-1,2,4-triazol-3-yl)-2-(1H-indol-3- | | |
| | yl)ethyl)pyrrolidine-2-carboxamide | | |
| 47 | (R)—N-((R)-1-(4-(4-methoxybenzyl)-5-phenethyl- | 548.3 | 548.9 |
| | 4H-1,2,4-triazol-3-yl)-2-(1H-indol-3- | | |
| | yl)ethyl)pyrrolidine-2-carboxamide | | |
| 48 | (S)—N-((R)-1-(4-(4-methoxybenzyl)-5-phenethyl- | 562.3 | 563.0 |
| | 4H-1,2,4-triazol-3-yl)-2-(1H-indol-3- | | |
| | yl)ethyl)piperidine-2-carboxamide | | |
| 49 | (R)—N-((R)-1-(4-(4-methoxybenzyl)-5-phenethyl- | 562.3 | 562.9 |
| | 4H-1,2,4-triazol-3-yl)-2-(1H-indol-3- | | |
| | yl)ethyl)piperidine-2-carboxamide | | |
| 50 | (R)—N-(1-(4-(4-methoxybenzyl)-5-phenethyl-4H- | 508.3 | 508.9 |
| | 1,2,4-triazol-3-yl)-2-(1H-indol-3-yl)ethyl)-2- | | |
| | aminoacetamide | | |
| 51 | (R)—N-(1-(4-(4-methoxybenzyl)-5-phenethyl-4H- | 570.3 | 571.2 |
| | 1,2,4-triazol-3-yl)-2-(1H-indol-3-yl)ethyl)-2- | | |
| | ($$yridine-2-yl)acetamide | | |
| 52 | N-((R)-1-(4-(4-methoxybenzyl)-5-phenethyl-4H- | 570.3 | 570.9 |
| | 1,2,4-triazol-3-yl)-2-(1H-indol-3-yl)ethyl)-2- | | |
| | ($$yridine-4-yl)acetamide | | |
| 53 | (R)—N-(1-(4-(4-methoxybenzyl)-5-phenethyl-4H- | 561.3 | 562.4 |
| | 1,2,4-triazol-3-yl)-2-(1H-indol-3- | | |
| | yl)ethyl)cyclohexanecarboxamide | | |
| 54 | (R)—N-(1-(5-(2-(1H-indol-3-yl)ethyl)-4-benzyl-4H- | 571.3 | 572.5 |
| | 1,2,4-triazol-3-yl)-2-(1H-indol-3-yl)ethyl)piperidine- | | |
| | 4-carboxamide | | |
| 55 | (R)—N-(1-(5-(2-(1H-indol-3-yl)ethyl)-4-benzyl-4H- | 571.3 | 572.4 |
| | 1,2,4-triazol-3-yl)-2-(1H-indol-3-yl)ethyl)piperidine- | | |
| | 3-carboxamide | | |
| 56 | (R)—N-(1-(4-(4-methoxybenzyl)-5-phenethyl-4H- | 522.3 | 523.3 |
| | 1,2,4-triazol-3-yl)-2-(1H-indol-3-yl)ethyl)-3- | | |
| | aminopropanamide | | |
| 57 | (S)—N-((R)-1-(4-(4-methoxybenzyl)-5-phenethyl- | 522.3 | 523.3 |
| | 4H-1,2,4-triazol-3-yl)-2-(1H-indol-3-yl)ethyl)-2- | | |
| | aminopropanamide | | |
| 58 | (R)—N-(1-(4-(4-methoxybenzyl)-5-phenethyl-4H- | 570.3 | 571.2 |
| | 1,2,4-triazol-3-yl)-2-(1H-indol-3-yl)ethyl)-2-(pyridin- | | |
| | 3-yl)acetamide | | |
| 59 | (R)—N-(1-(4-(4-methoxybenzyl)-5-phenethyl-4H- | 584.3 | 585.3 |
| | 1,2,4-triazol-3-yl)-2-(1H-indol-3-yl)ethyl)-3-(pyridin- | | |
| | 3-yl)propanamide | | |
| 60 | (R)—N-(1-(5-(2-(1H-indol-3-yl)ethyl)-4-benzyl-4H- | 579.3 | 580.2 |
| | 1,2,4-triazol-3-yl)-2-(1H-indol-3-yl)ethyl)-2-(pyridin- | | |
| | 2-yl)acetamide | | |
| 61 | (R)—N-(1-(5-(2-(1H-indol-3-yl)ethyl)-4-(2,4- | 639.3 | 640.5 |
| | dimethoxybenzyl)-4H-1,2,4-triazol-3-yl)-2-(1H- | | |
| | indol-3-yl)ethyl)-2-(pyridin-2-yl)acetamide | | |
| 62 | (R)—N-(1-(4-(2,4-dimethoxybenzyl)-5-phenethyl- | 592.3 | 593.3 |
| | 4H-1,2,4-triazol-3-yl)-2-(1H-indol-3- | | |
| | yl)ethyl)piperidine-4-carboxamide | | |
| 63 | (R)—N-((R)-1-(4-(2,4-dimethoxybenzyl)-5- | 592.3 | 593.3 |
| | phenethyl-4H-1,2,4-triazol-3-yl)-2-(1H-indol-3- | | |
| | yl)ethyl)piperidine-2-carboxamide | | |
| 64 | (R)—N-(1-(4-(2,4-dimethoxybenzyl)-5-phenethyl- | 586.3 | 587.2 |
| | 4H-1,2,4-triazol-3-yl)-2-(1H-indol-3- | | |
| | yl)ethyl)picolinamide | | |
| 65 | (R)—N-(1-(4-(2,4-dimethoxybenzyl)-5-phenethyl- | 586.3 | 587.2 |
| | 4H-1,2,4-triazol-3-yl)-2-(1H-indol-3- | | |
| | yl)ethyl)isonicotinamide | | |
| 66 | (R)—N-(1-(4-(2,4-dimethoxybenzyl)-5-phenethyl- | 587.3 | 588.2 |
| | 4H-1,2,4-triazol-3-yl)-2-(1H-indol-3- | | |
| | yl)ethyl)pyrazine-2-carboxamide | | |
| 67 | (R)—N-1-(4-(2,4-dimethoxybenzyl)-5-phenethyl-4H- | 593.3 | 594.2 |
| | 1,2,4-triazol-3-yl)-2-(1H-indol-3-yl)ethyl)piperazine- | | |
| | 2-carboxamide | | |
| 68 | (S)—N-((R)-1-(4-(2,4-dimethoxybenzyl)-5- | 578.3 | 579.4 |
| | phenethyl-4H-1,2,4-triazol-3-yl)-2-(1H-indol-3- | | |
| | yl)ethyl)pyrrolidine-2-carboxamide | | |
| 69 | (R)—N-(1-(5-(2-(1H-indol-3-yl)ethyl)-4-(2,4- | 577.3 | 578.0 |
| | dimethoxybenzyl)-4H-1,2,4-triazol-3-yl)-2-(1H- | | |
| | indol-3-yl)ethyl)-2-aminoacetamide | | |
| 70 | (S)—N-((R)-1-(5-(2-(1H-indol-3-yl)ethyl)-4-(2,4- | 617.3 | 618.0 |
| | dimethoxybenzyl)-4H-1,2,4-triazol-3-yl)-2-(1H- | | |
| | indol-3-yl)ethyl)pyrrolidine-2-carboxamide | | |
| 71 | (R)—N-(1-(5-(2-(1H-indol-3-yl)ethyl)-4-(2,4- | 626.3 | 627.2 |
| | dimethoxybenzyl)-4H-1,2,4-triazol-3-yl)-2-(1H- | | |
| | indol-3-yl)ethyl)pyrazine-2-carboxamide | | |
| 72 | (R)—N-1-(5-(2-(1H-indol-3-yl)ethyl)-4-(2,4- | 632.3 | 633.2 |
| | dimethoxybenzyl)-4H-1,2,4-triazol-3-yl)-2-(1H- | | |
| | indol-3-yl)ethyl)piperazine-2-carboxamide | | |
| 73 | (R)—N-(1-(5-(2-(1H-indol-3-yl)ethyl)-4-(2,4- | 625.3 | 626.3 |
| | dimethoxybenzyl)-4H-1,2,4-triazol-3-yl)-2-(1H- | | |
| | indol-3-yl)ethyl)picolinamide | | |
| 74 | (R)-1-(5-(2-(1H-indol-3-yl)ethyl)-4-(2,4- | 520.3 | 520.8 |
| | dimethoxybenzyl)-4H-1,2,4-triazol-3-yl)-2-(1H- | | |
| | indol-3-yl)ethanamine | | |
| 75 | (R)—N-(1-(4-(2,4-dimethoxybenzyl)-5-phenethyl- | 538.3 | 539.3 |
| | 4H-1,2,4-triazol-3-yl)-2-(1H-indol-3-yl)ethyl)-2- | | |
| | aminoacetamide | | |
| 76 | (R)—N-(1-(4-(4-methoxybenzyl)-5-phenethyl-4H- | 557.3 | 558.0 |
| | 1,2,4-triazol-3-yl)-2-(1H-indol-3-yl)ethyl)pyrazine-2- | | |
| | carboxamide | | |
| 77 | (R)—N-(1-(4-(4-methoxybenzyl)-5-phenethyl-4H- | 556.3 | 556.9 |
| | 1,2,4-triazol-3-yl)-2-(1H-indol-3- | | |
| | yl)ethyl)isonicotinamide | | |
| 78 | (R)—N-1-(4-(4-methoxybenzyl)-5-phenethyl-4H- | 563.3 | 564.0 |
| | 1,2,4-triazol-3-yl)-2-(1H-indol-3-yl)ethyl)piperazine- | | |
| | 2-carboxamide | | |
| 79 | (R)—N-(1-(4-(4-methoxybenzyl)-5-phenethyl-4H- | 556.3 | 557.3 |
| | 1,2,4-triazol-3-yl)-2-(1H-indol-3- | | |
| | yl)ethyl)picolinamide | | |
| 80 | (R)—N-(1-(5-(2-(1H-indol-3-yl)ethyl)-4-(4- | 595.3 | 596.2 |
| | methoxybenzyl)-4H-1,2,4-triazol-3-yl)-2-(1H-indol- | | |
| | 3-yl)ethyl)picolinamide | | |
| 81 | (R)—N-1-(5-(2-(1H-indol-3-yl)ethyl)-4-(4- | 602.3 | 603.3 |
| | methoxybenzyl)-4H-1,2,4-triazol-3-yl)-2-(1H-indol- | | |
| | 3-yl)ethyl)piperazine-2-carboxamide | | |
| 82 | (R)—N-(1-(4-(4-ethylbenzyl)-5-phenethyl-4H-1,2,4- | 568.3 | 569.3 |
| | triazol-3-yl)-2-(1H-indol-3-yl)ethyl)-2-($$yridine-2- | | |
| | yl)acetamide | | |
| 83 | (R)—N-(1-(4-(4-ethylbenzyl)-5-phenethyl-4H-1,2,4- | 560.3 | 561.3 |
| | triazol-3-yl)-2-(1H-indol-3-yl)ethyl)piperidine-4- | | |
| | carboxamide | | |
| 84 | (R)—N-1-(4-(4-ethylbenzyl)-5-phenethyl-4H-1,2,4- | 561.3 | 562.2 |
| | triazol-3-yl)-2-(1H-indol-3-yl)ethyl)piperazine-2- | | |
| | carboxamide | | |
| 85 | (R)—N-(1-(4-(4-ethylbenzyl)-5-phenethyl-4H-1,2,4- | 555.3 | 556.2 |
| | triazol-3-yl)-2-(1H-indol-3-yl)ethyl)pyrazine-2- | | |
| | carboxamide | | |
| 86 | (R)—N-(1-(5-(2-(1H-indol-3-yl)ethyl)-4-(2,4- | 645.3 | 646.2 |
| | dimethoxybenzyl)-4H-1,2,4-triazol-3-yl)-2-(1H- | | |
| | indol-3-yl)ethyl)-2-cis- | | |
| | aminocyclohexanecarboxamide | | |
| 87 | (S)—N-((R)-1-(5-(2-(1H-indol-3-yl)ethyl)-4-(4- | 601.3 | 601.9 |
| | methoxybenzyl)-4H-1,2,4-triazol-3-yl)-2-(1H-indol- | | |
| | 3-yl)ethyl)piperidine-3-carboxamide | | |
| 88 | (R)—N-((R)-1-(5-(2-(1H-indol-3-yl)ethyl)-4-(4- | 601.3 | 602.2 |
| | methoxybenzyl)-4H-1,2,4-triazol-3-yl)-2-(1H-indol- | | |
| | 3-yl)ethyl)piperidine-2-carboxamide | | |
| 89 | (S)—N-((R)-1-(5-(2-(1H-indol-3-yl)ethyl)-4-(4- | 587.3 | 588.2 |
| | methoxybenzyl)-4H-1,2,4-triazol-3-yl)-2-(1H-indol- | | |
| | 3-yl)ethyl)pyrrolidine-2-carboxamide | | |
| 90 | (R)—N-((R)-1-(5-(2-(1H-indol-3-yl)ethyl)-4-(4- | 587.3 | 588.0 |
| | methoxybenzyl)-4H-1,2,4-triazol-3-yl)-2-(1H-indol- | | |
| | 3-yl)ethyl)pyrrolidine-2-carboxamide | | |
| 91 | (R)—N-(1-(5-(2-(1H-indol-3-yl)ethyl)-4-(4- | 609.3 | 610.0 |
| | methoxybenzyl)-4H-1,2,4-triazol-3-yl)-2-(1H-indol- | | |
| | 3-yl)ethyl)-2-(pyridin-2-yl)acetamide | | |
| 92 | (R)—N-(1-(5-(2-(1H-indol-3-yl)ethyl)-4-(4- | 623.2 | 624.1 |
| | bromobenzyl)-4H-1,2,4-triazol-3-yl)-2-(1H-indol-3- | | |
| | yl)ethyl)-2-amino-2-methylpropanamide | | |
| 93 | (R)—N-(1-(5-(2-(1H-indol-3-yl)ethyl)-4-(4- | 536.3 | 536.9 |
| | methoxybenzyl)-4H-1,2,4-triazol-3-yl)-2- | | |
| | phenylethyl)-2-amino-2-methylpropanamide | | |
| 94 | (R)—N-(2-(1H-indol-3-yl)-1-(5-phenethyl-4- | 538.3 | 538.8 |
| | (thiophen-2-ylmethyl)-4H-1,2,4-triazol-3- | | |
| | yl)ethyl)piperidine-4-carboxamide | | |
| 95 | (R)—N-(1-(4-(2-(1H-indol-3-yl)ethyl)-4H-1,2,4- | 455.2 | 456.2 |
| | triazol-3-yl)-2-(1H-indol-3-yl)ethyl)-2-amino-2- | | |
| | methylpropanamide | | |
| 96 | (R)—N-(1-(5-((1H-indol-3-yl)methyl)-4-methyl-4H- | 455.2 | 456.4 |
| | 1,2,4-triazol-3-yl)-2-(1H-indol-3-yl)ethyl)-2-amino- | | |
| | 2-methylpropanamide | | |
| 97 | (R)—N-(1-(5-(2-(1H-indol-3-yl)ethyl)-4-methyl-4H- | 469.3 | 470.2 |
| | 1,2,4-triazol-3-yl)-2-(1H-indol-3-yl)ethyl)-2-amino- | | |
| | 2-methylpropanamide | | |
| 98 | (R)—N-(1-(5-((1H-indol-3-yl)methyl)-4H-1,2,4- | 441.3 | 442.1 |
| | triazol-3-yl)-2-(1H-indol-3-yl)ethyl)-2-amino-2- | | |
| | methylpropanamide | | |
| 99 | (R)—N-(1-(5-((1H-indol-3-yl)methyl)-4-(2,4- | 591.3 | 592.1 |
| | dimethoxybenzyl)-4H-1,2,4-triazol-3-yl)-2-(1H- | | |
| | indol-3-yl)ethyl)-2-amino-2-methylpropanamide | | |
| 100 | (R)—N-(1-(4-(2,4-dimethoxybenzyl)-5-methyl-4H- | 476.3 | 477.4 |
| | 1,2,4-triazol-3-yl)-2-(1H-indol-3-yl)ethyl)-2-amino- | | |
| | 2-methylpropanamide | | |
| 101 | (R)—N-(1-(5-((1H-indol-3-yl)methyl)-4-(4- | 561.3 | 562.3 |
| | methoxybenzyl)-4H-1,2,4-triazol-3-yl)-2-(1H-indol- | | |
| | 3-yl)ethyl)-2-amino-2-methylpropanamide | | |
| 102 | (R)—N-(1-(4-(2,4-dimethoxybenzyl)-5-benzyl-4H- | 552.3 | 553.2 |
| | 1,2,4-triazol-3-yl)-2-(1H-indol-3-yl)ethyl)-2-amino- | | |
| | 2-methylpropanamide | | |
| 103 | (R)—N-(1-(5-(3-(1H-indol-3-yl)propyl)-4-(2,4- | 619.3 | 620.2 |
| | dimethoxybenzyl)-4H-1,2,4-triazol-3-yl)-2-(1H- | | |
| | indol-3-yl)ethyl)-2-amino-2-methylpropanamide | | |
| 104 | (R)—N-(1-(5-((1H-indol-3-yl)methyl)-4-phenethyl- | 545.3 | 546.4 |
| | 4H-1,2,4-triazol-3-yl)-2-(1H-indol-3-yl)ethyl)-2- | | |
| | amino-2-methylpropanamide | | |
| 105 | (R)—N-(1-(5-benzyl-4-phenethyl-4H-1,2,4-triazol-3- | 506.3 | 507.4 |
| | yl)-2-(1H-indol-3-yl)ethyl)-2-amino-2- | | |
| | methylpropanamide | | |
| 106 | (R)—N-(1-(5-benzyl-4-(2,2-diphenylethyl)-4H-1,2,4- | 582.3 | 583.3 |
| | triazol-3-yl)-2-(1H-indol-3-yl)ethyl)-2-amino-2- | | |
| | methylpropanamide | | |
| 107 | (R)—N-(1-(5-(2-(1H-indol-3-yl)ethyl)-4-(2,2- | 635.3 | 636.4 |
| | diphenylethyl)-4H-1,2,4-triazol-3-yl)-2-(1H-indol-3- | | |
| | yl)ethyl)-2-amino-2-methylpropanamide | | |
| 108 | (R)—N-(1-(4-(3,5-dimethoxybenzyl)-5-benzyl-4H- | 552.3 | 553.3 |
| | 1,2,4-triazol-3-yl)-2-(1H-indol-3-yl)ethyl)-2-amino- | | |
| | 2-methylpropanamide | | |
| 109 | (R)—N-(1-(4,5-dibenzyl-4H-1,2,4-triazol-3-yl)-2-(1H- | 492.3 | 493.3 |
| | indol-3-yl)ethyl)-2-amino-2-methylpropanamide | | |
| 110 | (R)—N-(1-(5-benzyl-4-hexyl-4H-1,2,4-triazol-3-yl)-2- | 486.3 | 487.4 |
| | (1H-indol-3-yl)ethyl)-2-amino-2- | | |
| | methylpropanamide | | |
| 111 | (R)—N-(1-(4-(2-(1H-indol-3-yl)ethyl)-5-benzyl-4H- | 545.3 | 546.2 |
| | 1,2,4-triazol-3-yl)-2-(1H-indol-3-yl)ethyl)-2-amino- | | |
| | 2-methylpropanamide | | |
| 112 | (S)—N-(1-(4-(2,4-dimethoxybenzyl)-5-benzyl-4H- | 552.3 | 553.1 |
| | 1,2,4-triazol-3-yl)-2-(1H-indol-3-yl)ethyl)-2-amino- | | |
| | 2-methylpropanamide | | |
| 113 | (R)—N-(1-(4-(3,5-dimethoxybenzyl)-5-phenethyl- | 566.3 | 567.3 |
| | 4H-1,2,4-triazol-3-yl)-2-(1H-indol-3-yl)ethyl)-2- | | |
| | amino-2-methylpropanamide | | |
| 114 | (R)—N-(1-(4-(4-bromobenzyl)-5-benzyl-4H-1,2,4- | 570.2 | 571.0 |
| | triazol-3-yl)-2-(1H-indol-3-yl)ethyl)-2-amino-2- | | |
| | methylpropanamide | | |
| 115 | (R)—N-(1-(4-(2-methoxybenzyl)-5-benzyl-4H-1,2,4- | 522.3 | 523.0 |
| | triazol-3-yl)-2-(1H-indol-3-yl)ethyl)-2-amino-2- | | |
| | methylpropanamide | | |
| 116 | (S)—N-(1-(4-(2,4-dimethoxybenzyl)-5-phenethyl-4H- | 566.3 | 567.0 |
| | 1,2,4-triazol-3-yl)-2-(1H-indol-3-yl)ethyl)-2-amino- | | |
| | 2-methylpropanamide | | |
| 117 | (R)—N-(1-(4,5-diphenethyl-4H-1,2,4-triazol-3-yl)-2- | 520.3 | 521.1 |
| | (1H-indol-3-yl)ethyl)-2-amino-2- | | |
| | methylpropanamide | | |
| 118 | (R)—N-(1-(4-(3,4-dichlorobenzyl)-5-benzyl-4H- | 560.2 | 561.3 |
| | 1,2,4-triazol-3-yl)-2-(1H-indol-3-yl)ethyl)-2-amino- | | |
| | 2-methylpropanamide | | |
| 119 | (R)-1-(5-(2-(1H-indol-3-yl)ethyl)-4-(2,4- | 451.3 | 452.1 |
| | dimethoxybenzyl)-4H-1,2,4-triazol-3-yl)-2-(1H- | | |
| | indol-3-yl)ethanamine | | |
| 120 | (R)—N-(1-(4-(4-methoxybenzyl)-5-benzyl-4H-1,2,4- | 483.3 | 484.0 |
| | triazol-3-yl)-2-phenylethyl)-2-amino-2- | | |
| | methylpropanamide | | |
| 121 | (R)—N-(1-(4-(4-fluorobenzyl)-5-phenethyl-4H-1,2,4- | 524.3 | 524.9 |
| | triazol-3-yl)-2-(1H-indol-3-yl)ethyl)-2-amino-2- | | |
| | methylpropanamide | | |
| 122 | (R)—N-(1-(4-(3,4-dichlorobenzyl)-5-phenethyl-4H- | 574.2 | 574.9 |
| | 1,2,4-triazol-3-yl)-2-(1H-indol-3-yl)ethyl)-2-amino- | | |
| | 2-methylpropanamide | | |
| 124 | (R)—N-(1-(4-(4-methylbenzyl)-5-benzyl-4H-1,2,4- | 506.3 | 507.3 |
| | triazol-3-yl)-2-(1H-indol-3-yl)ethyl)-2-amino-2- | | |
| | methylpropanamide | | |
| 125 | (S)—N-(1-(4-(4-methoxybenzyl)-5-(3-phenylpropyl)- | 550.3 | 551.3 |
| | 4H-1,2,4-triazol-3-yl)-2-(1H-indol-3-yl)ethyl)-2- | | |
| | amino-2-methylpropanamide | | |
| 126 | (S)—N-(1-(4-(4-methoxybenzyl)-5-benzyl-1,2,4- | 522.3 | 523.4 |
| | triazol-3-yl)-2-(1H-indol-3-yl)ethyl)-2-amino-2- | | |
| | methylpropanamide | | |
| 128 | (R)—N-(1-(4-(4-nitrobenzyl)-5-phenethyl-4H-1,2,4- | 551.3 | 551.9 |
| | triazol-3-yl)-2-(1H-indol-3-yl)ethyl)-2-amino-2- | | |
| | methylpropanamide | | |
| 129 | (S)—N-(1-(4-(4-methoxybenzyl)-5-phenethyl-4H- | 536.3 | 537.0 |
| | 1,2,4-triazol-3-yl)-2-(1H-indol-3-yl)ethyl)-2-amino- | | |
| | 2-methylpropanamide | | |
| 130 | (R)—N-(1-(4-(4-methoxyphenethyl)-5-phenethyl-4H- | 550.3 | 551.0 |
| | 1,2,4-triazol-3-yl)-2-(1H-indol-3-yl)ethyl)-2-amino- | | |
| | 2-methylpropanamide | | |
| 131 | (R)—N-(2-(1H-indol-3-yl)-1-(5-phenethyl-4- | 512.2 | 512.8 |
| | (thiophen-2-ylmethyl)-4H-1,2,4-triazol-3-yl)ethyl)-2- | | |
| | amino-2-methylpropanamide | | |
| 132 | (R)—N-(2-(1H-indol-3-yl)-1-(5-phenethyl-4-(pyridin- | 507.3 | 508.4 |
| | 2-ylmethyl)-4H-1,2,4-triazol-3-yl)ethyl)-2-amino-2- | | |
| | methylpropanamide | | |
| 133 | (R)—N-(-2-(1H-indol-3-yl)-1-(5-phenethyl-4-(pyridin- | 533.3 | 534.0 |
| | 2-ylmethyl)-4H-1,2,4-triazol-3-yl)ethyl)piperidine-3- | | |
| | carboxamide | | |
| 134 | (S)—N-((R)-1-(4-(4-ethylbenzyl)-5-phenethyl-4H- | 546.3 | 547.3 |
| | 1,2,4-triazol-3-yl)-2-(1H-indol-3-yl)ethyl)pyrrolidine- | | |
| | 2-carboxamide | | |
| 135 | N-((R)-1-(4-(4-ethylbenzyl)-5-phenethyl-4H-1,2,4- | 506.3 | 507.3 |
| | triazol-3-yl)-2-(1H-indol-3-yl)ethyl)-2- | | |
| | aminoacetamide | | |
| 136 | N-((R)-1-(5-(2-(1H-indol-3-yl)ethyl)-4-(4- | 609.3 | 610.4 |
| | methoxybenzyl)-4H-1,2,4-triazol-3-yl)-2-(1H-indol- | | |
| | 3-yl)ethyl)-2-(pyridin-4-yl)acetamide | | |
| 137 | (2R)—N-((R)-1-(4-(4-ethylbenzyl)-5-phenethyl-4H- | 560.3 | 561.3 |
| | 1,2,4-triazol-3-yl)-2-(1H-indol-3-yl)ethyl)piperidine- | | |
| | 2-carboxamide | | |
| 138 | N-((R)-1-(4-(4-ethylbenzyl)-5-phenethyl-4H-1,2,4- | 554.3 | 555.3 |
| | triazol-3-yl)-2-(1H-indol-3-yl)ethyl)picolinamide | | |
| 139 | N-((R)-1-(4-(4-ethylbenzyl)-5-phenethyl-4H-1,2,4- | 569.3 | 570.3 |
| | triazol-3-yl)-2-(1H-indol-3-yl)ethyl)-2- | | |
| | aminopyridine-3-carboxamide | | |
| 140 | (2S)—N-((R)-1-(4-(4-ethylbenzyl)-5-phenethyl-4H- | 520.3 | 521.2 |
| | 1,2,4-triazol-3-yl)-2-(1H-indol-3-yl)ethyl)-2- | | |
| | aminopropanamide | | |
| 141 | N-((R)-1-(4-(4-ethylbenzyl)-5-phenethyl-4H-1,2,4- | 554.3 | 555.4 |
| | triazol-3-yl)-2-(1H-indol-3-yl)ethyl)isonicotinamide | | |
| 142 | N-((R)-2-(1H-indol-3-yl)-1-(5-phenethyl-4-phenyl- | 518.3 | 519.3 |
| | 4H-1,2,4-triazol-3-yl)ethyl)piperidine-4- | | |
| | carboxamide | | |
| 143 | (2S)—N-((R)-2-(1H-indol-3-yl)-1-(5-phenethyl-4- | 504.3 | 505.1 |
| | phenyl-4H-1,2,4-triazol-3-yl)ethyl)pyrrolidine-2- | | |
| | carboxamide | | |
| 144 | N-((R)-2-(1H-indol-3-yl)-1-(5-phenethyl-4-phenyl- | 464.3 | 465.1 |
| | 4H-1,2,4-triazol-3-yl)ethyl)-2-aminoacetamide | | |
| 145 | N-((R)-2-(1H-indol-3-yl)-1-(5-phenethyl-4-phenyl- | 526.3 | 527.2 |
| | 4H-1,2,4-triazol-3-yl)ethyl)-2-(pyridin-2- | | |
| | yl)acetamide | | |
| 146 | N-((R)-2-(1H-indol-3-yl)-1-(5-phenethyl-4-phenyl- | 512.3 | 513.4 |
| | 4H-1,2,4-triazol-3-yl)ethyl)picolinamide | | |
| 147 | N-((R)-1-(5-(2-(1H-indol-3-yl)ethyl)-4-(4- | 579.3 | 580.1 |
| | ethylphenyl)-4H-1,2,4-triazol-3-yl)-2-(1H-indol-3- | | |
| | yl)ethyl)picolinamide | | |
| 148 | N-((R)-1-(5-(2-(1H-indol-3-yl)ethyl)-4-(4- | 593.3 | 594.2 |
| | ethylphenyl)-4H-1,2,4-triazol-3-yl)-2-(1H-indol-3- | | |
| | yl)ethyl)-2-(pyridin-2-yl)acetamide | | |
| 149 | N-((R)-1-(5-(2-(1H-indol-3-yl)ethyl)-4-(4- | 531.3 | 532.2 |
| | ethylphenyl)-4H-1,2,4-triazol-3-yl)-2-(1H-indol-3- | | |
| | yl)ethyl)-2-aminoacetamide | | |
| 150 | (2S)—N-((R)-1-(5-(2-(1H-indol-3-yl)ethyl)-4-(4- | 571.3 | 572.4 |
| | ethylphenyl)-4H-1,2,4-triazol-3-yl)-2-(1H-indol-3- | | |
| | yl)ethyl)pyrrolidine-2-carboxamide | | |
| 152 | N-((R)-1-(5-(2-(1H-indol-3-yl)ethyl)-4-(4- | 547.3 | 548.0 |
| | methoxybenzyl)-4H-1,2,4-triazol-3-yl)-2-(1H-indol- | | |
| | 3-yl)ethyl)-2-aminoacetamide | | |
| 153 | N-((R)-1-(4-(4-methoxybenzyl)-5-phenethyl-4H- | 576.3 | 577.2 |
| | 1,2,4-triazol-3-yl)-2-(1H-indol-3-yl)ethyl)-2-trans- | | |
| | aminocyclohexanecarboxamide | | |
| 154 | N-((R)-1-(4-(4-ethylbenzyl)-5-phenethyl-4H-1,2,4- | 568.3 | 569.3 |
| | triazol-3-yl)-2-(1H-indol-3-yl)ethyl)-2-(pyridin-3- | | |
| | yl)acetamide | | |
| 155 | (3S)—N-((R)-1-(4-(4-ethylbenzyl)-5-phenethyl-4H- | 560.3 | 561.5 |
| | 1,2,4-triazol-3-yl)-2-(1H-indol-3-yl)ethyl)piperidine- | | |
| | 3-carboxamide | | |
| 156 | N-((R)-1-(4-(4-ethylbenzyl)-5-phenethyl-4H-1,2,4- | 568.3 | 569.1 |
| | triazol-3-yl)-2-(1H-indol-3-yl)ethyl)-2- | | |
| | aminobenzamide | | |
| 157 | N-((R)-1-(5-(2-(1H-indol-3-yl)ethyl)-4-phenyl-4H- | 551.3 | 552.2 |
| | 1,2,4-triazol-3-yl)-2-(1H-indol-3- | | |
| | yl)ethyl)picolinamide | | |
| 158 | N-((R)-1-(5-(2-(1H-indol-3-yl)ethyl)-4-phenyl-4H- | 557.3 | 558.1 |
| | 1,2,4-triazol-3-yl)-2-(1H-indol-3-yl)ethyl)piperidine- | | |
| | 4-carboxamide | | |
| 159 | N-((R)-2-(1H-indol-3-yl)-1-(4-(2,4- | 572.3 | 573.4 |
| | dimethoxyphenyl)-5-phenethyl-4H-1,2,4-triazol-3- | | |
| | yl)ethyl)picolinamide | | |
| 160 | N-((R)-2-(1H-indol-3-yl)-1-(4-(2,4- | 586.3 | 587.3 |
| | dimethoxyphenyl)-5-phenethyl-4H-1,2,4-triazol-3- | | |
| | yl)ethyl)-2-(pyridin-2-yl)acetamide | | |
| 161 | N-((R)-2-(1H-indol-3-yl)-1-(4-(2,4- | 573.3 | 574.2 |
| | dimethoxyphenyl)-5-phenethyl-4H-1,2,4-triazol-3- | | |
| | yl)ethyl)pyrazine-2-carboxamide | | |
| 162 | N-((R)-2-(1H-indol-3-yl)-1-(4-(2,4- | 524.3 | 525.3 |
| | dimethoxyphenyl)-5-phenethyl-4H-1,2,4-triazol-3- | | |
| | yl)ethyl)-2-aminoacetamide | | |
| 163 | N-((R)-2-(1H-indol-3-yl)-1-(4-(2,4- | 578.3 | 579.4 |
| | dimethoxyphenyl)-5-phenethyl-4H-1,2,4-triazol-3- | | |
| | yl)ethyl)piperidine-4-carboxamide | | |
| 164 | N-((R)-1-(5-benzyl-4-((pyridin-2-yl)methyl)-4H- | 513.3 | 514.3 |
| | 1,2,4-triazol-3-yl)-2-(1H-indol-3- | | |
| | yl)ethyl)picolinamide | | |
| 165 | N-((R)-1-(5-benzyl-4-((pyridin-2-yl)methyl)-4H- | 465.3 | 466.4 |
| | 1,2,4-triazol-3-yl)-2-(1H-indol-3-yl)ethyl)-2-amino- | | |
| | acetamide | | |
| 166 | N-((R)-1-(5-benzyl-4-((pyridin-2-yl)methyl)-4H- | 519.3 | 520.2 |
| | 1,2,4-triazol-3-yl)-2-(1H-indol-3-yl)ethyl)piperidine- | | |
| | 4-carboxamide | | |
| 167 | N-((R)-1-(5-benzyl-4-((pyridin-4-yl)methyl)-4H- | 493.3 | 494.3 |
| | 1,2,4-triazol-3-yl)-2-(1H-indol-3-yl)ethyl)-2-amino- | | |
| | 2-methylpropanamide | | |
| 168 | N-((R)-1-(5-(4-methoxybenzyl)-4-phenethyl-4H- | 536.3 | 537.3 |
| | 1,2,4-triazol-3-yl)-2-(1H-indol-3-yl)ethyl)-2-amino- | | |
| | 2-methylpropanamide | | |
| 169 | N-((R)-1-(5-benzyl-4-((pyridin-4-yl)methyl)-4H- | 513.3 | 514.2 |
| | 1,2,4-triazol-3-yl)-2-(1H-indol-3- | | |
| | yl)ethyl)picolinamide | | |
| 170 | N-((R)-1-(5-benzyl-4-((pyridin-4-yl)methyl)-4H- | 465.3 | 466.1 |
| | 1,2,4-triazol-3-yl)-2-(1H-indol-3-yl)ethyl)2-amino- | | |
| | acetamide | | |
| 171 | (R)-benzyl-3-(2-aminoisobutyramido)-3-(5-(2-(1H- | 594.3 | 595.2 |
| | indol-3-yl)ethyl)-4-(4-methoxybenzyl)-4H-1,2,4- | | |
| | triazol-3-yl)-propanoate | | |
| 172 | N-((R)-1-(5-benzyl-4-((pyridin-3-yl)methyl)-4H- | 493.3 | 494.3 |
| | 1,2,4-triazol-3-yl)-2-(1H-indol-3-yl)ethyl)-2-amino- | | |
| | 2-methylpropanamide | | |
| 173 | N-((R)-1-(4-benzyl-5-phenethyl-4H-1,2,4-triazol-3- | 506.3 | 507.3 |
| | yl)-2-(1H-indol-3-yl)ethyl)-2-amino-2- | | |
| | methylpropanamide | | |
| 174 | N-((R)-2-(1H-indol-3-yl)-1-(4-methyl-5-phenethyl- | 450.3 | 451.3 |
| | 4H-1,2,4-triazol-3-yl)ethyl)picolinamide | | |
| 175 | N-((R)-1-(5-(2-(1H-indol-3-yl)ethyl)-4-phenyl-4H- | 531.3 | 532.4 |
| | 1,2,4-triazol-3-yl)-2-(1H-indol-3-yl)ethyl)-2-amino- | | |
| | 2-methylpropanamide | | |
| 176 | N-((R)-1-(4-(4-methoxybenzyl)-5-phenethyl-4H- | 555.3 | 556.2 |
| | 1,2,4-triazol-3-yl)-2-(1H-indol-3-yl)ethyl)benzamide | | |
| 177 | (R)-1-(4-(2,4-dimethoxybenzyl)-5-phenethyl-4H- | 635.3 | 637.0 |
| | 1,2,4-triazol-3-yl)-2-(1H-indol-3-yl)-N- | | |
| | phenylmethanesulfonylamine | | |
| 178 | (R)-1-(4-(2,4-dimethoxybenzyl)-5-phenethyl-4H- | 635.3 | 636.3 |
| | 1,2,4-triazol-3-yl)-2-(1H-indol-3-yl)-N- | | |
| | tosylethanamine | | |
| 179 | N-((R)-1-(4-(2,4-dimethoxybenzyl)-5-phenethyl- | 566.3 | 567.2 |
| | 4H-1,2,4-triazol-3-yl)-2-(1H-indol-3-yl)ethyl)-2- | | |
| | amino-2-methylpropanamide | | |
| 180 | N-1-((R)-1-(4-(2,4-dimethoxybenzyl)-5-phenethyl- | 524.3 | 525.2 |
| | 4H-1,2,4-triazol-3-yl)-2-(1H-indol-3-yl)ethyl)ethane- | | |
| | 1,2-diamine | | |
| 181 | N-((R)-1-(4-((furan-2-yl)methyl)-5-phenethyl-4H- | 496.3 | 497.1 |
| | 1,2,4-triazol-3-yl)-2-(1H-indol-3-yl)ethyl)-2-amino- | | |
| | 2-methylpropanamide | | |
| 182 | N-((R)-1-(4-((furan-2-yl)methyl)-5-phenethyl-4H- | 516.3 | 517.1 |
| | 1,2,4-triazol-3-yl)-2-(1H-indol-3- | | |
| | yl)ethyl)picolinamide | | |
| 183 | N-((R)-1-(4-((furan-2-yl)methyl)-5-phenethyl-4H- | 522.3 | 523.2 |
| | 1,2,4-triazol-3-yl)-2-(1H-indol-3-yl)ethyl)piperidine- | | |
| | 4-carboxamide | | |
| 184 | N-((R)-1-(4-(4-methoxybenzyl)-5-phenethyl-4H- | 563.3 | 564.2 |
| | 1,2,4-triazol-3-yl)-2-(1H-indol-3-yl)ethyl)- | | |
| | tetrahydro-2H-pyran-4-carboxamide | | |
| 185 | N-((R)-1-(5-((1H-indol-3-yl)methyl)-4-(3- | 561.3 | 562.2 |
| | methoxybenzyl)-4H-1,2,4-triazol-3-yl)-2-(1H-indol- | | |
| | 3-yl)ethyl)-2-amino-2-methylpropanamide | | |
| 186 | (2S)—N-((R)-1-(4-(4-methoxybenzyl)-5-phenethyl- | 598.3 | 599.1 |
| | 4H-1,2,4-triazol-3-yl)-2-(1H-indol-3-yl)ethyl)-2- | | |
| | amino-3-phenylpropanamide | | |
| 187 | (R)-1-(5-(2-(1H-indol-3-yl)ethyl)-4-(2,4- | 674.3 | 675.0 |
| | dimethoxybenzyl)-4H-1,2,4-triazol-3-yl)-2-(1H- | | |
| | indol-3-yl)-N-tosylethanamine | | |
| 188 | N-((R)-1-(4-(2,4-dimethoxybenzyl)-5-phenethyl- | 626.3 | 627.3 |
| | 4H-1,2,4-triazol-3-yl)-2-(1H-indol-3-yl)ethyl)-4- | | |
| | azidobenzamide | | |
| 189 | N-benzyl-(R)-1-(4-(2,4-dimethoxybenzyl)-5- | 571.3 | 572.3 |
| | phenethyl-4H-1,2,4-triazol-3-yl)-2-(1H-indol-3- | | |
| | yl)ethanamine | | |
| 190 | (2S)—N-((R)-1-(5-(2-(1H-indol-3-yl)ethyl)-4-(4- | 585.3 | 586.2 |
| | methoxybenzyl)-4H-1,2,4-triazol-3-yl)-2-(1H-indol- | | |
| | 3-yl)ethyl)-2,5-dihydro-1H-pyrrole-2-carboxamide | | |

### II) RT-PCR Analysis of Total RNA Extracts from Primary Adipocytes and Differentiated 3T3-L1 Cells

Total RNA was extracted from primary mouse adipocytes or differentiated 3T3-L1 cells (ATCC CL-173) with Trizol LS reagent (Invitrogen, cat. No. 15596-018) according to the manufacturer's instructions. Total RNA were treated with DNase I Amplification Grade (Invitrogen, cat. No. 18068-015) according to manufacturer's instructions. cDNA was synthesized from 1 μg of DNase treated total RNA with random primers (250 ng/μl, Invitrogen, cat. No. 48190-011) and 20 U M-MLV Reverse Transcriptase (Invitrogen, cat. No. 28025-013) at 37° C. for 2 h in a total volume of 20 μl containing 50 mM Tris-HCl pH 8.3, 75 mM KCl, 3 mM MgCl₂, 10 mM DTT and 0.5 mM dNTP. After sample denaturation at 95° C. for 5 min, cDNAs were amplified in a volume of 50 μL with 2 μl of reverse transcription reaction and 0.2 μM of each specific primer using 2.5 U of Taq DNA polymerase (Invitrogen, cat. No. 18038-042). Cycling conditions for GPR103A were 35 cycles at 94° C. for 45 sec; 60° C. for 45 sec and 72° C. for 1 min; for GPR103B, 35 cycles at 94° C. for 45 sec; 59.5° C. for 45 sec and 72° C. for 1 min; for QRFP, 35 cycles at 94° C. for 45 sec; 60° C. for 45 sec and 72° C. for 1 min; and for RNA 18S, 26 cycles at 94° C. for 45 sec; 62.5° C. for 45 sec and 72° C. for 1.5 min. PCR products were separated on 1.5% agarose gel, and the relative signal intensity was measured with an image analyzer (Typhoon, Amersham). The primer sequences and the expected product size were as follows: GPR103A, sense primer: 5′-TCT TTG GCA ACT CTC TGG TCA TC-3′ (SEQ ID NO:6), GPR103A, antisense primer: 5′-CTT CGG GTA GTG TAC TGC CAC T-3′ (304 bp) (SEQ ID NO:7); GPR103B, sense primer: 5′-CGA TAT CM GTG GTG TGA ACA GCC-3′ (SEQ ID NO:8), GPR103B, antisense primer: 5′-GGG TCT CTT GTA GCC CAG GT-3′ (299 bp) (SEQ ID NO:9); QRFP, sense primer: 5′-TCT GCC GTC CTT ACC ATC TCA-3′ (SEQ ID NO:10), QRFP, antisense primer: 5′-TCT CAG GAC TGT CCC AM GGA G-3′ (142 bp) (SEQ ID NO:11) and RNA 18S, sense primer 5′-GGA CCA GAG CGA AAG CAT TTG CC-3′ (SEQ ID NO:12), RNA 18S, anti-sense primer (TCA ATC TCG GGT GGC TGA ACG C-3′ (496 bp) (SEQ ID NO:13). All values were normalized against RNA 18S.

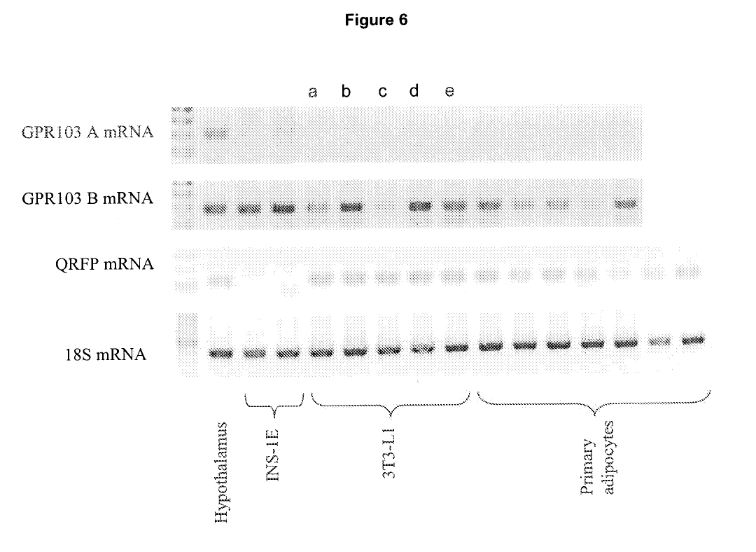

FIG. 6 shows the analysis of the mRNA expression of GPR103 receptors and QRFP peptide in differentiated 3T3-L1 cells and primary adipocytes from mice. From the analysis, it is clear that only GPR103B mRNA is present in differentiated 3T3-L1 cells and in primary adipocytes as follows: a) day 0, b) day 2, c) day 4, d) day 6 and e) day 8. Both primary adipocytes and differentiated 3T3-L1 cells also express QRFP mRNA. mRNA from hypothalamus was used as positive control as both GPR103A and GPR103B mRNA were found in this tissue (“Hypothalamus”). INS-1E is a pancreatic cell line which was found to express GPR103B (“INS-1E”).

### III) Study of Anti-Inhibitory Effects of Isoproterenol-Induced Lipolysis in an Adipocyte Cell Line Model

The effectiveness of compounds of the invention to antagonize the QRFP-induced inhibition of isoproterenol-induced lipolysis was investigated using an adipocyte cell line model.

### Preparation of Differentiated 3T3-L1 Cells

Cell Culture. Prior to differentiation, 3T3-L1 cells (ATCC CL-173 ATCC) (passage 6) were seeded in 150 mm Polystyrene plates (Corning) and grown in Dulbecco's modified Eagle's medium (DMEM) supplemented with 10% calf serum and 100 units/mL of penicillin/streptomycin in a 10% CO₂ incubator at 37° C.

At 95% confluence, cells were passed and seeded in 12-wells Costar plate (Corning 3513) in DMEM supplemented with 10% calf serum.

Upon 95% confluence, differentiation was induced by culturing cells with DMEM supplemented with 10% fetal bovine serum, antibiotics, insulin (1 μug/mL), dexamethasone (1 μM) and isobutylmethylxanthine (0.5 mM) for 4 days. After the differentiation step, cells were then cultured in DMEM with 10% FBS containing 167 nM insulin for 2 days. Cells were then were maintained in DMEM with 10% FBS for 2 days before lipolysis study.

### Lipolysis Study:

All the cellular assays were performed in triplicate for each time point if not otherwise specified.

Differentiated 3T3-L1 cells were first washed with DMEM and incubated with DMEM containing 200 nM adenosine for 1 h at 37° C. The incubation medium was then removed. The lipolysis was induced by incubating the cells with DMEM supplemented with 2% bovine serum albumin fatty acid free and adenosine deaminase (1 U/mL) containing 100 nM of isoproterenol for 90 min at 37° C. The incubation medium (600 μL) was collected for glycerol assay following triglyceride hydrolysis.

The inhibitory effect of QRFP on isoproterenol-induced lipolysis was documented by incubating the cells with DMEM supplemented with 2% bovine serum albumin and adenosine deaminase (1 U/mL) containing 100 nM of isoproterenol in presence of 10⁻¹², 10⁻¹⁰, 10⁻⁹, 10⁻⁸, 10⁻⁷ and 10⁻⁶ M QRFP-26 for 90 min at 37° C. After the incubation period, the incubation medium (600 μL) was collected for free glycerol measurement.

To document the antagonistic effect of compounds of the invention on the antilipolytic effect of QRFP, the dose-response curve of QRFP was carried out as described above in presence of selected compounds of the invention at 10⁻⁹ to 10⁻⁷ M. To document the net effect of QRFP and selected compounds of the invention on lipolysis induced by isoproterenol as control, cells were incubated in absence of isoproterenol in presence of QRFP or selected compounds of the invention at 10⁻⁶ M.

### Glycerol Assays:

Glycerol assays were performed using the free glycerol reagent (Sigma F 6428). The absorbance was read at 540 nm. Glycerol released was calculated as μmol/mg of total protein.

Lipolysis index was calculated as: (μmol of glycerol released/mg of protein of sample minus μmol of glycerol released/mg of protein in basal condition)/(μmol of glycerol released/mg of protein induced by 100 nM of isoproterenol minus μmol of glycerol released/mg of protein of basal condition).

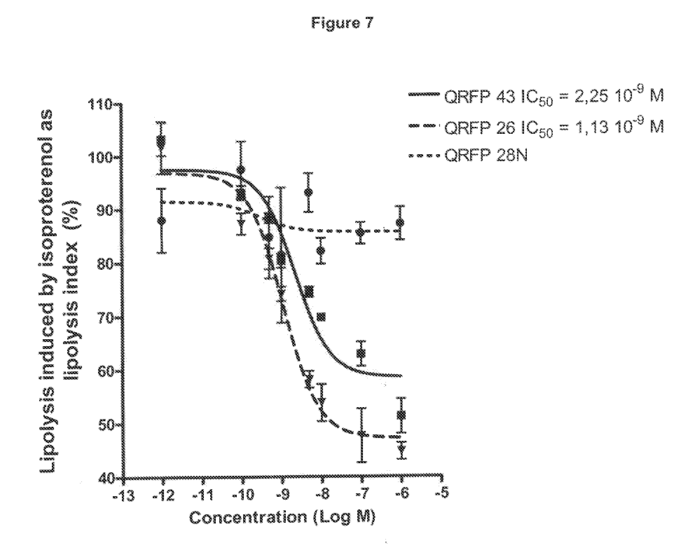

FIG. 7 shows the inhibitory effect of QRFP-43 and QRFP-26 on lipolysis induced by isoproterenol with IC₅₀ of 2.25×10⁻⁹ M and 1.13×10⁻⁹ M, respectively. QRFP-28N (the N-terminal fragment of QRFP-43), which displays no binding affinity to GRP103 is used as negative control.

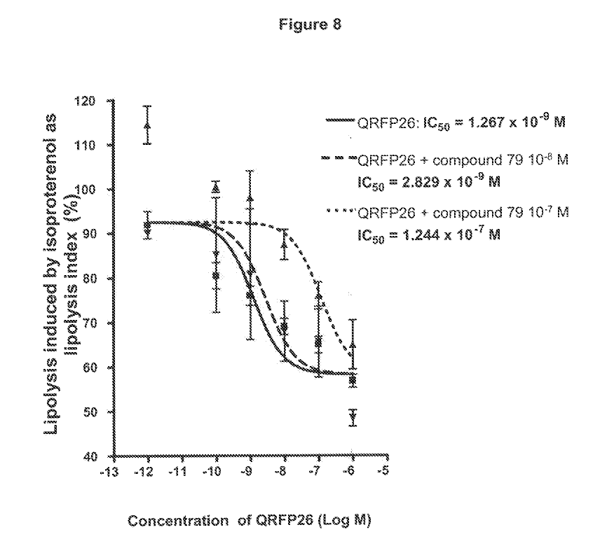

FIG. 8 shows that QRFP-26 induces an inhibitory effect on lipolysis induced by isoproterenol in a dose-dependant manner with IC₅₀ of 1.267×10⁻⁹ M. compound 79 at 10⁻⁹ M and 10⁻⁷ M displays an antagonist effect on the inhibitory effect of QRFP with a shift of IC⁵⁰ at 2.829×10⁻⁹ M and 1.244×10⁻⁷ M, respectively.

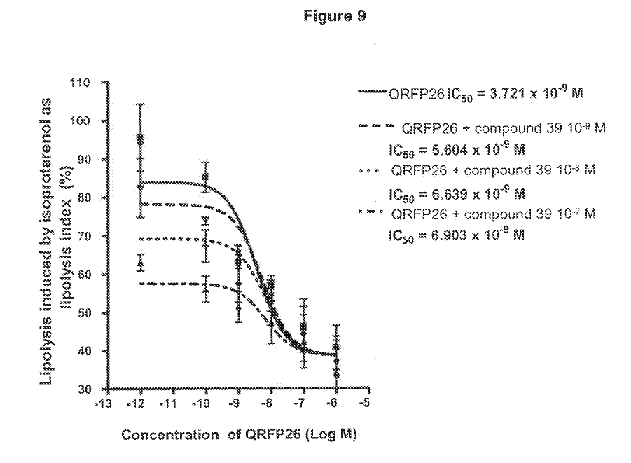

FIG. 9 shows that QRFP-26 induces an inhibitory effect on lipolysis induced by isoproterenol in a dose-dependant manner with IC₅₀ of 3.72×10⁻⁹ M. compound 39 at 10⁻⁹ M, 10⁻⁸ M and 10⁻⁷ M displays a non-competitive antagonist effect on the inhibitory effect of QRFP with a shift of IC₅₀ at 5.6×10⁻⁹ M, 6.6×10⁻⁹ M and 6.9×10⁻⁹ M, respectively.

### IV) Study of the Effect of Compounds of the Invention on Diet-Induced Obesity (DIO) in Mice

The effectiveness of selected compounds of the invention, i.e. their subchronic effects, on the energy balance of mice rendered obese by a high-fat diet was investigated.

Adult C57BL/6 male mice were fed ad libitum with either chow or a high-fat diet (60% of the calorie content from fat) for 5 weeks.

C57BL/6 mice fed with a high-fat diet readily develop obesity and represent a widely used model for investigating human obesity.

During the last two weeks under these regimen, mice were given daily subcutaneous injections of compound 9 or compound 50 (1.5 mg/kg per day, 6 mg/kg per day or 24 mg/kg per day). One-third of the dose was injected in the morning and two-third in the evening.

Body weight and food intake were monitored every day during the experiment. Body composition (lean and fat mass) was assessed using dual energy x-ray absorptiometry (DEXA) on day 0, 13 and 27. Blood was also sampled at sacrifice for the determination of plasma glucose, insulin and triglycerides.

Two weeks of treatment with 6 mg/kg per day and 24 mg/kg per day of compound 9 and of compound 50 significantly decreased body weight gain in mice. The reduced weight gain was accounted for by a reduction in the fat mass deposition. There was no effect of the drugs at any doses on the lean body mass. compound 9 at 6 mg/kg per day and 24 mg/kg per day and compound 50 at 24 mg/kg per day also reduced the size of the fat depots. The reduced fat depositions were not accompanied by a reduced food intake but by a diminished food efficiency [(body weight gain/food ingested)×100]. In addition, animals with reduced fat deposition also demonstrated diminished plasma insulin levels (for all data, refer to table 2).

The presented results provide clear evidence for the ability of selected compounds of the invention to reduce fat gain and improve insulin sensitivity in mice rendered obese by a high-fat diet. The effect on energy balance occurred through a reduced food efficiency demonstrating the ability of selected compounds of the invention to stimulate energy expenditure.

TABLE 2
Values of different variables measured in high-fat diet fed mice
injected sub-cutaneously with saline, compound 9 or compound
50 after a 2 week treatment period.

| | Chow | Saline | | | | | | |
|---|---|---|---|---|---|---|---|---|
| Parameters | control | control | Compound 9 | | | Compound 50 | | |
| Dose | — | — | 1.5 | 6.0 | 24 | 1.5 | 6.0 | 24 |
| (mg/kg per day) | | | | | | | | |
| Body weight gain | 0.5 | 1.9 | 1.03 | 0.20 | −0.54 | 1.61 | 1.44 | −0.04 |
| (g)* | | | | | | | | |
| Cumulative food | 959 | 819 | 793 | 809 | 790 | 852 | 813 | 801 |
| intake (kJ)* | | | | | | | | |
| Food efficiency | 0.52 | 2.31 | 1.30 | 0.25 | −0.68 | 1.88 | 1.77 | −0.05 |
| (mg/kJ) | | | | | | | | |
| Fat mass | 4.18 | 8.3 | 7.9 | 5.8 | 5.7 | 7.6 | 8.2 | 6.3 |
| (g) | | | | | | | | |
| Lean mass | 21.0 | 20.6 | 20.2 | 21.2 | 20.5 | 21.3 | 20.4 | 20.6 |
| (g) | | | | | | | | |
| Σ Fat depots | 467 | 1184 | 1137 | 791 | 792 | 1101 | 1192 | 902 |
| (mg) | | | | | | | | |
| Insulin | 78.0 | 73.4 | 63.8 | 57.4 | 52.0 | 66.3 | 64.8 | 60.3 |
| (pmol/l) | | | | | | | | |
| Glucose | 13.2 | 16.1 | 14.9 | 15.6 | 13.1 | 15.6 | 15.2 | 15.4 |
| (mmol/l) | | | | | | | | |

*Results correspond to the difference between the values at day 35 and day 21 (first day of administration).

### V) Assessment of Fasting-Induced Feeding Behavior (Food Intake) of Mice

The effectiveness of selected compounds of the invention on fasting-induced feeding behavior of mice was investigated.

The experiments were performed according to Asakawa A et al. (Asakawa A et al., Gut 2003, 52: 947-952) with the following technical details:

Preparation of compounds of the invention: dissolved in dimethylsulfoxide (DMSO) and then diluted in distilled water (i.e. 0.5, 1 and 10% DMSO for 0.5, 1 and 2 μmol/mouse, respectively);

Preparation of reference substance (positive control) sibutramine: dissolved in distilled water;

Control substance (negative vehicle control): 10% DMSO in distilled water (compounds of the invention); distilled water (sibutramine);

Route of administration: p.o.;

Pretreatment time: 30 min before presenting the food to the animals;

Volume of administration: 0.5 ml/mouse;

Animals: male C57BL/6J mice;

Body weight range: 18.3-21.4 g

Number per group: 8

The corresponding results are presented in table 3.

TABLE 3

| | | change of food intake - compounds | | | |
|---|---|---|---|---|---|
| | | versus vehicle | | | |
| | Oral | treated negative control | | | |
| Compound | dose (per mouse) | at 20 min | at 1 h | at 2 h | at 4 h |
| Sibutramine | 0.6 μmol | −63% | −66% | −46% | −21% |
| (positive control) | | | | | |
| 5 | 2 μmol | −56% | −58% | −53% | −51% |
| 11 | 2 μmol | −56% | −65% | −55% | −55% |
| 27 | 2 μmol | −57% | −58% | −48% | −38% |
| 28 | 2 μmol | −17% | −28% | −21% | −18% |
| 31 | 2 μmol | +50% | +20% | +16% | +9% |
| 37 | 2 μmol | −37% | −49% | −51% | −34% |
| 45 | 2 μmol | −36% | −28% | −33% | −34% |
| 48 | 2 μmol | −22% | −30% | −27% | −21% |
| 49 | 2 μmol | −59% | −48% | −39% | −23% |
| 50 | 2 μmol | −54% | −44% | −44% | −28% |
| 62 | 2 μmol | +56% | +68% | +50% | +26% |
| 66 | 2 μmol | −56% | −40% | −45% | −51% |
| 117 | 2 μmol | −31% | −24% | −11% | −6% |
| 118 | 2 μmol | −31% | −30% | −25% | −24% |
| 122 | 2 μmol | −52% | −42% | −28% | −16% |
| 142 | 2 μmol | −56% | −57% | −55% | −49% |
| 179 | 2 μmol | −25% | −39% | −40% | −38% |
| 190 | 2 μmol | −39% | −30% | −33% | −30% |

1. A method of modulating the activity of at least one G-protein coupled receptor (GPCR);with the proviso that the following G-protein coupled receptors (GPCR) are excluded: GHS receptors, GHS type 1 receptor, GHS-R 1a, GHS-R 1b, motilin receptor, motilin receptor la, neurotensin receptor, TRH receptor, GPR38 (FMI), GPR39 (FM2), FM3, GHS-R subtype, GHS binding site, cardiac GHS-R, and mammary GHS-R, comprising contacting the at least one G-protein coupled receptor with at least one compound according to formula (I) 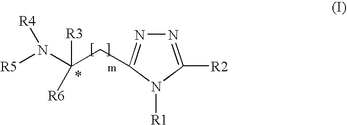
wherein:
R1 and R2 are independently selected from the group consisting of hydrogen, alkyl, alkenyl, alkynyl, cycloalkyl, cycloalkylalkyl, aryl, heteroaryl, arylalkyl, heteroarylalkyl, heterocyclyl, heterocyclylalkyl, alkylsulfonyl, arylsulfonyl, and arylalkylsulfonyl
which can be optionally substituted in the alkyl, cycloalkyl, cycloalkylalkyl, aryl, heteroaryl, arylalkyl, heteroarylalkyl, heterocyclyl and/or heterocyclylalkyl group by up to 3 substituents independently selected from the group consisting of halogen, —F, —Cl, —Br, —I, —N₃, —CN, —NR7R8, —OH, —NO₂, alkyl, aryl, arylalkyl, —O-alkyl, —O-aryl, —O-arylalkyl;
one of R3 and R4 is a hydrogen atom and the other of R3 and R4 is selected from the group consisting of hydrogen atom, alkyl, aryl, heteroaryl, arylalkyl, heteroarylalkyl, heterocyclyl, heterocyclylalkyl, -alkyl-O-aryl, -alkyl-O-arylalkyl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, -alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO—NH₂, -alkyl-CO—OH, -alkyl-NH₂, -alkyl-NH—C(NH)—NH₂, alkylsulfonyl, arylsulfonyl, arylalkylsulfonyl, alkyl-S-alkyl, and alkyl-S—H
which can be optionally substituted in the aryl, heteroaryl, arylalkyl, heteroarylalkyl, heterocyclyl and/or heterocyclylalkyl group by up to 3 substituents independently selected from the group consisting of halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably are selected from the group consisting of “arylalkyl, heteroarylalkyl, heterocyclylalkyl, -alkyl-O-aryl, -alkyl-O-arylalkyl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO—NH₂, -alkyl-CO—OH, -alkyl-NH₂, and -alkyl-NH—C(NH)—NH₂,
optionally being substituted in the aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
R5 is selected from the group consisting of hydrogen, alkyl, cycloalkyl, cycloalkylalkyl, aryl, heteroaryl, arylalkyl, heteroarylalkyl, heterocyclyl, heterocyclylalkyl, —CO-alkyl, —CO-cycloalkyl, —CO-cycloalkylalkyl, —CO-aryl, —CO-arylalkyl, —CO-heteroaryl, —CO-heteroarylalkyl, —CO-heterocyclyl, —CO-heterocyclylalkyl, —CO—C*(R9R10NH₂, —CO—CH₂—C*(R9R10)—NH₂, —CO—C*(R9R10)—CH₂—NH₂, alkylsulfonyl, arylsulfonyl, and arylalkylsulfonyl
which can be optionally substituted by up to 3 substituents independently selected from the group consisting of halogen, —F, —Cl, —Br, —I, —N₃, —CN, —NR7R8, —OH, —NO₂, alkyl, aryl, arylalkyl, —O-alkyl, —O-aryl, and —O-arylalkyl;
R6 is selected from the group consisting of hydrogen, alkyl, cycloalkyl, cycloalkylalkyl;
R7 and R8 are independently selected from the group consisting of hydrogen, alkyl, cycloalkyl, and cycloalkylalkyl;
R9 and R10 are independently selected from the group consisting of hydrogen, alkyl, a natural alpha-amino acid side chain, and an unnatural alpha-amino acid side chain and preferably are selected from the group consisting of “hydrogen atom, alkyl”;
m is 0, 1 or 2 and preferably is 0; and
* means a carbon atom of R or S configuration when chiral.

2. The method of claim 1, where in the compound of formula (I)
R3 is selected from the group consisting of -alkyl-CO-aryl, -alkyl-CO-arylalkyl, -alkyl-CO-heteroaryl, -alkyl-CO-heteroarylalkyl, -alkyl-CO-heterocyclyl, alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO—NH₂, -alkyl-CO—OH, -alkyl-NH—C(NH)—NH₂, alkyl-S-alkyl, and alkyl-S—H.

3. The method of claim 1, where in the compound of formula (I)
R4 is hydrogen;
R5 is selected from the group consisting of hydrogen atom, alkyl, cycloalkyl, cycloalkylalkyl, aryl, heteroaryl, arylalkyl, heteroarylalkyl, heterocyclyl, heterocyclylalkyl, alkylsulfonyl, arylsulfonyl, arylalkylsulfonyl, —CO-cycloalkyl, —CO-cycloalkyl-alkyl, —CO-aryl, —CO-arylalkyl, —CO-heteroaryl, —CO-heteroarylalkyl, —CO-heterocyclyl, and —CO-heterocyclylalkyl;
with the proviso that if R5 is —CO-heteroarylalkyl, heteroaryl is not imidazole; and with the proviso that if R5 is —CO-heterocyclyl and heterocyclyl contains only nitrogen atoms as heteroatoms, that at least two nitrogen atoms are contained in heterocyclyl; and
with the proviso that if R5 is —CO-heterocyclylalkyl and heterocyclyl contains only nitrogen atoms as heteroatoms that where one or two nitrogen atoms are contained in heterocyclyl no nitrogen atom is positioned at position 1 of heterocyclyl that is the atom directly linking heterocyclyl to the carbonyl group —CO—;
wherein the alkyl, cycloalkyl, cycloalkylalkyl, aryl, heteroaryl, arylalkyl, heteroarylalkyl, heterocyclyl, heterocyclylalkyl, alkylsulfonyl, arylsulfonyl, arylalkylsulfonyl, —CO-cycloalkyl, —CO-cycloalkylalkyl, —CO-aryl, —CO-arylalkyl, —CO-heteroaryl, —CO-heteroarylalkyl, —CO-heterocyclyl, and/or —CO-heterocyclylalkyl are optionally substituted by up to 3 substituents independently selected from the group consisting of halogen, —F, —Cl, —Br, —I, —N₃, —CN, —NR7R8, —OH, —NO₂, alkyl, aryl, arylalkyl, —O-alkyl, —O-aryl, and —O-arylalkyl;
with the proviso that if R5 is —CO-cycloalkyl or —CO-cycloalkylalkyl, R5 is not substituted with NR7R8 at position 1 of cycloalkyl, that is the C atom directly linking cycloalky” to the carbonyl group —CO— where R5 is —CO-cycloalkyl or to the alkyl where R5 is —CO-cycloalkylalkyl; and
with the proviso that if R5 is —CO-aryl or —CO-arylalkyl and aryl is phenyl/benzene and is only substituted with one substituent, this one substituent is not —NR7R8;
R6 is hydrogen;
R7 and R8 are independently selected from the group consisting of hydrogen atom, alkyl, cycloalkyl, and cycloalkylalkyl; and
m is 0, 1 or 2.

4. The method of claim 1, where in the compound of formula (I)
R1 is selected from the group consisting of hydrogen, methyl, (2-methoxyphenyl)-methyl, (3-methoxyphenyl)-methyl, (4-methoxyphenyl)-methyl, (3-methoxyphenyl)-ethyl, (4-methoxyphenyl)-ethyl, phenyl, phenyl-methyl, phenylethyl, (4-ethylphenyl)-methyl, (4-methylphenyl)-methyl, (4-fluorophenyl)-methyl, (4-bromophenyl)-methyl, (2,4-dimethoxyphenyl)-methyl, (3,5-dimethoxyphenyl)-methyl, 2,2-diphenyl-ethyl, naphthaline-1-yl-methyl, 1H-indole-3-yl-methyl, 2-(1H-indole-3-yl)-ethyl, 3-(1H-indole-3-yl)-propyl, 4-methyl-phenyl, 4-ethyl-phenyl, n-hexyl, (3,4-dichlorophenyl)-methyl, (4-nitro-phenyl)-methyl, (pyridine-2-yl)-methyl, (pyridine-3-yl)-methyl, (pyridine-4-yl)-methyl, (thiophene-2-yl)-methyl, (thiophene-3-yl)-methyl, (furan-2-yl)-methyl, and (furan-3-yl)-methyl;
R2 is selected from the group consisting of methyl, 1H-indole-3-yl-methyl, 2-(1H-indole-3-yl)-ethyl, 3-(1H-indole-3-yl)-propyl, 2-phenyl-ethyl, 3-phenyl-propyl, 4-phenyl-butyl, 2-methoxy-phenylmethyl, 3-methoxy-phenylmethyl, 4-methoxy-phenylmethyl, 2-methoxy-phenylethyl, 3-methoxy-phenylethyl, and 4-methoxy-phenylethyl;
R3 is selected from the group consisting of hydrogen, methyl, propan-2-yl, 2-methyl-propan-1-yl, butan-2-yl, butan-1-yl, —CH₂—SH, —(CH₂)₂—S—CH₃, 1H-indole-3-yl-methyl, phenyl-methyl, 2-phenyl-ethyl, —CH₂—O—CH₂-phenyl, —CH₂—CO—CH₂-phenyl, —(CH₂)₂—CO—CH₂-phenyl, —CH₂—C(O)O-phenyl, —(CH₂)₂—C(O)O-phenyl, hydroxy-methyl, 1-hydroxy-ethan-1-yl, —CH₂—CO—NH₂, —(CH₂)₂—CO—NH₂, (1-hydroxy-benzene-4-yl)-methyl, —CH₂—CO—OH, —(CH₂)₂—CO—OH, —(CH₂)₄—NH₂, (1H-imidazol-5-yl)-methyl, —(CH₂)₃—NH—C(NH)—NH₂, —(CH₂)₃—NH₂, and —(CH₂)₃—NH—CO—NH₂;
R4 is hydrogen;
R5 is selected from the group consisting of hydrogen, —CO—CH₂—NH₂ (Gly residue), —CO—CH₂—CH₂—NH₂ (beta-Ala residue), —CO—CHCH₃—NH₂ (D- and/or L-alpha-Ala residue), —CO-(pyrrolidine-2-yl) (D- and/or L-Pro residue), 2-amino-2-carbonyl-propane (2-amino-isobutyric acid/Aib residue), 4-carbonyl-1H-piperidine, 3-carbonyl-1H-piperidine, R-(3-carbonyl-1H-piperidine), S-(3-carbonyl-1H-piperidine), 2-carbonyl-1H-piperidine, R-(2-carbonyl-1H-piperidine), S-(2-carbonyl-1H-piperidine), 1-amino-2-carbonyl-benzene, carbonyl-cyclohexane, 2-acetyl-pyridine, 3-acetyl-pyridine, 4-acetyl-pyridine, 2-propionyl-pyridine, 3-propionyl-pyridine, 4-propionyl-pyridine, (R-1-amino)-2-carbonyl-cyclohexane, (S-1-amino)-2-carbonyl-cyclohexane, 2-carbonyl-1H-imidazole, 2-carbonyl-pyridine, 3-carbonyl-pyridine, 4-carbonyl-pyridine, 2-amino-3-carbonyl-pyridine, 2-carbonyl-pyrazine, 2-carbonyl-4-hydroxy-1H-pyrrolidine, 4-carbonyl-1H,3H-diazacyclohexane, methylsulfonyl, phenylsulfonyl, 1-carbonyl-1-amino-2-phenylethane, phenylmethyl, 1-carbonyl-4-azide-benzene, 2-carbonyl-2,5-dihydro-1H-pyrrole, 2-carbonyl-piperazine, 2-carbonyl-1H-pyrrolidine, 2-aminoethane, carbonyl-benzene, 2-carbonyl-pyrazine, 3-carbonyl-pyrazine, 4-carbonyl-oxacyclohexane, 4-methyl-phenylsulfonyl, and phenylmethyl-sulfonyl;
R6 is hydrogen; and
m is 0.

5. The method of claim 1, where in the compound of formula (I)
R3 is selected from the group consisting of —CH₂—CO—CH₂—phenyl, —(CH₂)₂—CO—CH₂-phenyl, —CH₂—CO—NH₂, —(CH₂)₂—CO—NH₂, —CH₂—CO—OH, —(CH₂)₂—CO—OH, —(CH₂)₃—NH—C(NH)—NH₂, and —CH₂—SH, —(CH₂)₂—S—CH₃.

6. The method of claim 1, where in the compound of formula (I)
R5 is selected from the group consisting of hydrogen, methylsulfonyl, phenylsulfonyl, carbonyl-cyclohexane, (R-1-amino)-2-carbonyl-cyclohexane, (S-1-amino)-2-carbonyl-cyclohexane, 2-carbonyl-pyridine, 3-carbonyl-pyridine, 4-carbonyl-pyridine, 2-acetyl-pyridine, 3-acetyl-pyridine, 4-acetyl-pyridine, 2-propionyl-pyridine, 3-propionyl-pyridine, 4-propionyl-pyridine, 2-amino-3-carbonyl-pyridine, 2-carbonyl-1H-imidazole, 2-carbonyl-pyrazine, and 4-carbonyl-1H,3H-diazacyclohexane.

7. The method of claim 1, where the compound is selected from the group consisting of:
compound 1 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,
compound 2 (R)-N-(1-(5-(2-(1H-indol-3-yl)ethyl)-4-phenethyl-4H-1,2,4-triazol-3-yl)-2-(1H-indol-3-yl)ethyl)-2-amino-2-methylpropanamide,
compound 3 (R)-N-(1-(5-(3-(1H-indol-3-yl)propyl)-4-phenethyl-4H-1,2,4-triazol-3-yl)-2-(1H-indol-3-yl)ethyl)-2-amino-2-methylpropanamide,
compound 4 (R)-N-(1-(5-benzyl-4-(naphthalen-1-ylmethyl)-4H-1,2,4-triazol-3-yl)-2-(1H-indol-3-yl)ethyl)-2-amino-2-methylpropanamide,
compound 5 (R)-N-(1-(5-(2-(1H-indol-3-yl)ethyl)-4-(naphthalen-1-ylmethyl)-4H-1,2,4-triazol-3-yl)-2-(1H-indol-3-yl)ethyl)-2-amino-2-methylpropanamide,
compound 6(R)-N-(1-(5-(2-(1H-indol-3-yl)ethyl)-4-(3-methoxybenzyl)-4H-1,2,4-triazol-3-yl)-2-(1H-indol-3-yl)ethyl)-2-amino-2-methylpropanamide,
compound 7 (R)-N-(1-(4-(3-methoxybenzyl)-5-benzyl-4H-1,2,4-triazol-3-yl)-2-(1H-indol-3-yl)ethyl)-2-amino-2-methylpropanamide,
compound 8 (R)-N-(1-(5-(2-(1H-indol-3-yl)ethyl)-4-benzyl-4H-1,2,4-triazol-3-yl)-2-(1H-indol-3-yl)ethyl)-2-amino-2-methylpropanamide,
compound 9 (R)-N-(1-(5-(3-(1H-indol-3-yl)propyl)-4-benzyl-4H-1,2,4-triazol-3-yl)-2-(1H-indol-3-yl)ethyl)-2-amino-2-methylpropanamide,
compound 10 (R)-N-(1-(5-(3-(1H-indol-3-yl)propyl)-4-(3-methoxybenzyl)-4H-1,2,4-triazol-3-yl)-2-(1H-indol-3-yl)ethyl)-2-amino-2-methylpropanamide,
compound 11 (R)-N-(1-(5-(3-(1H-indol-3-yl)propyl)-4-(naphthalen-1-ylmethyl)-4H-1,2,4-triazol-3-yl)-2-(1H-indol-3-yl)ethyl)-2-amino-2-methylpropanamide,
compound 12 (R)-N-(1-(5-(2-(1H-indol-3-yl)ethyl)-4-(4-methoxybenzyl)-4H-1,2,4-triazol-3-yl)-2-(1H-indol-3-yl)ethyl)-2-amino-2-methylpropanamide,
compound 13 (R)-N-(1-(4-(4-methoxybenzyl)-5-benzyl-4H-1,2,4-triazol-3-yl)-2-(1H-indol-3-yl)ethyl)-2-amino-2-methylpropanamide,
compound 14 (R)-N-(1-(5-(3-(1H-indol-3-yl)propyl)-4-(4-bromobenzyl)-4H-1,2,4-triazol-3-yl)-2-(1H-indol-3-yl)ethyl)-2-amino-2-methylpropanamide,
compound 15 (R)-N-(1-(5-(2-(1H-indol-3-yl)ethyl)-4-hexyl-4H-1,2,4-triazol-3-yl)-2-(1H-indol-3-yl)ethyl)-2-amino-2-methylpropanamide,
compound 16 (R)-N-(1-(5-(3-(1H-indol-3-yl)propyl)-4-hexyl-4H-1,2,4-triazol-3-yl)-2-(1H-indol-3-yl)ethyl)-2-amino-2-methylpropanamide,
compound 17 (R)-N-(1-(4,5-bis(2-(1H-indol-3-yl)ethyl)-4H-1,2,4-triazol-3-yl)-2-(1H-indol-3-yl)ethyl)-2-amino-2-methylpropanamide,
compound 18 (S)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,
compound 19 (R)-N-(1-(4-(3-methoxybenzyl)-5-phenethyl-4H-1,2,4-triazol-3-yl)-2-(1H-indol-3-yl)ethyl)-2-amino-2-methylpropanamide,
compound 20 (R)-N-(1-(4-(4-methoxybenzyl)-5-phenethyl-4H-1,2,4-triazol-3-yl)-2-(1H-indol-3-yl)ethyl)-2-amino-2-methylpropanamide,
compound 21 (R)-N-(1-(5-(2-(1H-indol-3-yl)ethyl)-4-(3,5-dimethoxybenzyl)-4H-1,2,4-triazol-3-yl)-2-(1H-indol-3-yl)ethyl)-2-amino-2-methylpropanamide,
compound 22 (R)-N-(1-(4-(4-methoxybenzyl)-5-(3-phenylpropyl)-4H-1,2,4-triazol-3-yl)-2-(1H-indol-3-yl)ethyl)-2-amino-2-methylpropanamide,
compound 23 (R)-N-(1-(5-(3-(1H-indol-3-yl)propyl)-4-(4-methoxybenzyl)-4H-1,2,4-triazol-3-yl)-2-(1H-indol-3-yl)ethyl)-2-amino-2-methylpropanamide,
compound 24 (R)-N-(1-(4-(2-(1H-indol-3-yl)ethyl)-5-(3-(1H-indol-3-yl)propyl)-4H-1,2,4-triazol-3-yl)-2-(1H-indol-3-yl)ethyl)-2-amino-2-methylpropanamide,
compound 25 (R)-N-(1-(5-(2-(1H-indol-3-yl)ethyl)-4-(2-methoxy)benzyl)-4H-1,2,4-triazol-3-yl)-2-(1H-indol-3-yl)ethyl)-2-amino-2-methylpropanamide,
compound 26 (R)-N-(1-(4-(2-methoxybenzyl)-5-phenethyl-4H-1,2,4-triazol-3-yl)-2-(1H-indol-3-yl)ethyl)-2-amino-2-methylpropanamide,
compound 27 (R)-N-(2-(1H-indol-3-yl)-1-(4-(naphthalen-1-ylmethyl)-5-phenethyl-4H-1,2,4-triazol-3-yl)ethyl)-2-amino-2-methylpropanamide,
compound 28 (R)-N-(1-(5-(2-(1H-indol-3-yl)ethyl)-4-(3,4-dichlorobenzyl)-4H-1,2,4-triazol-3-yl)-2-(1H-indol-3-yl)ethyl)-2-amino-2-methylpropanamide,
compound 29 (R)-N-(1-(5-(2-(1H-indol-3-yl)ethyl)-4-(4-fluorobenzyl)-4H-1,2,4-triazol-3-yl)-2-(1H-indol-3-yl)ethyl)-2-amino-2-methylpropanamide,
compound 30 (R)-N-(1-(4-(4-fluorobenzyl)-5-benzyl-4H-1,2,4-triazol-3-yl)-2-(1H-indol-3-yl)ethyl)-2-amino-2-methylpropanamide,
compound 31 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-4-carboxamide,
compound 32 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-3-carboxamide,
compound 33 (R)-N-(1-(4-(4-methylbenzyl)-5-(3-phenylpropyl)-4H-1,2,4-triazol-3-yl)-2-(1H-indol-3-yl)ethyl)-2-amino-2-methylpropanamide,
compound 34 (R)-N-(1-(5-(2-(1H-indol-3-yl)ethyl)-4-(4-methylbenzyl)-4H-1,2,4-triazol-3-yl)-2-(1H-indol-3-yl)ethyl)-2-amino-2-methylpropanamide,
compound 36 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-2-carboxamide,
compound 37 (R)-N-(1-(4-(4-methylbenzyl)-5-phenethyl-4H-1,2,4-triazol-3-yl)-2-(1H-indol-3-yl)ethyl)-2-amino-2-methylpropanamide,
compound 38 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminobenzamide,
compound 39 (R)-N-(1-(5-benzyl-4-(pyridin-2-ylmethyl)-4H-1,2,4-triazol-3-yl)-2-(1H-indol-³-yl)ethyl)-2-amino-2-methylpropanamide,
compound 40 (2S,4R)-N-((R)-1-(4-(4-methoxybenzyl)-5-phenethyl-4H-1,2,4-triazol-3-yl)-2-(1H-indol-3-yl)ethyl)-4-hydroxypyrrolidine-2-carboxamide,
compound 41 (S)-N-((R)-1-(4-(4-methoxybenzyl)-5-phenethyl-4H-1,2,4-triazol-3-yl)-2-(1H-indol-3-yl)ethyl)piperidine-3-carboxamide,
compound 42 (R)-N-((R)-1-(4-(4-methoxybenzyl)-5-phenethyl-4H-1,2,4-triazol-3-yl)-2-(1H-indol-3-yl)ethyl)piperidine-3-carboxamide,
compound 43 (R)-N-(1-(4-(4-ethylbenzyl)-5-phenethyl-4H-1,2,4-triazol-3-yl)-2-(1H-indol-3-yl)ethyl)-2-amino-2-methylpropanamide,
compound 44 (R)-N-(1-(4-(4-methoxybenzyl)-5-phenethyl-4H-1,2,4-triazol-3-yl)-2-(1H-indol-3-yl)ethyl)piperidine-4-carboxamide,
compound 45 (R)-N-(1-(5-(2-(1H-indol-3-yl)ethyl)-4-(4-methoxybenzyl)-4H-1,2,4-triazol-3-yl)-2-(1H-indol-3-yl)ethyl)piperidine-4-carboxamide,
compound 46 (S)-N-((R)-1-(4-(4-methoxybenzyl)-5-phenethyl-4H-1,2,4-triazol-3-yl)-2-(1H-indol-3-yl)ethyl)pyrrolidine-2-carboxamide,
compound 47 (R)-N-((R)-1-(4-(4-methoxybenzyl)-5-phenethyl-4H-1,2,4-triazol-3-yl)-2-(1H-indol-3-yl)ethyl)pyrrolidine-2-carboxamide,
compound 48 (S)-N-((R)-1-(4-(4-methoxybenzyl)-5-phenethyl-4H-1,2,4-triazol-3-yl)-2-(1H-indol-3-yl)ethyl)piperidine-2-carboxamide,
compound 49 (R)-N-((R)-1-(4-(4-methoxybenzyl)-5-phenethyl-4H-1,2,4-triazol-3-yl)-2-(1H-indol-³-yl)ethyl)piperidine-2-carboxamide,
compound 50 (R)-N-(1-(4-(4-methoxybenzyl)-5-phenethyl-4H-1,2,4-triazol-3-yl)-2-(1H-indol-3-yl)ethyl)-2-aminoacetamide,
compound 51 (R)-N-(1-(4-(4-methoxybenzyl)-5-phenethyl-4H-1,2,4-triazol-3-yl)-2-(1H-indol-3-yl)ethyl)-2-(pyridin-2-yl)acetamide,
compound 52 (R)-N-(1-(4-(4-methoxybenzyl)-5-phenethyl-4H-1,2,4-triazol-3-yl)-2-(1H-indol-3-yl)ethyl)-2-(pyridin-4-yl)acetamide,
compound 53 (R)-N-(1-(4-(4-methoxybenzyl)-5-phenethyl-4H-1,2,4-triazol-3-yl)-2-(1H-indol-3-yl)ethyl)cyclohexanecarboxamide,
compound 54 (R)-N-(1-(5-(2-(1H-indol-3-yl)ethyl)-4-benzyl-4H-1,2,4-triazol-3-yl)-2-(1H-indol-3-yl)ethyl)piperidine-4-carboxamide,
compound 55 (R)-N-(1-(5-(2-(1H-indol-3-yl)ethyl)-4-benzyl-4H-1,2,4-triazol-3-yl)-2-(1H-indol-3-yl)ethyl)piperidine-3-carboxamide,
compound 56 (R)-N-(1-(4-(4-methoxybenzyl)-5-phenethyl-4H-1,2,4-triazol-3-yl)-2-(1H-indol-3-yl)ethyl)-3-aminopropanamide,
compound 57 (S)-N-((R)-1-(4-(4-methoxybenzyl)-5-phenethyl-4H-1,2,4-triazol-3-yl)-2-(1H-indol-3-yl)ethyl)-2-aminopropanamide,
compound 58 (R)-N-(1-(4-(4-methoxybenzyl)-5-phenethyl-4H-1,2,4-triazol-3-yl)-2-(1H-indol-3-yl)ethyl)-2-(pyridin-3-yl)acetamide,
compound 59 (R)-N-(1-(4-(4-methoxybenzyl)-5-phenethyl-4H-1,2,4-triazol-3-yl)-2-(1H-indol-3-yl)ethyl)-3-(pyridin-3-yl)propanamide,
compound 60 (R)-N-(1-(5-(2-(1H-indol-3-yl)ethyl)-4-benzyl-4H-1,2,4-triazol-3-yl)-2-(1H-indol-3-yl)ethyl)-2-(pyridin-2-yl)acetamide,
compound 61 (R)-N-(1-(5-(2-(1H-indol-3-yl)ethyl)-4-(2,4-dimethoxybenzyl)-4H-1,2,4-triazol-3-yl)-2-(1H indol-3-yl)ethyl)-2-(pyridin-2-yl)acetamide,
compound 62 (R)-N-(1-(4-(2,4-dimethoxybenzyl)-5-phenethyl-4H-1,2,4-triazol-3-yl)-2-(1H-indol-3-yl)ethyl)piperidine-4-carboxamide,
compound 63 (R)-N-((R)-1-(4-(2,4-dimethoxybenzyl)-5-phenethyl-4H-1,2,4-triazol-3-yl)-2-(1H-indol-3-yl)ethyl)piperidine-2-carboxamide,
compound 64 (R)-N-(1-(4-(2,4-dimethoxybenzyl)-5-phenethyl-4H-1,2,4-triazol-3-yl)-2-(1H-indol-3-yl)ethyl)picolinamide,
compound 65 (R)-N-(1-(4-(2,4-dimethoxybenzyl)-5-phenethyl-4H-1,2,4-triazol-3-yl)-2-(1H-indol-3-yl)ethyl)isonicotinamide,
compound 66 (R)-N-(1-(4-(2,4-dimethoxybenzyl)-5-phenethyl-4H-1,2,4-triazol-3-yl)-2-(1H-indol-3-yl)ethyl)pyrazine-2-carboxamide,
compound 67 (R)-N-1-(4-(2,4-dimethoxybenzyl)-5-phenethyl-4H-1,2,4-triazol-3-yl)-2-(1H-indol-3-yl)ethyl)piperazine-2-carboxamide,
compound 68 (S)-N-((R)-1-(4-(2,4-dimethoxybenzyl)-5-phenethyl-4H-1,2,4-triazol-3-yl)-2-(1H-indol-3-yl)ethyl)pyrrolidine-2-carboxamide,
compound 69 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminoacetamide,
compound 70 (S)-N-((R)-1-(5-(2-(1H-indol-3-yl)ethyl)-4-(2,4-dimethoxybenzyl)-4H-1,2,4-triazol-3-yl)-2-(1H-indol-3-yl)ethyl)pyrrolidine-2-carboxamide,
compound 71 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)pyrazine-2-carboxamide,
compound 72 (R)-N-1-(5-(2-(1H-indol-3-yl)ethyl)-4-(2,4-dimethoxybenzyl)-4H-1,2, 4-triazol-3-yl)-2-(1H-indol-3-yl)ethyl)pi perazine-2-carboxamide,
compound 73 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)picolinamide,
compound 74 (R)-1-(5-(2-(1H-indol-3-yl)ethyl)-4-(2,4-dimethoxybenzyl)-4H-1,2,4-triazol-3-yl)-2-(1H-indol-3-yl)ethanamine,
compound 75 (R)-N-(1-(4-(2,4-dimethoxybenzyl)-5-phenethyl-4H-1,2,4-triazol-3-yl)-2-(1H-indol-3-yl)ethyl)-2-aminoacetamide,
compound 76 (R)-N-(1-(4-(4-methoxybenzyl)-5-phenethyl-4H-1,2,4-triazol-3-yl)-2-(1H-indol-3-yl)ethyl)pyrazine-2-carboxamide,
compound 77 (R)-N-(1-(4-(4-methoxybenzyl)-5-phenethyl-4H-1,2,4-triazol-3-yl)-2-(1H-indol-3-yl)ethyl)isonicotinamide,
compound 78 (R)-N-1-(4-(4-methoxybenzyl)-5-phenethyl-4H-1,2,4-triazol-3-yl)-2-(1H-indol-3-yl)ethyl)piperazine-2-carboxamide,
compound 79 (R)-N-(1-(4-(4-methoxybenzyl)-5-phenethyl-4H-1,2,4-triazol-3-yl)-2-(1H-indol-3-yl)ethyl)picolinamide,
compound 80 (R)-N-(1-(5-(2-(1H-indol-3-yl)ethyl)-4-(4-methoxybenzyl)-4H-1,2,4-triazol-3-yl)-2-(1H-indol-3-yl)ethyl)picolinamide,
compound 81 (R)-N-1-(5-(2-(1H-indol-3-yl)ethyl)-4-(4-methoxybenzyl)-4H-1,2,4-triazol-3-yl)-2-(1H-indol-3-yl)ethyl)piperazine-2-carboxamide,
compound 82 (R)-N-(1-(4-(4-ethylbenzyl)-5-phenethyl-4H-1,2,4-triazol-3-yl)-2-(1H-indol-3-yl)ethyl)-2-(pyridin-2-yl)acetamide,
compound 83 (R)-N-(1-(4-(4-ethylbenzyl)-5-phenethyl-4H-1,2,4-triazol-3-yl)-2-(1H-indol-3-yl)ethyl)piperidine-4-carboxamide,
compound 84 (R)-N-1-(4-(4-ethylbenzyl)-5-phenethyl-4H-1,2,4-triazol-3-yl)-2-(1H-indol-3-yl)ethyl)piperazine-2-carboxamide,
compound 85 (R)-N-(1-(4-(4-ethylbenzyl)-5-phenethyl-4H-1,2,4-triazol-3-yl)-2-(1H-indol-3-yl)ethyl)pyrazine-2-carboxamide,
compound 86 (R)-N-(1-(5-(2-(1H-indol-3-yl)ethyl)-4-(2,4-dimethoxybenzyl)-4H-1,2,4-triazol-3-yl)-2-(1H-indol-3-yl)ethyl)-2-cis-aminocyclohexanecarboxamide,
compound 87 (S)-N-((R)-1-(5-(2-(1H-indol-3-yl)ethyl)-4-(4-methoxybenzyl)-4H-1,2,4-triazol-3-yl)-2-(1H-indol-3-yl)ethyl)piperidine-3-carboxamide,
compound 88 (R)-N-((R)-1-(5-(2-(1H-indol-3-yl)ethyl)-4-(4-methoxybenzyl)-4H-1,2,4-triazol-3-yl)-2-(1H-indol-3-yl)ethyl)piperidine-2-carboxamide,
compound 89 (S)-N-((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,
compound 90 (R)-N-((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,
compound 91 (R)-N-(1-(5-(2-(1H-indol-3-yl)ethyl)-4-(4-methoxybenzyl)-4H-1,2,4-triazol-3-yl)-2-(1H-indol-3-yl)ethyl)-2-(pyridin-2-yl)acetamide,
compound 92 (R)-N-(1-(5-(2-(1H-indol-3-yl)ethyl)-4-(4-bromobenzyl)-4H-1,2,4-triazol-3-yl)-2-(1H-indol-³-yl)ethyl)-2-amino-2-methylpropanamide,
compound 93 (R)-N-(1-(5-(2-(1H-indol-3-yl)ethyl)-4-(4-methoxybenzyl)-4H-1,2,4-triazol-3-yl)-2-phenylethyl)-2-amino-2-methylpropanamide,
compound 94 (R)-N-(2-(1H-indol-3-yl)-1-(5-phenethyl-4-(thiophen-2-ylmethyl)-4H-1,2,4-triazol-3-yl)ethyl)piperidine-4-carboxamide,
compound 95 (R)-N-(1-(4-(2-(1H-indol-3-yl)ethyl)-4H-1,2,4-triazol-3-yl)-2-(1H-indol-3-yl)ethyl)-2-amino-2-methylpropanamide,
compound 96 (R)-N-(1-(5-((1H-indol-3-yl)methyl)-4-methyl-4H-1,2,4-triazol-3-yl)-2-(1H-indol-3-yl)ethyl)-2-amino-2-methylpropanamide,
compound 97 (R)-N-(1-(5-(2-(1H-indol-3-yl)ethyl)-4-methyl-4H-1,2,4-triazol-3-yl)-2-(1H-indol-3-yl)ethyl)-2-amino-2-methylpropanamide,
compound 98 (R)-N-(1-(5-((1H-indol-3-yl)methyl)-4H-1,2,4-triazol-3-yl)-2-(1H-indol-3-yl)ethyl)-2-amino-2-methylpropanamide,
compound 99 (R)-N-(1-(5-((1H-indol-3-yl)methyl)-4-(2,4-dimethoxybenzyl)-4H-1,2,4-triazol-3-yl)-2-(1H-indol-3-yl)ethyl)-2-amino-2-methylpropanamide,
compound 100 (R)-N-(1-(4-(2,4-dimethoxybenzyl)-5-methyl-4H-1,2,4-triazol-3-yl)-2-(1H-indol-3-yl)ethyl)-2-amino-2-methylpropanamide,
compound 101 (R)-N-(1-(5-((1H-indol-3-yl)methyl)-4-(4-methoxybenzyl)-4H-1,2,4-triazol-3-yl)-2-(1H-indol-3-yl)ethyl)-2-amino-2-methylpropanamide,
compound 102 (R)-N-(1-(4-(2,4-dimethoxybenzyl)-5-benzyl-4H-1,2,4-triazol-3-yl)-2-(1H-indol-3-yl)ethyl)-2-amino-2-methylpropanamide,
compound 103 (R)-N-(1-(5-(3-(1H-indol-3-yl)propyl)-4-(2,4-dimethoxybenzyl)-4H-1,2,4-triazol-3-yl)-2-(1H-indol-3-yl)ethyl )-2-amino-2-methylpropanamide,
compound 104 (R)-N-(1-(5-((1H-indol-3-yl)methyl)-4-phenethyl-4H-1,2,4-triazol-3-yl)-2-(1H-indol-3-yl)ethyl)-2-amino-2-methylpropanamide,
compound 105 (R)-N-(1-(5-benzyl-4-phenethyl-4H-1,2,4-triazol-3-yl)-2-(1H-indol-3-yl)ethyl)-2-amino-2-methylpropanamide,
compound 106 (R)-N-(1-(5-benzyl-4-(2,2-diphenylethyl)-4H-1,2,4-triazol-3-yl)-2-(1H-indol-3-yl)ethyl)-2-amino-2-methylpropanamide,
compound 107 (R)-N-(1-(5-(2-(1H-indol-3-yl)ethyl)-4-(2,2-diphenylethyl)-4H-1,2,4-triazol-3-yl)-2-(1H-indol-3-yl)ethyl)-2-amino-2-methylpropanamide,
compound 108 (R)-N-(1-(4-(3,5-dimethoxybenzyl)-5-benzyl-4H-1,2,4-triazol-3-yl)-2-(1H-indol-3-yl)ethyl )-2-amino-2-methylpropanamide,
compound 109 (R)-N-(1-(4,5-dibenzyl-4H-1,2,4-triazol-3-yl)-2-(1H-indol-3-yl)ethyl)-2-amino-2-methylpropanamide,
compound 110 (R)-N-(1-(5-benzyl-4-hexyl-4H-1,2,4-triazol-3-yl)-2-(1H-indol-3-yl)ethyl)-2-amino-2-methylpropanamide,
compound 111 (R)-N-(1-(4-(2-(1H-indol-3-yl)ethyl)-5-benzyl-4H-1,2,4-triazol-3-yl)-2-(1H-indol-3-yl)ethyl)-2-amino-2-methylpropanamide,
compound 112 (S)-N-(1-(4-(2,4-dimethoxybenzyl)-5-benzyl-4H-1,2,4-triazol-3-yl)-2-(1H-indol-3-yl)ethyl)-2-amino-2-methylpropanamide,
compound 113 (R)-N-(1-(4-(3,5-dimethoxybenzyl)-5-phenethyl-4H-1,2,4-triazol-3-yl)-2-(1H-indol-3-yl)ethyl)-2-amino-2-methylpropanamide,
compound 114 (R)-N-(1-(4-(4-bromobenzyl)-5-benzyl-4H-1,2,4-triazol-3-yl)-2-(1H-indol-3-yl)ethyl)-2-amino-2-methylpropanamide,
compound 115 (R)-N-(1-(4-(2-methoxybenzyl)-5-benzyl-4H-1,2,4-triazol-3-yl)-2-(1H-indol-3-yl)ethyl)-2-amino-2-methylpropanamide,
compound 116 (S)-N-(1-(4-(2,4-dimethoxybenzyl)-5-phenethyl-4H-1,2,4-triazol-3-yl)-2-(1H-indol-3-yl)ethyl)-2-amino-2-methylpropanamide,
compound 117 (R)-N-(1-(4,5-diphenethyl-4H-1,2,4-triazol-3-yl)-2-(1H-indol-3-yl)ethyl)-2-amino-2-methylpropanamide,
compound 118 (R)-N-(1-(4-(3,4-dichlorobenzyl)-5-benzyl-4H-1,2,4-triazol-3-yl)-2-(1H-indol-3-yl)ethyl)-2-amino-2-methylpropanamide,
compound 119 (R)-1-(5-(2-(1H-indol-3-yl)ethyl)-4-(2,4-dimethoxybenzyl)-4H-1,2,4-triazol-3-yl)-2-(1H-indol-3-yl)ethanamine,
compound 120 (R)-N-(1-(4-(4-methoxybenzyl)-5-benzyl-4H-1,2,4-triazol-3-yl)-2-phenylethyl)-2-amino-2-methylpropanamide,
compound 121 (R)-N-(1-(4-(4-fluorobenzyl)-5-phenethyl-4H-1,2,4-triazol-3-yl)-2-(1H-indol-3-yl)ethyl)-2-amino-2-methylpropanamide,
compound 122 (R)-N-(1-(4-(3,4-dichlorobenzyl)-5-phenethyl-4H-1,2,4-triazol-3-yl)-2-(1H-indol-3-yl)ethyl)-2-amino-2-methylpropanamide,
compound 124 (R)-N-(1-(4-(4-methylbenzyl)-5-benzyl-4H-1,2,4-triazol-3-yl)-2-(1H-indol-3-yl)ethyl)-2-amino-2-methylpropanamide,
compound 125 (S)-N-(1-(4-(4-methoxybenzyl)-5-(3-phenylpropyl)-4H-1,2,4-triazol-3-yl)-2-(1H-indol-3-yl)ethyl)-2-amino-2-methylpropanamide,
compound 126 (S)-N-(1-(4-(4-methoxybenzyl)-5-benzyl-4H-1,2,4-triazol-3-yl)-2-(1H-indol-3-yl)ethyl)-2-amino-2-methylpropanamide,
compound 128 N-((R)-1-(4-(4-nitrobenzyl)-5-phenethyl-4H-1,2,4-triazol-3-yl)-2-(1H-indol-3-yl)ethyl)-2-amino-2-methylpropanamide,
compound 129 (S)-N-(1-(4-(4-methoxybenzyl)-5-phenethyl-4H-1,2,4-triazol-3-yl)-2-(1H-indol-3-yl)ethyl)-2-amino-2-methylpropanamide,
compound 130 (R)-N-(1-(4-(4-methoxyphenethyl)-5-phenethyl-4H-1,2,4-triazol-3-yl)-2-(1H-indol-3-yl)ethyl)-2-amino-2-methylpropanamide,
compound 131 (R)-N-(2-(1H-indol-3-yl)-1-(5-phenethyl-4-(thiophen-2-ylmethyl)-4H-1,2,4-triazol-3-yl)ethyl)-2-amino-2-methylpropanamide,
compound 132 (R)-N-(2-(1H-indol-3-yl)-1-(5-phenethyl-4-(pyridin-2-ylmethyl)-4H-1,2,4-triazol-3-yl)ethyl)-2-amino-2-methylpropanamide,
compound 133 (R)-N-(2-(1H-indol-3-yl)-1-(5-phenethyl-4-(pyridin-2-ylmethyl)-4H-1,2,4-triazol-3-yl)ethyl)piperidine-3-carboxamide,
compound 134 (S)-N-((R)-1-(4-(4-ethylbenzyl)-5-phenethyl-4H-1,2,4-triazol-3-yl)-2-(1H-indol-3-yl)ethyl)pyrrolidine-2-carboxamide,
compound 135 N-((R)-1-(4-(4-ethylbenzyl)-5-phenethyl-4H-1,2,4-triazol-3-yl)-2-(1H-indol-3-yl)ethyl)-2-aminoacetamide,
compound 136 N-((R)-1-(5-(2-(1H-indol-3-yl)ethyl)-4-(4-methoxybenzyl)-4H-1,2,4-triazol-3-yl)-2-(1H-indol-3-yl)ethyl)-2-(pyridin-4-yl)acetamide,
compound 137 (2R)-N-((R)-1-(4-(4-ethylbenzyl)-5-phenethyl-4H-1,2,4-triazol-3-yl)-2-(1H-indol-3-yl)ethyl)piperidine-2-carboxamide,
compound 138 N-((R)-1-(4-(4-ethylbenzyl)-5-phenethyl-4H-1,2,4-triazol-3-yl)-2-(1H-indol-3-yl)ethyl)picolinamide,
compound 139 N-((R)-1-(4-(4-ethylbenzyl)-5-phenethyl-4H-1,2,4-triazol-3-yl)-2-(1H-indol-3-yl)ethyl)-2-aminopyridine-3-carboxamide,
compound 140 (2S)-N-((R)-1-(4-(4-ethylbenzyl)-5-phenethyl-4H-1,2,4-triazol-3-yl)-2-(1H-indol-3-yl)ethyl)-2-aminopropanamide,
compound 141 N-((R)-1-(4-(4-ethylbenzyl)-5-phenethyl-4H-1,2,4-triazol-3-yl)-2-(1H-indol-3-yl)ethyl)isonicotinamide,
compound 142 N-((R)-2-(1H-indol-3-yl)-1-(5-phenethyl-4-phenyl-4H-1,2,4-triazol-3-yl)ethyl)piperidine-4-carboxamide,
compound 143 (2S)-N-((R)-2-(1H-indol-3-yl)-1-(5-phenethyl-4-phenyl-4H-1,2,4-triazol-3-yl)ethyl)pyrrolidine-2-carboxamide,
compound 144 N-((R)-2-(1H-indol-3-yl)-1-(5-phenethyl-4-phenyl-4H-1,2,4-triazol-3-yl)ethyl)-2-aminoacetamide,
compound 145 N-((R)-2-(1H-indol-3-yl)-1-(5-phenethyl-4-phenyl-4H-1,2,4-triazol-3-yl)ethyl)-2-(pyridin-2-yl)acetamide,
compound 146 N-((R)-2-(1H-indol-3-yl)-1-(5-phenethyl-4-phenyl-4H-1,2,4-triazol-3-yl)ethyl)picolinamide,
compound 147 N-((R)-1-(5-(2-(1H-indol-3-yl)ethyl)-4-(4-ethylphenyl)-4H-1,2,4-triazol-3-yl)-2-(1H-indol-3-yl)ethyl)picolinamide,
compound 148 N-((R)-1-(5-(2-(1H-indol-3-yl)ethyl)-4-(4-ethylphenyl)-4H-1,2,4-triazol-3-yl)-2-(1H-indol-3-yl)ethyl)-2-(pyridin-2-yl)acetamide,
compound 149 N-((R)-1-(5-(2-(1H-indol-3-yl)ethyl)-4-(4-ethylphenyl)-4H-1,2,4-triazol-3-yl)-2-(1H-indol-3-yl)ethyl)-2-aminoacetamide,
compound 150 (2S)-N-((R)-1-(5-(2-(1H-indol-3-yl)ethyl)-4-(4-ethylphenyl)-4H-1,2,4-triazol-3-yl)-2-(1H-indol-3-yl)ethyl)pyrrolidine-2-carboxamide,
compound 152 N-((R)-1-(5-(2-(1H-indol-3-yl)ethyl)-4-(4-methoxybenzyl)-4H-1,2,4-triazol-3-yl)-2-(1H-indol-3-yl)ethyl)-2-aminoacetamide,
compound 153 N-((R)-1-(4-(4-methoxybenzyl)-5-phenethyl-4H-1,2,4-triazol-3-yl)-2-(1H-indol-3-yl)ethyl)-2-trans-aminocyclohexanecarboxamide,
compound 154 N-((R)-1-(4-(4-ethylbenzyl)-5-phenethyl-4H-1,2,4-triazol-3-yl)-2-(1H-indol-3-yl)ethyl)-2-(pyridin-3-yl)acetamide,
compound 155 (3S)-N-((R)-1-(4-(4-ethylbenzyl)-5-phenethyl-4H-1,2,4-triazol-3-yl)-2-(1H-indol-3-yl)ethyl)piperidine-3-carboxamide,
compound 156 N-((R)-1-(4-(4-ethylbenzyl)-5-phenethyl-4H-1,2,4-triazol-3-yl)-2-(1H-indol-3-yl)ethyl)-2-aminobenzamide,
compound 157 N-((R)-1-(5-(2-(1H-indol-3-yl)ethyl)-4-phenyl-4H-1,2,4-triazol-3-yl)-2-(1H-indol-3-yl)ethyl)picolinamide,
compound 158 N-((R)-1-(5-(2-(1H-indol-3-yl)ethyl)-4-phenyl-4H-1,2,4-triazol-3-yl)-2-(1H-indol-3-yl)ethyl)piperidine-4-carboxamide,
compound 159 N-((R)-2-(1H-indol-3-yl)-1-(4-(2,4-dimethoxyphenyl)-5-phenethyl-4H-1,2,4-triazol-3-yl)ethyl)picolinamide,
compound 160 N-((R)-2-(1H-indol-3-yl)-1-(4-(2,4-dimethoxyphenyl)-5-phenethyl-4H-1,2,4-triazol-3-yl)ethyl)-2-(pyridin-2-yl)acetamide,
compound 161 N-((R)-2-(1H-indol-3-yl)-1-(4-(2,4-dimethoxyphenyl)-5-phenethyl-4H-1,2,4-triazol-3-yl)ethyl)pyrazine-2-carboxamide,
compound 162 N-((R)-2-(1H-indol-3-yl)-1-(4-(2,4-dimethoxyphenyl)-5-phenethyl-4H-1,2,4-triazol-3-yl)ethyl)-2-aminoacetamide,
compound 163 N-((R)-2-(1H-indol-3-yl)-1-(4-(2,4-dimethoxyphenyl)-5-phenethyl-4H-1,2,4-triazol-3-yl)ethyl)piperidine-4-carboxamide,
compound 164 N-((R)-1-(5-benzyl-4-((pyridin-2-yl)methyl)-4H-1,2,4-triazol-3-yl)-2-(1H-indol-3-yl)ethyl)picolinamide,
compound 165 N-((R)-1-(5-benzyl-4-((pyridin-2-yl)methyl)-4H-1,2,4-triazol-3-yl)-2-(1H-indol-3-yl)ethyl)-2-amino-acetamide,
compound 166 N-((R)-1-(5-benzyl-4-((pyridin-2-yl)methyl)-4H-1,2,4-triazol-3-yl)-2-(1H-indol-3-yl)ethyl)piperidine-4-carboxamide,
compound 167 N-((R)-1-(5-benzyl-4-((pyridin-4-yl)methyl)-4H-1,2,4-triazol-3-yl)-2-(1H-indol-3-yl)ethyl)-2-amino-2-methylpropanamide,
compound 168 N-((R)-1-(5-(4-methoxybenzyl)-4-phenethyl-4H-1,2,4-triazol-3-yl)-2-(1H-indol-3-yl)ethyl)-2-amino-2-methylpropanamide,
compound 169 N-((R)-1-(5-benzyl-4-((pyridin-4-yl)methyl)-4H-1,2,4-triazol-3-yl)-2-(1H-indol-3-yl)ethyl)picolinamide,
compound 170 N-((R)-1-(5-benzyl-4-((pyridin-4-yl)methyl)-4H-1,2,4-triazol-3-yl)-2-(1H-indol-3-yl)ethyl)2-amino-acetamide,
compound 171 (R)-benzyl-3-(2-aminoisobutyramido)-3-(5-(2-(1H-indol-3-yl)ethyl)-4-(4-methoxybenzyl)-4H-1,2,4-triazol-3-yl)-propanoate,
compound 172 N-((R)-1-(5-benzyl-4-((pyridin-3-yl)methyl)-4H-1,2,4-triazol-3-yl)-2-(1H-indol-3-yl)ethyl)-2-amino-2-methylpropanamide,
compound 173 N-((R)-1-(4-benzyl-5-phenethyl-4H-1,2,4-triazol-3-yl)-2-(1H-indol-3-yl)ethyl)-2-amino-2-methylpropanamide,
compound 174 N-((R)-2-(1H-indol-3-yl)-1-(4-methyl-5-phenethyl-4H-1,2,4-triazol-3-yl)ethyl)picolinamide,
compound 175 N-((R)-1-(5-(2-(1H-indol-3-yl)ethyl)-4-phenyl-4H-1,2,4-triazol-3-yl)-2-(1H-indol-3-yl)ethyl)-2-amino-2-methylpropanamide,
compound 176 N-((R)-1-(4-(4-methoxybenzyl)-5-phenethyl-4H-1,2,4-triazol-3-yl)-2-(1H-indol-3-yl)ethyl)benzamide,
compound 177 (R)-1-(4-(2,4-dimethoxybenzyl)-5-phenethyl-4H-1,2,4-triazol-3-yl)-2-(1H-indol-3-yl)-N-phenylmethanesulfonylamine,
compound 178 (R)-1-(4-(2,4-dimethoxybenzyl)-5-phenethyl-4H-1,2,4-triazol-3-yl)-2-(1H-indol-3-yl)-N-tosylethanamine,
compound 179 N-((R)-1-(4-(2,4-dimethoxybenzyl)-5-phenethyl-4H-1,2,4-triazol-3-yl)-2-(1H-indol-3-yl)ethyl)-2-amino-2-methylpropanamide,
compound 180 N-1-((R)-1-(4-(2,4-dimethoxybenzyl)-5-phenethyl-4H-1,2,4-triazol-3-yl)-2-(1H-indol-3-yl)ethyl)ethane-1,2-diamine,
compound 181 N-((R)-1-(4-((furan-2-yl)methyl)-5-phenethyl-4H-1,2,4-triazol-3-yl)-2-(1H-indol-3-yl)ethyl)-2-amino-2-methylpropanamide,
compound 182 N-((R)-1-(4-((furan-2-yl)methyl)-5-phenethyl-4H-1,2,4-triazol-3-yl)-2-(1H-indol-3-yl)ethyl)picolinamide,
compound 183 N-((R)-1-(4-((furan-2-yl)methyl)-5-phenethyl-4H-1,2,4-triazol-3-yl)-2-(1H-indol-3-yl)ethyl)piperidine-4-carboxamide,
compound 184 N-((R)-1-(4-(4-methoxybenzyl)-5-phenethyl-4H-1,2,4-triazol-3-yl)-2-(1H-indol-3-yl)ethyl)-tetrahydro-2H-pyran-4-carboxamide,
compound 185 N-((R)-1-(5-((1H-indol-3-yl)methyl)-4-(3-methoxybenzyl)-4H-1,2,4-triazol-3-yl)-2-(1H-indol-3-yl)ethyl)-2-amino-2-methylpropanamide,
compound 186 (2S)-N-((R)-1-(4-(4-methoxybenzyl)-5-phenethyl-4H-1,2,4-triazol-3-yl)-2-(1H-indol-3-yl)ethyl)-2-amino-3-phenylpropanamide,
compound 187 (R)-1-(5-(2-(1H-indol-3-yl)ethyl)-4-(2,4-dimethoxybenzyl)-4H-1,2,4-triazol-3-yl)-2-(1H-indol-3-yl)-N-tosylethanamine,
compound 188 N-((R)-1-(4-(2,4-dimethoxybenzyl)-5-phenethyl-4H-1,2,4-triazol-3-yl)-2-(1H-indol-3-yl)ethyl)-4-azidobenzamide,
compound 189 N-benzyl-(R)-1-(4-(2,4-dimethoxybenzyl)-5-phenethyl-4H-1,2,4-triazol-3-yl)-2-(1H-indol-3-yl)ethanamine, and
compound 190 (2S)-N-((R)-1-(5-(2-(1H-indol-3-yl)ethyl)-4-(4-methoxybenzyl)-4H-1,2,4-triazol-3-yl)-2-(1H-indol-3-yl)ethyl)-2, 5-dihydro-1H-pyrrole-2-carboxamide.

8. The method of claim 1, wherein modulating the activity of one or more G-protein coupled receptors comprises treating a condition in a mammal mediated by the at least one G-protein coupled receptor (GPCR) and wherein the at least one compound is administered as a medicament to the mammal.

9. The method of claim 1, wherein the compound is a G-protein coupled receptor (GPCR) antagonist.

10. The method of claim 1, wherein the compound is a G-protein coupled receptor (GPCR) agonist.

11. The method of claim 1, wherein the at least one G-protein coupled receptor (GPCR) is selected from the group consisting of
5-hydroxytryptamine (serotonin) receptor, 5-hydroxytryptamine (serotonin) receptor 1A, 5-hydroxytryptamine (serotonin) receptor 1B, 5-hydroxytryptamine (serotonin) receptor 1D, 5-hydroxytryptamine (serotonin) receptor 1E, 5-hydroxytryptamine (serotonin) receptor 1F, 5-hydroxytryptamine (serotonin) receptor 2A, 5-hydroxytryptamine (serotonin) receptor 2B, 5-hydroxytryptamine (serotonin) receptor 2C, 5-hydroxytryptamine (serotonin) receptor 4, 5-hydroxytryptamine (serotonin) receptor 5A, 5-hydroxytryptamine (serotonin) receptor 6, 5-hydroxytryptamine (serotonin) receptor 7, muscarinic cholinergic receptor, muscarinic cholinergic receptor 1, muscarinic cholinergic receptor 2, muscarinic cholinergic receptor 3, muscarinic cholinergic receptor 4, muscarinic cholinergic receptor 5, Alpha-adrenergic receptor, Alpha-1-adrenergic receptor, Alpha-1A-adrenergic receptor, Alpha-1B-adrenergic receptor, Alpha-1D-adrenergic receptor, Alpha-2-adrenergic receptor, Alpha-2A-adrenergic receptor, Alpha-2B-adrenergic receptor, Alpha-2C-adrenergic receptor, Beta-adrenergic receptor, Beta-1-adrenergic receptor, Beta-2-adrenergic receptor, Beta-3-adrenergic receptor, angiotensin II receptor, angiotensin II receptor type 1, angiotensin II receptor type 2, neuromedin B receptor, gastrin-releasing peptide receptor, bombesin-like receptor 3, calcitonin receptor, calcitonin receptor-like, cannabinoid receptor, cannabinoid receptor 1, cannabinoid receptor 2, cholecystokinin receptor, cholecystokinin A receptor, cholecystokinin B receptor, corticotropin releasing hormone receptor, corticotropin releasing hormone receptor 1, corticotropin releasing hormone receptor 2, dopamine receptor, dopamine receptor D1, dopamine receptor D2, dopamine receptor D3, dopamine receptor D4, dopamine receptor D5, glucagon-like peptide receptor, glucagon-like peptide 1 receptor, glucagon-like peptide 2 receptor, follicle stimulating hormone receptor, histamine receptor, histamine receptor H1, histamine receptor H2, histamine receptor H3, histamine receptor H4, endothelial differentiation lysophosphatidic acid G-protein-coupled receptor, endothelial differentiation lysophosphatidic acid G-protein-coupled receptor 2, endothelial differentiation lysophosphatidic acid G-protein-coupled receptor 4, endothelial differentiation lysophosphatidic acid G-protein-coupled receptor 7, melanin-concentrating hormone receptor, melanin-concentrating hormone receptor 1, melanin-concentrating hormone receptor 2, melanocortin receptor, melanocortin 1 receptor (alpha melanocyte stimulating hormone receptor), melanocortin 2 receptor (adrenocorticotropic hormone receptor), melanocortin 3 receptor, melanocortin 4 receptor, melanocortin 5 receptor, neuropeptide FF receptor, neuropeptide FF receptor 1, neuropeptide FF receptor 2, neuropeptides B/W receptor, neuropeptides B/W receptor 1, neuropeptides B/W receptor 2, neuropeptide Y receptor, neuropeptide Y receptor Y1, neuropeptide Y receptor Y2, neuropeptide Y receptor Y4 (pancreatic polypeptide receptor 1), neuropeptide Y receptor Y5, opioid receptor, opioid receptor delta 1, opioid receptor kappa 1, opioid receptor mu 1, opiate receptor-like 1, hypocretin (orexin) receptor, hypocretin (orexin) receptor 1, hypocretin (orexin) receptor 2, G protein-coupled receptor 103 (GPR103), G protein-coupled receptor 103A (GPR103A), G protein-coupled receptor 103B (GPR103B), prolactin releasing hormone receptor, somatostatin receptor, somatostatin receptor 1, somatostatin receptor 2, somatostatin receptor 3, somatostatin receptor 4, somatostatin receptor 5, galanin receptor, galanin receptor 1, galanin receptor 2, galanin receptor 3, cortistatin receptor, cortistatin receptor MAS-related GPR member X1, cortistatin receptor MAS-related GPR member X2, cortistatin receptor MAS-related GPR member X3, and cortistatin receptor MAS-related GPR member X4.

12. The method of claim 11, wherein the at least one G-protein coupled receptor (GPCR) is selected from the group consisting of
5-hydroxytryptamine (serotonin) receptor 2C, muscarinic cholinergic receptor 3, Beta-3-adrenergic receptor, angiotensin II receptor type 1, angiotensin II receptor type 2, gastrin-releasing peptide receptor, bombesin-like receptor 3, calcitonin receptor, calcitonin receptor-like, cannabinoid receptor 1, cannabinoid receptor 2, cholecystokinin A receptor, cholecystokinin B receptor, corticotropin releasing hormone receptor 1, corticotropin releasing hormone receptor 2, dopamine receptor D1, dopamine receptor D2, dopamine receptor D3, dopamine receptor D4, dopamine receptor D5, glucagon-like peptide 1 receptor, follicle stimulating hormone receptor, histamine receptor H1, histamine receptor H3, endothelial differentiation lysophosphatidic acid G-protein-coupled receptor 2, melanin-concentrating hormone receptor 1, melanocortin 1 receptor (alpha melanocyte stimulating hormone receptor), melanocortin 3 receptor, melanocortin 4 receptor, neuropeptide FF receptor 1, neuropeptide FF receptor 2, neuropeptides B/W receptor 1, neuropeptides B/W receptor 2, neuropeptide Y receptor Y1, neuropeptide Y receptor Y2, neuropeptide Y receptor Y5, opioid receptor kappa 1, hypocretin (orexin) receptor 1, hypocretin (orexin) receptor 2, G protein-coupled receptor 103 (GPR103), G protein-coupled receptor 103A (GPR103A), G protein-coupled receptor 103B (GPR103B), prolactin releasing hormone receptor and/or galanin receptor 1” and most preferably is selected from the group consisting of: “cholecystokinin A receptor, cholecystokinin B receptor, neuropeptide FF receptor 1, neuropeptide FF receptor 2, neuropeptide Y receptor Y1, neuropeptide Y receptor Y2, neuropeptide Y receptor Y5, hypocretin (orexin) receptor 1, hypocretin (orexin) receptor 2, G protein-coupled receptor 103 (GPR103), G protein-coupled receptor 103A (GPR103A), G protein-coupled receptor 103B (GPR103B), galanin receptor 1 and cortistatin receptor MAS-related GPR member X2.

13. The method of claim 8, wherein the condition is mediated by the at least one G-protein coupled receptor (GPCR) and at least one other receptor selected from the group consisting of: GHS receptors, GHS type 1 receptor, GHS-R 1a, GHS-R 1b, motilin receptor, motilin receptor la, neurotensin receptor, TRH receptor, GPR38 (FM1), GPR39 (FM2), FM3, GHS-R subtype, GHS binding site, cardiac GHS-R, and mammary GHS-R.

14. The method of claim 13, where the treatment is achieved by modulating one or more of the other receptors.

15. The method of claim 14, where the compound is an antagonist of the one or more other receptors.

16. The method of claim 14, where the compound is an agonist of the one or more other receptors.

17. The method of claim 8, wherein the condition is selected from the group consisting of metabolic disorders, lipid metabolism, disorders of lipid metabolism, adipogenesis, adiposity, anorexia, body weight gain, body weight reduction, bulimia, cachexia, diabetes, diabetes type I, diabetes type II, energy balance, energy homeostasis, food intake, hyperlipidemia, hyperphagia, insulin resistance in the heart, insulin resistance in type 2 diabetic patients, insulin resistance including NIDDM, metabolic homeostasis maintenance; obesity, overweight, short-, medium- and/or long-term regulation of energy balance, short-, medium- and long-term regulation (stimulation and/or inhibition) of food intake.

18. The method of claim 8, wherein the condition is selected from the group consisting of premature death, diabetes, type 2 diabetes, heart disease, heart attack, congestive heart failure, stroke, hypertension (high blood pressure), gallbladder disease, osteoarthritis, sleep apnea, asthma, breathing problems, endometrial cancer, colon cancer, kidney cancer, gallbladder cancer, postmenopausal breast cancer, hypercholesterolemia (high blood cholesterol), insulin resistance, complications of pregnancy, menstrual irregularities, hirsutism, stress incontinence, increased surgical risk, psychological disorders such as depression” and preferably are selected from the group consisting of “adipogenesis, adiposity, cachexia, diabetes, diabetes type I, diabetes type II, energy balance, energy homeostasis, food intake, hyperlipidemia, hyperphagia, obesity, short-, medium- and/or long-term regulation of energy balance, short-, medium- and long-term regulation (stimulation and/or inhibition) of food intake.

19. The method of claim 8, wherein the medicament comprises at least one additional pharmacologically active substance.

20. The method of claim 8, wherein the medicament is applied before and/or during and/or after treatment with at least one additional pharmacologically active substance.

21. The method of claim 19, wherein the at least one additional pharmacologically active substance is selected from the group consisting of GHS receptor agonist and/or antagonist, GHS type 1 receptor agonist and/or antagonist, GHS-R 1a agonist and/or antagonist, GHS-R 1b agonist and/or antagonist, motilin receptor agonist and/or antagonist, motilin receptor 1a agonist and/or antagonist, neurotensin receptor agonist and/or antagonist, TRH receptor agonist and/or antagonist, GPR38 (FM1) agonist and/or antagonist, GPR39 (FM2) agonist and/or antagonist, FM3 agonist and/or antagonist, GHS-R subtype agonist and/or antagonist, GHS binding site agonist and/or antagonist, cardiac GHS-R agonist and/or antagonist, mammary GHS-R agonist and/or antagonist, endocannabinoid receptor agonist and/or antagonist, CB1 receptor agonist and/or antagonist, rimonabant [1H-Pyrazole-3-carboxamide, 5-(4-chlorophenyl)-1-(2,4-dichlorophenyl)-4-methyl-N-1-piperidinyl-, monohydrochloride], sibutramine [1-(4-chlorophenyl)-N,N-dimethyl-alpha-(2-methylpropyl)-cyclobutane-methanamine], and sibutramide hydrochloride monohydrate.

22. The method of claim 1, where in the compound of formula (I) R1 and R2 are independently selected from the group consisting of alkyl, aryl, heteroaryl, arylalkyl, and heteroarylalkyl
optionally being substituted by up to 3 substituents independently selected from the group consisting of halogen, —F, —Cl, —Br, —I, —N₃, —CN, —NR7R8, —OH, —NO₂, alkyl, aryl, arylalkyl, —O-alkyl, —O-aryl, and —O-arylalkyl.

23. The method of claim 1, where in the compound of formula (I) one of radicals R3 and R4 is substituted in the aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl.

24. The method of claim 1, where in the compound of formula (I) R5 is selected from the group consisting of hydrogen, —CO-alkyl, —CO-cycloalkyl, —CO-aryl, —CO-heteroaryl, —CO-arylalkyl, —CO-heteroarylalkyl, —CO-heterocyclyl, —CO—C*(R9R10)-NH₂, —CO—CH₂—C*(R9R10)-NH₂, and —CO—C*(R9R10)-CH₂—NH₂,
optionally being substituted by up to 3 substituents independently selected from the group consisting of halogen, —F, —Cl, —Br, —I, —N₃, —CN, —NR7R8, —OH, —NO₂, alkyl, aryl, arylalkyl, —O-alkyl, —O-aryl, and —O-arylalkyl.

25. The method of claim 1, where in the compound of formula (I) R6 is hydrogen.

26. The method of claim 1, where in the compound of formula (I) R7 and R8 are hydrogen.

27. The method of claim 1, where in the compound of formula (I) R9 and R10 are independently selected from the group consisting of hydrogen atom and alkyl.

28. The method of claim 1, where in the compound of formula (I) m is 0.

29. The method of claim 2, where in the compound of formula (I) R3 is selected from the group consisting of -alkyl-CO-arylalkyl, -alkyl-C(O)O-arylalkyl, -alkyl-CO—NH₂, and -alkyl-CO—OH.

30. The method of claim 3, where in the compound of formula (I) R7 and R8 are hydrogen.

31. The method of claim 3, where in the compound of formula (I) m is 0.

32. The method of claim 4, where in the compound of formula (I) R3 is selected from the group consisting of 1H-indole-3-yl-methyl, —CH₂—CO—CH₂-phenyl, —(CH₂)₂—CO—CH₂-phenyl, —CH₂—C(O)O-phenyl, and —(CH₂)₂—C(O)O-phenyl.
